(19) United States
(12) Patent Application Publication
(10) Pub. No.: US 2026/0284074 A1
(43) Pub. Date: Sep. 24, 2026

(54) ENT1 Inhibitors in Combination with Checkpoint Inhibitors

(71) Applicant: iTeos Belgium SA, Gosselies (BE)

(72) Inventors: Theodore J. Sanders, Arquennes (BE); Erica Joke Katelijne Heleen Houthuys, Halle (BE); Reece Gerrad Marillier, Caumont-Gistoux (BE); Lucas Chable, Gosselies (BE); Maura Rossetti, Waterloo (BE)

(73) Assignee: iTeos Belgium SA, Gosselies (BE)

(21) Appl. No.: 19/674,668

(22) Filed: May 12, 2026

Related U.S. Application Data
(63) Continuation of application No. PCT/IB2024/061280, filed on Nov. 13, 2024.
(60) Provisional application No. 63/598,899, filed on Nov. 14, 2023.
(60) Provisional application No. 63/562,953, filed on Mar. 8, 2024.
(60) Provisional application No. 63/568,078, filed on Mar. 21, 2024.

(51) Int. Cl. A61K 31/551; A61K 31/522; A61K 39/00; A61K 39/395; A61P 35/00

(52) U.S. Cl. CPC A61K 31/551; A61K 31/522; A61K 39/3955; A61P 35/00; A61K 2039/505

(57) ABSTRACT

Disclosed herein are combinations, compositions, and kits comprising an ENT1 inhibitor and a checkpoint inhibitor. In some embodiments, the ENT1 inhibitor is a compound of Formula (I) or a pharmaceutically acceptable salt, hydrate, or solvate thereof. In some embodiments, the checkpoint inhibitor is a PD1 inhibitor, a PD-L1 inhibitor, a LAG-3 inhibitor, and a CTLA-4 inhibitor. Also disclosed herein are methods comprising administering an ENT1 inhibitor and a checkpoint inhibitor as well as uses of an ENT1 inhibitor and a checkpoint inhibitor in the manufacture of a medicament. In some embodiments, the combinations, compositions, kits, methods, and uses further comprise an adenosine receptor antagonist.

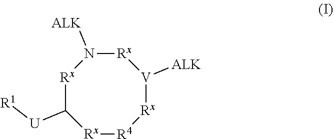

### CROSS REFERENCE TO RELATED APPLICATIONS

This application is a continuation of International Application No. PCT/IB2024/061280, filed Nov. 13, 2024, which claims priority to U.S. Application No. 63/568,078, filed Mar. 21, 2024, U.S. Application No. 63/562,953, filed Mar. 8, 2024, and U.S. Application No. 63/598,899, filed Nov. 14, 2023, the entire contents of which are incorporated by reference herein for all purposes.

### FIELD

Disclosed herein are combinations, compositions, and kits comprising an ENT1 inhibitor and a checkpoint inhibitor. Also disclosed herein are methods comprising administering an ENT1 inhibitor and a checkpoint inhibitor as well as uses of an ENT1 inhibitor and a checkpoint inhibitor in the manufacture of a medicament. In some embodiments, the combinations, compositions, kits, methods, and uses further comprise an adenosine receptor antagonist.

In some embodiments, the checkpoint inhibitor is a PD1 inhibitor, a PD-L1 inhibitor, a LAG-3 inhibitor, and a CTLA-4 inhibitor. In some embodiments, the method is a method of reversing adenosine-mediated suppression of T cell proliferation and/or viability. In some embodiments, the method is a method of reversing adenosine-mediated suppression of antigen-specific killing activity of T cells. In some embodiments, the method is a method of reversing adenosine-mediated suppression of production of cytokines. In some embodiments, the method is a method of treating cancer. In some embodiments, the use is in the manufacture of a medicament for the treatment of cancer.

### BACKGROUND

The profound benefit of immune checkpoint blockade for cancer therapy is restricted to limited subsets of patients with specific cancers. Many factors contribute to primary and acquired resistance to immune checkpoint blockade, including local accumulation of immunosuppressive metabolites such as the nucleoside adenosine and downstream adenosine receptor signaling. Pharmacological inhibition of adenosine generation and signaling are active areas of clinical investigation.

The equilibrative nucleoside transporter (ENT) family, also known as SLC29, is a group of plasmalemmal transport proteins which transport nucleosides into cells. There are four known ENTs, designated ENT1, ENT2, ENT3, and ENT4.

One of the endogenous substrates for ENTs is adenosine, a potent physiological and pharmacological regulator of numerous functions. Cellular signaling by adenosine occurs through four known G-protein-coupled adenosine receptors A1, A_{2A}, A2B, and A3. By influencing the concentration of adenosine available to these receptors, ENTs fulfill important regulatory roles in different physiological processes, such as modulation of coronary blood flow, inflammation, and neurotransmission (Griffith D A and Jarvis S M, Biochim Biophys Acta, 1996, 1286, 153-181; Shryock J C and Belardinelli L, Am J Cardiol, 1997, 79(12A), 2-10; Anderson C M et al., J Neurochem, 1999, 73, 867-873).

Adenosine is a potent immunosuppressive metabolite that is often found elevated in the extracellular tumor microenvironment (TME) (Blay J et al., Cancer Res, 1997, 57, 2602-2605). Extracellular adenosine is generated mainly by the conversion of ATP by the ectonucleotidases CD39 and CD73 (Stagg J and Smyth M J, Oncogene, 2010, 2, 5346-5358). Adenosine activates four G-protein-coupled receptor subtypes (A1, A_{2A}, A2B, and A3). In particular, activation of the A_{2A} receptor is believed to be the main driver of innate and adaptive immune cell suppression leading to suppression of antitumor immune responses (Ohta and Sitkovsky, Nature, 2001, 414, 916-920; Stagg and Smyth, Oncogene, 2010, 2, 5346-5358; Antonioli L et al., Nature Reviews Cancer, 2013, 13, 842-857; Cekic C and Linden J, Nature Reviews, Immunology, 2016, 16, 177-192; Allard B et al., Curr Op Pharmacol, 2016, 29, 7-16; Vijayan D et al., Nature Reviews Cancer, 2017, 17, 709-724).

Applicant previously showed that adenosine as well as ATP profoundly suppressed T cell proliferation and cytokine secretion (IL-2), and strongly reduced T cell viability. See WO 2020/065036. Adenosine- and ATP-mediated suppression of T cell viability and proliferation were successfully restored using ENT inhibitors. Moreover, the use of ENT inhibitors with an adenosine receptor antagonist restored not only adenosine and ATP-mediated suppression of T cell viability and proliferation, but also restored T cell cytokine secretion. These results show that ENT inhibitors, either alone or in combination with an adenosine receptor antagonist, may be useful for the treatment of cancer.

The disclosure herein shows the combination of ENT1 antagonists and checkpoint inhibitors, and optionally adenosine receptor antagonists, for the treatment of cancer.

### SUMMARY

In some embodiments, provided herein is a combination of an ENT1 inhibitor and a checkpoint inhibitor.

In some embodiments, provided herein is a pharmaceutical composition comprising an ENT1 inhibitor and a checkpoint inhibitor.

In some embodiments, provided herein is a kit of parts comprising an ENT1 inhibitor and a checkpoint inhibitor.

In some embodiments, provided herein is a method of reversing adenosine-mediated suppression of T cell proliferation and/or viability, comprising administering an ENT1 inhibitor and a checkpoint inhibitor. In some embodiments, provided herein is a method of reversing adenosine-mediated suppression of antigen-specific killing activity of T cells, comprising administering an ENT1 inhibitor and a checkpoint inhibitor. In some embodiments, provided herein is a method of reversing adenosine-mediated suppression of production of cytokines.

In some embodiments, provided herein is a method of treating cancer comprising administering to a subject in need thereof an ENT1 inhibitor and a checkpoint inhibitor. In some embodiments, provided herein is a use of an ENT1 inhibitor and a checkpoint inhibitor in the manufacture of a medicament for the treatment of cancer.

In some embodiments, the combinations, compositions, kits, methods, and uses further comprise an adenosine receptor antagonist.

In some embodiments, the checkpoint inhibitor is not an anti-TIGIT antibody.

In some embodiments, the checkpoint inhibitor is chosen from a PD1 inhibitor, a PD-L1 inhibitor, and a CTLA-4 inhibitor.

In some embodiments, the checkpoint inhibitor is a PD1 inhibitor.

In some embodiments, the checkpoint inhibitor is selected from pembrolizumab, nivolumab, atezolizumab, dostarlimab, durvalumab, avelumab, and opdualag.

In some embodiments, the ENT1 inhibitor is a compound of Formula (I):

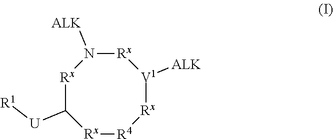

or a pharmaceutically acceptable salt, hydrate, or solvate thereof,
   wherein
R¹ is selected from the group consisting of

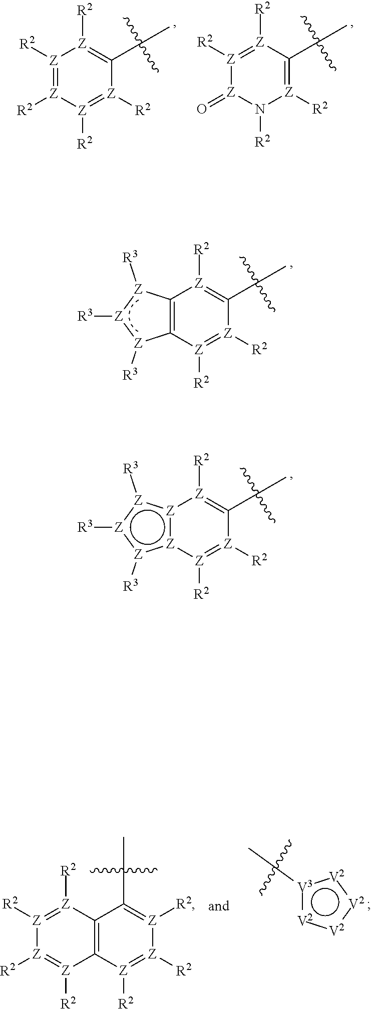

each R² is independently selected from the group consisting of absent, halogen, —NHR³, —OR³, —R³, —C(O)R³, —CO₂R³, C(O)N(R³)₂, —CH₂C(O)N(R³)₂, —S(O)₂R³, and —CN;
or two instances of R² are taken together with the atoms on which they are attached to form a heterocyclyl or heteroaryl ring;
each R³ is independently selected from absent, —H, oxo, ALK, phenyl, heterocyclyl, and heteroaryl;
R⁴ is selected from the group consisting of

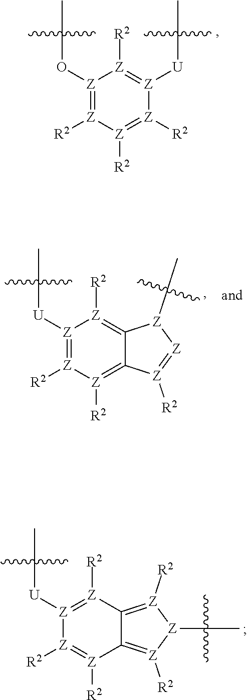

each U is independently selected from the group consisting of —C(O)—, alkylene, —O—, —N(R³)—, —C(O)O—, —C(O)N(R³)—, and

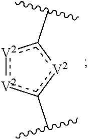

each R^{x} is independently selected from alkylene;
V¹ is selected from —C(R³)— and —N—;
each V² is independently selected from —C(R³)═, —N(R³)—, —N═, and —O—;
V³ is selected from —C═ and —N—; and
Z is C or N,
   wherein ALK is unsubstituted alkyl or substituted alkyl, or two instances of ALK may be joined together with their intervening atoms to form a cycloalkyl or heterocyclyl ring.

In some embodiments, the ENT1 inhibitor is a compound of Formula (II):

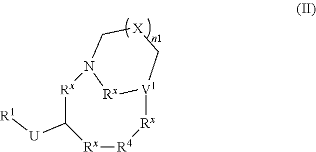

or a pharmaceutically acceptable salt, hydrate, or solvate thereof,
   wherein
R¹ is selected from the group consisting of ALK, cycloalkyl, heterocyclyl,

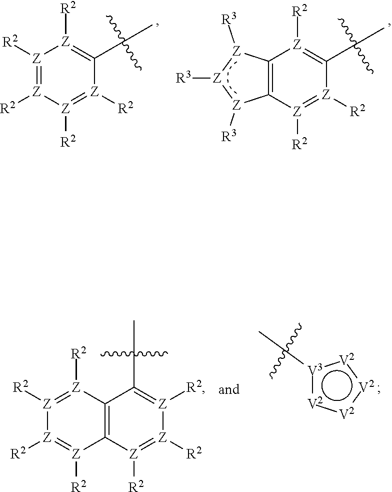

each R² is independently selected from the group consisting of absent, halogen, —OR³, —R³, —CO₂R³, C(O)N(R³)₂, —CH₂C(O)N(R³)₂, —S(O)₂R³, and —CN;
or two instances of R² are taken together with the atoms on which they are attached to form a heterocyclyl or heteroaryl ring;
each R³ is independently selected from absent, —H, ALK, phenyl, and heteroaryl;
R⁴ is

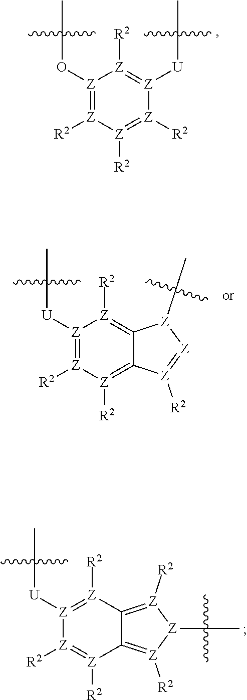

X is selected from the group consisting of —CH₂—, —CHF—, and —CF₂—; each U is independently selected from the group consisting of —O—, —N(R³)—, —C(O)O—, —C(O)N(R³)—,

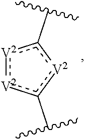

—C(O)—, —O—N═C(H)— and alkylene;
each R^{x} is independently selected from alkylene;
V¹ is selected from —C(R³)— and —N—;
each V² is independently selected from —C(R³)═, —N(R³)—, —N═, and —O—;
V³ is selected from —C═ and —N—;
each Z is independently C or N; and
n¹ is a number of 0 or 1,
   wherein ALK is unsubstituted alkyl or substituted alkyl, or two instances of ALK may be joined together with their intervening atoms to form a cycloalkyl or heterocyclyl ring.

In some embodiments, the ENT1 inhibitor is a compound of Formula (IIa):

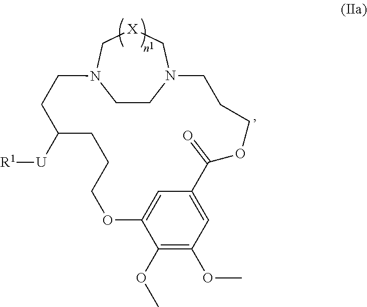

or a pharmaceutically acceptable salt, hydrate, or solvate thereof,
   wherein X is CH₂, CHF, or CF₂.

In some embodiments, in a compound of Formula (I), (II), or (IIa), R¹ is

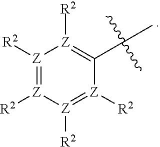

In some embodiments, in a compound of Formula (I), (II), or (IIa), R¹ is

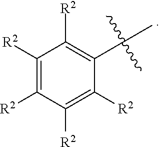

In some embodiments, the compound is a compound of Formula (IIb):

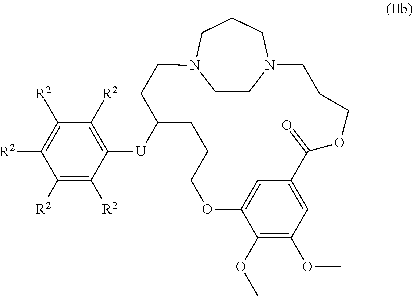

or a pharmaceutically acceptable salt, hydrate, or solvate thereof.

In some embodiments, in a compound of Formula (I), (II), (IIa), or (IIb), U is —C(O)O—.

In some embodiments, in a compound of Formula (I) or (II), R⁴ is

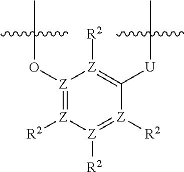

and the U in R⁴ is —C(O)O— or —C(O)NR³—.

In some embodiments, the compound is a compound of Formula (IIa1):

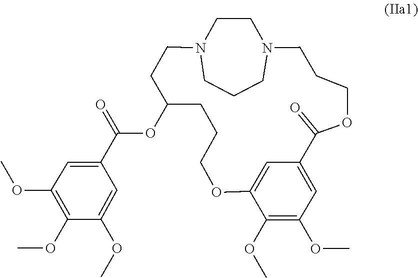

or a pharmaceutically acceptable salt, hydrate, or solvate thereof.

In some embodiments, the compound is selected from:
(12S)-7⁴,7⁵-dimethoxy-6-oxo-5,8-dioxa-1(1,4)-diazepana-7(1,3)-benzenacyclotetradecaphane-12-yl 3,4,5-trimethoxybenzoate
(12R)-7⁴,7⁵-dimethoxy-6-oxo-5,8-dioxa-1(1,4)-diazepana-7(1,3)-benzenacyclotetradecaphane-12-yl 3,4,5-trimethoxybenzoate
16,16-difluoro-7⁴,7⁵-dimethoxy-6-oxo-5,8-dioxa-1(1,4)-diazepana-7(1,3)-benzenacyclotetradecaphane-12-yl 3,4,5-trimethoxybenzoate
(12S)-16,16-difluoro-7⁴,7⁵-dimethoxy-6-oxo-5,8-dioxa-1(1,4)-diazepana-7(1,3)-benzenacyclotetradecaphane-12-yl 3,4,5-trimethoxybenzoate
(12R)-16,16-difluoro-7⁴,7⁵-dimethoxy-6-oxo-5,8-dioxa-1(1,4)-diazepana-7(1,3)-benzenacyclotetradecaphane-12-yl 3,4,5-trimethoxybenzoate
N-(7⁴,7⁵)-dimethoxy-6-oxo-5,8-dioxa-1(1,4)-diazepana-7(1,3)-benzenacyclotetradecaphane-12-yl)-3,4,5-trimethoxybenzamide
7⁴,7⁵-dimethoxy-6-oxo-8-oxa-5-aza-1(1,4)-diazepana-7(1,3)-benzenacyclotetradecaphane-12-yl 3,4,5-trimethoxybenzoate
(12S)-7⁴,7⁵-dimethoxy-6-oxo-8-oxa-5-aza-1(1,4)-diazepana-7(1,3)-benzenacyclotetradecaphane-12-yl 3,4,5-trimethoxybenzoate
(12R)-7⁴,7⁵-dimethoxy-6-oxo-8-oxa-5-aza-1(1,4)-diazepana-7(1,3)-benzenacyclotetradecaphane-12-yl 3,4,5-trimethoxybenzoate
7⁴,7⁵-dimethoxy-5-methyl-6-oxo-8-oxa-5-aza-1(1,4)-diazepana-7(1,3)-benzenacyclotetradecaphane-12-yl 3,4,5-trimethoxybenzoate
(12S)-7⁴,7⁵-dimethoxy-5-methyl-6-oxo-8-oxa-5-aza-1(1,4)-diazepana-7(1,3)-benzenacyclotetradecaphane-12-yl 3,4,5-trimethoxybenzoate
(12R)-7⁴,7⁵-dimethoxy-5-methyl-6-oxo-8-oxa-5-aza-1(1,4)-diazepana-7(1,3)-benzenacyclotetradecaphane-12-yl 3,4,5-trimethoxybenzoate
(11R)-7⁴,7⁵-dimethoxy-6-oxo-5-aza-1(1,4)-diazepana-7(1,3)-benzenacyclotridecaphane-11-yl 3,4,5-trimethoxybenzoate
(10S)-14-chloro-2-oxo-11H-3-aza-1(6,1)-indazola-7(1,4)-diazepanacyclotridecaphane-10-yl 3,4,5-trimethoxybenzoate
(10R)-14-chloro-2-oxo-11H-3-aza-1(6,1)-indazola-7(1,4)-diazepanacyclotridecaphane-10-yl 3,4,5-trimethoxybenzoate
(12S)-6-oxo-5,8-dioxa-1(1,4)-diazepana-7(1,3)-benzenacyclotetradecaphane-12-yl 3,4,5-trimethoxybenzoate
(12R)-6-oxo-5,8-dioxa-1(1,4)-diazepana-7(1,3)-benzenacyclotetradecaphane-12-yl 3,4,5-trimethoxybenzoate
(12S)-6-oxo-8-oxa-5-aza-1(1,4)-diazepana-7(1,3)-benzenacyclotetradecaphane-12-yl benzoate
(12R)-6-oxo-8-oxa-5-aza-1(1,4)-diazepana-7(1,3)-benzenacyclotetradecaphane-12-yl benzoate
7⁴,7⁵-dichloro-6-oxo-5,8-dioxa-1(1,4)-diazepana-7(1,3)-benzenacyclotetradecaphane-12-yl 3,4,5-trimethoxybenzoate
(12S)-7⁴,7⁵-dichloro-6-oxo-5,8-dioxa-1(1,4)-diazepana-7(1,3)-benzenacyclotetradecaphane-12-yl 3,4,5-trimethoxybenzoate
(12R)-7⁴,7⁵-dichloro-6-oxo-5,8-dioxa-1(1,4)-diazepana-7(1,3)-benzenacyclotetradecaphane-12-yl 3,4,5-trimethoxybenzoate
7⁵-carbamoyl-7⁴-chloro-6-oxo-5,8-dioxa-1(1,4)-diazepana-7(1,3)-benzenacyclotetradecaphane-12-yl 3,4,5-trimethoxybenzoate
(11Z,16E,10S)-14-chloro-2-oxo-12H-3-aza-1(6,2)-indazola-7(1,4)-diazepanacyclotridecaphane-10-yl 3,4,5-trimethoxybenzoate
(11Z,16E,10R)-14-chloro-2-oxo-12H-3-aza-1(6,2)-indazola-7(1,4)-diazepanacyclotridecaphane-10-yl 3,4,5-trimethoxybenzoate
(12S)-7⁴-carbamoyl-7⁵-chloro-6-oxo-8-oxa-5-aza-1(1,4)-diazepana-7(1,3)-benzenacyclotetradecaphane-12-yl 3,4,5-trimethoxybenzoate
(12R)-7⁴-carbamoyl-7⁵-chloro-6-oxo-8-oxa-5-aza-1(1,4)-diazepana-7(1,3)-benzenacyclotetradecaphane-12-yl 3,4,5-trimethoxybenzoate
7⁴-bromo-7⁵-chloro-6-oxo-8-oxa-5-aza-1(1,4)-diazepana-7(1,3)-benzenacyclotetradecaphane-12-yl 3,4,5-trimethoxybenzoate
7⁵-chloro-7⁴-cyano-6-oxo-8-oxa-5-aza-1(1,4)-diazepana-7(1,3)-benzenacyclotetradecaphane-12-yl 3,4,5-trimethoxybenzoate
(12R)-7⁴,7⁵-dimethoxy-6-oxo-5,8-dioxa-1(1,4)-diazepana-7(1,3)-benzenacyclotetradecaphane-12-yl benzoate
(12R)-7⁴,7⁵-dimethoxy-6-oxo-5,8-dioxa-1(1,4)-diazepana-7(1,3)-benzenacyclotetradecaphane-12-yl benzoate
(12S)-7⁴,7⁵-dimethoxy-6-oxo-5,8-dioxa-1(1,4)-diazepana-7(1,3)-benzenacyclotetradecaphane-12-yl benzoate
(Z)-benzaldehyde O-(7⁴,7⁵-dimethoxy-6-oxo-5,8-dioxa-1(1,4)-diazepana-7(1,3)-benzenacyclotetradecaphane-12-yl) oxime
12-hydroxy-7⁴,7⁵-dimethoxy-5,8-dioxa-1(1,4)-diazepana-7(1,3)-benzenacyclotetradecaphan-6-one
7⁴,7⁵-dimethoxy-6-oxo-5,8-dioxa-1(1,4)-diazepana-7(1,3)-benzenacyclotetradecaphane-12-yl 4-hydroxybenzoate
7⁴,7⁵-dimethoxy-6-oxo-5,8-dioxa-1(1,4)-diazepana-7(1,3)-benzenacyclotetradecaphane-12-yl 4-fluorobenzoate
7⁴,7⁵-dimethoxy-6-oxo-5,8-dioxa-1(1,4)-diazepana-7(1,3)-benzenacyclotetradecaphane-12-yl 4-isopropoxybenzoate
7⁴,7⁵-dimethoxy-6-oxo-5,8-dioxa-1(1,4)-diazepana-7(1,3)-benzenacyclotetradecaphane-12-yl 3-(trifluoromethyl)benzoate
7⁴,7⁵-dimethoxy-6-oxo-5,8-dioxa-1(1,4)-diazepana-7(1,3)-benzenacyclotetradecaphane-12-yl 3-(methylsulfonyl)benzoate
7⁴,7⁵-dimethoxy-6-oxo-5,8-dioxa-1(1,4)-diazepana-7(1,3)-benzenacyclotetradecaphane-12-yl 3-phenoxybenzoate
7⁴,7⁵-dimethoxy-6-oxo-5,8-dioxa-1(1,4)-diazepana-7(1,3)-benzenacyclotetradecaphane-12-yl 2-fluorobenzoate
7⁴,7⁵-dimethoxy-6-oxo-5,8-dioxa-1(1,4)-diazepana-7(1,3)-benzenacyclotetradecaphane-12-yl 4-bromo-3-cyanobenzoate
7⁴,7⁵-dimethoxy-6-oxo-5,8-dioxa-1(1,4)-diazepana-7(1,3)-benzenacyclotetradecaphane-12-yl 3-methyl-5-(trifluoromethyl)benzoate
7⁴,7⁵-dimethoxy-6-oxo-5,8-dioxa-1(1,4)-diazepana-7(1,3)-benzenacyclotetradecaphane-12-yl 2-fluoro-4-methoxybenzoate
7⁴,7⁵-dimethoxy-6-oxo-5,8-dioxa-1(1,4)-diazepana-7(1,3)-benzenacyclotetradecaphane-12-yl 4-methoxy-2-(trifluoromethoxy)benzoate
7⁴,7⁵-dimethoxy-6-oxo-5,8-dioxa-1(1,4)-diazepana-7(1,3)-benzenacyclotetradecaphane-12-yl picolinate
7⁴,7⁵-dimethoxy-6-oxo-5,8-dioxa-1(1,4)-diazepana-7(1,3)-benzenacyclotetradecaphane-12-yl nicotinate
7⁴,7⁵-dimethoxy-6-oxo-5,8-dioxa-1(1,4)-diazepana-7(1,3)-benzenacyclotetradecaphane-12-yl pyrazine-2-carboxylate
7⁴,7⁵-dimethoxy-6-oxo-5,8-dioxa-1(1,4)-diazepana-7(1,3)-benzenacyclotetradecaphane-12-yl 6-hydroxynicotinate
7⁴,7⁵-dimethoxy-6-oxo-5,8-dioxa-1(1,4)-diazepana-7(1,3)-benzenacyclotetradecaphane-12-yl quinoline-5-carboxylate
7⁴,7⁵-dimethoxy-6-oxo-5,8-dioxa-1(1,4)-diazepana-7(1,3)-benzenacyclotetradecaphane-12-yl oxazole-4-carboxylate
7⁴,7⁵-dimethoxy-6-oxo-5,8-dioxa-1(1,4)-diazepana-7(1,3)-benzenacyclotetradecaphane-12-yl 1H-1,2,3-triazole-4-carboxylate
7⁴,7⁵-dimethoxy-6-oxo-5,8-dioxa-1(1,4)-diazepana-7(1,3)-benzenacyclotetradecaphane-12-yl acetate
7⁴,7⁵-dimethoxy-6-oxo-5,8-dioxa-1(1,4)-diazepana-7(1,3)-benzenacyclotetradecaphane-12-yl cyclopropanecarboxylate
7⁴,7⁵-dimethoxy-6-oxo-5,8-dioxa-1(1,4)-diazepana-7(1,3)-benzenacyclotetradecaphane-12-yl 3-methylbutanoate
7⁴,7⁵-dimethoxy-6-oxo-5,8-dioxa-1(1,4)-diazepana-7(1,3)-benzenacyclotetradecaphane-12-yl 4,4,4-trifluorobutanoate
7⁴,7⁵-dimethoxy-6-oxo-5,8-dioxa-1(1,4)-diazepana-7(1,3)-benzenacyclotetradecaphane-12-yl cyclohexanecarboxylate
7⁴,7⁵-dimethoxy-6-oxo-5,8-dioxa-1(1,4)-diazepana-7(1,3)-benzenacyclotetradecaphane-12-yl 1-methylpiperidine-4-carboxylate
7⁴,7⁵-dimethoxy-6-oxo-5,8-dioxa-1(1,4)-diazepana-7(1,3)-benzenacyclotetradecaphane-12-yl 3,3-dimethylcyclobutane-1-carboxylate
7⁴,7⁵-dimethoxy-6-oxo-5,8-dioxa-1(1,4)-diazepana-7(1,3)-benzenacyclotetradecaphane-12-yl 2-(oxetan-3-yl)acetate
7⁴,7⁵-dimethoxy-6-oxo-5,8-dioxa-1(1,4)-diazepana-7(1,3)-benzenacyclotetradecaphane-12-yl (1R,5S,6R)-3-oxabicyclo[3.1.0]hexane-6-carboxylate
7⁴,7⁵-dimethoxy-6-oxo-5,8-dioxa-1(1,4)-diazepana-7(1,3)-benzenacyclotetradecaphane-12-yl 5-oxopyrrolidine-3-carboxylate
7⁴,7⁵-dimethoxy-6-oxo-5,8-dioxa-1(1,4)-diazepana-7(1,3)-benzenacyclotetradecaphane-12-yl 1-benzyl-5-oxopyrrolidine-3-carboxylate
7⁴,7⁵-dimethoxy-6-oxo-5,8-dioxa-1(1,4)-diazepana-7(1,3)-benzenacyclotetradecaphane-12-yl 4-methoxycyclohexane-1-carboxylate
7⁴,7⁵-dimethoxy-6-oxo-5,8-dioxa-1(1,4)-diazepana-7(1,3)-benzenacyclotetradecaphane-12-yl 2,6-difluorobenzoate
7⁴,7⁵-dimethoxy-6-oxo-5,8-dioxa-1(1,4)-diazepana-7(1,3)-benzenacyclotetradecaphane-12-yl 4-(trifluoromethoxy)benzoate
7⁴,7⁵-dimethoxy-6-oxo-5,8-dioxa-1(1,4)-diazepana-7(1,3)-benzenacyclotetradecaphane-12-yl 3-cyanobenzoate
7⁴,7⁵-dimethoxy-6-oxo-5,8-dioxa-1(1,4)-diazepana-7(1,3)-benzenacyclotetradecaphane-12-yl 2-oxo-1,2,3,4-tetrahydroquinoline-6-carboxylate
7⁴,7⁵-dimethoxy-6-oxo-5,8-dioxa-1(1,4)-diazepana-7(1,3)-benzenacyclotetradecaphane-12-yl 3-(difluoromethoxy)benzoate
7⁴,7⁵-dimethoxy-6-oxo-5,8-dioxa-1(1,4)-diazepana-7(1,3)-benzenacyclotetradecaphane-12-yl 3,5-dichlorobenzoate
7⁴,7⁵-dimethoxy-6-oxo-5,8-dioxa-1(1,4)-diazepana-7(1,3)-benzenacyclotetradecaphane-12-yl 3,4-dichlorobenzoate
7⁴,7⁵-dimethoxy-6-oxo-5,8-dioxa-1(1,4)-diazepana-7(1,3)-benzenacyclotetradecaphane-12-yl 2,3-dichlorobenzoate
7⁴,7⁵-dimethoxy-6-oxo-5,8-dioxa-1(1,4)-diazepana-7(1,3)-benzenacyclotetradecaphane-12-yl 2-chloro-6-fluoro-3-methylbenzoate
7⁴,7⁵-dimethoxy-6-oxo-5,8-dioxa-1(1,4)-diazepana-7(1,3)-benzenacyclotetradecaphane-12-yl 3-fluoro-5-(trifluoromethyl)benzoate
7⁴,7⁵-dimethoxy-6-oxo-5,8-dioxa-1(1,4)-diazepana-7(1,3)-benzenacyclotetradecaphane-12-yl 4-fluoro-3-(trifluoromethyl)benzoate
7⁴,7⁵-dimethoxy-6-oxo-5,8-dioxa-1(1,4)-diazepana-7(1,3)-benzenacyclotetradecaphane-12-yl 4-cyano-3-fluorobenzoate
7⁴,7⁵-dimethoxy-6-oxo-5,8-dioxa-1(1,4)-diazepana-7(1,3)-benzenacyclotetradecaphane-12-yl 4-(trifluoromethyl)benzoate
7⁴,7⁵-dimethoxy-6-oxo-5,8-dioxa-1(1,4)-diazepana-7(1,3)-benzenacyclotetradecaphane-12-yl 3,5-difluorobenzoate
7⁴,7⁵-dimethoxy-6-oxo-5,8-dioxa-1(1,4)-diazepana-7(1,3)-benzenacyclotetradecaphane-12-yl 3,4-difluorobenzoate
7⁴,7⁵-dimethoxy-6-oxo-5,8-dioxa-1(1,4)-diazepana-7(1,3)-benzenacyclotetradecaphane-12-yl 3-cyano-4-fluorobenzoate
7⁴,7⁵-dimethoxy-6-oxo-5,8-dioxa-1(1,4)-diazepana-7(1,3)-benzenacyclotetradecaphane-12-yl 4-cyanobenzoate
7⁴,7⁵-dimethoxy-6-oxo-5,8-dioxa-1(1,4)-diazepana-7(1,3)-benzenacyclotetradecaphane-12-yl 3-chloro-4-fluorobenzoate
7⁴,7⁵-dimethoxy-6-oxo-5,8-dioxa-1(1,4)-diazepana-7(1,3)-benzenacyclotetradecaphane-12-yl 1-methyl-1H-benzo[d]imidazole-5-carboxylate
7⁴,7⁵-dimethoxy-6-oxo-5,8-dioxa-1(1,4)-diazepana-7(1,3)-benzenacyclotetradecaphane-12-yl 4-(oxazol-5-yl)benzoate
7⁴,7⁵-dimethoxy-6-oxo-5,8-dioxa-1(1,4)-diazepana-7(1,3)-benzenacyclotetradecaphane-12-yl 4,5-dichloro-2-fluorobenzoate
7⁴,7⁵-dimethoxy-6-oxo-5,8-dioxa-1(1,4)-diazepana-7(1,3)-benzenacyclotetradecaphane-12-yl 3,4,5-triethoxybenzoate
7⁴,7⁵-dimethoxy-6-oxo-5,8-dioxa-1(1,4)-diazepana-7(1,3)-benzenacyclotetradecaphane-12-yl 3-methoxypropanoate
7⁴,7⁵-dimethoxy-6-oxo-5,8-dioxa-1(1,4)-diazepana-7(1,3)-benzenacyclotetradecaphane-12-yl 3-(1H-pyrazol-1-yl)propanoate
7⁴,7⁵-dimethoxy-6-oxo-5,8-dioxa-1(1,4)-diazepana-7(1,3)-benzenacyclotetradecaphane-12-yl 3-cyanopropanoate
7⁴,7⁵-dimethoxy-6-oxo-5,8-dioxa-1(1,4)-diazepana-7(1,3)-benzenacyclotetradecaphane-12-yl 4-cyanobutanoate
7⁴,7⁵-dimethoxy-6-oxo-5,8-dioxa-1(1,4)-diazepana-7(1,3)-benzenacyclotetradecaphane-12-yl 4-acetamidobutanoate
7⁴,7⁵-dimethoxy-6-oxo-5,8-dioxa-1(1,4)-diazepana-7(1,3)-benzenacyclotetradecaphane-12-yl 3-(1H-tetrazol-1-yl)propanoate
7⁴,7⁵-dimethoxy-6-oxo-5,8-dioxa-1(1,4)-diazepana-7(1,3)-benzenacyclotetradecaphane-12-yl 4-(dimethylamino)-4-oxobutanoate
7⁴,7⁵-dimethoxy-6-oxo-5,8-dioxa-1(1,4)-diazepana-7(1,3)-benzenacyclotetradecaphane-12-yl 3-acetamidopropanoate
7⁴,7⁵-dimethoxy-6-oxo-5,8-dioxa-1(1,4)-diazepana-7(1,3)-benzenacyclotetradecaphane-12-yl 4-(methylamino)-4-oxobutanoate
7⁴,7⁵-dimethoxy-6-oxo-5,8-dioxa-1(1,4)-diazepana-7(1,3)-benzenacyclotetradecaphane-12-yl 3-(1H-1,2,4-triazol-1-yl)propanoate
7⁴,7⁵-dimethoxy-6-oxo-5,8-dioxa-1(1,4)-diazepana-7(1,3)-benzenacyclotetradecaphane-12-yl 4-morpholino-4-oxobutanoate
7⁴,7⁵-dimethoxy-6-oxo-5,8-dioxa-1(1,4)-diazepana-7(1,3)-benzenacyclotetradecaphane-12-yl 3-(4-fluorophenoxy)propanoate
7⁴,7⁵-dimethoxy-6-oxo-5,8-dioxa-1(1,4)-diazepana-7(1,3)-benzenacyclotetradecaphane-12-yl 4,4-difluorocyclohexane-1-carboxylate
7⁴,7⁵-dimethoxy-6-oxo-5,8-dioxa-1(1,4)-diazepana-7(1,3)-benzenacyclotetradecaphane-12-yl 4-(trifluoromethyl)cyclohexane-1-carboxylate
7⁴,7⁵-dimethoxy-6-oxo-5,8-dioxa-1(1,4)-diazepana-7(1,3)-benzenacyclotetradecaphane-12-yl 3-(2,5-dioxopyrrolidin-1-yl)propanoate
7⁴,7⁵-dimethoxy-6-oxo-5,8-dioxa-1(1,4)-diazepana-7(1,3)-benzenacyclotetradecaphane-12-yl 3-methoxycyclohexane-1-carboxylate
7⁴,7⁵-dimethoxy-6-oxo-8-oxa-5-aza-1(1,4)-diazepana-7(1,3)-benzenacyclotetradecaphane-12-yl benzoate
(E)-benzaldehyde O-(7⁴,7⁵-dimethoxy-6-oxo-8-oxa-5-aza-1(1,4)-diazepana-7(1,3)-benzenacyclotetradecaphane-12-yl) oxime
(E)-benzaldehyde O-((12R)-7⁴,7⁵-dimethoxy-6-oxo-8-oxa-5-aza-1(1,4)-diazepana-7(1,3)-benzenacyclotetradecaphane-12-yl) oxime
(E)-benzaldehyde O-((12S)-7⁴,7⁵-dimethoxy-6-oxo-8-oxa-5-aza-1(1,4)-diazepana-7(1,3)-benzenacyclotetradecaphane-12-yl) oxime
12-hydroxy-7⁴,7⁵-dimethoxy-8-oxa-5-aza-1(1,4)-diazepana-7(1,3)-benzenacyclotetradecaphan-6-one
(12R)-12-hydroxy-7⁴,7⁵-dimethoxy-5,8-dioxa-1(1,4)-diazepana-7(1,3)-benzenacyclotetradecaphan-6-one
(12S)-12-hydroxy-7⁴,7⁵-dimethoxy-5,8-dioxa-1(1,4)-diazepana-7(1,3)-benzenacyclotetradecaphan-6-one
7⁴,7⁵-dimethoxy-12-(5-phenyl-2H-tetrazol-2-yl)-5,8-dioxa-1(1,4)-diazepana-7(1,3)-benzenacyclotetradecaphan-6-one
7⁴,7⁵-dimethoxy-12-(4-phenyl-1H-1,2,3-triazol-1-yl)-5,8-dioxa-1(1,4)-diazepana-7(1,3)-benzenacyclotetradecaphan-6-one
7⁴,7⁵-dimethoxy-12-(5-phenyl-1H-tetrazol-1-yl)-5,8-dioxa-1(1,4)-diazepana-7(1,3)-benzenacyclotetradecaphan-6-one,
   and pharmaceutically acceptable salts, hydrates, or solvates thereof.

In some embodiments, the ENT1 inhibitor is selected from (12R)-7⁴,7⁵-dimethoxy-6-oxo-8-oxa-5-aza-1(1,4)-diazepana-7(1,3)-benzenacyclotetradecaphane-12-yl 3,4,5-trimethoxybenzoate (i.e., Compound 1, structure below)

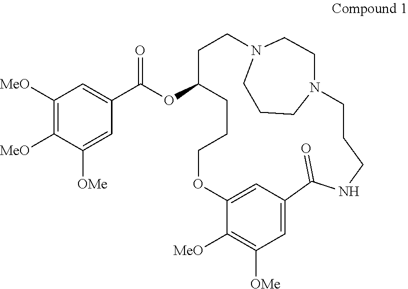 [SMILES: COc1cc(C(=O)O[C@@H]2CCCOc3cc(cc(OC)c3OC)C(=O)NCCCN3CCCN(CC2)CC3)cc(OC)c1OC]

and pharmaceutically acceptable salts, hydrates, and solvates thereof.

In some embodiments, the ENT1 inhibitor is selected from Compound 1 and pharmaceutically acceptable salts thereof.

In some embodiments, the ENT1 inhibitor is Compound 1 hydrogen sulfate or a hydrate or solvate thereof.

In some embodiments, the ENT1 inhibitor is Compound 1 di(hydrogen sulfate) or a hydrate or solvate thereof. In some embodiments, the Compound 1 di(hydrogen sulfate) or a hydrate or solvate thereof is crystalline. In some embodiments, the crystalline Compound 1 di(hydrogen sulfate) or a hydrate or solvate thereof is crystalline Form 2 Compound 1 di(hydrogen sulfate) trihydrate.

In some embodiments, the ENT1 inhibitor is administered prior to, concomitant with, or subsequent to the administration of the checkpoint inhibitor. In some embodiments, the ENT1 inhibitor is administered prior to, concomitant with, or subsequent to the administration of the checkpoint inhibitor and an adenosine receptor antagonist.

In some embodiments, the cancer is characterized by a high concentration of adenosine in the tumor microenvironment (TME).

In some embodiments, the cancer is selected from breast, carcinoid, cervical, colorectal, endometrial, glioma, head and neck, liver, lung, melanoma, ovarian, pancreatic, prostate, renal, gastric, thyroid and urothelial cancers.

In some embodiments, the cancer is breast cancer. In some embodiments, the breast cancer is triple negative breast cancer.

In some embodiments, the cancer is a solid-tumor cancer.

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

### BRIEF DESCRIPTION OF THE DRAWINGS

FIG. 1A shows an XRPD pattern of crystalline Form 2 Compound 1 di(hydrogen sulfate) trihydrate.

FIG. 1B shows a peak picked XRPD pattern of crystalline Form 2 Compound 1 di(hydrogen sulfate) trihydrate.

FIG. 2 shows XRPD patterns of crystalline Form 2 Compound 1 di(hydrogen sulfate) trihydrate as prepared in Example 1—damp and dried samples—compared to an exemplary pattern for crystalline Form 2 Compound 1 di(hydrogen sulfate) trihydrate.

FIG. 3 shows TG/DSC results of crystalline Form 2 Compound 1 di(hydrogen sulfate) trihydrate prepared in Example 1.

FIG. 4 shows a DSC thermogram of crystalline Form 2 Compound 1 di(hydrogen sulfate) trihydrate prepared in Example 1.

FIG. 5 shows a ¹H-NMR spectrum of dissolved crystalline Form 2 Compound 1 di(hydrogen sulfate) trihydrate prepared in Example 1, compared to an exemplary spectrum of dissolved Compound 1 (free base).

FIG. 6 shows a DVS isothermal plot for crystalline Form 2 Compound 1 di(hydrogen sulfate) trihydrate prepared in Example 1.

FIG. 7 shows a DVS kinetic plot for crystalline Form 2 Compound 1 di(hydrogen sulfate) trihydrate prepared in Example 1.

FIG. 8 shows an XRPD patterns of crystalline Form 2 Compound 1 di(hydrogen sulfate) trihydrate prepared according to Example 2, compared to an exemplary pattern of crystalline Form 2 Compound 1 di(hydrogen sulfate) trihydrate.

FIG. 9 shows a Raman Spectrum of a sample of crystalline Form 2 Compound 1 di(hydrogen sulfate) trihydrate prepared according to Example 2.

FIG. 10 shows XRPD patterns of crystalline Form 2 Compound 1 di(hydrogen sulfate) trihydrate prepared according to Example 3—damp, dried, and after moisture equilibration samples—compared to an exemplary XRPD pattern and an exemplary simulated single crystal XRPD of crystalline Form 2 Compound 1 di(hydrogen sulfate) trihydrate.

FIG. 11 shows PLM images of crystalline Form 2 Compound 1 di(hydrogen sulfate) trihydrate samples prepared according to Example 3—damp, dried, and after moisture equilibration.

FIG. 12 shows an overlay of Particle Size Distributions of crystalline Form 2 Compound 1 di(hydrogen sulfate) trihydrate prepared according to Example 3 at 4.33% and 6.07% water contents.

FIG. 13 shows TG/DSC data of crystalline Form 2 Compound 1 di(hydrogen sulfate) trihydrate prepared according to Example 3.

FIG. 14 shows a DSC thermogram of crystalline Form 2 Compound 1 di(hydrogen sulfate) trihydrate prepared according to Example 3.

FIG. 15 shows a ¹H-NMR spectrum of dissolved crystalline Form 2 Compound 1 di(hydrogen sulfate) trihydrate prepared according to Example 3.

FIG. 16 shows an FT-Infrared spectrum of crystalline Form 2 Compound 1 di(hydrogen sulfate) trihydrate prepared according to Example 3.

FIG. 17 shows a Single Crystal Structure Drawing of crystalline Form 2 Compound 1 di(hydrogen sulfate) trihydrate at 100 K—orientation 1.

FIG. 18 shows Single Crystal Structure Drawing of crystalline Form 2 Compound 1 di(hydrogen sulfate) trihydrate at 100 K—orientation 2.

FIG. 19 shows simulated XRPD patterns for crystalline Form 2 Compound 1 di(hydrogen sulfate) trihydrate from crystallographic data obtained at 100 K and 295 K.

FIG. 20 shows stacked plots of simulated XRPD patterns from crystallographic data obtained at 100 K and 295 K (bottom two patterns) along with the experimental XRPD for crystalline Form 2 Compound 1 di(hydrogen sulfate) trihydrate at 298 K (top pattern).

FIG. 21 shows XRPD patterns taken during the experiments relating to crystalline Compound 1 as described in Example 5, compared to exemplary patterns of Compound 1 Form 1 and Compound 1 Form 2.

FIG. 22 shows an XRPD pattern of crystalline Form 1 Compound 1, as prepared in Example 5.

FIG. 23 shows an XRPD pattern of crystalline Form 2 Compound 1, as prepared in Example 5.

FIG. 24 shows XRPD patterns of the results of the DVS experiment described in Example 5 on crystalline Form 1 Compound 1 compared to the input material.

FIG. 25 an XRPD pattern of amorphous Compound 1 di(hydrogen sulfate) prepared according to Example 6B.

FIG. 26 shows the results of the experiment described in Example 7. Compound 1 is the ENT1 inhibitor (12R)-7⁴,7⁵-dimethoxy-6-oxo-8-oxa-5-aza-1(1,4)-diazepana-7(1,3)-benzenacyclotetradecaphane-12-yl 3,4,5-trimethoxybenzoate. The anti-PD-1 agent used was nivolumab. The vehicle was a solution of 2.5% dimethyl sulfoxide (DMSO), 10% solutol HS15, and 87.5% phosphate-buffered saline (PBS). The data is representative of 8 mice per group.

FIG. 27A shows the results of an experiment whereby human T cells were activated for 2 days in the presence of Compound 1 or dilazep, then washed and used in a [³H]adenosine uptake experiment. The results indicate that Compound 1 inhibits the uptake of adenosine into human T cells with an IC50 value of 0.2 nM as compared with 1.2 nM for dilazep.

FIG. 27B shows the results of an experiment whereby human T cells were activated in the presence of 100 μM ATP and dilazep or Compound 1 and proliferation was monitored by CarboxyFluoroscein Succinimidyl Ester (CFSE) dilution. The results indicate that Compound 1 restores T cell proliferation under these conditions with an IC50 value of 0.06 nM as compared with 2.4 nM for dilazep under these conditions.

FIG. 27C shows the results of ENT1 transport inhibition assay experiment demonstrating that Compound 1 inhibits ENT1-mediated uridine transport with nanomolar potency.

FIGS. 28A, 28B, and 28C show the results of an experiment whereby human tumors were dissociated, and cells were CFSE-labelled and stimulated with CD3/CD28 microbeads and IL-2 in the presence of 500 μM of ATP as a source of adenosine. FIGS. 28A and 28B show data of Compound 1-mediated rescue of CD8+ T cell proliferation and TNF, IFNγ, and granzyme B, respectively, from dissociated tumor cell suspension cultures. Symbols represent each donor, bars are group mean±s.d. One-way RM ANOVA with Tukey's multiple comparisons test. The results indicate that ATP restricted T cell proliferation was partially reversed by Compound 1 alone and in combination with anti-PD-1 (FIG. 28A). TNF, IFNγ, and granzyme B levels were also assessed in culture supernatants and demonstrated to be suppressed by ATP but restored in the presence of Compound 1 (FIG. 28B). FIG. 28C shows Compound 1 suppresses ENT1 expression.

FIGS. 29A and 29B show the results of an experiment whereby human T cells were activated in the presence of 300 μM ATP and Compound 1, anti-PD-1, or Compound 1 and anti-PD-1. The results indicate that Compound 1 restores CMV pp65 peptide-specific T cell expansion in the presence of ATP (300 M) as a source of adenosine, resulting in a restoration of antigen-specific killing activity, respectively.

### DESCRIPTION OF THE EMBODIMENTS

### I. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art. Standard techniques may be used for chemical synthesis and chemical analysis. As appropriate, procedures involving the use of commercially available kits and reagents are generally carried out in accordance with manufacturer-defined protocols and conditions unless otherwise noted.

For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001.

Unless otherwise indicated, the following terms have the following meanings:

As used herein, the singular forms “a,” “an,” and “the” include the plural referents unless the context clearly indicates otherwise. The terms “include,” “such as,” and the like are intended to convey inclusion without limitation, unless otherwise specifically indicated.

As used herein, the term “comprising” also specifically includes embodiments “consisting of” and “consisting essentially of” the recited elements, unless specifically indicated otherwise.

The term “about” indicates and encompasses an indicated value and a range above and below that value. In certain embodiments, the term “about” indicates the designated value±10%, ±5%, or ±1%. In certain embodiments, where applicable, the term “about” indicates the designated value(s)±one standard deviation of that value(s).

The terms “adenosine A_{2A} receptor,” “A_{2A} receptor,” “A_{2A}R,” and “A_{2A}R” are used interchangeably to refer to a cell surface adenosine receptor with adenosine as the endogenous ligand. In humans, A_{2A}R is encoded by the ADORA2A gene.

The term “aldehyde” refers to a group —CHO.

The term “alkenyl” refers to unsaturated hydrocarbyl group, which may be linear or branched, comprising one or more carbon-carbon double bonds. Suitable alkenyl groups comprise between 2 and 6 carbon atoms, for example between 2 and 4 carbon atoms, such as between 2 and 3 carbon atoms. Examples of alkenyl groups are ethenyl, 2-propenyl, 2-butenyl, 3-butenyl, 2-pentenyl and its isomers, 2-hexenyl and its isomers, 2,4-pentadienyl and the like.

The term “alkenylcarbonyl” refers to a group —(C═O)-alkenyl wherein alkenyl is as herein defined.

The term “alkenylcarbonylamino” refers to a group —NH—(C═O)-alkenyl wherein alkenyl is as herein defined.

The term “alkoxy” refers to a group —O-alkyl wherein alkyl is as herein defined.

The term “ALK” or “Alk” or “alk” refers to an alkyl group (hydrocarbyl radical of formula CₙH₂ₙ₊₁ wherein n is a number greater than or equal to 1) or an alkyl group substituted by, for example, one to four substituents, such as, halo, trifluoromethyl, trifluoromethoxy, hydroxy, alkoxy, cycloalkoxy, heterocyclooxy, oxo, alkanoyl, aryloxy, alkanoyloxy, amino, alkylamino, arylamino, aralkylamino, cycloalkylamino, heterocycloamino, disubstituted amines in which the 2 amino substituents are selected from alkyl, aryl or aralkyl, alkanoylamino, aroylamino, aralkanoylamino, substituted alkanoylamino, substituted arylamino, substituted aralkanoylamino, thiol, alkylthio, arylthio, aralkylthio, cycloalkylthio, heterocyclothio, alkylthiono, arylthiono, aralkylthiono, alkylsulfonyl, arylsulfonyl, aralkylsulfonyl, sulfonamido (e.g. SO₂NH₂), substituted sulfonamido, nitro, cyano, carboxy, carbamyl (e.g. CONH₂), substituted carbamyl (e.g. CONH alkyl, CONH aryl, CONH aralkyl or cases where there are two substituents on the nitrogen selected from alkyl, aryl or aralkyl), alkoxycarbonyl, aryl, substituted aryl, guanidino and heterocyclos, such as, indolyl, imidazolyl, furyl, thienyl, thiazolyl, pyrrolidyl, pyridyl, pyrimidyl and the like. Where noted above, when the substituent is further substituted, it may be substituted with halogen, alkyl, alkoxy, aryl or aralkyl. In some embodiments, ALK is optionally substituted C₁-C₈ alkyl.

In some embodiments, an alkyl group is substituted by OH, alkoxy, CF₃, and/or NR₂.

Generally, alkyl groups comprise from 1 to 8 carbon atoms, for example, from 1 to 6 carbon atoms. Alkyl groups may be linear or branched. Suitable alkyl groups include methyl, ethyl, propyl, butyl, pentyl, hexyl, heptyl, and octyl.

The term “alkylaminoalkyl” refers to a group -alkyl-NH-alkyl wherein alkyl is as herein defined.

The term “alkylaminoalkylaminocarbonyl” refers to a group —(C═O)—NH-alkyl-NH-alkyl wherein alkyl is as herein defined.

The term “(alkylaminoalkyl)(alkyl)aminocarbonyl” refers to a group —(C═O)—NR¹R² wherein R¹ is an alkyl group and R² is a -alkyl-NH-alkyl group, wherein alkyl is as herein defined.

The term “alkylaminoalkylcarbonyl” refers to a group —(C═O)-alkyl-NH-alkyl wherein alkyl is as herein defined.

The term “alkylcarbonyl” refers to a group —(C═O)-alkyl wherein alkyl is as herein defined.

The term “alkylcarbonylamine” refers to a group —NH—(C═O)-alkyl wherein alkyl is as herein defined.

The term “alkylcarbonyloxyalkyl” refers to a group -alkyl-O—(C═O)-alkyl wherein alkyl is as herein defined.

The term “alkylheteroaryl” refers to any heteroaryl substituted by an alkyl group wherein alkyl is as herein defined.

The term “alkyloxyalkyl” refers to a group -alkyl-O-alkyl wherein alkyl is as herein defined.

The term “alkyloxycarbonyl” refers to a group —(C═O)—O-alkyl wherein alkyl is as herein defined.

The term “alkylsulfonyl” refers to a group —SO₂-alkyl wherein alkyl is as herein defined.

The term “alkylsulfonylaminoalkyl” refers to a group -alkyl-NH—SO₂-alkyl wherein alkyl is as herein defined.

The term “alkylsulfonealkyl” refers to a group -alkyl-SO₂-alkyl wherein alkyl is as herein defined.

The term “alkylsulfonimidoyl” refers to a group —S(═O)(═NH)-alkyl wherein alkyl is as herein defined.

The term “alkylsulfoxide” refers to a group —(S═O)-alkyl wherein alkyl is as herein defined.

The term “alkylsulfoxidealkyl” refers to a group -alkyl-SO-alkyl wherein alkyl is as herein defined.

The term “alkylene,” as used herein, refers to an alkyl group, as defined above, wherein one of the alkyl group's hydrogen atoms has been replaced with a bond. Alkylene groups possess two points of attachment. Non-limiting examples of alkylene groups include —CH₂—, —CH₂CH₂—, —CH₂CH₂CH₂—, —CH₂CH₂CH₂CH₂—, —CH(CH₃)CH₂CH₂—, —CH(CH₃)— and CH₂CH(CH₃)CH₂—. In one embodiment, an alkylene group has from 1 to about 6 carbon atoms. In another embodiment, an alkylene group has from about 3 to about 5 carbon atoms. In another embodiment, an alkylene group is branched. In another embodiment, an alkylene group is linear. In one embodiment, an alkylene group is —CH₂—. In one embodiment, at least one hydrogen atom of an alkylene group is substituted by a substituent such as halo, trifluoromethyl, trifluoromethoxy, hydroxy, alkoxy, cycloalkoxy, heterocyclooxy, oxo, alkanoyl, aryloxy, alkanoyloxy, amino, alkylamino, arylamino, aralkylamino, cycloalkylamino, heterocycloamino, disubstituted amines in which the 2 amino substituents are selected from alkyl, aryl or aralkyl, alkanoylamino, aroylamino, aralkanoylamino, substituted alkanoylamino, substituted arylamino, substituted aralkanoylamino, thiol, alkylthio, arylthio, aralkylthio, cycloalkylthio, heterocyclothio, alkylthiono, arylthiono, aralkylthiono, alkylsulfonyl, arylsulfonyl, aralkylsulfonyl, sulfonamido (e.g. SO₂NH₂), substituted sulfonamido, nitro, cyano, carboxy, carbamyl (e.g. CONH₂), substituted carbamyl (e.g. CONH alkyl, CONH aryl, CONH aralkyl or cases where there are two substituents on the nitrogen selected from alkyl, aryl or aralkyl), alkoxycarbonyl, aryl, substituted aryl, guanidino and heterocyclos, such as, indolyl, imidazolyl, furyl, thienyl, thiazolyl, pyrrolidyl, pyridyl, pyrimidyl and the like. Where noted above, when the substituent is further substituted, it may be substituted with halogen, alkyl, alkoxy, aryl or aralkyl. In another embodiment, at least one hydrogen atom of an alkylene group is substituted by —OH, alkoxy, —CF₃, or —NR₂.

The term “alkyne” refers to a class of monovalent unsaturated hydrocarbyl groups, wherein the unsaturation arises from the presence of one or more carbon-carbon triple bonds. Alkynyl groups typically have the same number of carbon atoms as described above in relation to alkyl groups. Non-limiting examples of alkynyl groups are ethynyl, 2-propynyl, 2-butynyl, 3-butynyl, 2-pentynyl and its isomers, 2-hexynyl and its isomers and the like.

The term “alkynealkyl” refers to a group -alkyl-alkyne wherein alkyl and alkyne are as herein defined.

The term “amino” refers to a group —NH₂.

The term “aminoalkyl” refers to a group -alkyl-NH₂ wherein alkyl is as herein defined.

The term “aminoalkylaminocarbonyl” refers to a group —(C═O)—NH-alkyl-NH₂ wherein alkyl is as herein defined.

The term “aminoalkylcarbonylamino” refers to a group —NH—(C═O)-alkyl-NH₂ wherein alkyl is as herein defined.

The term “aminocarbonyl” or “aminocarboxy” refers to a group —(C═O)—NH₂.

The term “(aminocarbonylalkyl)(alkyl)amino” refers to a group —NR¹R² wherein R¹ is an alkyl group and R² is a -alkyl-(C═O)—NH₂ group, wherein alkyl is as herein defined.

The term “aminocarbonylalkylamino” refers to a group —NH-alkyl-(C═O)—NH₂ wherein alkyl is as herein defined.

The term “aminosulfonyl” refers to a group —SO₂—NH₂.

The term “aryl” refers to a polyunsaturated, aromatic hydrocarbyl group having a single ring (i.e. phenyl) or multiple aromatic rings fused together (e.g. naphtyl), typically containing 5 to 12 atoms; for example, 5 to 10 atoms. In some embodiments, the aryl is a 5- or 6-membered aryl. Non-limiting examples of aryl include phenyl and naphthalenyl.

The term “arylalkyl” refers to a group -alkyl-aryl wherein alkyl and aryl are as herein defined.

The term “aryloxyalkyl” refers to a group -alkyl-O-aryl wherein alkyl and aryl are as herein defined.

The term “carbonyl” refers to a group —(C═O)—.

The term “carbonylamino” refers to a group —NH—(C═O)—.

The term “cycloalkyl” refers to a cyclic alkyl group, that is to say, a monovalent, saturated, or unsaturated hydrocarbyl group having 1 or more cyclic structures. Cycloalkyl includes monocyclic or bicyclic hydrocarbyl groups. Cycloalkyl groups may comprise 3 or more carbon atoms in the ring and, in some embodiments, comprise from 3 to 10 carbon atoms, for example from 3 to 8 carbon atoms. In some embodiments, cycloalkyl is a 5- or 6-membered cycloalkyl. Examples of cycloalkyl groups include but are not limited to cyclopropyl, cyclobutyl, cyclopentyl, and cyclohexyl.

The term “cycloalkyloxy” refers to a group —O-cycloalkyl wherein cycloalkyl is as herein defined.

The term “dialkylamino” refers to a group —NR¹R² wherein R¹ and R² are both independently alkyl group as herein defined.

The term “dialkylaminoalkyl” refers to a group -alkyl-NR¹R² wherein R¹ and R² are both independently alkyl group, as herein defined.

The term “dialkylaminoalkylaminocarbonyl” refers to a group —(C═O)—NH-alkyl-NR¹R² wherein R¹ and R² are both alkyl group, as herein defined.

The term “dialkylaminoalkylcarbonyl” refers to a group —(C═O)-alkyl-NR¹R² wherein R¹ and R² are both alkyl group, as herein defined.

The term “dihydroxyalkyl” refers to a group alkyl is as herein defined substituted by two hydroxyl (—OH) groups.

The term “halo” or “halogen” refers to fluoro, chloro, bromo, or iodo.

The term “haloalkyl” refers to an alkyl group in which one or more hydrogen atom is replaced by a halogen atom.

The term “haloalkyloxy” refers to a group —O-haloalkyl wherein alkyl is as herein defined.

The term “heteroaryl” refers to an aryl group as herein defined wherein at least one carbon atom is replaced with a heteroatom. In some embodiments, it refers to 5 to 12 carbon-atom aromatic single rings or ring systems containing 2 rings which are fused together, in some instances containing 5 to 6 atoms, in which one or more carbon atoms is replaced by a heteroatom such as an oxygen, nitrogen and/or sulfur atoms where the nitrogen and sulfur heteroatoms may optionally be oxidized and the nitrogen heteroatoms may optionally be quaternized. Non-limiting examples of heteroaryls include: pyrrolyl, furanyl, thiophenyl, pyrazolyl, imidazolyl, oxazolyl, isoxazolyl, thiazolyl, isothiazolyl, triazolyl, oxadiazolyl, thiadiazolyl, pyridinyl, pyridazinyl, pyrimidinyl, and pyrazinyl.

The term “heteroarylalkyl” refers to a group -alkyl-heteroaryl wherein alkyl and heteroaryl are as herein defined.

The term “heterocyclyl” or “heterocycle” refers to non-aromatic, fully saturated or partially unsaturated cyclic groups (for example, 3 to 7 member monocyclic, 7 to 11 member bicyclic, or containing a total of 3 to 10 ring atoms) which have at least one heteroatom in at least one carbon atom-containing ring. In some embodiments, the heterocyclyl is a 5- or 6-membered heterocyclyl. Each ring of the heterocyclic group containing a heteroatom may have 1, 2, 3 or 4 heteroatoms selected from, for example, nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system, where valence allows. The rings of multi-ring heterocycles may be fused, bridged and/or joined through one or more spiro atoms. Non limiting exemplary heterocyclic groups include piperidinyl, piperazinyl, azetidinyl, azocanyl, diazepanyl, diazocanyl, morpholin-4-yl, oxazepanyl, pyrrolidinyl, thiomorpholin-4-yl, tetrahydrofuranyl, tetrahydropyranyl,aziridinyl, oxiranyl, thiiranyl, 2-imidazolinyl, pyrazolidinyl imidazolidinyl, isoxazolinyl, oxazolidinyl, isoxazolidinyl, thiazolidinyl, isothiazolidinyl, succinimidyl, 3H-indolyl, indolinyl, isoindolinyl, 2H-pyrrolyl, 1-pyrrolinyl, 2-pyrrolinyl, 3-pyrrolinyl, 4H-quinolizinyl, 2-oxopiperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, oxetanyl, thietanyl, 3-dioxolanyl, 1,4-dioxanyl, 2,5-dioximidazolidinyl, 2-oxopiperidinyl, 2-oxopyrrolodinyl, indolinyl, tetrahydrothiophenyl, tetrahydroquinolinyl, tetrahydroisoquinolinyl, 1-oxido-1-thiomorpholin-4-yl, 1-dioxido-1-thiomorpholin-4-yl, 1,3-dioxolanyl, 1,4-oxathianyl, 1,4-dithianyl, 1,3,5-trioxanyl, 1H-pyrrolizinyl, tetrahydro-1,1-dioxothiophenyl, N-formylpiperazinyl, dihydrotriazolopyrazine, dihydroimidazopyrazine, hexahydropyrrolopyrrole, and hexahydropyrrolopyrazine.

The term “heterocyclylalkyl” refers to a group -alkyl-heterocyclyl wherein alkyl and heterocyclyl are as herein defined.

The term “heterocyclylalkylaminocarbonyl” refers to a group —(C═O)—NH-alkyl-heterocyclyl, wherein alkyl and heterocyclyl are as herein defined.

The term “(heterocyclyl)(alkyl)aminoalkyl” refers to a group -alkyl-NR¹R² wherein R¹ is an alkyl group and R² is a heterocyclyl group, wherein alkyl and heterocyclyl are as herein defined.

The term “heterocyclylalkyloxyalkyl” refers to a group -alkyl-O-alkyl-heterocyclyl wherein alkyl and heterocyclyl are as herein defined.

The term “heterocyclylcarbonyl” refers to a group —(C═O)-heterocyclyl wherein heterocyclyl is as herein defined.

The term “heterocyclyloxy” refers to a group —O-heterocyclyl wherein heterocyclyl is as herein defined.

The term “heterocyclylsulfonyl” refers to a group —SO₂-heterocyclyl wherein heterocyclyl is as herein defined.

The term “hydroxy” or “hydroxyl” refers to a group —OH.

The term “hydroxyalkyl” refers to a group -alkyl-OH wherein alkyl is as herein defined.

The term “hydroxyalkylaminoalkyl” refers to a group -alkyl-NH-alkyl-OH wherein alkyl is as herein defined.

The term “hydroxycarbonyl” refers to a group —C(═O)—OH wherein carbonyl is as herein defined. In other words, “hydroxycarbonyl” corresponds to a carboxylic acid group.

The term “oxo” refers to a ═O substituent.

The term “sulfonylamino” refers to a group —NH—SO₂.

The term “administration”, or a variant thereof (e.g. “administering”), means providing the active agent or active ingredient, alone or as part of a pharmaceutically acceptable composition, to the patient in whom/which the condition, symptom, or disease is to be treated or prevented.

The term “chemotherapy” refers to a type of cancer treatment that uses one or more anti-cancer drugs (chemotherapeutic agents) as part of a standardized chemotherapy regimen. Chemotherapy may be given with a curative intent or it may aim to prolong life or to reduce symptoms. Chemotherapeutic agents are for example selected from anticancer alkylating agents, anticancer antimetabolites, anticancer antibiotics, plant-derived anticancer agents, anticancer platinum coordination compounds and any combination thereof.

The term “hormone therapy” refers to the use of hormones in medical treatment. In one embodiment, the hormone therapy is oncologic hormone therapy.

The term “radiation therapy” refers to a method of treatment of cancer employing various radiations such as X-ray, gamma-ray, neutron ray, electron beam, proton beam and radiation sources. It is used as part of cancer treatment to control or kill malignant cells. Radiation therapy may be curative in a number of types of cancer if they are localized to one area of the body. It may also be used as part of adjuvant therapy, to prevent tumor recurrence after surgery to remove a primary malignant tumor. The three main divisions of radiation therapy are: external beam radiation therapy (EBRT or XRT); brachytherapy or sealed source radiation therapy; and systemic radioisotope therapy (RIT) or unsealed source radiotherapy.

The term “immunotherapy” refers to a therapy aiming at inducing and/or enhancing an immune response towards a specific target, for example towards cancer cells. Immunotherapy may involve the use of CAR-T cell therapy, checkpoint inhibitors, checkpoint agonists (also called T-cell agonists), IDO inhibitors, PI3K inhibitors, adenosine receptor inhibitors, adenosine-producing enzymes inhibitors, adoptive transfer, therapeutic vaccines, and combinations thereof.

The term “patient” refers to a mammal, such as a human, who/which is awaiting the receipt of, or is receiving medical care, or was/is/will be the object of a medical procedure or is monitored for the development or progression of a disease, such as a cancer.

The expression “pharmaceutically acceptable” refers to compounds, salts, compositions, dosage forms, and other materials which are useful in preparing a pharmaceutical composition that is suitable for veterinary or human pharmaceutical use.

The expression “pharmaceutically acceptable carrier” or “pharmaceutically acceptable excipient” or “excipient” includes any and all solvents, dispersion media, coatings, antibacterial and antifungal agents, isotonic and absorption delaying agents and the like. The use of such media and agents for pharmaceutically active substances is known in the art. Except insofar as any conventional media or agent is incompatible with the active ingredient, its use in the compositions disclosed herein is contemplated. Additional active ingredients can also be incorporated into the compositions.

As used herein, the term “dose” or “dosage” is defined as an amount of a therapeutic agent that is administered at a given point. The dose or dosage can be an amount sufficient to achieve or at least partially achieve a desired effect, but such a desired effect may not be visible or detectable. The terms “therapeutically effective amount” or “effective amount” or “therapeutically effective dose” or dose of a compound or a composition refer to that amount of the compound or the composition that results in reduction or inhibition of symptoms or a prolongation of survival in a subject (such as a human patient). The results may require multiple doses of the compound or the composition. A therapeutically effective amount may be administered prior to the onset of a disease or disorder for a prophylactic or preventive action. Alternatively, or additionally, a therapeutically effective amount may be administered after initiation of a disease or disorder for a therapeutic action. In one embodiment, the disease or disorder is cancer.

The term “subject” refers to a mammal, such as a human. In one embodiment, the subject is diagnosed with cancer. In one embodiment, the subject is a patient, such as a human patient, who/which is awaiting the receipt of, or is receiving, medical care or was/is/will be the subject of a medical procedure or is monitored for the development or progression of a disease, such as a cancer. In one embodiment, the subject is a human patient who is treated and/or monitored for the development or progression of a cancer. In one embodiment, the subject is a male. In another embodiment, the subject is a female. In one embodiment, the subject is an adult. In another embodiment, the subject is a child.

The term “and/or” used herein is to be taken to mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

### II. Combinations, Compositions, Kits, Methods and Uses

Disclosed herein are combinations, compositions, and kits comprising an ENT1 inhibitor and a checkpoint inhibitor. Disclosed herein are methods and uses comprising an ENT1 inhibitor and a checkpoint inhibitor. Disclosed herein are methods and uses comprising a combination, composition, or kit as described herein.

### A. ENT1 Inhibitors

Applicant observed that activated human T cells took up [3H]adenosine, and this uptake was reduced in the presence of the selective ENT1 inhibitor dilazep, confirming that ENT1 is the dominant transporter involved in adenosine uptake in T cells. These results indicate that T cells increase ENT1 expression upon activation, which could allow uptake of adenosine present in the extracellular environment.

Applicant engrafted Syngeneic cancer cell lines into wild type (WT) mice and mice in which ENT1 was genetically deleted (ENT1-knock-out [KO]). A delay in tumor growth rate was observed for MCA205 (fibrosarcoma), MC38 (colon adenocarcinoma), and Pan02 (pancreatic ductal adenocarcinoma [PDAC]) cells when implanted subcutaneously in ENT1-KO compared to WT mice. Growth of tumors derived from PDAC cells derived from tumors generated in the autochthonous KPC mouse model was also significantly reduced in ENT1-KO versus WT mice. This suppression of tumor growth was associated with an increased proportion of CD8+ T cells within the tumor microenvironment. Quantitative mass spectrometry imaging reveals adenosine concentrations in human tumors up to the range of 100 μM—levels significantly greater than previously demonstrated and consistent with suppression of T cell responses. Accordingly, deletion of ENT1 may lead to potent control of tumor growth in syngeneic mouse models including KPC, a poorly immunogenic model of pancreatic ductal adenocarcinoma, and is associated with increased CD8+ T cell frequency, proliferation and cytokine production within tumors. Furthermore, ENT1 expression was observed by flow cytometry on human tumor-infiltrating CD8+ T cells.

In one embodiment, Compound 1 or a pharmaceutically acceptable salt, hydrate, or solvate thereof inhibits adenosine uptake into activated human T cells with sub-nanomolar potency. In some embodiments, the Compound 1 or a pharmaceutically acceptable salt, hydrate, or solvate thereof is Compound 1 di(hydrogen sulfate).

In one embodiment, Compound 1 or a pharmaceutically acceptable salt, hydrate, or solvate thereof dose-dependently restores T cell proliferation in the presence of ATP acting as a source of adenosine. In some embodiments, the Compound 1 or a pharmaceutically acceptable salt, hydrate, or solvate thereof is Compound 1 di(hydrogen sulfate).

Any ENT1 inhibitor may be used in the combinations, compositions, kits, methods, and uses disclosed herein. In some embodiments, the ENT1 inhibitor is a small molecule, a nucleic acid, a peptide, or an antibody.

In some embodiments, the ENT1 inhibitor is an ENT1 inhibitor such as those disclosed in WO 2021/170797, WO 2021/204896, and WO 2023/059739.

In some embodiments, the ENT1 inhibitor is a compound of Formula (I):

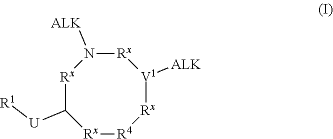

or a pharmaceutically acceptable salt, hydrate, or solvate thereof,
   wherein
R¹ is selected from the group consisting of

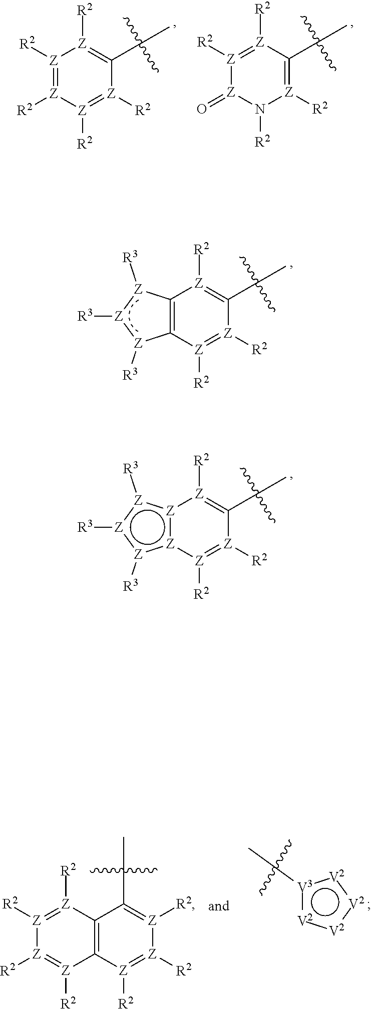

each R² is independently selected from the group consisting of absent, halogen, —NR³, —OR³, —R³, —C(O)R³, —CO₂R³, C(O)N(R³)₂, —CH₂C(O)N(R³)₂, —S(O)₂R³, and —CN;
or two instances of R² are taken together with the atoms on which they are attached to form a heterocyclyl or heteroaryl ring;
each R³ is independently selected from absent, —H, oxo, ALK, phenyl, heterocyclyl, and heteroaryl;
R⁴ is selected from the group consisting of

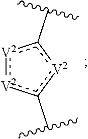

each U is independently selected from the group consisting of —C(O)—, alkylene, —O—, —N(R³)—, —C(O)O—, —C(O)N(R³)—, and

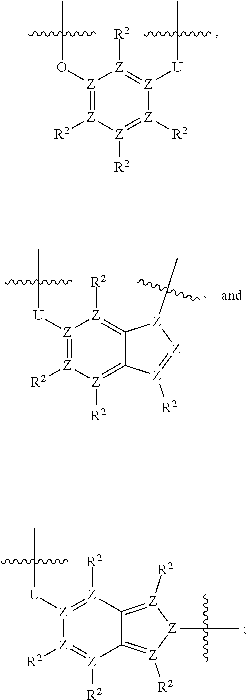

each R^{x} is independently selected from alkylene;
V¹ is selected from —C(R³)— and —N—;
each V² is independently selected from —C(R³)═, —N(R³)—, —N═, and —O—;
V³ is selected from —C═ and —N—; and
Z is C or N,
   wherein ALK is unsubstituted alkyl or substituted alkyl, or two instances of ALK may be joined together with their intervening atoms to form a cycloalkyl or heterocyclyl ring.

In some embodiments, the ENT1 inhibitor is a compound of Formula (II):

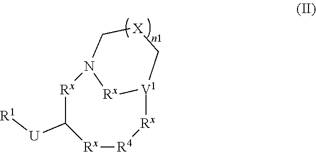

or a pharmaceutically acceptable salt, hydrate, or solvate thereof,
   wherein
R¹ is selected from the group consisting of ALK, cycloalkyl, heterocyclyl,

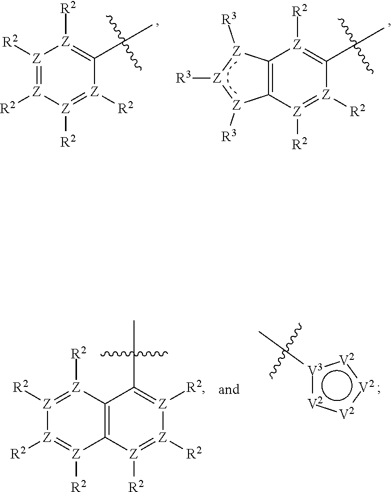

each R² is independently selected from the group consisting of absent, halogen, —OR³, —R³, —CO₂R³, C(O)N(R³)₂, —CH₂C(O)N(R³)₂, —S(O)₂R³, and —CN;
or two instances of R² are taken together with the atoms on which they are attached to form a heterocyclyl or heteroaryl ring;
each R³ is independently selected from absent, —H, ALK, phenyl, and heteroaryl;
R⁴ is

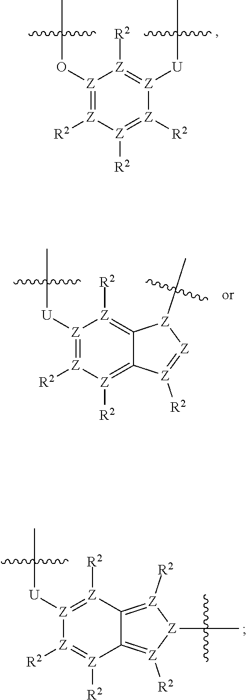

X is selected from the group consisting of —CH₂—, —CHF—, and —CF₂—;
each U is independently selected from the group consisting of —O—, —N(R³)—, —C(O)O—, —C(O)N(R³)—,

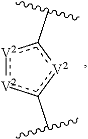

—C(O)—, —O—N═C(H)— and alkylene;
each R^{x} is independently selected from alkylene;
V¹ is selected from —C(R³)— and —N—;
each V² is independently selected from —C(R³)═, —N(R³)—, —N═, and —O—;
V³ is selected from —C═ and —N—;
each Z is independently C or N; and
n¹ is a number of 0 or 1,
   wherein ALK is unsubstituted alkyl or substituted alkyl, or two instances of ALK may be joined together with their intervening atoms to form a cycloalkyl or heterocyclyl ring.

In some embodiments, the ENT1 inhibitor is a compound of Formula (IIa):

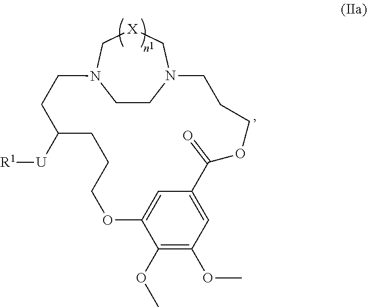

or a pharmaceutically acceptable salt, hydrate, or solvate thereof,
wherein X is CH₂, CHF, or CF₂.

In some embodiments, in a compound of Formula (I), (II), or (IIa), R¹ is

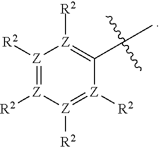

In some embodiments, in a compound of Formula (I), (II), or (IIa), R¹ is

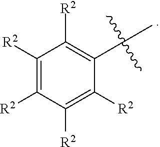

In some embodiments, the ENT1 inhibitor is a compound of Formula (IIb):

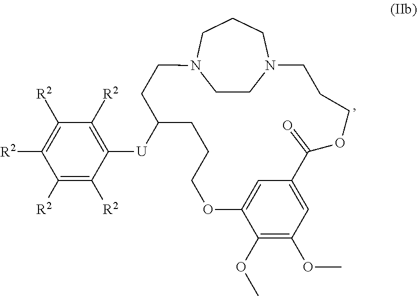

or a pharmaceutically acceptable salt, hydrate, or solvate thereof.

In some embodiments, in a compound of Formula (I), (II), (IIa), or (IIb), U is —C(O)O—.

In some embodiments, in a compound of Formula (I) or (II), R⁴ is

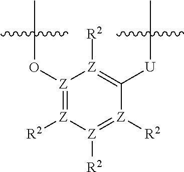

and the U in R⁴ is —C(O)O— or —C(O)NR³—. In some embodiments, U in R⁴ is —C(O)O—. In some embodiments, U in R⁴ is —C(O)NR³—.

In some embodiments, the ENT1 inhibitor is a compound of Formula (IIa1):

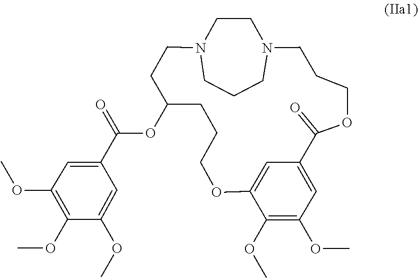

or a pharmaceutically acceptable salt, hydrate, or solvate thereof.

In some embodiments, the ENT1 inhibitor is selected from (12R)-7⁴,7⁵-dimethoxy-6-oxo-8-oxa-5-aza-1(1,4)-diazepana-7(1,3)-benzenacyclotetradecaphane-12-yl 3,4,5-trimethoxybenzoate (also known as Compound 1, structure below) and pharmaceutically acceptable salts, hydrates, or solvates thereof.

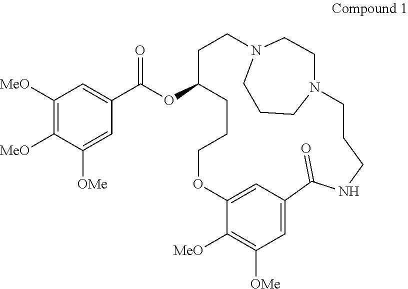 [SMILES: COc1cc(C(=O)O[C@@H]2CCCOc3cc(cc(OC)c3OC)C(=O)NCCCN3CCCN(CC2)CC3)cc(OC)c1OC]

In some embodiments, the ENT1 inhibitor is selected from (12R)-7⁴,7⁵-dimethoxy-6-oxo-8-oxa-5-aza-1(1,4)-diazepana-7(1,3)-benzenacyclotetradecaphane-12-yl 3,4,5-trimethoxybenzoate (Compound 1) and pharmaceutically acceptable salts, hydrates, or solvates thereof.

In some embodiments, the ENT1 inhibitor is a hydrogen sulfate of Compound 1 (“Compound 1 hydrogen sulfate”) or a hydrate or solvate thereof. In some embodiments, the hydrogen sulfate of Compound 1 or a hydrate or solvate thereof is a hydrate. In some embodiments, the hydrogen sulfate of Compound 1 or a hydrate or solvate thereof is a di(hydrogen sulfate) (“Compound 1 di(hydrogen sulfate)”) or a hydrate or solvate thereof, and in some embodiments, the hydrogen sulfate of Compound 1 di(hydrogen sulfate) or a hydrate or solvate thereof is a trihydrate.

In some embodiments, the Compound 1 hydrogen sulfate or a hydrate or solvate thereof is a crystalline hydrogen sulfate such as a crystalline mono(hydrogen sulfate) or a crystalline di(hydrogen sulfate) or a hydrate or solvate thereof.

In some embodiments, the ENT1 inhibitor is a Compound 1 hydrogen sulfate or a hydrate or solvate thereof, including crystalline Form 2 Compound 1 di(hydrogen sulfate) trihydrate, having an X-ray powder diffraction pattern comprising a peak at about 4.9° 2θ. In some embodiments, the Compound 1 hydrogen sulfate or hydrate or solvate thereof, including crystalline Form 2 Compound 1 di(hydrogen sulfate) trihydrate, has an X-ray powder diffraction pattern comprising peaks at about 4.9° 2θ and about 9.9° 2θ. In some embodiments, the Compound 1 hydrogen sulfate or hydrate or solvate thereof, including crystalline Form 2 Compound 1 di(hydrogen sulfate) trihydrate, has an X-ray powder diffraction pattern comprising peaks at about 4.9° 2θ and about 11.7° 2θ. In some embodiments, the Compound 1 hydrogen sulfate or hydrate or solvate thereof, including crystalline Form 2 Compound 1 di(hydrogen sulfate) trihydrate, has an X-ray powder diffraction pattern comprising peaks at about 4.9° 2θ and about 15.0° 2θ. In some embodiments, the Compound 1 hydrogen sulfate or hydrate or solvate thereof, including crystalline Form 2 Compound 1 di(hydrogen sulfate) trihydrate, has an X-ray powder diffraction pattern comprising peaks at about 4.9° 2θ, about 11.7° 2θ, and about 15.0° 2θ.

In some embodiments, the ENT1 inhibitor is a Compound 1 hydrogen sulfate or hydrate or solvate thereof, including crystalline Form 2 Compound 1 di(hydrogen sulfate) trihydrate, having an X-ray powder diffraction pattern comprising one or more peaks chosen from about 4.9° 2θ, about 9.9° 2θ about 10.2° 2θ, about 11.7° 2θ, about 12.7° 2θ, about 14.4° 2θ, about 15.0° 2θ, about 15.7° 2θ, about 19.0° 2θ, and about 19.6° 2θ.

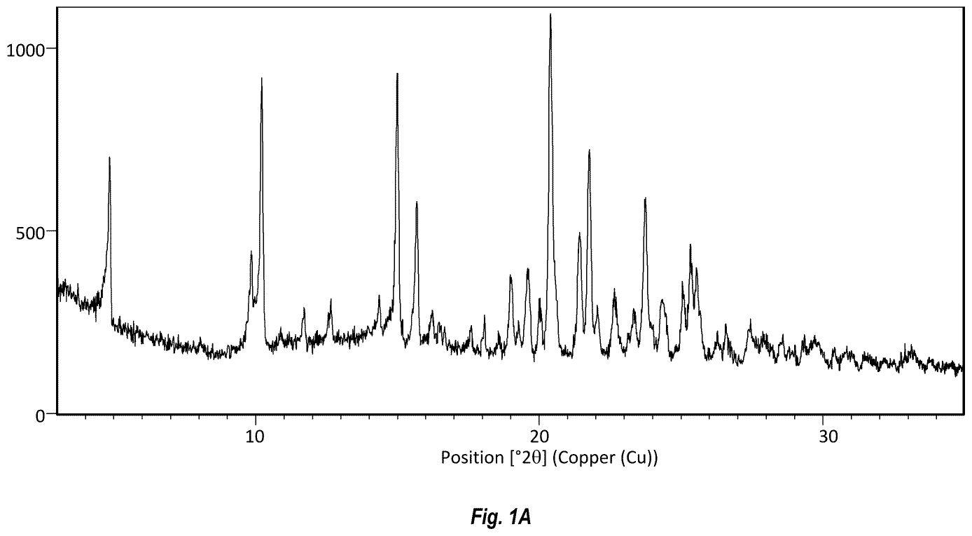

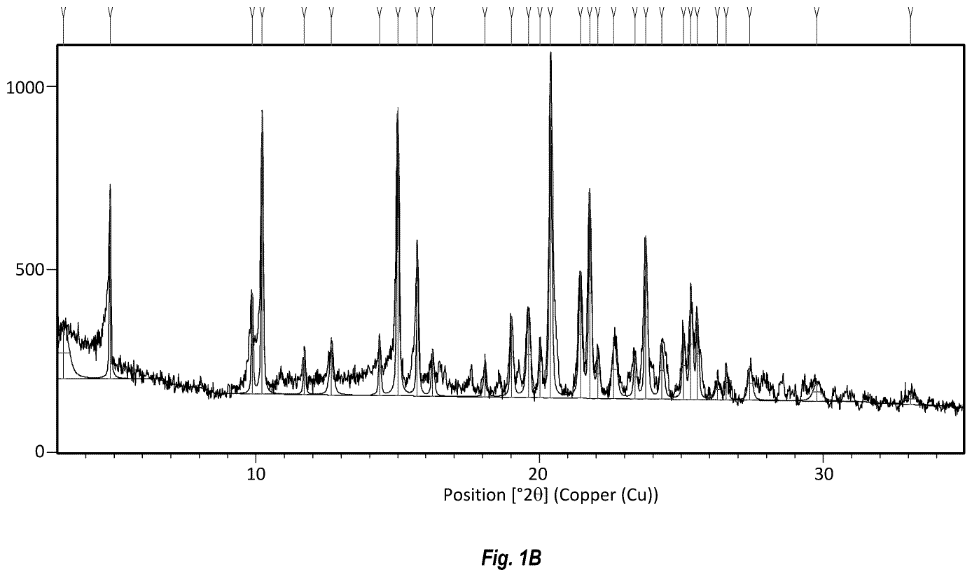

In some embodiments, the ENT1 inhibitor is a Compound 1 hydrogen sulfate or hydrate or solvate thereof, including crystalline Form 2 Compound 1 di(hydrogen sulfate) trihydrate, having an X-ray powder diffraction pattern comprising a peak at about 4.9° 2θ and one or more peaks chosen from peaks at about 10.2° 2θ and about 15.0° 2θ. In some embodiments, the Compound 1 hydrogen sulfate or hydrate or solvate, including crystalline Form 2 Compound 1 di(hydrogen sulfate) trihydrate, has an X-ray powder diffraction pattern substantially the same as that of FIG. 1A.

In some embodiments, the ENT1 inhibitor is crystalline Form 2 Compound 1 di(hydrogen sulfate) trihydrate. Crystalline Form 2 Compound 1 di(hydrogen sulfate) trihydrate can be prepared in a number of different ways. The XRPD diffraction patterns of FIGS. 1A and 1B are, as with the other XRPD patterns of crystalline Form 2 Compound 1 di(hydrogen sulfate) trihydrate disclosed herein, exemplary patterns of crystalline Form 2 Compound 1 di(hydrogen sulfate) trihydrate.

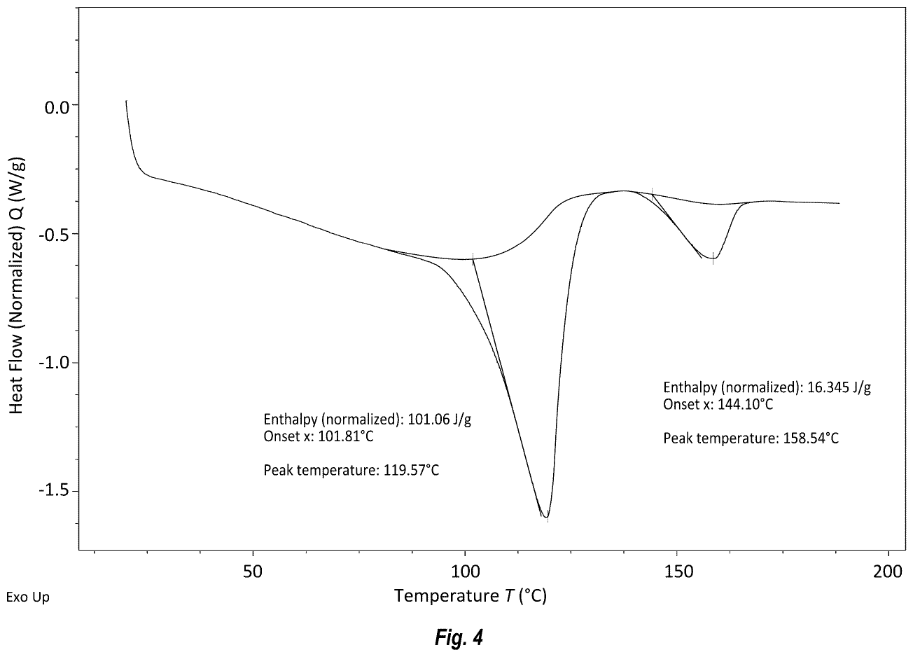

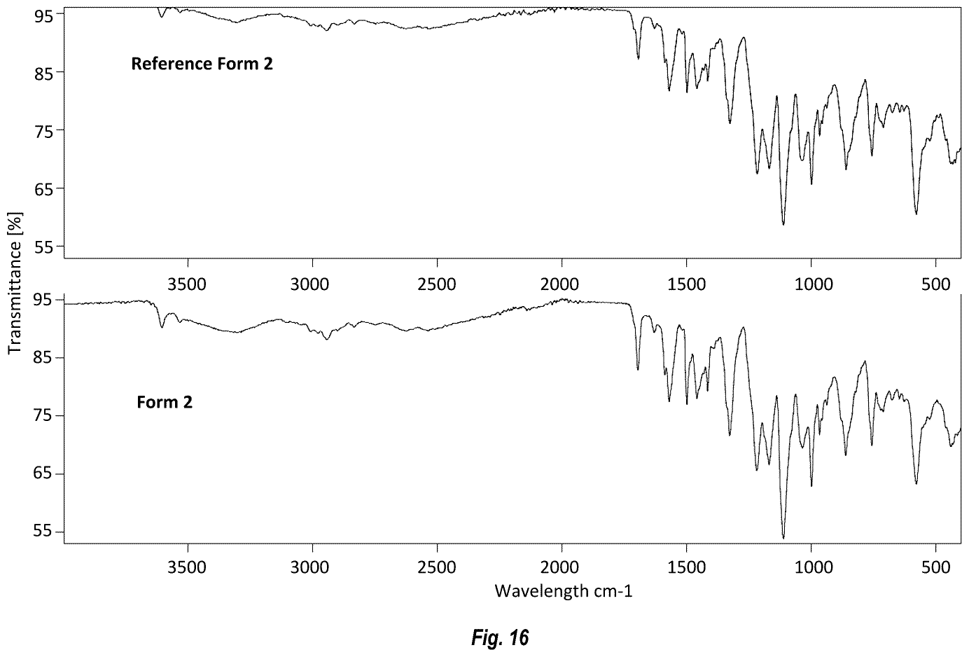

In some embodiments, the ENT1 inhibitor is a Compound 1 hydrogen sulfate or hydrate or solvate thereof, including crystalline Form 2 Compound 1 di(hydrogen sulfate) trihydrate, having an FT-IR spectrum substantially the same as that of FIG. 16. In some embodiments, the Compound 1 hydrogen sulfate or hydrate or solvate thereof, including crystalline Form 2 Compound 1 di(hydrogen sulfate) trihydrate, has a melting onset as measured by DSC in a sealed aluminum pan with a pierced lid of about 144° C. In some embodiments, the Compound 1 hydrogen sulfate or hydrate or solvate thereof, including crystalline Form 2 Compound 1 di(hydrogen sulfate) trihydrate, has a DSC thermogram substantially the same as that of FIG. 4.

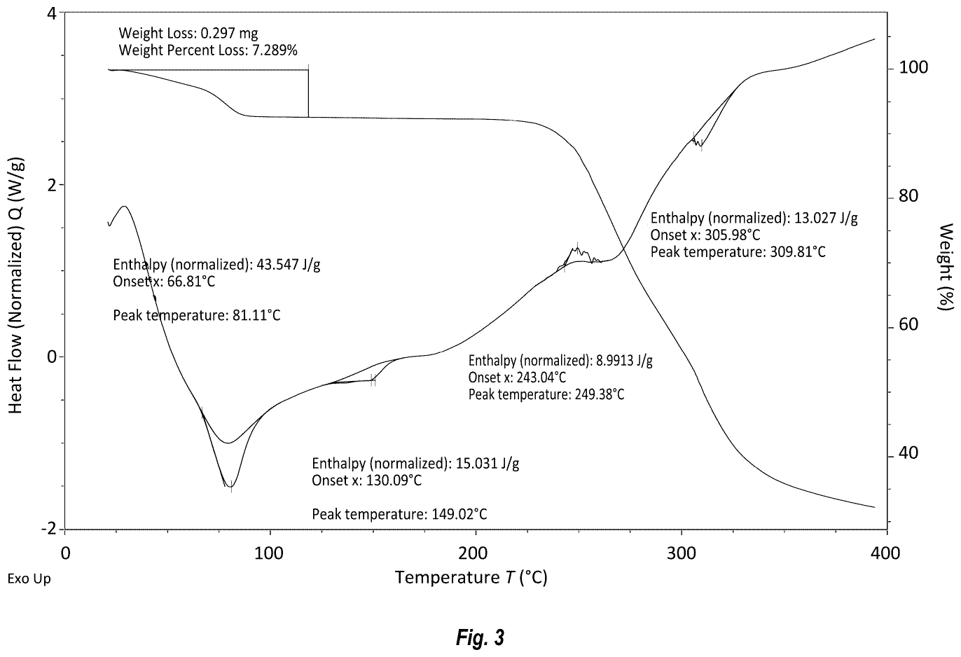

In some embodiments, the ENT1 inhibitor is a Compound 1 hydrogen sulfate or hydrate or solvate thereof, including crystalline Form 2 Compound 1 di(hydrogen sulfate) trihydrate, exhibiting a mass loss of between about 5% wt and about 9% wt upon heating from about 20° C. to about 130° C. In some embodiments, the Compound 1 hydrogen sulfate or hydrate or solvate thereof, including crystalline Form 2 Compound 1 di(hydrogen sulfate) trihydrate, exhibits a weight loss of about 6% wt upon heating from about 20° C. to about 130° C. In some embodiments, the Compound 1 hydrogen sulfate or hydrate or solvate thereof, including crystalline Form 2 Compound 1 di(hydrogen sulfate) trihydrate, exhibits a weight loss of about 7% wt upon heating from about 20° C. to about 130° C. In some embodiments, the Compound 1 hydrogen sulfate or hydrate or solvate thereof, including crystalline Form 2 Compound 1 di(hydrogen sulfate) trihydrate, has a TGA thermogram substantially the same as in FIG. 3.

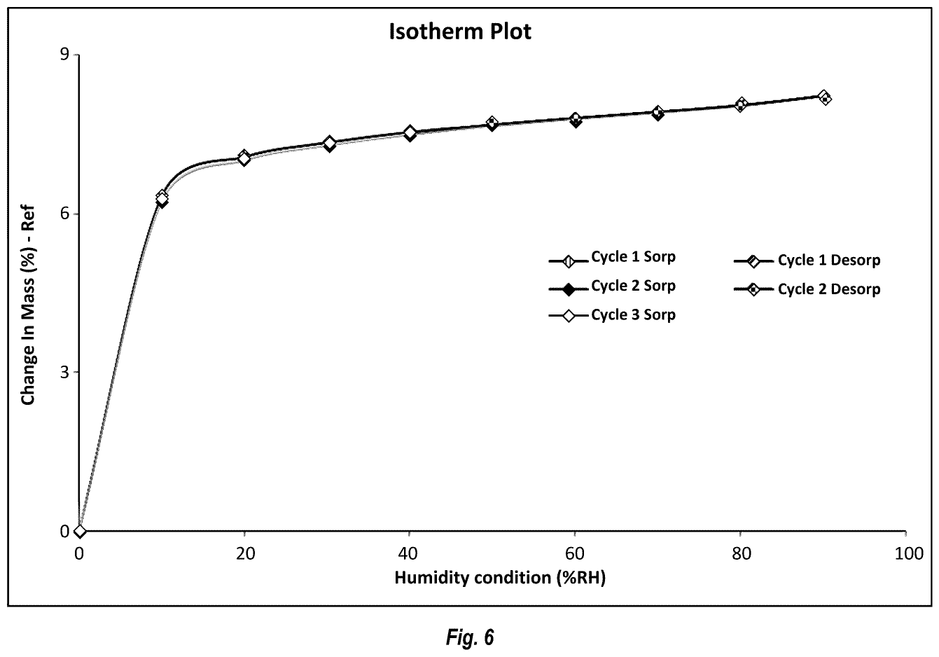

In some embodiments, the ENT1 inhibitor is a Compound 1 hydrogen sulfate or hydrate or solvate thereof, including crystalline Form 2 Compound 1 di(hydrogen sulfate) trihydrate, exhibiting a moisture uptake of between about 5% and about 7% between about 0% RH and about 10% RH. In some embodiments, the moisture uptake is between about 6% and about 6.5% between about 0% RH and about 10% RH. In some embodiments, the moisture uptake is about 6% between about 0% RH and about 10% RH. In some embodiments, the moisture uptake is about 7% between about 0% RH and about 10% RH. In some embodiments, the Compound 1 hydrogen sulfate or hydrate or solvate thereof, including crystalline Form 2 Compound 1 di(hydrogen sulfate) trihydrate, has an isotherm plot substantially the same as in FIG. 6.

In some embodiments, the ENT1 inhibitor is a Compound 1 hydrogen sulfate or hydrate or solvate thereof, including crystalline Form 2 Compound 1 di(hydrogen sulfate) trihydrate, having a water content of between about 4% and about 7%. In some embodiments, the water content is between about 6% and about 7%. In some embodiments, the water content is about 4%. In some embodiments, the water content is about 6%. In some embodiments, the water content is about 7%.

In some embodiments, the ENT1 inhibitor is a Compound 1 hydrogen sulfate or hydrate or solvate thereof, including crystalline Form 2 Compound 1 di(hydrogen sulfate) trihydrate, having a chemical purity of about 95%. In some embodiments, the chemical purity is about 970. In some embodiments, the chemical purity is 99%. In some embodiments, the chemical purity is greater than 99%.

In some embodiments, the ENT1 inhibitor is crystalline Form 2 Compound 1 di(hydrogen sulfate) trihydrate having an X-ray powder diffraction pattern comprising one or more peaks chosen from the peak list in Table 1 below (all peaks that can be or have been chosen from Table 1 are rounded to the nearest 0.1° 2θ). In some embodiments, the crystalline Form 2 Compound 1 di(hydrogen sulfate) trihydrate has an X-ray powder diffraction pattern comprising two or more peaks chosen from the peak list in Table 1 below (all peaks that can be or have been chosen from Table 1 are rounded to the nearest 0.1° 2θ). In some embodiments, the crystalline Form 2 Compound 1 di(hydrogen sulfate) trihydrate has an X-ray powder diffraction pattern comprising three or more peaks chosen from the peak list in Table 1 below (all peaks that can be or have been chosen from Table 1 are rounded to the nearest 0.1° 2θ).

TABLE 1
Peak list for crystalline Form 2 Compound
1 di(hydrogen sulfate) trihydrate

| | Pos. | d-spacing | Height | Rel. Int. |
|---|---|---|---|---|
| No. | [°2θ] | [Å] | [cts] | [%] |
| 1 | 3.2145 | 27.4858 | 144.12 | 16.34 |
| 2 | 4.8584 | 18.18883 | 503.65 | 57.11 |
| 3 | 9.8661 | 8.96524 | 282.03 | 31.98 |
| 4 | 10.2164 | 8.65865 | 784.06 | 88.9 |
| 5 | 11.6994 | 7.56419 | 119.98 | 13.6 |
| 6 | 12.6578 | 6.99355 | 140.57 | 15.94 |
| 7 | 14.3558 | 6.16994 | 159.37 | 18.07 |
| 8 | 15.0008 | 5.90607 | 803.41 | 91.1 |
| 9 | 15.6918 | 5.64753 | 424.18 | 48.1 |
| 10 | 16.2234 | 5.46363 | 127.56 | 14.46 |
| 11 | 18.0641 | 4.91085 | 105.13 | 11.92 |
| 12 | 19.0052 | 4.66973 | 225.18 | 25.53 |
| 13 | 19.5999 | 4.52935 | 237.59 | 26.94 |
| 14 | 20.019 | 4.43548 | 142.38 | 16.14 |
| 15 | 20.3701 | 4.35981 | 881.93 | 100 |
| 16 | 21.4304 | 4.14645 | 352.47 | 39.97 |
| 17 | 21.7663 | 4.08321 | 585.79 | 66.42 |
| 18 | 22.0553 | 4.03035 | 135.37 | 15.35 |
| 19 | 22.6211 | 3.93081 | 160.73 | 18.23 |
| 20 | 23.3558 | 3.8088 | 120.51 | 13.66 |
| 21 | 23.7449 | 3.74726 | 454.73 | 51.56 |
| 22 | 24.2988 | 3.66308 | 159.23 | 18.05 |
| 23 | 25.0687 | 3.55229 | 188.58 | 21.38 |
| 24 | 25.3332 | 3.51581 | 306.82 | 34.79 |
| 25 | 25.5499 | 3.48647 | 253.82 | 28.78 |
| 26 | 26.2676 | 3.39282 | 56.63 | 6.42 |
| 27 | 26.5834 | 3.35323 | 95.33 | 10.81 |
| 28 | 27.4053 | 3.2545 | 94.49 | 10.71 |
| 29 | 29.7707 | 3.00109 | 50.92 | 5.77 |
| 30 | 33.0781 | 2.70819 | 33 | 3.74 |

Below is the peak list and parameters used for a simulated XRPD diffractogram that was calculated for crystalline Form 2 Compound 1 di(hydrogen sulfate) trihydrate from the crystallographic analysis conducted at 295 K. Accordingly, in some embodiments, the ENT1 inhibitor is a Compound 1 hydrogen sulfate or a hydrate or solvate thereof, including crystalline Form 2 Compound 1 di(hydrogen sulfate) trihydrate, having an X-ray powder diffraction pattern comprising a peak at about 4.9° 2θ. In some embodiments, the Compound 1 hydrogen sulfate or hydrate or solvate thereof, including crystalline Form 2 Compound 1 di(hydrogen sulfate) trihydrate, has an X-ray powder diffraction pattern comprising peaks at about 4.9° 2θ and about 9.9° 2θ. In some embodiments, the Compound 1 hydrogen sulfate or hydrate or solvate thereof, including crystalline Form 2 Compound 1 di(hydrogen sulfate) trihydrate, has an X-ray powder diffraction pattern comprising peaks at about 4.9° 2θ and about 11.8° 2θ. In some embodiments, the Compound 1 hydrogen sulfate or hydrate or solvate thereof, including crystalline Form 2 Compound 1 di(hydrogen sulfate) trihydrate, has an X-ray powder diffraction pattern comprising peaks at about 4.9° 2θ and about 15.1° 2θ. In some embodiments, the Compound 1 hydrogen sulfate or hydrate or solvate thereof, including crystalline Form 2 Compound 1 di(hydrogen sulfate) trihydrate, has an X-ray powder diffraction pattern comprising peaks at about 4.9° 2θ, about 11.8° 2θ, and about 15.1° 2θ.

In some embodiments, the ENT1 inhibitor is a Compound 1 hydrogen sulfate or hydrate or solvate thereof, including crystalline Form 2 Compound 1 di(hydrogen sulfate) trihydrate, having an X-ray powder diffraction pattern comprising one or more peaks chosen from about 4.9° 2θ, about 9.9° 2θ, about 10.3° 2θ, about 11.8° 2θ, about 12.7° 2θ, about 14.4° 2θ, about 15.1° 2θ, about 15.7° 2θ, about 19.1° 2θ, and about 19.7° 2θ.

In some embodiments, the ENT1 inhibitor is a Compound 1 hydrogen sulfate or hydrate or solvate thereof, including crystalline Form 2 Compound 1 di(hydrogen sulfate) trihydrate, having an X-ray powder diffraction pattern comprising a peak at about 4.9° 2θ and one or more peaks chosen from peaks at about 10.3° 2θ and about 15.1° 2θ.

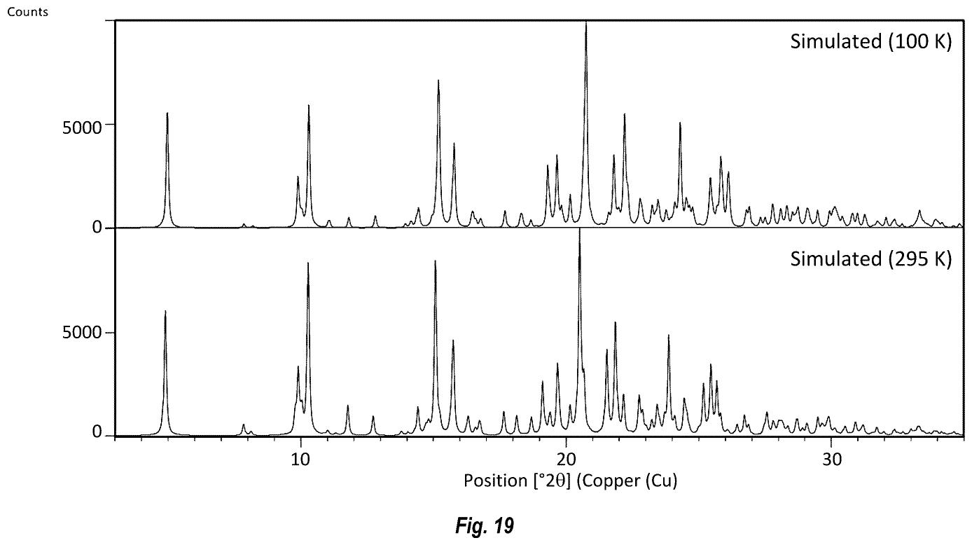

In some embodiments, the ENT1 inhibitor is a Compound 1 hydrogen sulfate or hydrate or solvate, including crystalline Form 2 Compound 1 di(hydrogen sulfate) trihydrate, having an X-ray powder diffraction pattern substantially the same as the bottom pattern of FIG. 19 (Simulated at 295 K).

In some embodiments, the ENT1 inhibitor is crystalline Form 2 Compound 1 di(hydrogen sulfate) trihydrate having an X-ray powder diffraction pattern comprising one or more peaks chosen from the peak list in Table 3 or 4 below (all peaks that can be or have been chosen from Table 3 or 4 are rounded to the nearest 0.1° 2θ). In some embodiments, the crystalline Form 2 Compound 1 di(hydrogen sulfate) trihydrate has an X-ray powder diffraction pattern comprising two or more peaks chosen from the peak list in Table 3 or 4 below (all peaks that can be or have been chosen from Table 3 or 4 are rounded to the nearest 0.1° 2θ). In some embodiments, the crystalline Form 2 Compound 1 di(hydrogen sulfate) trihydrate has an X-ray powder diffraction pattern comprising three or more peaks chosen from the peak list in Table 3 or 4 below (all peaks that can be or have been chosen from Table 3 or 4 are rounded to the nearest 0.1° 2θ).

TABLE 2
Crystallographic parameters and refinement indicators of Crystalline
Form 2 Compound 1 di(hydrogen sulfate) trihydrate (295 K)

| Empirical | C₃₃H₅₅N₃O₁₉S₂ | |
|---|---|---|
| formula | *the calculated formula differs from the | |
| | reported formula by two hydrogen atoms and one | |
| | oxygen atom that were not located | |
| Formula weight | 858.89 | g/mol |
| Temperature/K | 295 | |
| Crystal system | Triclinic | |
| Space group | P1 | |
| a/Å | 9.669 | (2) |
| b/Å | 12.439 | (3) |
| c/Å | 18.717 | (4) |
| α/° | 102.143 | (13) |
| β/° | 94.505 | (14) |
| γ/° | 110.746 | (12) |
| Volume/Å³ | 2029.0 | (8) |
| Z, Z′ | 2, 2 | |
| ρ_{calc} g/cm³ | 1.406 | |
| μ/mm⁻¹ | 1.896 | |
| F(000) | 910.0 | |
| Crystal size/mm³ | 0.26 × 0.18 × 0.1 | |
| Radiation | CuKα (λ = 1.54184) | |
| 2Θ range for | 4.898 to 133.718 | |
| data collection/° | | |
| Index ranges | −11 ≤ h ≤ 11, −14 ≤ k ≤ 14, −22 ≤ l ≤ 22 | |
| Reflections | 59429 | |
| collected | | |
| Independent | 13898 [Rᵢₙₜ = 0.0790, R_{sigma} = 0.0782] | |
| reflections | | |
| Data/restraints/ | 13898/9/1037 | |
| parameters | | |
| S | 1.141 | |
| Final R indexes | R₁ = 0.0835, wR₂ = 0.2430 | |
| [F² > 2σ (F²)] | | |
| Final R indexes | R₁ = 0.1304, wR₂ = 0.2767 | |
| [all data] | | |
| Δρmax, Δρmin/e | 0.75/−0.72 | |
| Å⁻³ | | |
| Flack parameter | 0.016 | (16) |

R₁ = (Σ |Fₒ| − |F_{c}|)/Σ |Fₒ|); wR₂ = {Σ [w(Fₒ² − F_{c}²)²]/Σ [w(Fₒ²)²]}^{1/2}; S = {Σ [w(Fₒ² − F_{c}²)²]/(n − p)}^{1/2.}

TABLE 3
Simulated XRPD 2θ diffractogram of Crystalline Form 2 Compound 1
di(hydrogen sulfate) trihydrate (295 K) (to 35° 2θ).

| | Pos. | d-spacing | Height | Rel. Int. |
|---|---|---|---|---|
| No. | [°2θ] | [Å] | [cts] | [%] |
| 1 | 4.8946 | 18.03960 | 6042.30 | 59.50 |
| 2 | 7.8547 | 11.24670 | 556.77 | 5.48 |
| 3 | 8.1269 | 10.87055 | 210.73 | 2.08 |
| 4 | 9.9142 | 8.91452 | 3372.58 | 33.21 |
| 5 | 10.2843 | 8.59445 | 8325.80 | 81.99 |
| 6 | 11.0213 | 8.02136 | 246.99 | 2.43 |
| 7 | 11.3260 | 7.80626 | 124.83 | 1.23 |
| 8 | 11.7769 | 7.50836 | 1449.36 | 14.27 |
| 9 | 12.7288 | 6.94891 | 946.25 | 9.32 |
| 10 | 13.7978 | 6.41286 | 208.45 | 2.05 |
| 11 | 14.0557 | 6.29576 | 175.68 | 1.73 |
| 12 | 14.4212 | 6.13703 | 1381.48 | 13.60 |
| 13 | 14.8161 | 5.97431 | 798.24 | 7.86 |
| 14 | 15.0866 | 5.86780 | 8503.98 | 83.74 |
| 15 | 15.7472 | 5.62311 | 4635.76 | 45.65 |
| 16 | 16.3067 | 5.43141 | 950.60 | 9.36 |
| 17 | 16.5804 | 5.34236 | 396.22 | 3.90 |
| 18 | 16.7518 | 5.28810 | 732.42 | 7.21 |
| 19 | 17.6648 | 5.01677 | 1162.56 | 11.45 |
| 20 | 18.1455 | 4.88495 | 977.39 | 9.63 |
| 21 | 18.7007 | 4.74114 | 895.86 | 8.82 |
| 22 | 19.1179 | 4.63861 | 2593.46 | 25.54 |
| 23 | 19.3951 | 4.57295 | 1135.31 | 11.18 |
| 24 | 19.6844 | 4.50638 | 3525.96 | 34.72 |
| 25 | 20.1541 | 4.40241 | 1482.37 | 14.60 |
| 26 | 20.5087 | 4.32709 | 10154.64 | 100.00 |
| 27 | 20.6883 | 4.28991 | 3063.49 | 30.17 |
| 28 | 21.5353 | 4.12307 | 4158.59 | 40.95 |
| 29 | 21.8691 | 4.06089 | 5594.20 | 55.09 |
| 30 | 22.1724 | 4.00602 | 2019.79 | 19.89 |
| 31 | 22.7572 | 3.90438 | 1932.14 | 19.03 |
| 32 | 23.2319 | 3.82567 | 774.16 | 7.62 |
| 33 | 23.4414 | 3.79193 | 1503.00 | 14.80 |
| 34 | 23.8808 | 3.72316 | 4836.63 | 47.63 |
| 35 | 24.0983 | 3.69005 | 976.79 | 9.62 |
| 36 | 24.4603 | 3.63624 | 1794.58 | 17.67 |
| 37 | 25.1790 | 3.53406 | 2515.82 | 24.78 |
| 38 | 25.4667 | 3.49479 | 3482.27 | 34.29 |
| 39 | 25.6807 | 3.46614 | 2622.15 | 25.82 |
| 40 | 25.8405 | 3.44507 | 1042.79 | 10.27 |
| 41 | 26.0935 | 3.41223 | 282.03 | 2.78 |
| 42 | 26.4560 | 3.36630 | 534.53 | 5.26 |
| 43 | 26.7225 | 3.33333 | 988.58 | 9.74 |
| 44 | 26.8890 | 3.31307 | 537.29 | 5.29 |
| 45 | 27.5852 | 3.23101 | 1124.78 | 11.08 |
| 46 | 27.8048 | 3.20599 | 721.02 | 7.10 |
| 47 | 28.0213 | 3.18171 | 737.19 | 7.26 |
| 48 | 28.1626 | 3.16606 | 700.32 | 6.90 |
| 49 | 28.3719 | 3.14318 | 450.70 | 4.44 |
| 50 | 28.7104 | 3.10689 | 799.44 | 7.87 |
| 51 | 28.9146 | 3.08541 | 359.05 | 3.54 |
| 52 | 29.0906 | 3.06714 | 581.41 | 5.73 |
| 53 | 29.4930 | 3.02620 | 888.83 | 8.75 |
| 54 | 29.6586 | 3.00969 | 595.93 | 5.87 |
| 55 | 29.9076 | 2.98519 | 897.31 | 8.84 |
| 56 | 30.1441 | 2.96231 | 360.12 | 3.55 |
| 57 | 30.5331 | 2.92544 | 448.44 | 4.42 |
| 58 | 30.9066 | 2.89094 | 637.94 | 6.28 |
| 59 | 31.2138 | 2.86318 | 500.03 | 4.92 |
| 60 | 31.7259 | 2.81813 | 403.18 | 3.97 |
| 61 | 31.9896 | 2.79550 | 180.41 | 1.78 |
| 62 | 32.3740 | 2.76318 | 292.01 | 2.88 |
| 63 | 32.7117 | 2.73542 | 173.43 | 1.71 |
| 64 | 32.9971 | 2.71240 | 310.93 | 3.06 |
| 65 | 33.3364 | 2.68557 | 441.08 | 4.34 |
| 66 | 33.9267 | 2.64018 | 201.06 | 1.98 |
| 67 | 34.1530 | 2.62320 | 165.81 | 1.63 |
| 68 | 34.6331 | 2.58793 | 175.24 | 1.73 |

TABLE 4
Simulated XRPD 2θ diffractogram of Crystalline Form 2 Compound
1 di(hydrogen sulfate) trihydrate (295 K) (20 most intense peaks).

| | Pos. | d-spacing | Height | Rel. Int. |
|---|---|---|---|---|
| No. | [°2θ] | [Å] | [cts] | [%] |
| 1 | 20.5087 | 4.32709 | 10154.64 | 100.00 |
| 2 | 15.0866 | 5.86780 | 8503.98 | 83.74 |
| 3 | 10.2843 | 8.59445 | 8325.80 | 81.99 |
| 4 | 4.8946 | 18.03960 | 6042.30 | 59.50 |
| 5 | 21.8691 | 4.06089 | 5594.20 | 55.09 |
| 6 | 23.8808 | 3.72316 | 4836.63 | 47.63 |
| 7 | 15.7472 | 5.62311 | 4635.76 | 45.65 |
| 8 | 21.5353 | 4.12307 | 4158.59 | 40.95 |
| 9 | 19.6844 | 4.50638 | 3525.96 | 34.72 |
| 10 | 25.4667 | 3.49479 | 3482.27 | 34.29 |
| 11 | 9.9142 | 8.91452 | 3372.58 | 33.21 |
| 12 | 20.6883 | 4.28991 | 3063.49 | 30.17 |
| 13 | 25.6807 | 3.46614 | 2622.15 | 25.82 |
| 14 | 19.1179 | 4.63861 | 2593.46 | 25.54 |
| 15 | 25.1790 | 3.53406 | 2515.82 | 24.78 |
| 16 | 22.1724 | 4.00602 | 2019.79 | 19.89 |
| 17 | 22.7572 | 3.90438 | 1932.14 | 19.03 |
| 18 | 24.4603 | 3.63624 | 1794.58 | 17.67 |
| 19 | 23.4414 | 3.79193 | 1503.00 | 14.80 |
| 20 | 20.1541 | 4.40241 | 1482.37 | 14.60 |

Below is the peak list and parameters used for a simulated XRPD diffractogram that was calculated for crystalline Form 2 Compound 1 di(hydrogen sulfate) trihydrate at 100 K. Accordingly, in some embodiments, the ENT1 inhibitor is crystalline Form 2 Compound 1 di(hydrogen sulfate) trihydrate having an X-ray powder diffraction pattern comprising one or more peaks chosen from the peak list in Table 6 or 7 below (all peaks that can be or have been chosen from Table 6 or 7 are rounded to the nearest 0.1° 2θ). In some embodiments, the crystalline Form 2 Compound 1 di(hydrogen sulfate) trihydrate has an X-ray powder diffraction pattern comprising two or more peaks chosen from the peak list in Table 6 or 7 below (all peaks that can be or have been chosen from Table 6 or 7 are rounded to the nearest 0.1° 2θ). In some embodiments, the crystalline Form 2 Compound 1 di(hydrogen sulfate) trihydrate has an X-ray powder diffraction pattern comprising three or more peaks chosen from the peak list in Table 6 or 7 below (all peaks that can be or have been chosen from Table 6 or 7 are rounded to the nearest 0.1° 2θ). In some embodiments, the Compound 1 hydrogen sulfate or hydrate or solvate, including crystalline Form 2 Compound 1 di(hydrogen sulfate) trihydrate, has an X-ray powder diffraction pattern substantially the same as the top pattern of FIG. 19 (Simulated at 100 K).

TABLE 5
Crystallographic parameters and refinement indicators of Crystalline
Form 2 Compound 1 di(hydrogen sulfate) trihydrate (100 K)

| Empirical formula | C₃₃H₅₇N₃O₂₀S₂ | |
|---|---|---|
| Formula weight | 879.93 | g/mol |
| Temperature/K | 100 | |
| Crystal system | Triclinic | |
| Space group | P1 | |
| a/Å | 9.6794 | (2) |
| b/Å | 12.3886 | (2) |
| c/Å | 18.4016 | (4) |
| α/° | 101.6708 | (8) |
| β/° | 94.7448 | (8) |
| γ/° | 110.7514 | (8) |
| Volume/Å³ | 1991.88 | (7) |
| Z, Z′ | 2, 2 | |
| ρ_{calc} g/cm³ | 1.467 | |
| μ/mm⁻¹ | 1.962 | |
| F(000) | 936.0 | |
| Crystal size/mm³ | 0.38 × 0.24 × 0.12 | |
| Radiation | CuKα (λ = 1.54178) | |
| 2Θ range for data | 4.974 to 144.29 | |
| collection/° | | |
| Index ranges | −10 ≤ h ≤ 11, −15 ≤ k ≤ 15, −22 ≤ l ≤ 2 | |
| Reflections collected | 97841 | |
| Independent reflections | 15198 [Rᵢₙₜ = 0.0299, R_{sigma} = 0.0202] | |
| Data/restraints/parameters | 15198/33/1139 | |
| S | 1.029 | |
| Final R indexes [F² > | R₁ = 0.0586, wR₂ = 0.1588 | |
| 2σ (F²)] | | |
| Final R indexes [all data] | R₁ = 0.0592, wR₂ = 0.1597 | |
| Δρmax, Δρmin/e Å⁻³ | 0.63/−1.17 | |
| Flack parameter | −0.008 | (5) |

R₁ = (Σ |Fₒ| − |F_{c}|)/Σ |Fₒ|); wR₂ = {Σ [w(Fₒ² − F_{c}²)²]/Σ [w(Fₒ²)²]}^{1/2}; S = {Σ [w(Fₒ² − F_{c}²)²]/(n − p)}^{1/2.}

TABLE 6
Simulated XRPD 2θ diffractogram of Crystalline Form 2 Compound 1
di(hydrogen sulfate) trihydrate (100 K) (to 35° 2θ).

| | Pos. | d-spacing | Height | Rel. Int. |
|---|---|---|---|---|
| No. | [°2θ] | [Å] | [cts] | [%] |
| 1 | 4.9708 | 17.76340 | 5692.39 | 57.05 |
| 2 | 7.8721 | 11.22177 | 192.87 | 1.93 |
| 3 | 8.2012 | 10.77221 | 118.89 | 1.19 |
| 4 | 9.9051 | 8.92269 | 2513.16 | 25.19 |
| 5 | 10.3093 | 8.57375 | 6074.58 | 60.88 |
| 6 | 11.0697 | 7.98639 | 381.20 | 3.82 |
| 7 | 11.8171 | 7.48292 | 506.02 | 5.07 |
| 8 | 12.8134 | 6.90323 | 597.90 | 5.99 |
| 9 | 13.9479 | 6.34418 | 211.75 | 2.12 |
| 10 | 14.1586 | 6.25023 | 346.39 | 3.47 |
| 11 | 14.4510 | 6.12442 | 967.78 | 9.70 |
| 12 | 15.2031 | 5.82311 | 7129.03 | 71.45 |
| 13 | 15.7809 | 5.61116 | 4079.65 | 40.89 |
| 14 | 16.4745 | 5.37646 | 794.87 | 7.97 |
| 15 | 16.7852 | 5.27763 | 453.42 | 4.54 |
| 16 | 17.7061 | 5.00516 | 847.97 | 8.50 |
| 17 | 18.3200 | 4.83881 | 709.84 | 7.11 |
| 18 | 18.6841 | 4.74533 | 393.29 | 3.94 |
| 19 | 19.3213 | 4.59023 | 3032.46 | 30.39 |
| 20 | 19.6644 | 4.51092 | 3526.60 | 35.34 |
| 21 | 19.8463 | 4.46998 | 1072.98 | 10.75 |
| 22 | 20.1614 | 4.40082 | 1610.14 | 16.14 |
| 23 | 20.7696 | 4.27331 | 9978.21 | 100.00 |
| 24 | 21.5995 | 4.11096 | 759.84 | 7.61 |
| 25 | 21.8102 | 4.07171 | 3594.54 | 36.02 |
| 26 | 22.2082 | 3.99964 | 5614.95 | 56.27 |
| 27 | 22.3481 | 3.97491 | 1892.70 | 18.97 |
| 28 | 22.7901 | 3.89882 | 1412.77 | 14.16 |
| 29 | 23.2451 | 3.82351 | 1120.21 | 11.23 |
| 30 | 23.4845 | 3.78508 | 1365.48 | 13.68 |
| 31 | 23.7730 | 3.73979 | 884.41 | 8.86 |
| 32 | 24.0898 | 3.69132 | 1251.47 | 12.54 |
| 33 | 24.3072 | 3.65880 | 5161.43 | 51.73 |
| 34 | 24.5353 | 3.62530 | 1465.70 | 14.69 |
| 35 | 24.7824 | 3.58971 | 996.51 | 9.99 |
| 36 | 25.4465 | 3.49751 | 2461.31 | 24.67 |
| 37 | 25.8398 | 3.44517 | 3425.07 | 34.33 |
| 38 | 26.1214 | 3.40866 | 2704.76 | 27.11 |
| 39 | 26.7881 | 3.32531 | 828.92 | 8.31 |
| 40 | 26.9212 | 3.30917 | 1036.14 | 10.38 |
| 41 | 27.3278 | 3.26086 | 516.98 | 5.18 |
| 42 | 27.5109 | 3.23957 | 527.00 | 5.28 |
| 43 | 27.7881 | 3.20787 | 1169.38 | 11.72 |
| 44 | 28.1012 | 3.17284 | 937.34 | 9.39 |
| 45 | 28.3241 | 3.14838 | 1081.95 | 10.84 |
| 46 | 28.5401 | 3.12505 | 773.11 | 7.75 |
| 47 | 28.7454 | 3.10318 | 1024.38 | 10.27 |
| 48 | 29.1078 | 3.06537 | 964.03 | 9.66 |
| 49 | 29.4874 | 3.02676 | 857.33 | 8.59 |
| 50 | 29.9255 | 2.98345 | 806.76 | 8.09 |
| 51 | 30.1457 | 2.96215 | 1038.18 | 10.40 |
| 52 | 30.4145 | 2.93659 | 577.92 | 5.79 |
| 53 | 30.7961 | 2.90106 | 714.10 | 7.16 |
| 54 | 30.9877 | 2.88356 | 724.52 | 7.26 |
| 55 | 31.2722 | 2.85797 | 663.29 | 6.65 |
| 56 | 31.7460 | 2.81639 | 339.03 | 3.40 |
| 57 | 32.0668 | 2.78894 | 523.95 | 5.25 |
| 58 | 32.3819 | 2.76252 | 429.15 | 4.30 |
| 59 | 32.6774 | 2.73821 | 209.40 | 2.10 |
| 60 | 33.3433 | 2.68503 | 859.27 | 8.61 |
| 61 | 33.9170 | 2.64091 | 408.11 | 4.09 |
| 62 | 34.1856 | 2.62078 | 272.79 | 2.73 |
| 63 | 34.6088 | 2.58969 | 110.44 | 1.11 |
| 64 | 34.8394 | 2.57308 | 228.50 | 2.29 |

TABLE 7
Simulated XRPD 2θ diffractogram of Crystalline Form 2 Compound
1 di(hydrogen sulfate) trihydrate (100 K) (20 most intense peaks).

| | Pos. | d-spacing | Height | Rel. Int. |
|---|---|---|---|---|
| No. | [°2θ] | [Å] | [cts] | [%] |
| 1 | 20.7696 | 4.27331 | 9978.21 | 100.00 |
| 2 | 15.2031 | 5.82311 | 7129.03 | 71.45 |
| 3 | 10.3093 | 8.57375 | 6074.58 | 60.88 |
| 4 | 4.9708 | 17.76340 | 5692.39 | 57.05 |
| 5 | 22.2082 | 3.99964 | 5614.95 | 56.27 |
| 6 | 24.3072 | 3.65880 | 5161.43 | 51.73 |
| 7 | 15.7809 | 5.61116 | 4079.65 | 40.89 |
| 8 | 21.8102 | 4.07171 | 3594.54 | 36.02 |
| 9 | 19.6644 | 4.51092 | 3526.60 | 35.34 |
| 10 | 25.8398 | 3.44517 | 3425.07 | 34.33 |
| 11 | 19.3213 | 4.59023 | 3032.46 | 30.39 |
| 12 | 26.1214 | 3.40866 | 2704.76 | 27.11 |
| 13 | 9.9051 | 8.92269 | 2513.16 | 25.19 |
| 14 | 25.4465 | 3.49751 | 2461.31 | 24.67 |
| 15 | 22.3481 | 3.97491 | 1892.70 | 18.97 |
| 16 | 20.1614 | 4.40082 | 1610.14 | 16.14 |
| 17 | 24.5353 | 3.62530 | 1465.70 | 14.69 |
| 18 | 22.7901 | 3.89882 | 1412.77 | 14.16 |
| 19 | 23.4845 | 3.78508 | 1365.48 | 13.68 |
| 20 | 24.0898 | 3.69132 | 1251.47 | 12.54 |

In some embodiments, the ENT1 inhibitor is crystalline Form 2 Compound 1 di(hydrogen sulfate) trihydrate. Crystalline Form 2 Compound 1 di(hydrogen sulfate) trihydrate contains one stereogenic center and is a single enantiomer with the R-configuration, as shown in Table 8. It contains three molecules of water, as shown in Table 8, and thus is a trihydrate.

TABLE 8

| | |
|---|---|
| Chemical Structure | 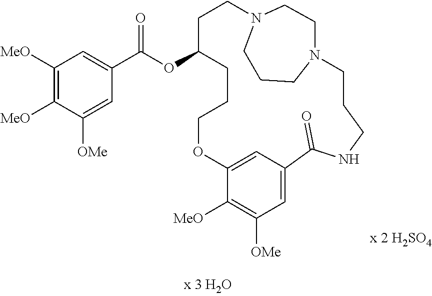 [SMILES: COc1cc(C(=O)O[C@@H]2CCCOc3cc(cc(OC)c3OC)C(=O)NCCCN3CCCN(CC2)CC3)cc(OC)c1OC] |
| Molecular formula | C₃₃H₄₇N₃O₉, 2 H₂SO₄, 3 H₂O (C₃₃H₅₁N₃0₁₇S₂, 3 H₂O) |
| Relative molecular | 629.75 (free base); 879.94 (salt: di(hydrogen sulfate)trihydrate) |
| mass (MW) | |

Crystalline Form 2 Compound 1 di(hydrogen sulfate) trihydrate has a single Compound 1 cation and at least one hydrogen sulfate anion and where the other sulfate moiety may be in the form of a second hydrogen sulfate anion or sulfuric acid (which would be neutral).

In some embodiments, the ENT1 inhibitor is selected from one of the compounds in Tables 9 or 10, or a pharmaceutically acceptable salt, hydrate, or solvate thereof.

TABLE 9

| Compound Structures | Chemical Name |
|---|---|
| 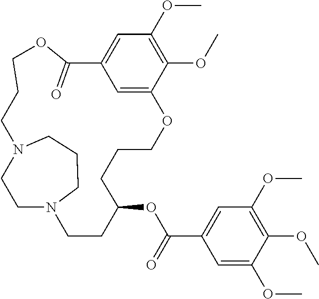 [SMILES: COc1cc(C(=O)O[C@@H]2CCCOc3cc(cc(OC)c3OC)C(=O)OCCCN3CCCN(CC2)CC3)cc(OC)c1OC] | (12R)-7⁴,7⁵-dimethoxy-6-oxo-5,8-dioxa-1(1,4)-diazepana- 7(1,3) benzenacyclotetradecaphane-12-yl 3,4,5- trimethoxybenzoate |
| 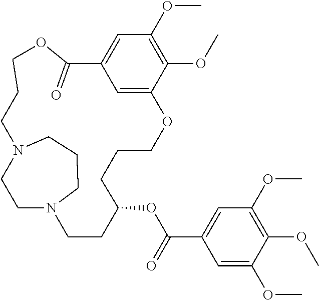 [SMILES: COc1cc(C(=O)O[C@H]2CCCOc3cc(cc(OC)c3OC)C(=O)OCCCN3CCCN(CC2)CC3)cc(OC)c1OC] | (12S)- 7⁴,7⁵-dimethoxy-6-oxo-5,8-dioxa-1(1,4)-diazepana- 7(1,3)-benzenacyclotetradecaphane-12-yl 3,4,5- trimethoxybenzoate |
| 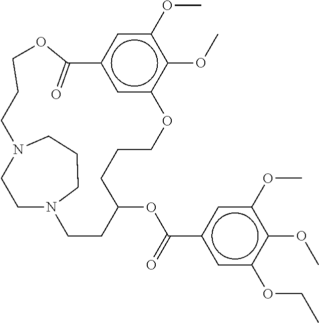 | 7⁴,7⁵-dimethoxy-6-oxo-5,8-dioxa-1(1,4)-diazepana-7(1,3)- benzenacyclotetradecaphane-12-yl 3-ethoxy-4,5- dimethoxybenzoate; |
| 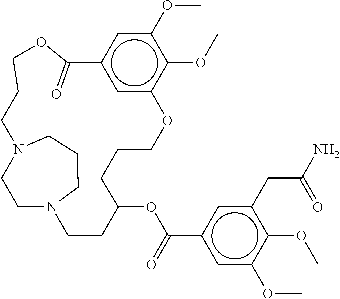 | 7⁴,7⁵-dimethoxy-6-oxo-5,8-dioxa-1(1,4)-diazepana-7(1,3)- benzenacyclotetradecaphane-12-yl 3-(2-amino-2-oxoethyl)- 4,5-dimethoxybenzoate; |
| 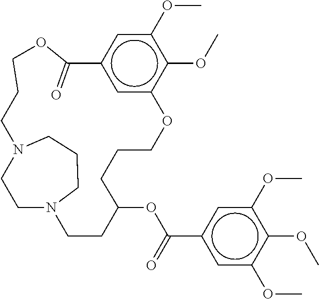 | 7⁴,7⁵-dimethoxy-6-oxo-5,8-dioxa-1(1,4)-diazepana-7(1,3)- benzenacyclotetradecaphane-12-yl 3,4,5 trimethoxybenzoate |
| 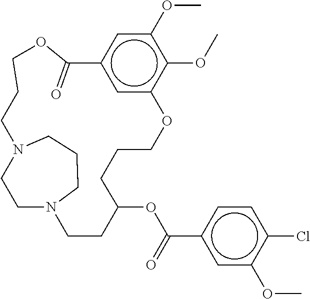 | 7⁴,7⁵-dimethoxy-6-oxo-5,8-dioxa-1(1,4)-diazepana-7(1,3)- benzenacyclotetradecaphane-12-yl 4-chloro-3- methoxybenzoate |
| 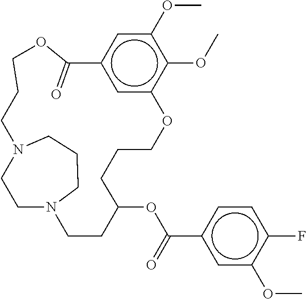 | 7⁴,7⁵-dimethoxy-6-oxo-5,8-dioxa-1(1,4)-diazepana-7(1,3)- benzenacyclotetradecaphane-12-yl 4-fluoro-3- methoxybenzoate |
| 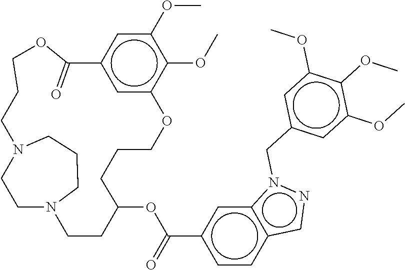 | 7⁴,7⁵-dimethoxy-6-oxo-5,8-dioxa-1(1,4)-diazepana-7(1,3)- benzenacyclotetradecaphane-12-yl 1-(3,4,5- trimethoxybenzyl)-1H-indazole-6-carboxylate |
| 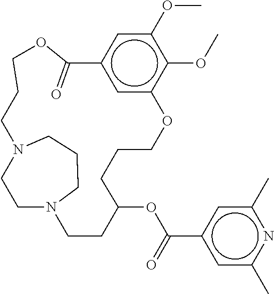 | 7⁴,7⁵-dimethoxy-6-oxo-5,8-dioxa-1(1,4)-diazepana-7(1,3)- benzenacyclotetradecaphane-12-yl 2,6-dimethylisonicotinate |
| 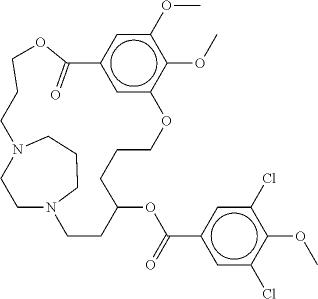 | 7⁴,7⁵-dimethoxy-6-oxo-5,8-dioxa-1(1,4)-diazepana-7(1,3)- benzenacyclotetradecaphane-12-yl 3,5-dichloro-4- methoxybenzoate |
| 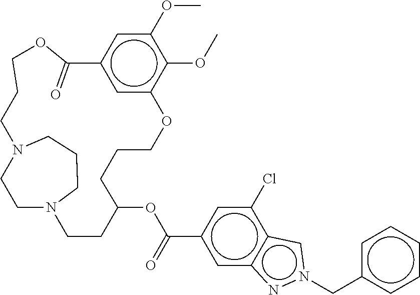 | 7⁴,7⁵-dimethoxy-6-oxo-5,8-dioxa-1(1,4)-diazepana-7(1,3)- benzenacyclotetradecaphane-12-yl 2-benzyl-4-chloro-2H- indazole-6-carboxylate |
| 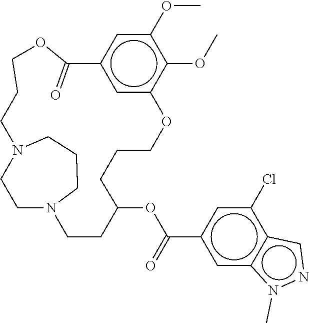 | 7⁴,7⁵-dimethoxy-6-oxo-5,8-dioxa-1(1,4)-diazepana-7(1,3)- benzenacyclotetradecaphane-12-yl 4-chloro-1-methyl-1H- indazole-6-carboxylate |
| 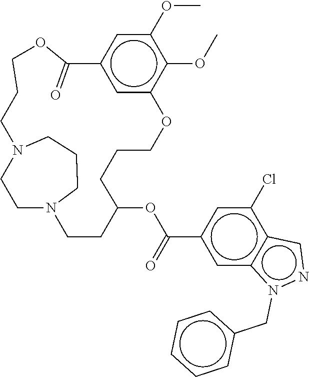 | 7⁴,7⁵-dimethoxy-6-oxo-5,8-dioxa-1(1,4)-diazepana-7(1,3)- benzenacyclotetradecaphane-12-yl 1-benzyl-4-chloro-1H- indazole-6-carboxylate |
| 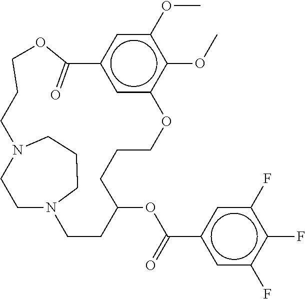 | 7⁴,7⁵-dimethoxy-6-oxo-5,8-dioxa-1(1,4)-diazepana-7(1,3)- benzenacyclotetradecaphane-12-yl 3,4,5-trifluorobenzoate |
| 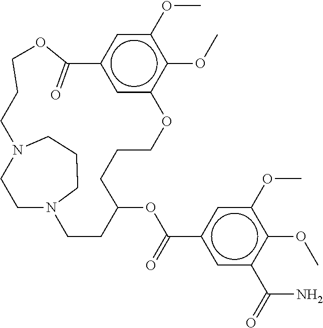 | 7⁴,7⁵-dimethoxy-6-oxo-5,8-dioxa-1(1,4)-diazepana-7(1,3)- benzenacyclotetradecaphane-12-yl 3-carbamoyl-4,5- dimethoxybenzoate |
| 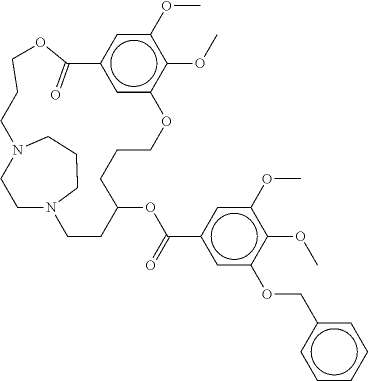 | 7⁴,7⁵-dimethoxy-6-oxo-5,8-dioxa-1(1,4)-diazepana-7(1,3)- benzenacyclotetradecaphane-12-yl 3-(benzyloxy)-4,5- dimethoxybenzoate |
| 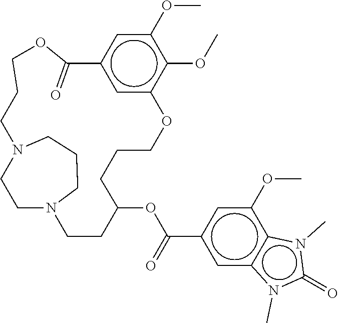 | 7⁴,7⁵-dimethoxy-6-oxo-5,8-dioxa-1(1,4)-diazepana-7(1,3)- benzenacyclotetradecaphane-12-yl 7-methoxy-1,3-dimethyl- 2-oxo-2,3-dihydro-1H-benzo[d]imidazole-5-carboxylate |
| 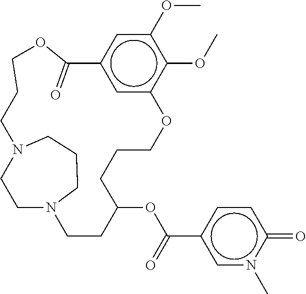 | 74, 75-dimethoxy-6-oxo-5,8-dioxa-1(1,4)-diazepana-7(1,3)- benzenacyclotetradecaphane-12-yl 1-methyl-6-oxo-1,6- dihydropyridine-3-carboxylate |
| 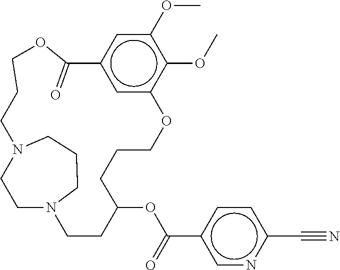 | 7⁴,7⁵-dimethoxy-6-oxo-5,8-dioxa-1(1,4)-diazepana-7(1,3)- benzenacyclotetradecaphane-12-yl 6-cyanonicotinate |
| 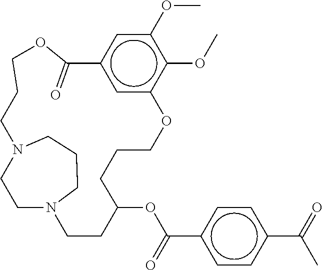 | 7⁴,7⁵-dimethoxy-6-oxo-5,8-dioxa-1(1,4)-diazepana-7(1,3)- benzenacyclotetradecaphane-12-yl 4-acetylbenzoate |
| 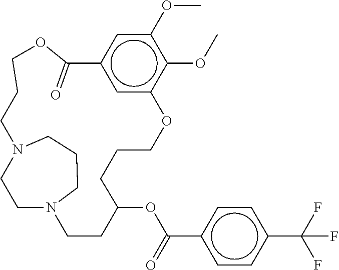 | 7⁴,7⁵-dimethoxy-6-oxo-5,8-dioxa-1(1,4)-diazepana-7(1,3)- benzenacyclotetradecaphane-12-yl 4- (trifluoromethyl)benzoate |
| 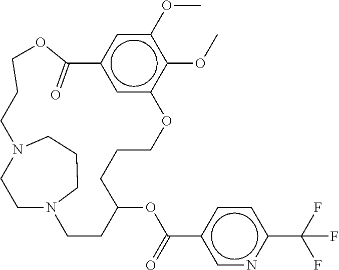 | 7⁴,7⁵-dimethoxy-6-oxo-5,8-dioxa-1(1,4)-diazepana-7(1,3)- benzenacyclotetradecaphane-12-yl 6- (trifluoromethyl)nicotinate |
| 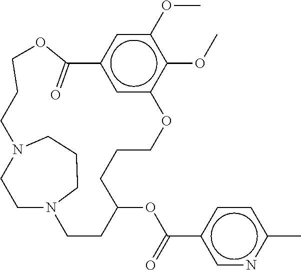 | 7⁴,7⁵-dimethoxy-6-oxo-5,8-dioxa-1(1,4)-diazepana-7(1,3)- benzenacyclotetradecaphane-12-yl 6-methylnicotinate |
| 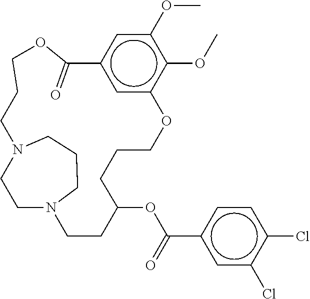 | 7⁴,7⁵-dimethoxy-6-oxo-5,8-dioxa-1(1,4)-diazepana-7(1,3)- benzenacyclotetradecaphane-12-yl 3,4-dichlorobenzoate |
| 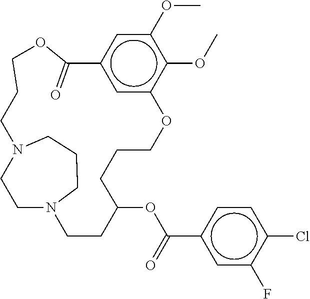 | 7⁴,7⁵-dimethoxy-6-oxo-5,8-dioxa-1(1,4)-diazepana-7(1,3)- benzenacyclotetradecaphane-12-yl 4-chloro-3- fluorobenzoate |
| 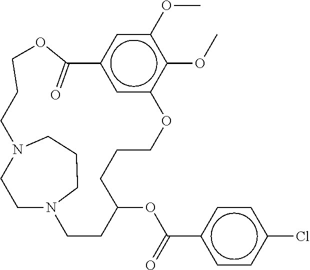 | 7⁴,7⁵-dimethoxy-6-oxo-5,8-dioxa-1(1,4)-diazepana-7(1,3)- benzenacyclotetradecaphane-12-yl 4-chlorobenzoate |
| 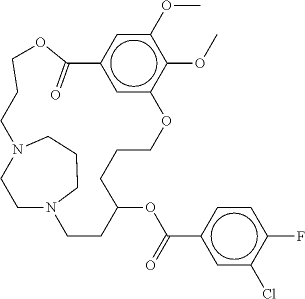 | 7⁴,7⁵-dimethoxy-6-oxo-5,8-dioxa-1(1,4)-diazepana-7(1,3)- benzenacyclotetradecaphane-12-yl 3-chloro-4- fluorobenzoate |
| 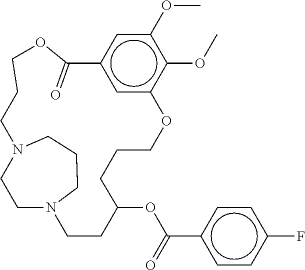 | 7⁴,7⁵-dimethoxy-6-oxo-5,8-dioxa-1(1,4)-diazepana-7(1,3)- benzenacyclotetradecaphane-12-yl 4-fluorobenzoate |
| 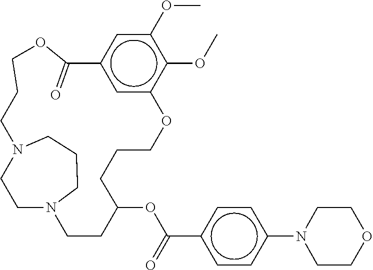 | 7⁴,7⁵-dimethoxy-6-oxo-5,8-dioxa-1(1,4)-diazepana-7(1,3)- benzenacyclotetradecaphane-12-yl 4-morpholinobenzoate |
| 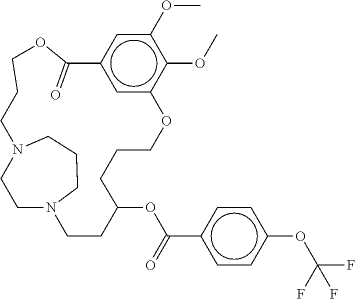 | 7⁴,7⁵-dimethoxy-6-oxo-5,8-dioxa-1(1,4)-diazepana-7(1,3)- benzenacyclotetradecaphane-12-yl 4- (trifluoromethoxy)benzoate |
| 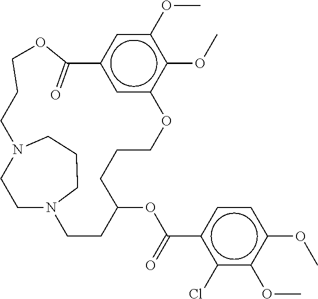 | 7⁴,7⁵-dimethoxy-6-oxo-5,8-dioxa-1(1,4)-diazepana-7(1,3)- benzenacyclotetradecaphane-12-yl 2-chloro-3,4- dimethoxybenzoate |
| 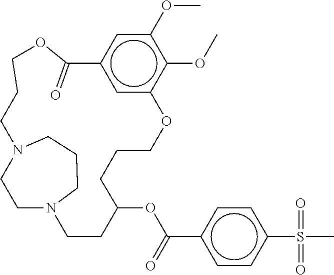 | 7⁴,7⁵-dimethoxy-6-oxo-5,8-dioxa-1(1,4)-diazepana-7(1,3)- benzenacyclotetradecaphane-12-yl 4- (methylsulfonyl)benzoate |
| 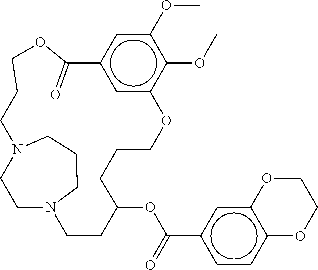 | 7⁴,7⁵-dimethoxy-6-oxo-5,8-dioxa-1(1,4)-diazepana-7(1,3)- benzenacyclotetradecaphane-12-yl 2,3- dihydrobenzo[b][1,4]dioxine-6-carboxylate |
| 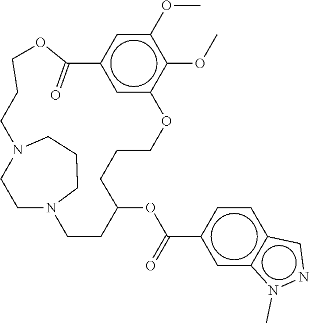 | 7⁴,7⁵-dimethoxy-6-oxo-5,8-dioxa-1(1,4)-diazepana-7(1,3)- benzenacyclotetradecaphane-12-yl 1-methyl-1H-indazole-6- carboxylate |
| 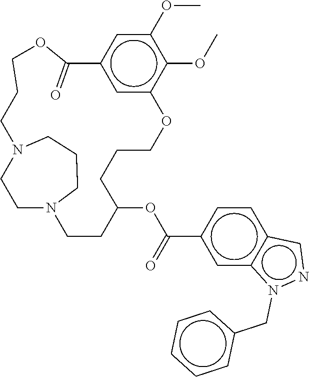 | 7⁴,7⁵-dimethoxy-6-oxo-5,8-dioxa-1(1,4)-diazepana-7(1,3)- benzenacyclotetradecaphane-12-yl 1-benzyl-1H-indazole-6- carboxylate |
| 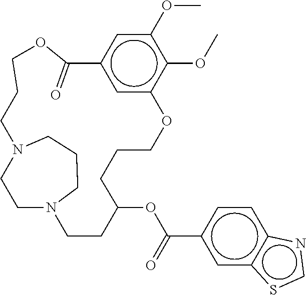 | 7⁴,7⁵-dimethoxy-6-oxo-5,8-dioxa-1(1,4)-diazepana-7(1,3)- benzenacyclotetradecaphane-12-yl benzo[d]thiazole-6- carboxylate |
| 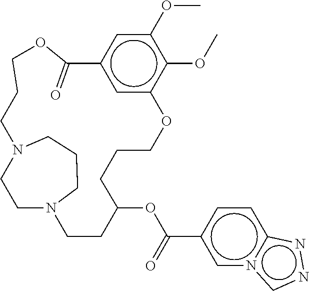 | 7⁴,7⁵-dimethoxy-6-oxo-5,8-dioxa-1(1,4)-diazepana-7(1,3)- benzenacyclotetradecaphane-12-yl [1,2,4]triazolo[4,3- alpyridine-6-carboxylate |
| 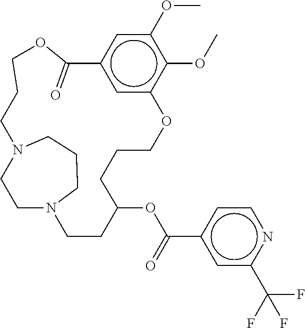 | 7⁴,7⁵-dimethoxy-6-oxo-5,8-dioxa-1(1,4)-diazepana-7(1,3)- benzenacyclotetradecaphane-12-yl 2- (trifluoromethyl)isonicotinate |
| 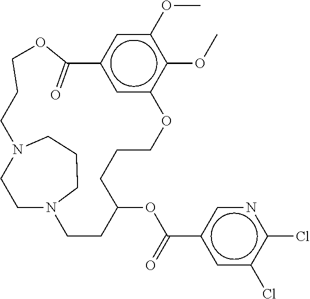 | 7⁴,7⁵-dimethoxy-6-oxo-5,8-dioxa-1(1,4)-diazepana-7(1,3)- benzenacyclotetradecaphane-12-yl 5,6-dichloronicotinate |
| 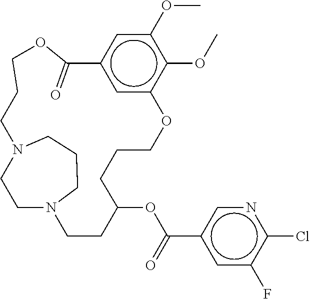 | 7⁴,7⁵-dimethoxy-6-oxo-5,8-dioxa-1(1,4)-diazepana-7(1,3)- benzenacyclotetradecaphane-12-yl 6-chloro-5- fluoronicotinate |
| 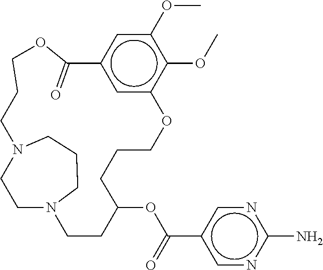 | 7⁴,7⁵-dimethoxy-6-oxo-5,8-dioxa-1(1,4)-diazepana-7(1,3)- benzenacyclotetradecaphane-12-yl 2-aminopyrimidine-5- carboxylate |
| 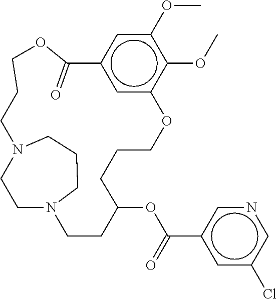 | 7⁴,7⁵-dimethoxy-6-oxo-5,8-dioxa-1(1,4)-diazepana-7(1,3)- benzenacyclotetradecaphane-12-yl 5-chloronicotinate |
| 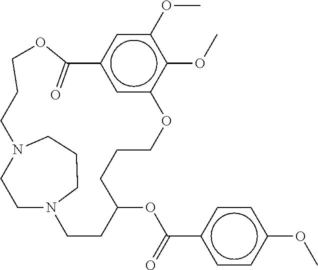 | 7⁴,7⁵-dimethoxy-6-oxo-5,8-dioxa-1(1,4)-diazepana-7(1,3)- benzenacyclotetradecaphane-12-yl 4-methoxybenzoate |
| 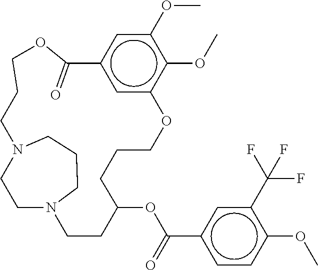 | 74, 75-dimethoxy-6-oxo-5,8-dioxa-1(1,4)-diazepana-7(1,3)- benzenacyclotetradecaphane-12-yl 4-methoxy-3- (trifluoromethyl)benzoate |
| 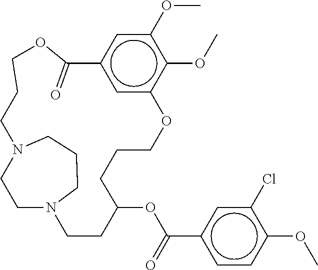 | 7⁴,7⁵-dimethoxy-6-oxo-5,8-dioxa-1(1,4)-diazepana-7(1,3)- benzenacyclotetradecaphane-12-yl 3-chloro-4- methoxybenzoate |
| 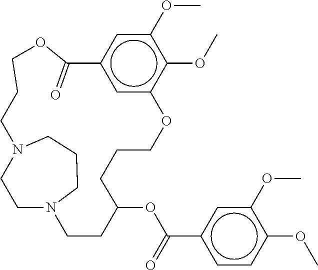 | 7⁴,7⁵-dimethoxy-6-oxo-5,8-dioxa-1(1,4)-diazepana-7(1,3)- benzenacyclotetradecaphane-12-yl 3,4-dimethoxybenzoate |
| 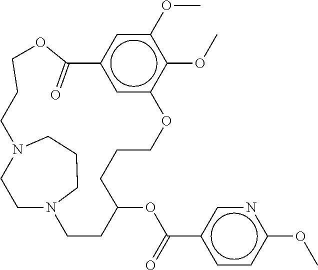 | 7⁴,7⁵-dimethoxy-6-oxo-5,8-dioxa-1(1,4)-diazepana-7(1,3)- benzenacyclotetradecaphane-12-yl 6-methoxynicotinate |
| 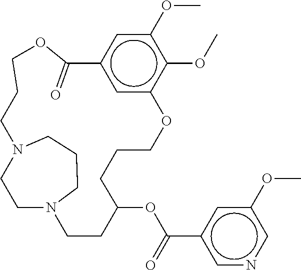 | 7⁴,7⁵-dimethoxy-6-oxo-5,8-dioxa-1(1,4)-diazepana-7(1,3)- benzenacyclotetradecaphane-12-yl 5-methoxynicotinate |
| 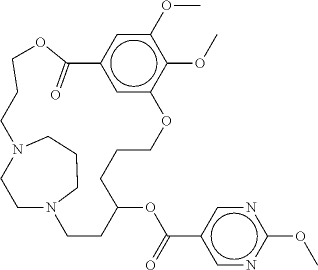 | 7⁴,7⁵-dimethoxy-6-oxo-5,8-dioxa-1(1,4)-diazepana-7(1,3)- benzenacyclotetradecaphane-12-yl 2-methoxypyrimidine-5- carboxylate |
| 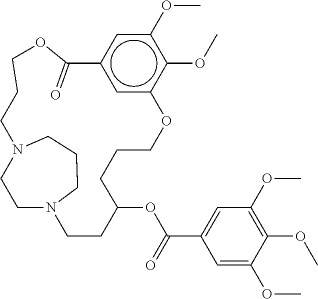 | 16-fluoro-7⁴,7⁵-dimethoxy-6-oxo-5,8-dioxa-1(1,4)- diazepana-7(1,3)-benzenacyclotetradecaphane-12-yl 3,4,5- trimethoxybenzoate |
| 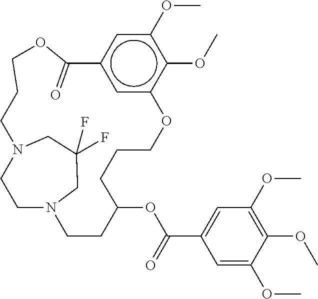 | 16,16-difluoro-7⁴,7⁵-dimethoxy-6-oxo-5,8-dioxa-1(1,4)- diazepana-7(1,3)-benzenacyclotetradecaphane-12-yl 3,4,5- trimethoxybenzoate |
| 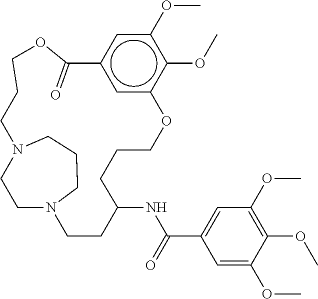 | N-(7⁴,7⁵-dimethoxy-6-oxo-5,8-dioxa-1(1,4)-diazepana- 7(1,3)-benzenacyclotetradecaphane-12-yl)-3,4,5- trimethoxybenzamide |
| 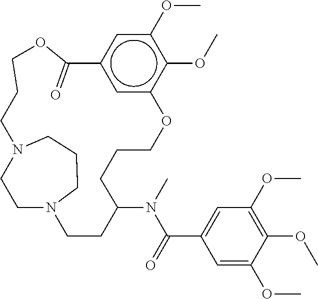 | N-(7⁴,7⁵-dimethoxy-6-oxo-5,8-dioxa-1(1,4)-diazepana- 7(1,3)-benzenacyclotetradecaphane-12-yl)-3,4,5-trimethoxy- N-methylbenzamide |
| 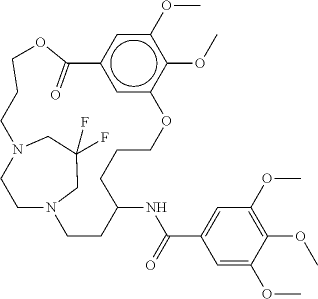 | N-(16,16-difluoro-7⁴,7⁵-dimethoxy-6-oxo-5,8-dioxa-1(1,4)- diazepana-7(1,3)-benzenacyclotetradecaphane-12-yl)-3,4,5- trimethoxybenzamide |
| 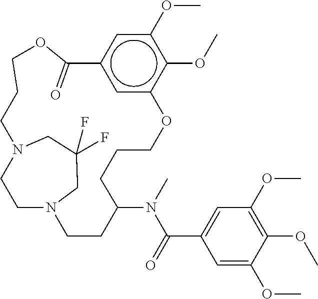 | N-(16,16-difluoro-7⁴,7⁵-dimethoxy-6-oxo-5,8-dioxa-1(1,4)- diazepana-7(1,3)-benzenacyclotetradecaphane-12-yl)-3,4,5- trimethoxy-N-methylbenzamide |
| 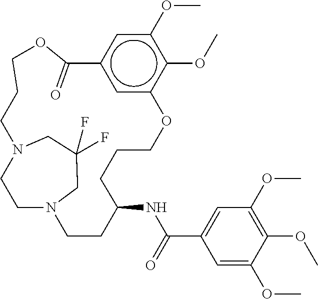 | N-((12R)-16,16-difluoro-7⁴,7⁵-dimethoxy-6-oxo-5,8-dioxa- 1(1,4)-diazepana-7(1,3) benzenacyclotetradecaphane-12-yl)- 3,4,5-trimethoxybenzamide |
| 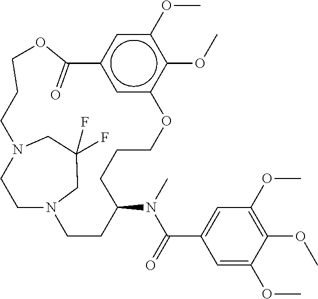 | N-((12R)-16,16-difluoro-7⁴,7⁵-dimethoxy-6-oxo-5,8-dioxa- 1(1,4)-diazepana-7(1,3) benzenacyclotetradecaphane-12-yl)- 3,4,5-trimethoxy-N-methylbenzamide |
| 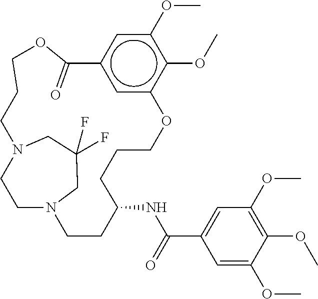 | N-((12S)-16,16-difluoro-7⁴,7⁵-dimethoxy-6-oxo-5,8-dioxa- 1(1,4)-diazepana-7(1,3)-benzenacyclotetradecaphane-12-yl)- 3,4,5-trimethoxybenzamide |
| 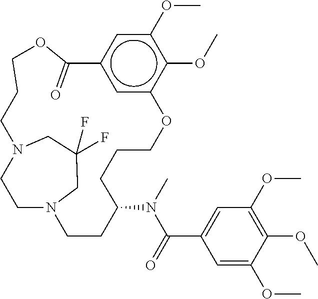 | N-((12S)-16,16-difluoro-7⁴,7⁵-dimethoxy-6-oxo-5,8-dioxa- 1(1,4)-diazepana-7(1,3)-benzenacyclotetradecaphane-12-yl)- 3,4,5-trimethoxy-N-methylbenzamide |

TABLE 10

| STRUCTURE | Name |
|---|---|
| 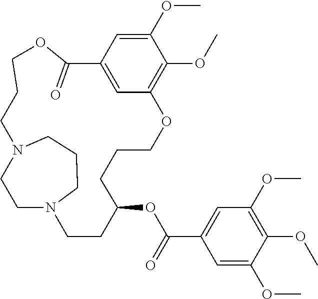 [SMILES: COc1cc(C(=O)O[C@@H]2CCCOc3cc(cc(OC)c3OC)C(=O)OCCCN3CCCN(CC2)CC3)cc(OC)c1OC] | (12R)-7⁴,7⁵-dimethoxy-6-oxo-5,8-dioxa- 1(1,4)-diazepana-7(1,3)- benzenacyclotetradecaphane-12-yl 3,4,5- trimethoxybenzoate |
| 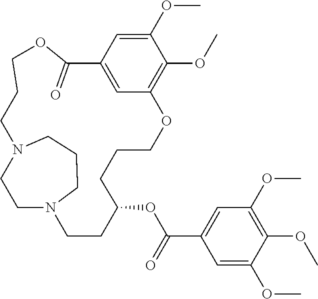 [SMILES: COc1cc(C(=O)O[C@H]2CCCOc3cc(cc(OC)c3OC)C(=O)OCCCN3CCCN(CC2)CC3)cc(OC)c1OC] | (12S)-7⁴,7⁵-dimethoxy-6-oxo-5,8-dioxa- 1(1,4)-diazepana-7(1,3)- benzenacyclotetradecaphane-12-yl 3,4,5- trimethoxybenzoate |
| 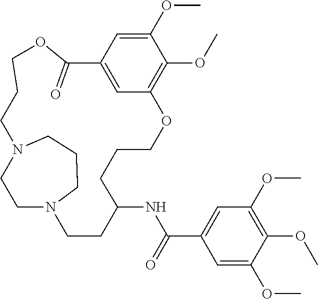 [SMILES: COc1cc(C(=O)NC2CCCOc3cc(cc(OC)c3OC)C(=O)OCCCN3CCCN(CC2)CC3)cc(OC)c1OC] | N-(7⁴,7⁵-dimethoxy-6-oxo-5,8-dioxa- 1(1,4)-diazepana-7(1,3)- benzenacyclotetradecaphane-12-yl)- 3,4,5-trimethoxybenzamide |
| 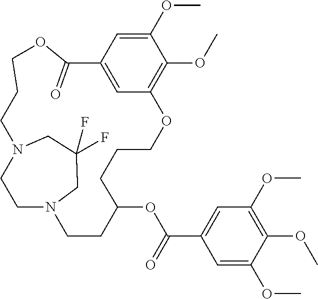 [SMILES: COc1cc(C(=O)OC2CCCOc3cc(cc(OC)c3OC)C(=O)OCCCN3CCN(CC2)CC(F)(F)C3)cc(OC)c1OC] | 16,16-difluoro-7⁴,7⁵-dimethoxy-6-oxo- 5,8-dioxa-1(1,4)-diazepana-7(1,3)- benzenacyclotetradecaphane-12-yl 3,4,5- trimethoxybenzoate |
| 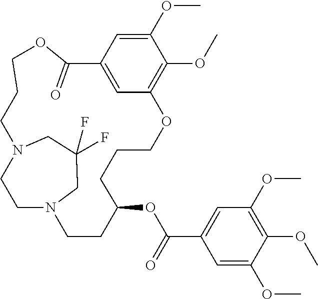 [SMILES: COc1cc(C(=O)O[C@@H]2CCCOc3cc(cc(OC)c3OC)C(=O)OCCCN3CCN(CC2)CC(F)(F)C3)cc(OC)c1OC] | (12R)-16,16-difluoro-7⁴,7⁵-dimethoxy-6- oxo-5,8-dioxa-1(1,4)-diazepana-7(1,3)- benzenacyclotetradecaphane-12-yl 3,4,5- trimethoxybenzoate |
| 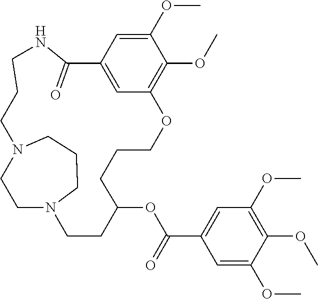 [SMILES: COc1cc(C(=O)OC2CCCOc3cc(cc(OC)c3OC)C(=O)NCCCN3CCCN(CC2)CC3)cc(OC)c1OC] | 7⁴,7⁵-dimethoxy-6-oxo-8-oxa-5-aza- 1(1,4)-diazepana-7(1,3)- benzenacyclotetradecaphane-12-yl 3,4,5- trimethoxybenzoate |
| 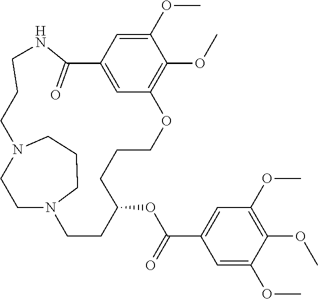 [SMILES: COc1cc(C(=O)O[C@H]2CCCOc3cc(cc(OC)c3OC)C(=O)NCCCN3CCCN(CC2)CC3)cc(OC)c1OC] | (12S)-7⁴,7⁵-dimethoxy-6-oxo-8-oxa-5- aza-1(1,4)-diazepana-7(1,3)- benzenacyclotetradecaphane-12-yl 3,4,5- trimethoxybenzoate |
| 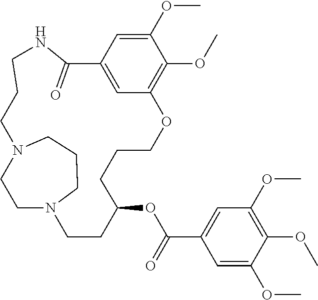 [SMILES: COc1cc(C(=O)O[C@@H]2CCCOc3cc(cc(OC)c3OC)C(=O)NCCCN3CCCN(CC2)CC3)cc(OC)c1OC] | (12R)-7⁴,7⁵-dimethoxy-6-oxo-8-oxa-5- aza-1(1,4)-diazepana-7(1,3)- benzenacyclotetradecaphane-12-yl 3,4,5- trimethoxybenzoate |
| 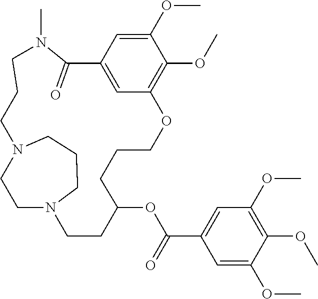 [SMILES: COc1cc(C(=O)OC2CCCOc3cc(cc(OC)c3OC)C(=O)N(C)CCCN3CCCN(CC2)CC3)cc(OC)c1OC] | 7⁴,7⁵-dimethoxy-5-methyl-6-oxo-8-oxa- 5-aza-1(1,4)-diazepana-7(1,3)- benzenacyclotetradecaphane-12-yl 3,4,5- trimethoxybenzoate |
| 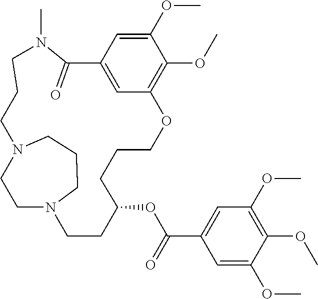 [SMILES: COc1cc(C(=O)O[C@H]2CCCOc3cc(cc(OC)c3OC)C(=O)N(C)CCCN3CCCN(CC2)CC3)cc(OC)c1OC] | (12S)-7⁴,7⁵-dimethoxy-5-methyl-6-oxo- 8-oxa-5-aza-1(1,4)-diazepana-7(1,3)- benzenacyclotetradecaphane-12-yl 3,4,5- trimethoxybenzoate |
| 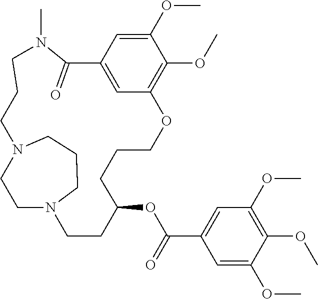 [SMILES: COc1cc(C(=O)O[C@@H]2CCCOc3cc(cc(OC)c3OC)C(=O)N(C)CCCN3CCCN(CC2)CC3)cc(OC)c1OC] | (12R)-7⁴,7⁵-dimethoxy-5-methyl-6-oxo- 8-oxa-5-aza-1(1,4)-diazepana-7(1,3)- benzenacyclotetradecaphane-12-yl 3,4,5- trimethoxybenzoate |
| 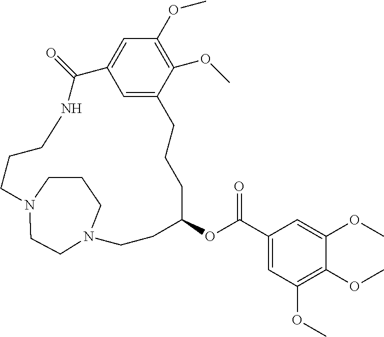 [SMILES: COc1cc2cc(c1OC)CCC[C@@H](OC(=O)c1cc(OC)c(OC)c(OC)c1)CCN1CCCN(CCCNC2=O)CC1] | (11R)-7⁴,7⁵-dimethoxy-6-oxo-5-aza- 1(1,4)-diazepana-7(1,3)- benzenacyclotridecaphane-11-yl 3,4,5- trimethoxybenzoate |
| 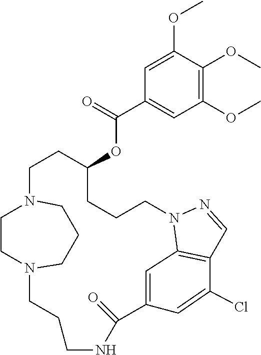 [SMILES: COc1cc(C(=O)O[C@H]2CCCn3ncc4c(Cl)cc(cc43)C(=O)NCCCN3CCCN(CC2)CC3)cc(OC)c1OC] | (10S)-14-chloro-2-oxo-11H-3-aza- 1(6,1)-indazola-7(1,4)- diazepanacyclotridecaphane-10-yl 3,4,5- trimethoxybenzoate |
| 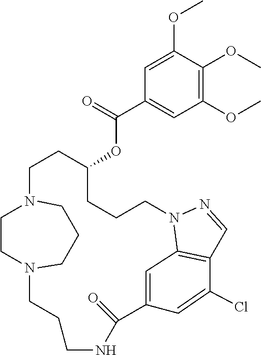 [SMILES: COc1cc(C(=O)O[C@@H]2CCCn3ncc4c(Cl)cc(cc43)C(=O)NCCCN3CCCN(CC2)CC3)cc(OC)c1OC] | (10R)-14-chloro-2-oxo-11H-3-aza- 1(6,1)-indazola-7(1,4)- diazepanacyclotridecaphane-10-yl 3,4,5- trimethoxybenzoate |
| 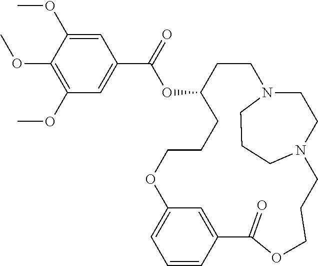 [SMILES: COc1cc(C(=O)O[C@H]2CCCOc3cccc(c3)C(=O)OCCCN3CCCN(CC2)CC3)cc(OC)c1OC] | (12S)-6-oxo-5,8-dioxa-1(1,4)-diazepana- 7(1,3)-benzenacyclotetradecaphane-12-yl 3,4,5-trimethoxybenzoate |
| 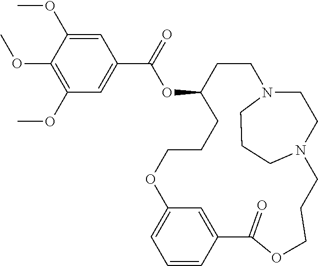 [SMILES: COc1cc(C(=O)O[C@@H]2CCCOc3cccc(c3)C(=O)OCCCN3CCCN(CC2)CC3)cc(OC)c1OC] | (12R)-6-oxo-5,8-dioxa-1(1,4)-diazepana- 7(1,3)-benzenacyclotetradecaphane-12-yl 3,4,5-trimethoxybenzoate |
| 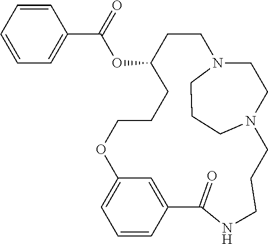 [SMILES: O=C1NCCCN2CCCN(CC[C@@H](OC(=O)c3ccccc3)CCCOc3cccc1c3)CC2] | (12S)-6-oxo-8-oxa-5-aza-1(1,4)- diazepana-7(1,3)- benzenacyclotetradecaphane-12-yl benzoate |
| 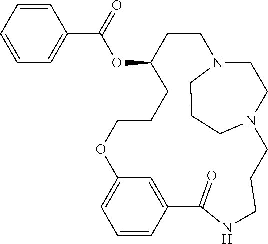 [SMILES: O=C1NCCCN2CCCN(CC[C@H](OC(=O)c3ccccc3)CCCOc3cccc1c3)CC2] | (12R)-6-oxo-8-oxa-5-aza-1(1,4)- diazepana-7(1,3)- benzenacyclotetradecaphane-12-yl benzoate |
| 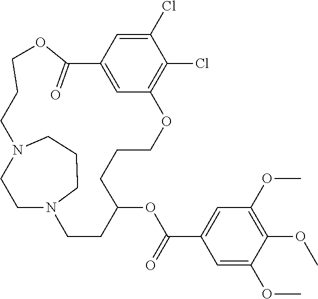 [SMILES: COc1cc(C(=O)OC2CCCOc3cc(cc(Cl)c3Cl)C(=O)OCCCN3CCCN(CC2)CC3)cc(OC)c1OC] | 7⁴,7⁵-dichloro-6-oxo-5,8-dioxa-1(1,4)- diazepana-7(1,3)- benzenacyclotetradecaphane-12-yl 3,4,5- trimethoxybenzoate |
| 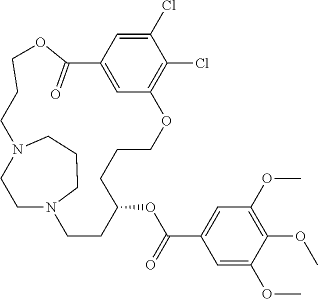 [SMILES: COc1cc(C(=O)O[C@H]2CCCOc3cc(cc(Cl)c3Cl)C(=O)OCCCN3CCCN(CC2)CC3)cc(OC)c1OC] | (12S)-7⁴,7⁵-dichloro-6-oxo-5,8-dioxa- 1(1,4)-diazepana-7(1,3)- benzenacyclotetradecaphane-12-yl 3,4,5- trimethoxybenzoate |
| 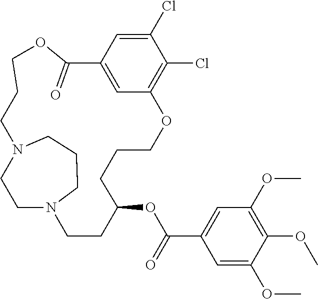 [SMILES: COc1cc(C(=O)O[C@@H]2CCCOc3cc(cc(Cl)c3Cl)C(=O)OCCCN3CCCN(CC2)CC3)cc(OC)c1OC] | (12R)-7⁴,7⁵-dichloro-6-oxo-5,8-dioxa- 1(1,4)-diazepana-7(1,3)- benzenacyclotetradecaphane-12-yl 3,4,5- trimethoxybenzoate |
| 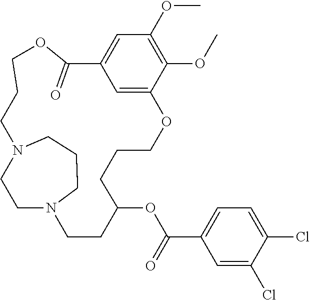 [SMILES: COc1cc2cc(c1OC)OCCCC(OC(=O)c1ccc(Cl)c(Cl)c1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 3,4- dichlorobenzoate |
| 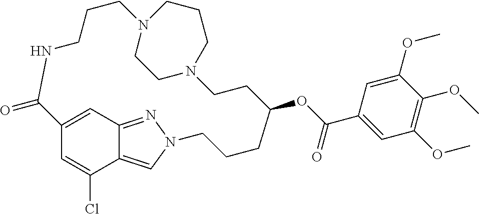 [SMILES: COc1cc(C(=O)O[C@H]2CCCn3cc4c(Cl)cc(cc4n3)C(=O)NCCCN3CCCN(CC2)CC3)cc(OC)c1OC] | (11Z,16E,10S)-14-chloro-2-oxo-12H-3- aza-1(6,2)-indazola-7(1,4)- diazepanacyclotridecaphane-10-yl 3,4,5- trimethoxybenzoate |
| 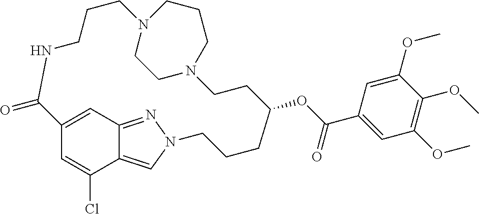 [SMILES: COc1cc(C(=O)O[C@@H]2CCCn3cc4c(Cl)cc(cc4n3)C(=O)NCCCN3CCCN(CC2)CC3)cc(OC)c1OC] | (11Z,16E,10R)-14-chloro-2-oxo-12H-3- aza-1(6,2)-indazola-7(1,4)- diazepanacyclotridecaphane-10-yl 3,4,5- trimethoxybenzoate |
| 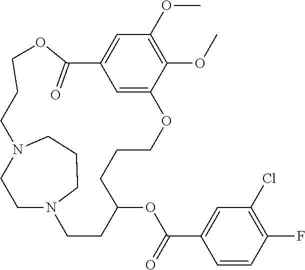 [SMILES: COc1cc2cc(c1OC)OCCCC(OC(=O)c1ccc(F)c(Cl)c1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 3- chloro-4-fluorobenzoate |
| 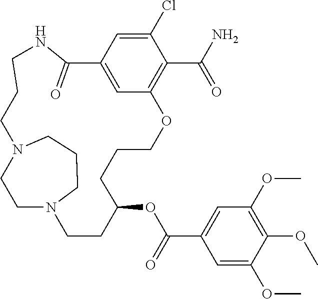 [SMILES: COc1cc(C(=O)O[C@@H]2CCCOc3cc(cc(Cl)c3C(N)=O)C(=O)NCCCN3CCCN(CC2)CC3)cc(OC)c1OC] | (12R)-7⁴-carbamoyl-7⁵-chloro-6-oxo-8- oxa-5-aza-1(1,4)-diazepana-7(1,3)- benzenacyclotetradecaphane-12-yl 3,4,5- trimethoxybenzoate |
| 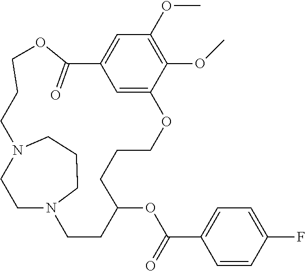 [SMILES: COc1cc2cc(c1OC)OCCCC(OC(=O)c1ccc(F)cc1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 4- fluorobenzoate |
| 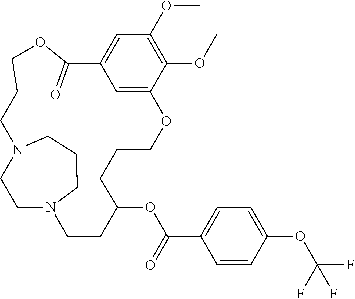 [SMILES: COc1cc2cc(c1OC)OCCCC(OC(=O)c1ccc(OC(F)(F)F)cc1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 4- (trifluoromethoxy)benzoate |
| 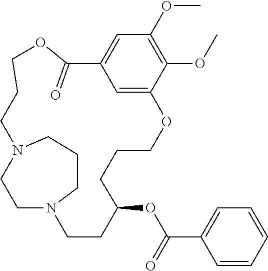 [SMILES: COc1cc2cc(c1OC)OCCC[C@@H](OC(=O)c1ccccc1)CCN1CCCN(CCCOC2=O)CC1] | (12R)-7⁴,7⁵-dimethoxy-6-oxo-5,8-dioxa- 1(1,4)-diazepana-7(1,3)- benzenacyclotetradecaphane-12-yl benzoate |
| 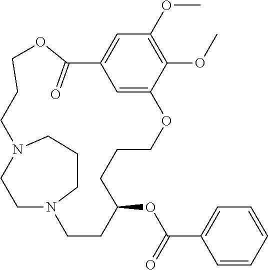 [SMILES: COc1cc2cc(c1OC)OCCC[C@@H](OC(=O)c1ccccc1)CCN1CCCN(CCCOC2=O)CC1] | (12R)-7⁴,7⁵-dimethoxy-6-oxo-5,8-dioxa- 1(1,4)-diazepana-7(1,3)- benzenacyclotetradecaphane-12-yl benzoate |
| 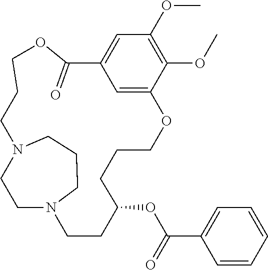 [SMILES: COc1cc2cc(c1OC)OCCC[C@H](OC(=O)c1ccccc1)CCN1CCCN(CCCOC2=O)CC1] | (12S)-7⁴,7⁵-dimethoxy-6-oxo-5,8-dioxa- 1(1,4)-diazepana-7(1,3)- benzenacyclotetradecaphane-12-yl benzoate |
| 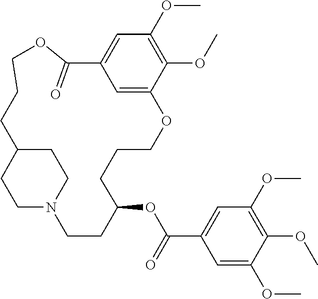 [SMILES: COc1cc(C(=O)O[C@@H]2CCCOc3cc(cc(OC)c3OC)C(=O)OCCCC3CCN(CC3)CC2)cc(OC)c1OC] | (R)-7⁴,7⁵-dimethoxy-6-oxo-5,8-dioxa- 1(4,1)-piperidina-7(1,3)- benzenacyclotetradecaphane-12-yl 3,4,5- trimethoxybenzoate |
| 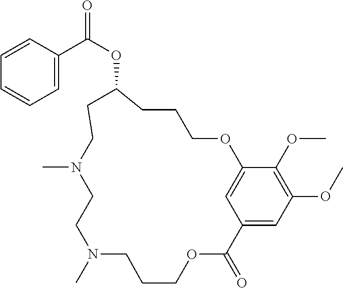 [SMILES: COc1cc2cc(c1OC)OCCC[C@@H](OC(=O)c1ccccc1)CCN(C)CCN(C)CCCOC2=O] | (R)-15,16-dimethoxy-9,12-dimethyl-17- oxo-2,16-dioxa-9,12-diaza-1(1,3)- benzenacycloheptadecaphane-6-yl benzoate |
| 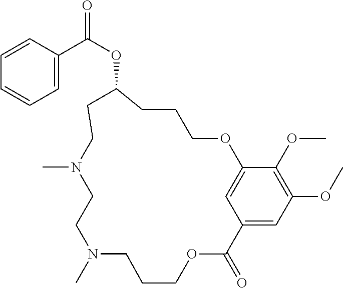 [SMILES: COc1cc2cc(c1OC)OCCC[C@@H](OC(=O)c1ccccc1)CCN(C)CCN(C)CCCOC2=O] | (R)-15,16-dimethoxy-9,12-dimethyl-17- oxo-2,16-dioxa-9,12-diaza-1(1,3)- benzenacycloheptadecaphane-6-yl benzoate |
| 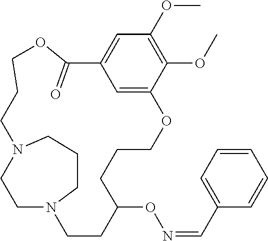 [SMILES: COc1cc2cc(c1OC)OCCCC(O/N=C\c1ccccc1)CCN1CCCN(CCCOC2=O)CC1] | (Z)-benzaldehyde O-(7⁴,7⁵-dimethoxy-6- oxo-5,8-dioxa-1(1,4)-diazepana-7(1,3)- benzenacyclotetradecaphane-12-yl) oxime |
| 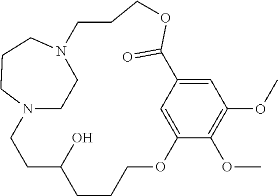 [SMILES: COc1cc2cc(c1OC)OCCCC(O)CCN1CCCN(CCCOC2=O)CC1] | 12-hydroxy-7⁴,7⁵-dimethoxy-5,8-dioxa- 1(1,4)-diazepana-7(1,3)- benzenacyclotetradecaphan-6-one |
| 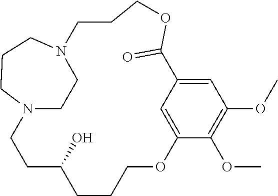 [SMILES: COc1cc2cc(c1OC)OCCC[C@@H](O)CCN1CCCN(CCCOC2=O)CC1] | (12R)-12-hydroxy-7⁴,7⁵-dimethoxy-5,8- dioxa-1(1,4)-diazepana-7(1,3)- benzenacyclotetradecaphan-6-one |
| 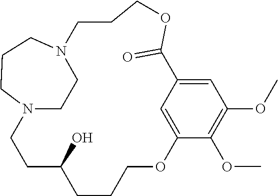 [SMILES: COc1cc2cc(c1OC)OCCC[C@H](O)CCN1CCCN(CCCOC2=O)CC1] | (12S)-12-hydroxy-7⁴,7⁵-dimethoxy-5,8- dioxa-1(1,4)-diazepana-7(1,3)- benzenacyclotetradecaphan-6-one |
| 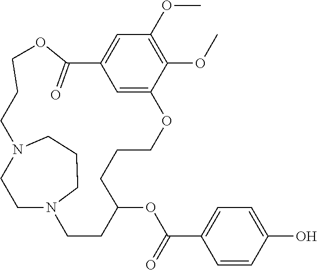 [SMILES: COc1cc2cc(c1OC)OCCCC(OC(=O)c1ccc(O)cc1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 4- hydroxybenzoate |
| 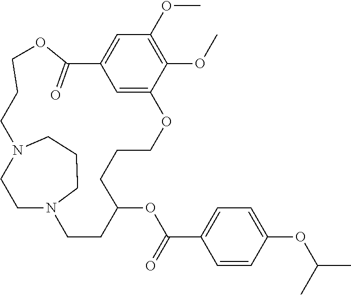 [SMILES: COc1cc2cc(c1OC)OCCCC(OC(=O)c1ccc(OC(C)C)cc1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 4- isopropoxybenzoate |
| 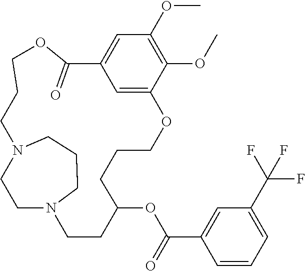 [SMILES: COc1cc2cc(c1OC)OCCCC(OC(=O)c1cccc(C(F)(F)F)c1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 3- (trifluoromethyl)benzoate |
| 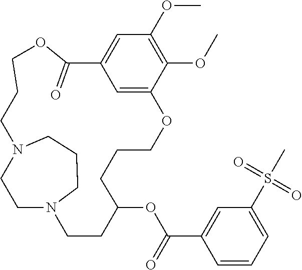 [SMILES: COc1cc2cc(c1OC)OCCCC(OC(=O)c1cccc(S(C)(=O)=O)c1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 3- (methylsulfonyl)benzoate |
| 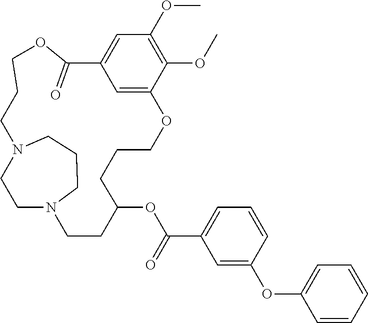 [SMILES: COc1cc2cc(c1OC)OCCCC(OC(=O)c1cccc(Oc3ccccc3)c1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 3- phenoxybenzoate |
| 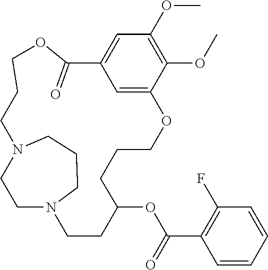 [SMILES: COc1cc2cc(c1OC)OCCCC(OC(=O)c1ccccc1F)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 2- fluorobenzoate |
| 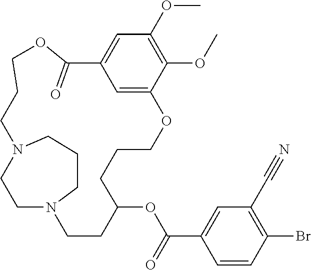 [SMILES: COc1cc2cc(c1OC)OCCCC(OC(=O)c1ccc(Br)c(C#N)c1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 4- bromo-3-cyanobenzoate |
| 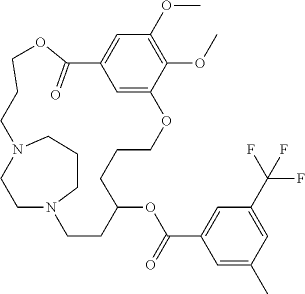 [SMILES: COc1cc2cc(c1OC)OCCCC(OC(=O)c1cc(C)cc(C(F)(F)F)c1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 3- methyl-5-(trifluoromethyl)benzoate |
| 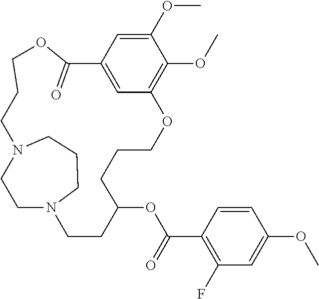 [SMILES: COc1ccc(C(=O)OC2CCCOc3cc(cc(OC)c3OC)C(=O)OCCCN3CCCN(CC2)CC3)c(F)c1] | 7⁴,7⁵-dimethoxy-6-oxo-5,8-dioxa-1(1,4)- diazepana-7(1,3)- benzenacyclotetradecaphane-12-yl 2- fluoro-4-methoxybenzoate |
| 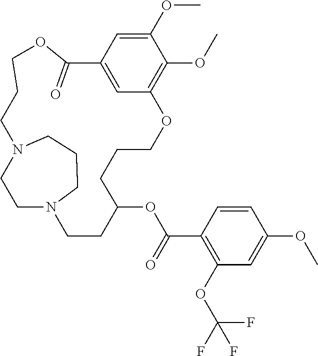 [SMILES: COc1ccc(C(=O)OC2CCCOc3cc(cc(OC)c3OC)C(=O)OCCCN3CCCN(CC2)CC3)c(OC(F)(F)F)c1] | 7⁴,7⁵-dimethoxy-6-oxo-5,8-dioxa-1(1,4)- diazepana-7(1,3)- benzenacyclotetradecaphane-12-yl 4- methoxy-2-(trifluoromethoxy)benzoate |
| 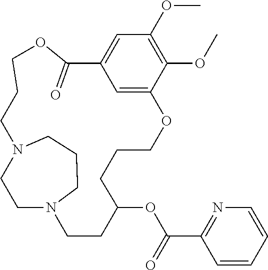 [SMILES: COc1cc2cc(c1OC)OCCCC(OC(=O)c1ccccn1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl picolinate |
| 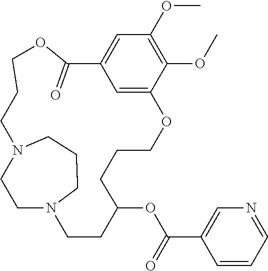 [SMILES: COc1cc2cc(c1OC)OCCCC(OC(=O)c1cccnc1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl nicotinate |
| 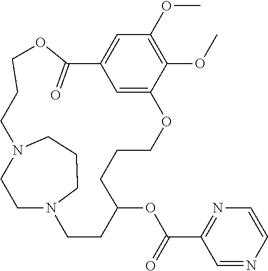 [SMILES: COc1cc2cc(c1OC)OCCCC(OC(=O)c1cnccn1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl pyrazine-2-carboxylate |
| 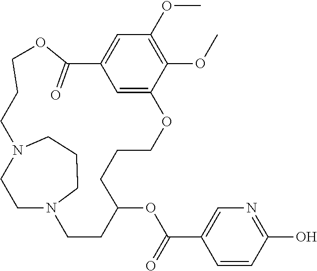 [SMILES: COc1cc2cc(c1OC)OCCCC(OC(=O)c1ccc(O)nc1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 6- hydroxynicotinate |
| 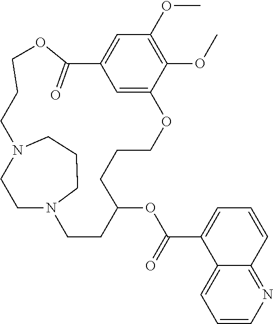 [SMILES: COc1cc2cc(c1OC)OCCCC(OC(=O)c1cccc3ncccc13)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl quinoline-5-carboxylate |
| 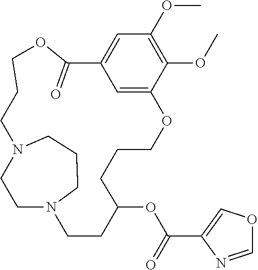 [SMILES: COc1cc2cc(c1OC)OCCCC(OC(=O)c1cocn1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl oxazole-4-carboxylate |
| 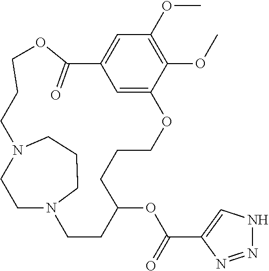 [SMILES: COc1cc2cc(c1OC)OCCCC(OC(=O)c1c[nH]nn1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 1H- 1,2,3-triazole-4-carboxylate |
| 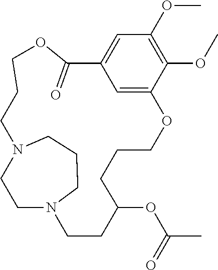 [SMILES: COc1cc2cc(c1OC)OCCCC(OC(C)=O)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl acetate |
| 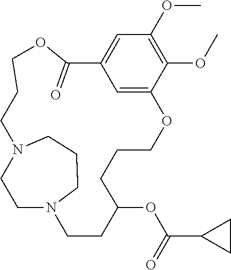 [SMILES: COc1cc2cc(c1OC)OCCCC(OC(=O)C1CC1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl cyclopropanecarboxylate |
| 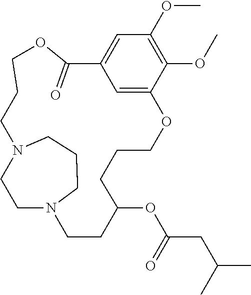 [SMILES: COc1cc2cc(c1OC)OCCCC(OC(=O)CC(C)C)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 3- methylbutanoate |
| 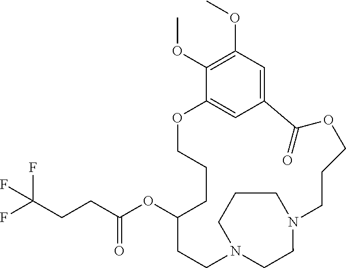 [SMILES: COc1cc2cc(c1OC)OCCCC(OC(=O)CCC(F)(F)F)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 4,4,4- trifluorobutanoate |
| 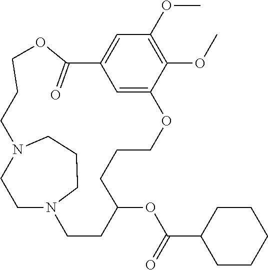 [SMILES: COc1cc2cc(c1OC)OCCCC(OC(=O)C1CCCCC1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl cyclohexanecarboxylate |
| 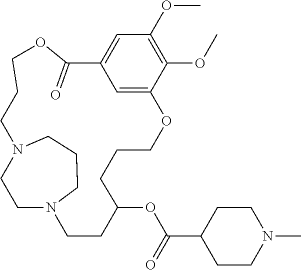 [SMILES: COc1cc2cc(c1OC)OCCCC(OC(=O)C1CCN(C)CC1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 1- methylpiperidine-4-carboxylate |
| 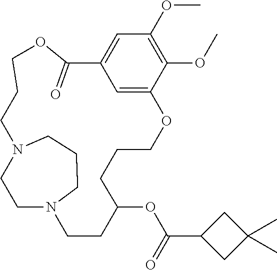 [SMILES: COc1cc2cc(c1OC)OCCCC(OC(=O)C1CC(C)(C)C1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 3,3- dimethylcyclobutane-1-carboxylate |
| 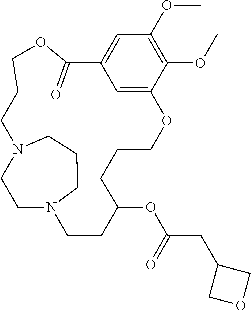 [SMILES: COc1cc2cc(c1OC)OCCCC(OC(=O)CC1COC1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 2- (oxetan-3-yl)acetate |
| 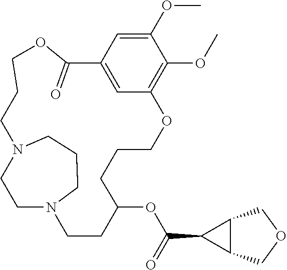 [SMILES: COc1cc2cc(c1OC)OCCCC(OC(=O)[C@H]1[C@@H]3COC[C@@H]31)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl (1R,5S,6r)-3-oxabicyclo[3.1.0]hexane-6- carboxylate |
| 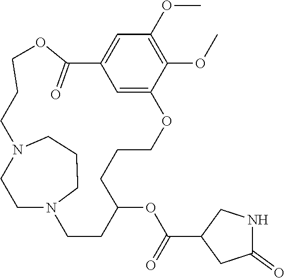 [SMILES: COc1cc2cc(c1OC)OCCCC(OC(=O)C1CNC(=O)C1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 5- oxopyrrolidine-3-carboxylate |
| 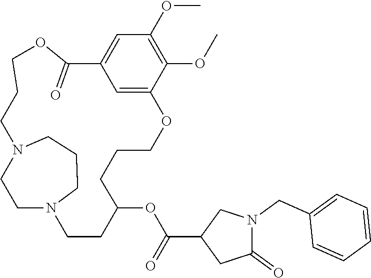 [SMILES: COc1cc2cc(c1OC)OCCCC(OC(=O)C1CC(=O)N(Cc3ccccc3)C1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 1- benzyl-5-oxopyrrolidine-3-carboxylate |
| 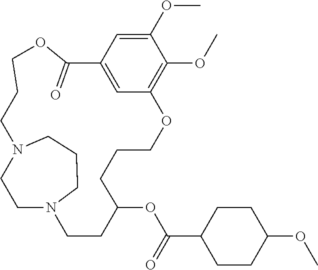 [SMILES: COc1cc2cc(c1OC)OCCCC(OC(=O)C1CCC(OC)CC1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 4- methoxycyclohexane-1-carboxylate |
| 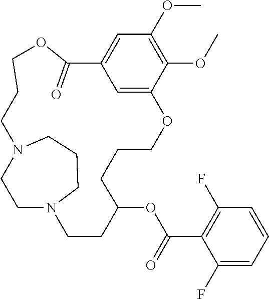 [SMILES: COc1cc2cc(c1OC)OCCCC(OC(=O)c1c(F)cccc1F)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 2,6- difluorobenzoate |
| 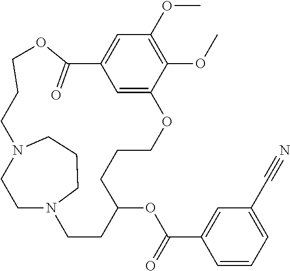 [SMILES: COc1cc2cc(c1OC)OCCCC(OC(=O)c1cccc(C#N)c1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 3- cyanobenzoate |
| 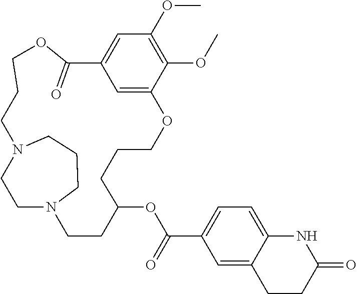 [SMILES: COc1cc2cc(c1OC)OCCCC(OC(=O)c1ccc3c(c1)CCC(=O)N3)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 2- oxo-1,2,3,4-tetrahydroquinoline-6- carboxylate |
| 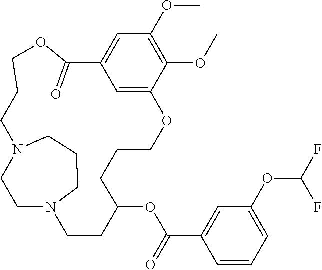 [SMILES: COc1cc2cc(c1OC)OCCCC(OC(=O)c1cccc(OC(F)F)c1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 3- (difluoromethoxy)benzoate |
| 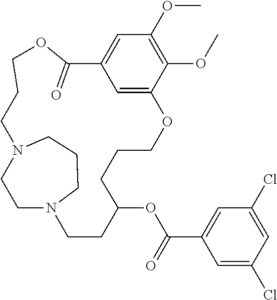 [SMILES: COc1cc2cc(c1OC)OCCCC(OC(=O)c1cc(Cl)cc(Cl)c1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 3,5- dichlorobenzoate |
| 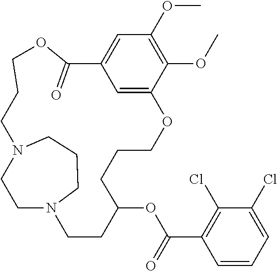 [SMILES: COc1cc2cc(c1OC)OCCCC(OC(=O)c1cccc(Cl)c1Cl)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 2,3- dichlorobenzoate |
| 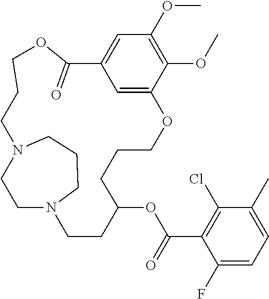 [SMILES: COc1cc2cc(c1OC)OCCCC(OC(=O)c1c(F)ccc(C)c1Cl)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 2- chloro-6-fluoro-3-methylbenzoate |
| 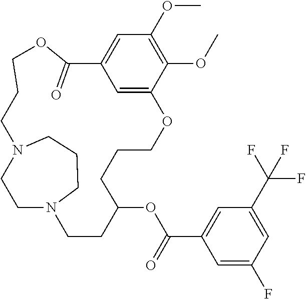 [SMILES: COc1cc2cc(c1OC)OCCCC(OC(=O)c1cc(F)cc(C(F)(F)F)c1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 3- fluoro-5-(trifluoromethyl)benzoate |
| 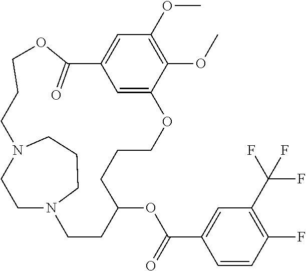 [SMILES: COc1cc2cc(c1OC)OCCCC(OC(=O)c1ccc(F)c(C(F)(F)F)c1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 4- fluoro-3-(trifluoromethyl)benzoate |
| 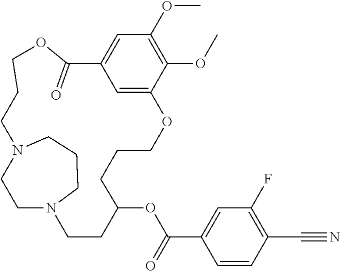 [SMILES: COc1cc2cc(c1OC)OCCCC(OC(=O)c1ccc(C#N)c(F)c1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 4- cyano-3-fluorobenzoate |
| 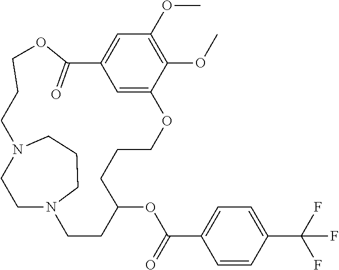 [SMILES: COc1cc2cc(c1OC)OCCCC(OC(=O)c1ccc(C(F)(F)F)cc1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 4- (trifluoromethyl)benzoate |
| 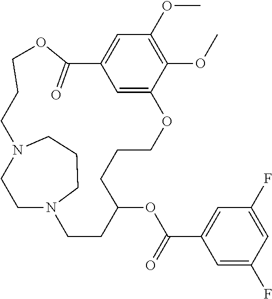 [SMILES: COc1cc2cc(c1OC)OCCCC(OC(=O)c1cc(F)cc(F)c1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 3,5- difluorobenzoate |
| 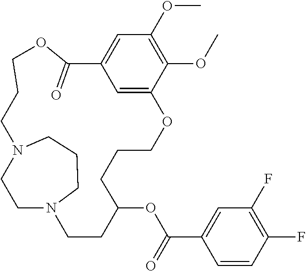 [SMILES: COc1cc2cc(c1OC)OCCCC(OC(=O)c1ccc(F)c(F)c1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 3,4- difluorobenzoate |
| 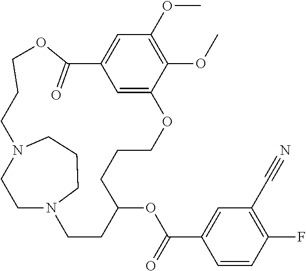 [SMILES: COc1cc2cc(c1OC)OCCCC(OC(=O)c1ccc(F)c(C#N)c1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 3- cyano-4-fluorobenzoate |
| 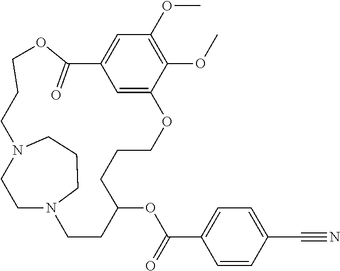 [SMILES: COc1cc2cc(c1OC)OCCCC(OC(=O)c1ccc(C#N)cc1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 4- cyanobenzoate |
| 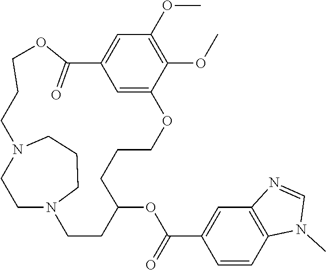 [SMILES: COc1cc2cc(c1OC)OCCCC(OC(=O)c1ccc3c(c1)ncn3C)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 1- methyl-1H-benzo[d]imidazole-5- carboxylate |
| 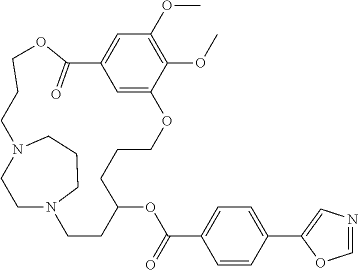 [SMILES: COc1cc2cc(c1OC)OCCCC(OC(=O)c1ccc(-c3cnco3)cc1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 4- (oxazol-5-yl)benzoate |
| 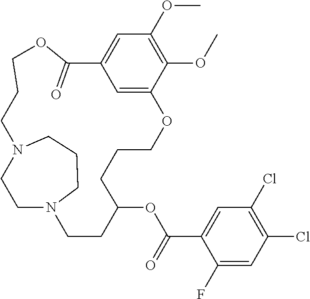 [SMILES: COc1cc2cc(c1OC)OCCCC(OC(=O)c1cc(Cl)c(Cl)cc1F)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 4,5- dichloro-2-fluorobenzoate |
| 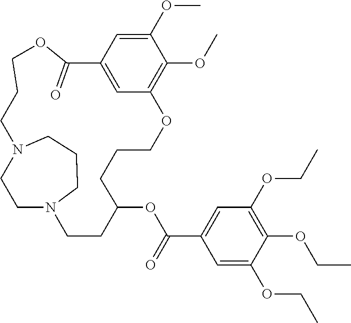 [SMILES: CCOc1cc(C(=O)OC2CCCOc3cc(cc(OC)c3OC)C(=O)OCCCN3CCCN(CC2)CC3)cc(OCC)c1OCC] | 7⁴,7⁵-dimethoxy-6-oxo-5,8-dioxa-1(1,4)- diazepana-7(1,3)- benzenacyclotetradecaphane-12-yl 3,4,5- triethoxybenzoate |
| 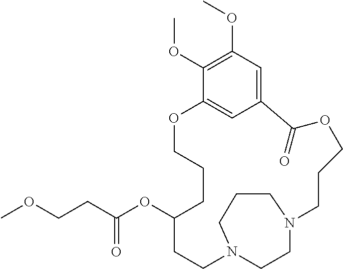 [SMILES: COCCC(=O)OC1CCCOc2cc(cc(OC)c2OC)C(=O)OCCCN2CCCN(CC1)CC2] | 7⁴,7⁵-dimethoxy-6-oxo-5,8-dioxa-1(1,4)- diazepana-7(1,3)- benzenacyclotetradecaphane-12-yl 3- methoxypropanoate |
| 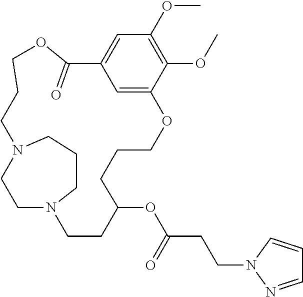 [SMILES: COc1cc2cc(c1OC)OCCCC(OC(=O)CCn1cccn1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 3- (1H-pyrazol-1-yl)propanoate |
| 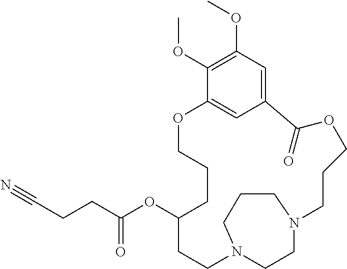 [SMILES: COc1cc2cc(c1OC)OCCCC(OC(=O)CCC#N)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 3- cyanopropanoate |
| 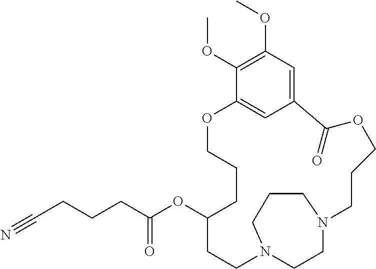 [SMILES: COc1cc2cc(c1OC)OCCCC(OC(=O)CCCC#N)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 4- cyanobutanoate |
| 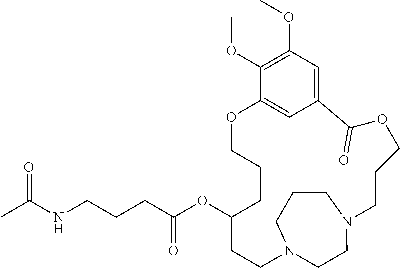 [SMILES: COc1cc2cc(c1OC)OCCCC(OC(=O)CCCNC(C)=O)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 4- acetamidobutanoate |
| 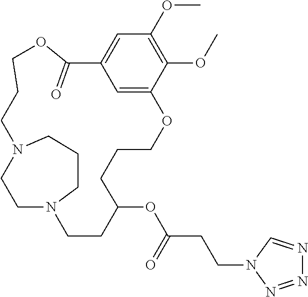 [SMILES: COc1cc2cc(c1OC)OCCCC(OC(=O)CCn1cnnn1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 3- (1H-tetrazol-1-yl)propanoate |
| 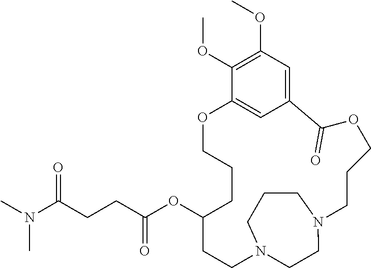 [SMILES: COc1cc2cc(c1OC)OCCCC(OC(=O)CCC(=O)N(C)C)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 4- (dimethylamino)-4-oxobutanoate |
| 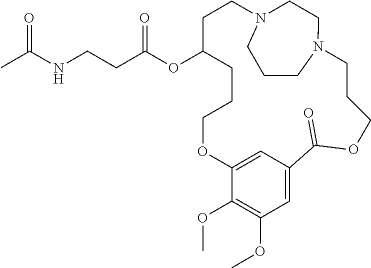 [SMILES: COc1cc2cc(c1OC)OCCCC(OC(=O)CCNC(C)=O)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 3- acetamidopropanoate |
| 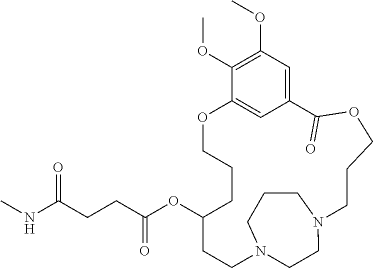 [SMILES: CNC(=O)CCC(=O)OC1CCCOc2cc(cc(OC)c2OC)C(=O)OCCCN2CCCN(CC1)CC2] | 7⁴,7⁵-dimethoxy-6-oxo-5,8-dioxa-1(1,4)- diazepana-7(1,3)- benzenacyclotetradecaphane-12-yl 4- (methylamino)-4-oxobutanoate |
| 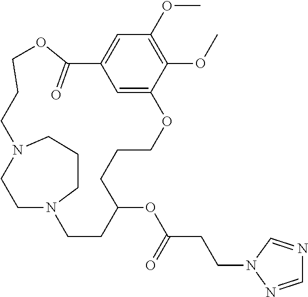 [SMILES: COc1cc2cc(c1OC)OCCCC(OC(=O)CCn1cncn1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 3- (1H-1,2,4-triazol-1-yl)propanoate |
| 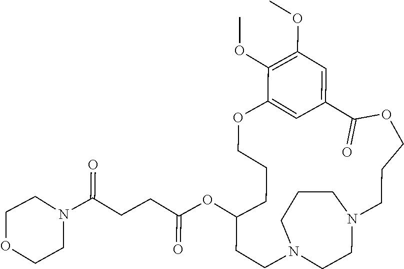 [SMILES: COc1cc2cc(c1OC)OCCCC(OC(=O)CCC(=O)N1CCOCC1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 4- morpholino-4-oxobutanoate |
| 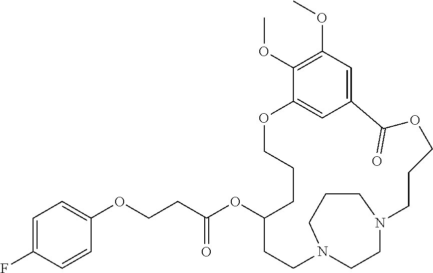 [SMILES: COc1cc2cc(c1OC)OCCCC(OC(=O)CCOc1ccc(F)cc1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 3-(4- fluorophenoxy)propanoate |
| 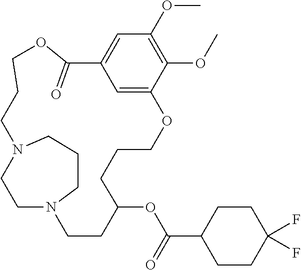 [SMILES: COc1cc2cc(c1OC)OCCCC(OC(=O)C1CCC(F)(F)CC1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 4,4- difluorocyclohexane-1-carboxylate |
| 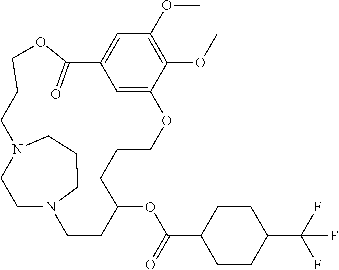 [SMILES: COc1cc2cc(c1OC)OCCCC(OC(=O)C1CCC(C(F)(F)F)CC1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 4- (trifluoromethyl)cyclohexane-1- carboxylate |
| 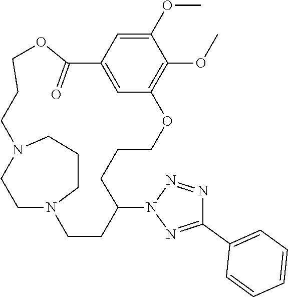 [SMILES: COc1cc2cc(c1OC)OCCCC(n1nnc(-c3ccccc3)n1)CCN1CCCN(CCCOC2=O)CC1] | 7⁴,7⁵-dimethoxy-12-(5-phenyl-2H- tetrazol-2-yl)-5,8-dioxa-1(1,4)- diazepana-7(1,3)- benzenacyclotetradecaphan-6-one |
| 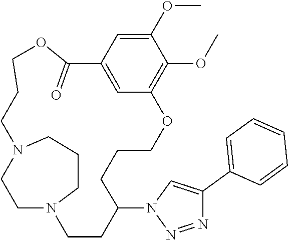 [SMILES: COc1cc2cc(c1OC)OCCCC(n1cc(-c3ccccc3)nn1)CCN1CCCN(CCCOC2=O)CC1] | 7⁴,7⁵-dimethoxy-12-(4-phenyl-1H-1,2,3- triazol-1-yl)-5,8-dioxa-1(1,4)-diazepana- 7(1,3)-benzenacyclotetradecaphan-6-one |
| 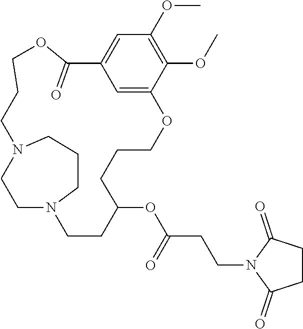 [SMILES: COc1cc2cc(c1OC)OCCCC(OC(=O)CCN1C(=O)CCC1=O)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 3- (2,5-dioxopyrrolidin-1-yl)propanoate |
| 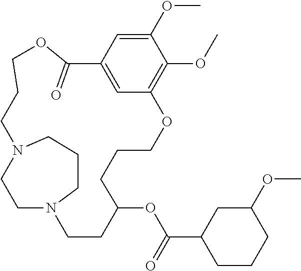 [SMILES: COc1cc2cc(c1OC)OCCCC(OC(=O)C1CCCC(OC)C1)CCN1CCCN(CCCOC2=O)CC1] | 7⁴,7⁵-dimethoxy-6-oxo-5,8-dioxa-1(1,4)- diazepana-7(1,3)- benzenacyclotetradecaphane-12-yl 3- methoxycyclohexane-1-carboxylate |
| 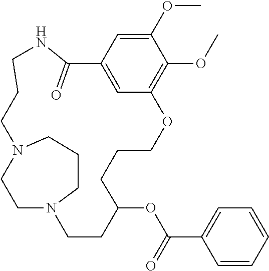 [SMILES: COc1cc2cc(c1OC)OCCCC(OC(=O)c1ccccc1)CCN1CCCN(CCCNC2=O)CC1] | 7⁴,7⁵-dimethoxy-6-oxo-8-oxa-5-aza- 1(1,4)-diazepana-7(1,3)- benzenacyclotetradecaphane-12-yl benzoate |
| 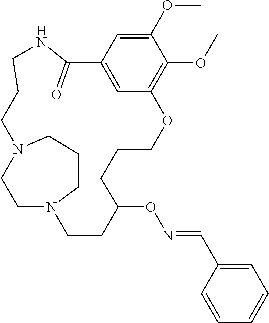 [SMILES: COc1cc2cc(c1OC)OCCCC(O/N=C/c1ccccc1)CCN1CCCN(CCCNC2=O)CC1] | (E)-benzaldehyde O-(7⁴,7⁵-dimethoxy-6- oxo-8-oxa-5-aza-1(1,4)-diazepana- 7(1,3)-benzenacyclotetradecaphane-12- yl)oxime |
| 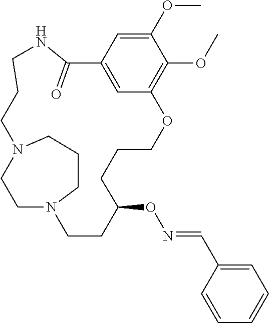 [SMILES: COc1cc2cc(c1OC)OCCC[C@@H](O/N=C/c1ccccc1)CCN1CCCN(CCCNC2=O)CC1] | (E)-benzaldehyde O-((12R)-7⁴,7⁵- dimethoxy-6-oxo-8-oxa-5-aza-1(1,4)- diazepana-7(1,3)- benzenacyclotetradecaphane-12-yl) oxime |
| 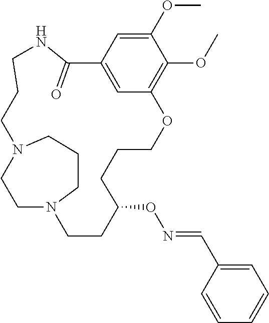 [SMILES: COc1cc2cc(c1OC)OCCC[C@H](O/N=C/c1ccccc1)CCN1CCCN(CCCNC2=O)CC1] | (E)-benzaldehyde O-((12S)-7⁴,7⁵- dimethoxy-6-oxo-8-oxa-5-aza-1(1,4)- diazepana-7(1,3)- benzenacyclotetradecaphane-12-yl) oxime |
| 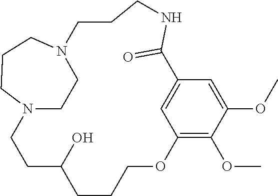 [SMILES: COc1cc2cc(c1OC)OCCCC(O)CCN1CCCN(CCCNC2=O)CC1] | 12-hydroxy-7⁴,7⁵-dimethoxy-8-oxa-5- aza-1(1,4)-diazepana-7(1,3)- benzenacyclotetradecaphan-6-one |
| 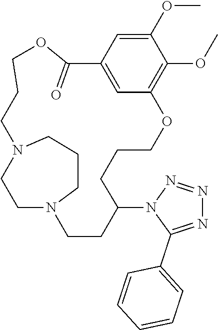 [SMILES: COc1cc2cc(c1OC)OCCCC(n1nnnc1-c1ccccc1)CCN1CCCN(CCCOC2=O)CC1] | 7⁴,7⁵-dimethoxy-12-(5-phenyl-1H- tetrazol-1-yl)-5,8-dioxa-1(1,4)- diazepana-7(1,3)- benzenacyclotetradecaphan-6-one |
| 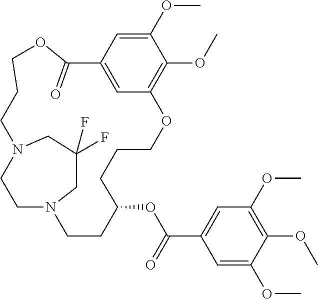 [SMILES: COc1cc(C(=O)O[C@H]2CCCOc3cc(cc(OC)c3OC)C(=O)OCCCN3CCN(CC2)CC(F)(F)C3)cc(OC)c1OC] | (12S)-16,16-difluoro-7⁴,7⁵-dimethoxy-6- oxo-5,8-dioxa-1(1,4)-diazepana-7(1,3)- benzenacyclotetradecaphane-12-yl 3,4,5- trimethoxybenzoate |
| 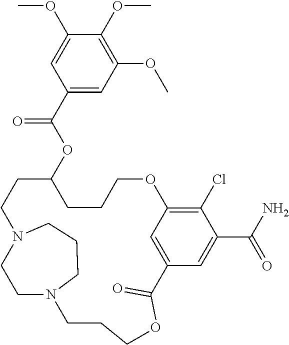 [SMILES: COc1cc(C(=O)OC2CCCOc3cc(cc(C(N)=O)c3Cl)C(=O)OCCCN3CCCN(CC2)CC3)cc(OC)c1OC] | 7⁵-carbamoyl-7⁴-chloro-6-oxo-5,8-dioxa- 1(1,4)-diazepana-7(1,3)- benzenacyclotetradecaphane-12-yl 3,4,5- trimethoxybenzoate |
| 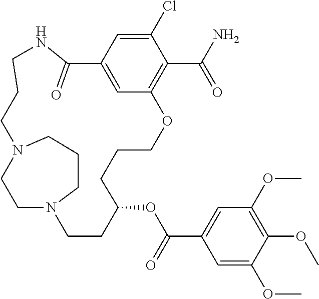 [SMILES: COc1cc(C(=O)O[C@H]2CCCOc3cc(cc(Cl)c3C(N)=O)C(=O)NCCCN3CCCN(CC2)CC3)cc(OC)c1OC] | (12S)-7⁴-carbamoyl-7⁵-chloro-6-oxo-8- oxa-5-aza-1(1,4)-diazepana-7(1,3)- benzenacyclotetradecaphane-12-yl 3,4,5- trimethoxybenzoate |
| 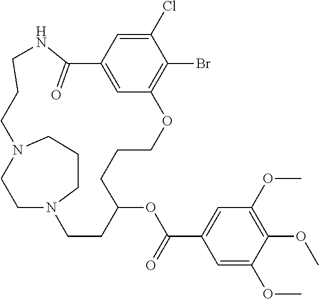 [SMILES: COc1cc(C(=O)OC2CCCOc3cc(cc(Cl)c3Br)C(=O)NCCCN3CCCN(CC2)CC3)cc(OC)c1OC] | 7⁴-bromo-7⁵-chloro-6-oxo-8-oxa-5-aza- 1(1,4)-diazepana-7(1,3)- benzenacyclotetradecaphane-12-yl 3,4,5- trimethoxybenzoate |
| 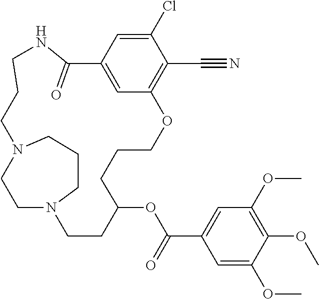 [SMILES: COc1cc(C(=O)OC2CCCOc3cc(cc(Cl)c3C#N)C(=O)NCCCN3CCCN(CC2)CC3)cc(OC)c1OC] | 7⁵-chloro-7⁴-cyano-6-oxo-8-oxa-5-aza- 1(1,4)-diazepana-7(1,3)- benzenacyclotetradecaphane-12-yl 3,4,5- trimethoxybenzoate |
| 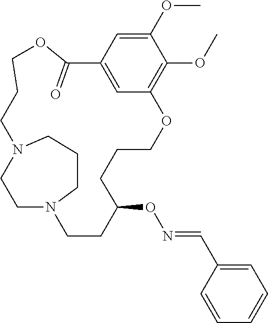 [SMILES: COc1cc2cc(c1OC)OCCC[C@@H](O/N=C/c1ccccc1)CCN1CCCN(CCCOC2=O)CC1] | 7⁴,7⁵-dimethoxy-12-(5-phenyl-1H- tetrazol-1-yl)-5,8-dioxa-1(1,4)- diazepana-7(1,3)- benzenacyclotetradecaphan-6-one |
| 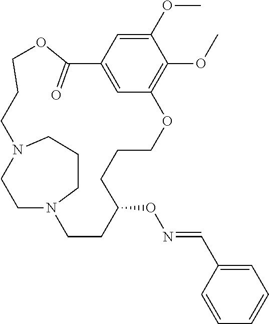 [SMILES: COc1cc2cc(c1OC)OCCC[C@H](O/N=C/c1ccccc1)CCN1CCCN(CCCOC2=O)CC1] | 7⁴,7⁵-dimethoxy-12-(5-phenyl-1H- tetrazol-1-yl)-5,8-dioxa-1(1,4)- diazepana-7(1,3)- benzenacyclotetradecaphan-6-one |

In some embodiments, the ENT1 inhibitor is selected from dilazep, dipyridamole, NBMPR (nitrobenzylthioinosine), draflazine, STI-571 (Gleevec), ticagrelor, soluflazine, mioflazine, decynium-22, lopinavir, quinidine, 8MDP, TC-T 6000, 5-iodotubercidin, cilostazol, and salts thereof and any mixture thereof. In one embodiment, the ENT1 inhibitor is selected from NBMPR, dipyridamole, dilazep, ticagrelor and salts thereof (including dilazep hydrochloride). In another embodiment, the ENT1 inhibitor is selected from dipyridamole, dilazep, ticagrelor and salts thereof (including dilazep hydrochloride). In one embodiment, the ENT1 inhibitor is NBMP or a salt thereof. In one embodiment, the ENT1 inhibitor is dipyridamole or a salt thereof. In one embodiment, the ENT1 inhibitor is dilazep or a salt thereof (including dilazep hydrochloride). In one embodiment, the ENT1 inhibitor is ticagrelor or a salt thereof.

In some embodiments, the ENT1 inhibitor is selected from dilazep, dipyridamole, NBMPR (nitrobenzylthioinosine), draflazine, STI-571 (Gleevec), ticagrelor, 8MDP, 5-iodotubercidin, cilostazol, and salts thereof and any mixture thereof. Examples of selective ENT1 inhibitors include NBMPR, STI-571 (Gleevec), ticagrelor, salts thereof and any mixture thereof.

### B. Checkpoint Inhibitors

Any checkpoint inhibitor may be used in the combinations, compositions, kits, methods, and uses disclosed herein.

In one embodiment, the checkpoint inhibitor is an immunotherapeutic agent.

In some embodiments, checkpoint inhibitors (CPI), which may also be referred to as immune checkpoint inhibitors (ICI), block the interactions between inhibitory receptors expressed on T cells and their ligands. As a cancer treatment, use of checkpoint inhibitor aims at preventing the activation of inhibitory receptors expressed on T cells by ligands expressed by a tumor. Use of checkpoint inhibitors thus aims at preventing inhibition of T cells present in the tumor, i.e., tumor infiltrating T cells, and thus at enhancing the subject immune response towards a tumor.

Examples of checkpoint inhibitors include, without being limited to:
inhibitors of the cell surface receptor PD-1 (programmed cell death protein 1), also known as CD279 (cluster differentiation 279);
inhibitors of the ligand PD-L1 (programmed death-ligand 1), also known as CD274 (cluster of differentiation 274) or B7-H1 (B7 homolog 1);
inhibitors of the cell surface receptor CTLA4 or CTLA-4 (cytotoxic T-lymphocyte-associated protein 4), also known as CD152 (cluster of differentiation 152);
inhibitors of LAG-3 (lymphocyte-activation gene 3), also known as CD223 (cluster differentiation 223);
inhibitors of TIM-3 (T-cell immunoglobulin and mucin-domain containing-3), also known as HAVCR2 (hepatitis A virus cellular receptor 2) or CD366 (cluster differentiation 366);
inhibitors of BTLA (B and T lymphocyte attenuator), also known as CD272 (cluster differentiation 272);
inhibitors of CEACAM-1 (carcinoembryonic antigen-related cell adhesion molecule 1) also known as CD66a (cluster differentiation 66a); and
inhibitors of GITR (glucocorticoid-induced TNFR-related protein) also known as TNFRSF18 (tumor necrosis factor receptor superfamily member 18) or AITR (activation-inducible TNFR family receptor).

In one embodiment, a checkpoint inhibitor is selected from inhibitors of PD-1, inhibitors of PD-L1, inhibitors of CTLA4, inhibitors of LAG-3, inhibitors of TIM-3, inhibitors of BTLA, inhibitors of CEACAM-1, inhibitors of GITR and any mixtures thereof.

In one embodiment, a checkpoint inhibitor is selected from inhibitors of PD-1, inhibitors of PD-L1, inhibitors of CTLA 4, and any mixtures thereof.

In one embodiment, a checkpoint inhibitor is selected from inhibitors of PD-1, inhibitors of PD-L1, inhibitors of CTLA 4, and any mixtures thereof.

In one embodiment, a checkpoint inhibitor is an inhibitor of PD-1, also referred to as an anti-PD-1. Inhibitors of PD-1 may include antibodies targeting PD-1, in particular monoclonal antibodies, and non-antibody inhibitors such as small molecule inhibitors.

Examples of inhibitors of PD-1 include, without being limited to, pembrolizumab, nivolumab, atezolizumab, dostarlimab, cemiplimab, tislelizumab, spartalizumab, ABBV-181, JNJ-63723283, BI 754091, MAG012, TSR-042, AGEN2034, and Opdualag™ (nivolumab and relatlimab-rmbw). Pembrolizumab is also known as MK-3475, MK03475, lambrolizumab, or SCH-900475. The trade name of pembrolizumab is Keytruda®. Nivolumab is also known as ONO-4538, BMS-936558, MDX1106, or GTPL7335. The trade name of nivolumab is Opdivo®. Cemiplimab is also known as REGN2810 or REGN-2810. Tislelizumab is also known as BGB-A317. Spartalizumab is also known as PDR001 or PDR-001.

In one embodiment, a checkpoint inhibitor is an inhibitor of PD-L1, also referred to as an anti-PD-L1. Inhibitors of PD-L1 may include antibodies targeting PD-L1, in particular monoclonal antibodies, and non-antibody inhibitors such as small molecule inhibitors.

Examples of inhibitors of PD-L1 include, without being limited to, avelumab, atezolizumab, durvalumab and LY3300054. Avelumab is also known as MSB0010718C, MSB-0010718C, MSB0010682, or MSB-0010682. The trade name of avelumab is Bavencio®. Atezolizumab is also known as MPDL3280A (clone YW243.55.S70), MPDL-3280A, RG-7446 or RG7446. The trade name of atezolizumab is Tecentriq®. Durvalumab is also known as MEDI4736 or MEDI-4736. The trade name of durvalumab is Imfinzi®.

In one embodiment, the checkpoint inhibitor is selected from pembrolizumab, nivolumab, atezolizumab, dostarlimab, cemiplimab, tislelizumab, spartalizumab, ABBV-181, JNJ-63723283, BI 754091, MAG012, TSR-042, AGEN2034, and Opdualag. In one embodiment, the checkpoint inhibitor is selected from avelumab, atezolizumab, durvalumab and LY3300054.

In one embodiment, the checkpoint inhibitor is selected from pembrolizumab, nivolumab, atezolizumab, dostarlimab, durvalumab, avelumab, and opdualag. In one embodiment, a checkpoint inhibitor is pembrolizumab. In one embodiment, a checkpoint inhibitor is nivolumab. In one embodiment, a checkpoint inhibitor is atezolizumab. In one embodiment, a checkpoint inhibitor is dostarlimab. In one embodiment, a checkpoint inhibitor is durvalumab. In one embodiment, a checkpoint inhibitor is avelumab. In one embodiment, a checkpoint inhibitor is opdualag.

In one embodiment, a checkpoint inhibitor is an inhibitor of CTLA-4, also referred to as an anti-CTLA-4.

Inhibitors of CTLA-4 may include antibodies targeting CTLA-4, in particular monoclonal antibodies, and non-antibody inhibitors such as small molecule inhibitors. Examples of inhibitors of CTLA-4 include, without being limited to, ipilimumab and tremelimumab. Ipilimumab is also known as BMS-734016, MDX-010, or MDX-101. The trade name of ipilimumab is Yervoy®. Tremelimumab is also known as ticilimumab, CP-675, or CP-675,206.

In one embodiment, at least one checkpoint inhibitor is selected from ipilimumab, tremelimumab, and any mixtures thereof.

### C. Adenosine Receptor Antagonists

Any adenosine receptor antagonist may be used in the combinations, compositions, kits, methods, and uses disclosed herein.

An adenosine receptor antagonist refers to a compound that, upon administration to a patient, results in inhibition or down-regulation of a biological activity associated with activation of an adenosine receptor in the patient, including any of the downstream biological effects otherwise resulting from the binding to an adenosine receptor of its natural ligand. Such adenosine receptor antagonists include any agent that can block activation of an adenosine receptor or any of the downstream biological effects of an adenosine receptor activation.

Adenosine receptors (or P1 receptors) are a class of purinergic G protein-coupled receptors with adenosine as endogenous ligand. There are four known types of adenosine receptors in humans: A₁, A_{2A}, A_{2B} and A₃; each is encoded by a different gene (ADOARA1, ADORA2A, ADORA2B, and ADORA3 respectively).

In one embodiment, an adenosine receptor antagonist is an antagonist of A₁ receptor, A_{2A} receptor, A_{2B} receptor, A₃ receptor or of a combination thereof.

In one embodiment, an adenosine receptor antagonist is an antagonist of A_{2A} receptor, A_{2B} receptor or of a combination thereof. In one embodiment, an adenosine receptor antagonist is an A_{2A} or A_{2B} receptor antagonist.

In one embodiment, an adenosine receptor antagonist is an antagonist of A_{2A} receptor (A_{2A}R antagonist). In one embodiment, the adenosine receptor antagonist is an antagonist of A_{2B} receptor (A_{2B}R antagonist).

In one embodiment, an adenosine receptor antagonist is an antagonist that is selective of A_{2A} receptor with respect to other adenosine receptors. In one embodiment, the adenosine receptor antagonist is an antagonist that is selective of A_{2A} receptor with respect to A_{2B} receptor.

In one embodiment, an adenosine receptor antagonist is an antagonist that is selective of A_{2B} receptor with respect to other adenosine receptors. In one embodiment, an adenosine receptor antagonist is an antagonist that is selective of A_{2B} receptor with respect to A_{2A} receptor.

### 1. A_{2A} Receptor Antagonists

An “A_{2A}R antagonist” refers to a compound that, upon administration to a subject, results in inhibition or down-regulation of a biological activity associated with activation of A_{2A} receptor in the patient, including any of the downstream biological effects otherwise resulting from the binding to A_{2A} receptor of its natural ligand. Such A_{2A}R antagonists include any agent that can block activation of A_{2A} receptor or any of the downstream biological effects of A_{2A} receptor activation.

In some embodiments, an A_{2A}R antagonist can be chosen from, but is not limited to, Preladenant (SCH-420,814), Vipadenant (BIIB-014), Tozadenant (SYK-115), ATL-444, Istradefylline (KW-6002), MSX-3, SCH-58261, SCH-412,348, SCH-442,416, ST-1535, Caffeine, VER-6623, VER-6947, VER-7835, ZM-241,385, and theophylline. In some embodiments, an A_{2A}R antagonist can be chosen from, but is not limited to, compounds disclosed in WO2018/178338, WO2011/121418, WO2009/156737, WO2011/095626 or WO2018/136700, WO 2023/059817, the contents of which are herein incorporated by reference.

In one embodiment, an A_{2A}R antagonist is a thiocarbamate disclosed in WO2018/178338 and/or WO 2023/059817. In some embodiments, an A_{2A}R antagonist is a compound of formula (I):

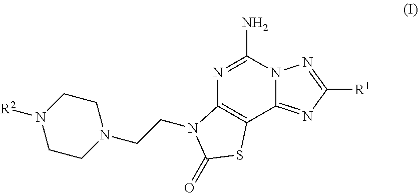

or a pharmaceutically acceptable salt or solvate thereof,
wherein
R¹ represents a 5- or 6-membered heteroaryl or 5- or 6-membered aryl, wherein the heteroaryl or aryl groups are optionally substituted by one or more substituent selected from C1-C6 alkyl (preferably methyl) and halo (preferably fluoro or chloro); preferably R¹ represents a 5-membered heteroaryl; more preferably R¹ a represents furyl;
R² represents a 6-membered aryl or 6-membered heteroaryl,
wherein the heteroaryl or aryl groups are optionally substituted by one or more substituent selected from halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino and alkylsulfonealkyl;
said substituents being optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl alkylsulfonyl and alkylsulfonealkyl;
or the heteroaryl or aryl groups are optionally substituted with two substituents that form together with the atoms to which they are attached a 5- or 6-membered aryl ring, a 5- or 6-membered heteroaryl ring, a 5- or 6-membered cycloalkyl ring or a 5- or 6-membered heterocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In one embodiment, an A_{2A}R antagonist is a compound of Formula (Ia):

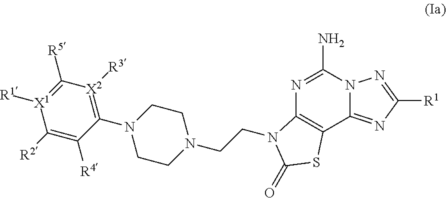

or a pharmaceutically acceptable salt or solvate thereof,
wherein:
R¹ represents a 5- or 6-membered heteroaryl or 5- or 6-membered aryl, wherein the heteroaryl or aryl groups are optionally substituted by one or more substituent selected from C1-C6 alkyl (preferably methyl) and halo (preferably fluoro or chloro); preferably R¹ represents a 5-membered heteroaryl; more preferably R¹ represents a furyl;
X¹ and X² represent each independently C or N;
R^{1′} is absent when X¹ is N; or when X¹ is C, R^{1′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino or alkylsulfonealkyl;
said R^{1′} being optionally substituted where appropriate by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;
R^{2′} represents H, halo, alkyl, heterocyclyl, alkoxy, cycloalkyloxy, heterocyclyloxy, carbonyl, alkylcarbonyl, aminocarbonyl, hydroxycarbonyl, heterocyclylcarbonyl, alkylsulfoxide, alkylsulfonyl, aminosulfonyl, heterocyclylsulfonyl, alkylsulfonimidoyl, carbonylamino, sulfonylamino, or alkylsulfonealkyl;
said R^{2′} being optionally substituted where appropriate by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;
or R^{1′} and R^{2′} form together with the atoms to which they are attached a 5- or 6-membered aryl ring, a 5- or 6-membered heteroaryl ring, a 5- or 6-membered cycloalkyl ring or a 5- or 6-membered heterocyclyl ring; optionally substituted by one or more substituent selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl;
R^{3′} is absent when X² is N; or when X² is C, R^{3′} represents H or halo, preferably H or F;
R^{4′} represents H or halo, preferably H or F; and
R^{5′} represents H or halo, preferably H or F.

In some embodiments, an A_{2A}R antagonist is a compound of Formula (Ia-1):

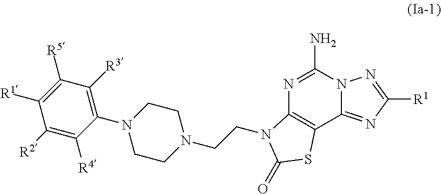

or a pharmaceutically acceptable salt or solvate thereof, wherein R¹, R^{1′}, R^{2′}, R^{3′}, R^{4′} and R^{5′} are as defined in Formula (Ia).

In some embodiments, an A_{2A}R antagonist is a compound of Formula (Ia-1a):

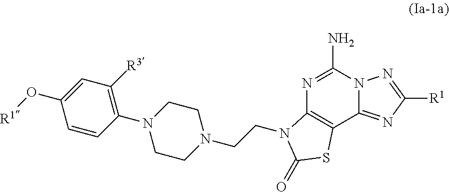

or a pharmaceutically acceptable salt or solvate thereof,
wherein:
R¹ and R^{3′} are as defined in Formula (Ia); and
R^{1′} represents an alkyl or heterocyclyl group substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In one embodiment, a A_{2A}R antagonist is a compound of Formula (Ia-1b):

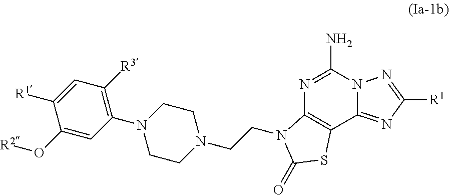

or a pharmaceutically acceptable salt or solvate thereof,
wherein:
R¹ and R^{3′} are as defined in Formula (Ia);
R^{1′} represents H or halo, preferably H or F; and
R^{2″} represents an alkyl or heterocyclyl group substituted by one or more group selected from oxo, halo, hydroxy, cyano, alkyl, alkenyl, aldehyde, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkyne, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxide, alkylsulfoxidealkyl, alkylsulfonyl and alkylsulfonealkyl.

In one embodiment, an A_{2A}R antagonist is a compound of Formula (Ia-1c) or (Ia-1d):

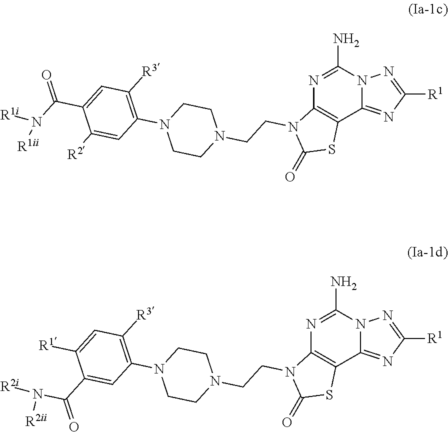

or a pharmaceutically acceptable salt or solvate thereof,
wherein:
R¹ and R^{3′} are as defined in Formula (Ia);
R^{1′} represents H or halo, preferably H or F;
R^{2′} represents H or halo, preferably H or F;
R¹ⁱ and R¹ⁱⁱ represent each independently hydrogen, hydroxy, alkyl, alkenyl, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynealkyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxidealkyl or alkylsulfonealkyl; and
R²ⁱ and R²ⁱⁱ represent each independently hydrogen, hydroxy, alkyl, alkenyl, heterocyclylalkyl, hydroxyalkyl, dihydroxyalkyl, hydroxyalkylaminoalkyl, aminoalkyl, alkylaminoalkyl, dialkylaminoalkyl, (heterocyclyl)(alkyl)aminoalkyl, heterocyclyl, heteroaryl, alkylheteroaryl, alkynealkyl, alkoxy, amino, dialkylamino, aminoalkylcarbonylamino, aminocarbonylalkylamino, (aminocarbonylalkyl)(alkyl)amino, alkenylcarbonylamino, hydroxycarbonyl, alkyloxycarbonyl, aminocarbonyl, aminoalkylaminocarbonyl, alkylaminoalkylaminocarbonyl, dialkylaminoalkylaminocarbonyl, heterocyclylalkylaminocarbonyl, (alkylaminoalkyl)(alkyl)aminocarbonyl, alkylaminoalkylcarbonyl, dialkylaminoalkylcarbonyl, heterocyclylcarbonyl, alkenylcarbonyl, alkynylcarbonyl, alkylsulfoxidealkyl or alkylsulfonealkyl.

In some embodiments, an A_{2A}R antagonist is a compound of Formulae (Ia-2) or (IIa-3):

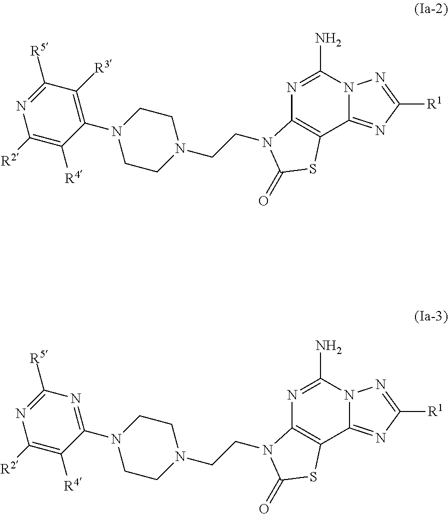

or a pharmaceutically acceptable salt or solvate thereof,
wherein R¹, R^{2′}, R^{3′}, R^{4′} and R^{5′} are as defined in Formula (Ia).

In some embodiments an A_{2A}R antagonist is a compound selected from the group consisting of:
3-(2-(4-(4-((1H-1,2,3-triazolo-4-yl)methoxy-2-fluorophenyl)piperazine-1-yl)ethyl)-5-amino-(8-(furan-2-yl)thiazolo[5,4-e][1,2,4]triazolo[1,5-c]pyrimidine-2(3H)-one;
5-((4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-3-fluorophenoxy)methyl)-1,3,4-oxadiazol-2(3H)-one;
5-amino-3-(2-(4-(3-fluoropyridin-4-yl)piperazin-1-yl)ethyl)-8-(furan-2-yl)thiazolo[5,4-e][1,2,4]triazolo[1,5-c]pyrimidin-2(3H)-one;
2-(5-(4-(2-(5-amino-8-(furan-2-yl)-2-oxothiazolo[5,4-e][1,2,4]triazolo [1,5-c]pyrimidin-3(2H)-yl)ethyl)piperazin-1-yl)-2,4-difluorophenoxy)acetamide;
(S)-5-amino-3-(2-(4-(2-fluoro-4-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2-fluoro-4-(2-(methylsulfinyl)ethoxy)phenyl)-piperazin-1-yl)ethyl)-8-(furan-2-yl)thiazolo[5,4-e][1,2,4]triazolo[1,5-c]pyrimidin-2(3H)-one;
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(−)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
5-amino-8-(furan-2-yl)-3-(2-(4-(4-(2-hydroxyethoxy) phenyl)piperazin-1-yl)ethyl)thiazolo[5,4-e][1,2,4]triazolo[1,5-c]pyrimidin-2(3H)-one;
2-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)phenoxy)acetic acid;
2-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)phenoxy)acetamide;
5-amino-3-(2-(4-(4-(2,3-dihydroxypropoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(4-(2-aminoethoxy)phenyl)piperazin-1-yl)ethyl)-8-(furan-2-yl)thiazolo[5,4-e][1,2,4]triazolo[1,5-c]pyrimidin-2(3H)-one;
4-(4-(2-(5-amino-8-(furan-2-yl)-2-oxothiazolo[5,4-e][1,2,4]triazolo[1,5-c]pyrimidin-3(2H)-yl) ethyl)piperazin-1-yl)benzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-methylbenzamide;
5-amino-8-(furan-2-yl)-3-(2-(4-(4-(2-morpholinoethoxy)phenyl)piperazin-1-yl)ethyl)thiazolo[5,4-e][1,2,4]triazolo[1,5-c]pyrimidin-2(3H)-one;
5-amino-3-(2-(4-(4-(2-(dimethylamino)ethoxy)phenyl)piperazin-1-yl)ethyl)-8-(furan-2-yl)thiazolo[5,4-e][1,2,4]triazolo[1,5-c]pyrimidin-2(3H)-one;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)benzenesulfonamide;
4-(4-(2-(5-amino-8-(furan-2-yl)-2-oxothiazolo[5,4-e][1,2,4]triazolo[1,5-c]pyrimidin-3(2H)-yl)ethyl) piperazin-1-yl)-N-methylbenzenesulfonamide;
5-amino-8-(furan-2-yl)-3-(2-(4-(4-(methylsulfonyl) phenyl)piperazin-1-yl)ethyl)thiazolo[5,4-e][1,2,4]triazolo[1,5-c]pyrimidin-2(3H)-one;
5-amino-8-(furan-2-yl)-3-(2-(4-(4-(methylsulfinyl) phenyl)piperazin-1-yl)ethyl)thiazolo[5,4-e][1,2,4]triazolo[1,5-c]pyrimidin-2(3H)-one;
3-(4-(2-(5-amino-8-(furan-2-yl)-2-oxothiazolo[5,4-e][1,2,4]triazolo[1,5-c]pyrimidin-3(2H)-yl)ethyl)piperazin-1-yl)benzamide;
5-amino-8-(furan-2-yl)-3-(2-(4-(3-(2-hydroxyethoxy) phenyl)piperazin-1-yl)ethyl)thiazolo[5,4-e][1,2,4]triazolo[1,5-c]pyrimidin-2(3H)-one;
5-amino-3-(2-(4-(2-fluoro-4-(2-oxo-2-(piperazin-1-yl)ethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(piperidin-4-ylmethoxy) phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(piperazine-1-carbonyl) phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(2-(piperazin-1-yl)ethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(piperazin-1-ylsulfonyl)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(methylsulfonyl)phenyl) piperazin-1-yl)ethyl)-8-(furan-2-yl)thiazolo[5,4-e][1,2,4]triazolo[1,5-c]pyrimidin-2(3H)-one;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-aminoethyl)-3-fluorobenzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylamino)ethyl) benzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-3-fluorobenzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-hydroxyethyl)benzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2,3-dihydroxypropyl)-3-fluorobenzamide;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)acetic acid;
2-(4-(4-(2-(5-amino-8-(furan-2-yl)-2-oxothiazolo [5,4-e][1,2,4]triazolo[1,5-c]pyrimidin-3(2H)-yl)ethyl) piperazin-1-yl)-3,5-difluorophenoxy) acetic acid;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)propanoic acid;
(S)-2-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-3-fluorophenoxy)propanoic acid;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-2-methylpropanoic acid;
3-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenyl)propanoic acid;
4-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-3-fluorophenoxy)butanoic acid;
2-(3-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,6-difluorophenoxy) acetic acid;
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy) acetic acid;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorobenzoic acid;
2-((2-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-3-fluorophenoxy)ethyl) amino)acetamide;
2-((2-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-3-fluorophenoxy)ethyl)(methyl)amino)acetamide;
5-amino-3-(2-(4-(2-fluoro-4-(piperidin-4-yloxy) phenyl)piperazin-1-yl) ethyl)-8-(furan-2-yl) thiazolo[5,4-e][1,2,4]triazolo[1,5-c]pyrimidin-2(3H)-one;
5-amino-3-(2-(4-(2-fluoro-4-(pyrrolidin-3-yloxy)phenyl)piperazin-1-yl)ethyl)-8-(furan-2-yl)thiazolo[5,4-e][1,2,4]triazolo[1,5-c]pyrimidin-2(3H)-one;
3-(2-(4-(4-((1H-1,2,4-triazol-3-yl)methoxy)-2-fluorophenyl)piperazin-1-yl)ethyl)-5-amino-8-(furan-2-yl)thiazolo[5,4-e][1,2,4]triazolo[1,5-c]pyrimidin-2(3H)-one;
2-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-3-fluorophenoxy)-N-(2-(methylamino)ethyl) acetamide;
2-(4-(4-(2-(5-amino-8-(furan-2-yl)-2-oxothiazolo [5,4-e][1,2,4]triazolo[1,5-c]pyrimidin-3(2H)-yl) ethyl)piperazin-1-yl)-3-fluorophenoxy)-N-(2-(dimethylamino)ethyl) acetamide;
2-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-3-fluorophenoxy)-N-(2-aminoethyl)acetamide;
(R)-2-(4-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-3-fluorophenoxy)propanoic acid;
2-(4-(4-(2-(5-amino-8-(furan-2-yl)-2-oxothiazolo [5,4-e][1,2,4]triazolo[1,5-c]pyrimidin-3(2H)-yl) ethyl)piperazin-1-yl)-3-fluorophenoxy)acetamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-methyl-N-(2-(methylamino)ethyl) benzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-3-fluoro-N-methylbenzamide;
(R)-4-(4-(2-(5-amino-8-(furan-2-yl)-2-oxothiazolo [5,4-e][1,2,4]triazolo[1,5-c]pyrimidin-3(2H)-yl) ethyl)piperazin-1-yl)-N-(1-(dimethylamino) propan-2-yl)-3-fluorobenzamide;
2-(4-(4-(2-(5-amino-8-(furan-2-yl)-2-oxothiazolo [5,4-e][1,2,4]triazolo[1,5-c]pyrimidin-3(2H)-yl) ethyl)piperazin-1-yl)-3-fluorophenoxy)-N-methyl-N-(2-(methylamino)ethyl) acetamide;
2-(5-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-2,4-difluorophenoxy)-2-methylpropanoic acid;
(S)-2-(5-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-2,4-difluorophenoxy) propanoic acid;
(R)-2-(5-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-2,4-difluorophenoxy) propanoic acid;
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)-N-(2-(methylamino)ethyl) acetamide;
2-(5-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-2,4-difluorophenoxy)-N-(2-(dimethylamino)ethyl) acetamide;
5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-2,4-difluoro-N-methylbenzamide;
4-(5-(4-(2-(5-amino-8-(furan-2-yl)-2-oxothiazolo [5,4-e][1,2,4]triazolo[1,5-c]pyrimidin-3(2H)-yl)ethyl)piperazin-1-yl)-2,4-difluorophenoxy) butanoic acid;
3-(2-(4-(5-((1H-tetrazol-5-yl)methoxy)-2,4-difluorophenyl)piperazin-1-yl)ethyl)-5-amino-8-(furan-2-yl)thiazolo[5,4-e][1,2,4]triazolo[1,5-c]pyrimidin-2(3H)-one;
5-amino-3-(2-(4-(2-fluoro-4-((1-methyl-1H-1,2,4-triazol-3-yl)methoxy) phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-((1-methyl-1H-1,2,4-triazol-3-yl) methoxy)phenyl)piperazin-1-yl)ethyl)-8-(furan-2-yl)thiazolo[5,4-e][1,2,4]triazolo[1,5-c]pyrimidin-2(3H)-one;
4-(4-(2-(5-amino-8-(furan-2-yl)-2-oxothiazolo[5,4-e][1,2,4]triazolo[1,5-c]pyrimidin-3(2H)-yl) ethyl)piperazin-1-yl)-3-fluoro-N-(2-(methyl (oxetan-3-yl)amino)ethyl) benzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-((2-hydroxyethyl)amino)ethyl)benzamide;
2-amino-N-(2-(4-(4-(2-(5-amino-8-(furan-2-yl)-2-oxothiazolo[5,4-e][1,2,4]triazolo[1,5-c]pyrimidin-3(2H)-yl) ethyl)piperazin-1-yl)-3-fluorophenoxy)ethyl) acetamide;
(S)-2-amino-N-(2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)ethyl)-3-methylbutanamide;
ethyl 2-(5-(4-(2-(5-amino-8-(furan-2-yl)-2-oxothiazolo[5,4-e][1,2,4]triazolo[1,5-c]pyrimidin-3(2H)-yl) ethyl)piperazin-1-yl)-2,4-difluorophenoxy) acetate;
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy) acetonitrile;
5-amino-8-(furan-2-yl)-3-(2-(4-(pyridin-4-yl) piperazin-1-yl)ethyl)thiazolo[5,4-e][1,2,4]triazolo[1,5-c]pyrimidin-2(3H)-one;
5-amino-8-(furan-2-yl)-3-(2-(4-(pyrimidin-4-yl)piperazin-1-yl)ethyl)thiazolo[5,4-e][1,2,4]triazolo[1,5-c]pyrimidin-2(3H)-one;
5-amino-3-(2-(4-(2,4-difluoro-5-(2-(methylsulfonyl)ethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(2-(methylsulfonyl)ethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(6-fluoro-2-oxoindolin-5-yl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(S-methylsulfonimidoyl)phenyl)piperazin-1-yl)ethyl)-8-(furan-2-yl)thiazolo[5,4-e][1,2,4]triazolo[1,5-c]pyrimidin-2(3H)-one;
5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2-(dimethylamino)ethyl)-2,4-difluorobenzamide;
5-amino-3-(2-(4-(5-fluoro-2-methylpyridin-4-yl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((3R,4R)-4-hydroxytetrahydrofuran-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((3S,4S)-4-hydroxytetrahydrofuran-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(2-hydroxy-2-methylpropoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(2-hydroxypropan-2-yl)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(3,3,3-trifluoro-2-hydroxypropoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-5-(2-hydroxyethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(morpholin-2-ylmethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(morpholin-3-ylmethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(((3S,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(((3S,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(((3R,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(((3S,4R)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
(S)-5-amino-3-(2-(4-(2,4-difluoro-5-((2-oxopyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2,4-difluoro-5-((2-oxopyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
2-(5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluorophenoxy)-N-(2-morpholinoethyl)acetamide;
5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-(morpholin-3-ylmethyl)benzamide;
5-amino-3-(2-(4-(2-fluoro-4-(morpholin-3-ylmethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(morpholin-2-ylmethoxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((3R,4R)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((3S,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((3R,4S)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((3S,4R)-4-fluoropyrrolidin-3-yl)oxy)phenyl)piperazin-1-yl)ethyl)-8-(furan-2-yl)thiazolo[5,4-e][1,2,4]triazolo[1,5-c]pyrimidin-2(3H)-one;
2-(4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluorophenoxy)-N-(2-morpholinoethyl)acetamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-morpholinoethyl)benzamide;
4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(morpholin-3-ylmethyl)benzamide;
5-amino-3-(2-(4-(4-(azetidin-3-yloxy)phenyl)piperazin-1-yl)ethyl)-8-(furan-2-yl)thiazolo[5,4-e][1,2,4]triazolo[1,5-c]pyrimidin-2(3H)-one;
(S)-5-amino-3-(2-(4-(2,4-difluoro-5-(methylsulfinyl)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2,4-difluoro-5-(methylsulfinyl)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(((1s,4s)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(((1r,4r)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one;
(S)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-(2-(methylsulfinyl)ethyl)benzamide;
(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-(2-(methylsulfinyl)ethyl)benzamide;
(S)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-methyl-N-(2-(methylsulfinyl)ethyl)benzamide;
(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-2,4-difluoro-N-methyl-N-(2-(methylsulfinyl)ethyl)benzamide;
5-amino-3-(2-(4-(2,4-difluoro-5-(1-oxidothiomorpholine-4-carbonyl)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2,4-difluoro-5-(1-oxidothiomorpholino)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2-fluoro-4-(methylsulfinyl)phenyl)piperazin-1-yl)ethyl)-8-(furan-2-yl)thiazolo[5,4-e][1,2,4]triazolo[1,5-c]pyrimidin-2(3H)-one;
(S)-5-amino-3-(2-(4-(2-fluoro-4-(methylsulfinyl)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((1s,4s)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(((1r,4r)-1-oxidotetrahydro-2H-thiopyran-4-yl)oxy)phenyl)piperazin-1-yl)ethyl)-8-(furan-2-yl)thiazolo[5,4-e][1,2,4]triazolo[1,5-c]pyrimidin-2(3H)-one;
(S)-4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylsulfinyl)ethyl)benzamide;
(R)-4-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-3-fluoro-N-(2-(methylsulfinyl)ethyl)benzamide;
5-amino-3-(2-(4-(2-fluoro-4-(1-oxidothiomorpholine-4-carbonyl)phenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(2-fluoro-4-(1-oxidothiomorpholino)phenyl)piperazin-1-yl)ethyl)-8-(furan-2-yl)thiazolo[5,4-e][1,2,4]triazolo[1,5-c]pyrimidin-2(3H)-one;
(S)-5-amino-3-(2-(4-(5-(2,3-dihydroxypropoxy)-2,4-difluorophenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(5-(2,3-dihydroxypropoxy)-2,4-difluorophenyl)piperazin-1-yl)ethyl)-8-(furan-2-yl)thiazolo[5,4-e][1,2,4]triazolo[1,5-c]pyrimidin-2(3H)-one;
(S)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2,3-dihydroxypropyl)-2,4-difluorobenzamide;
(R)-5-(4-(2-(5-amino-8-(furan-2-yl)-2-oxothiazolo[5,4-e][1,2,4]triazolo[1,5-c]pyrimidin-3(2H)-yl)ethyl)piperazin-1-yl)-N-(2,3-dihydroxypropyl)-2,4-difluorobenzamide;
5-amino-3-(2-(4-(4-(azetidin-3-yloxy)-2-fluorophenyl)piperazin-1-yl)ethyl)-8-(furan-2-yl)thiazolo[5,4-e][1,2,4]triazolo[1,5-c]pyrimidin-2(3H)-one;
5-amino-3-(2-(4-(5-(azetidin-3-yloxy)-2,4-difluorophenyl)piperazin-1-yl)ethyl)-8-(furan-2-yl)thiazolo[5,4-e][1,2,4]triazolo[1,5-c]pyrimidin-2(3H)-one; and
(S)-5-amino-3-(2-(4-(2,4-difluoro-5-(3-(methylsulfinyl)propoxy)phenyl)piperazin-1-yl)ethyl)-8-(furan-2-yl)thiazolo[5,4-e][1,2,4]triazolo[1,5-c]pyrimidin-2(3H)-one,
   or a pharmaceutically acceptable salt or solvate thereof.

In some embodiments, an A_{2A}R antagonist is selected from the group consisting of:
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(+)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one; and
(−)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one,
   or a pharmaceutically acceptable salt or solvate thereof.

In some embodiments, an A_{2A}R antagonist is selected from the group consisting of:
(R,S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one; and
(S)-5-amino-3-(2-(4-(2,4-difluoro-5-(2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one,
   or a pharmaceutically acceptable salt or solvate thereof.

In some embodiments, an A_{2A}R antagonist is (R,S)-5-amino-3-(2-(4-(2,4-difluoro-5-(92-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one or a pharmaceutically acceptable salt or solvate thereof.

In some embodiments, an A_{2A}R antagonist is (+)-5-amino-3-(2-(4-(2,4-difluoro-5-((2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one or a pharmaceutically acceptable salt or solvate thereof.

In some embodiments, an A_{2A}R antagonist is (−)-5-amino-3-(2-(4-(2,4-difluoro-5-((2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one or a pharmaceutically acceptable salt or solvate thereof.

In some embodiments, an A_{2A}R antagonist is (R)-5-amino-3-(2-(4-(2,4-difluoro-5-((2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one or a pharmaceutically acceptable salt or solvate thereof.

In some embodiments, an A_{2A}R antagonist is (S)-5-amino-3-(2-(4-(2,4-difluoro-5-((2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one or a pharmaceutically acceptable salt or solvate thereof.

In some embodiments, an A_{2A}R antagonist is (R)-5-amino-3-(2-(4-(2,4-difluoro-5-((2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one or a pharmaceutically acceptable salt thereof.

In some embodiments, an A_{2A}R antagonist is (S)-5-amino-3-(2-(4-(2,4-difluoro-5-((2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one or a pharmaceutically acceptable salt thereof. In some embodiments, the salt is a hydrochloride salt, for example, as disclosed in WO 2023/059817.

In some embodiments, an A_{2A}R antagonist is (R)-5-amino-3-(2-(4-(2,4-difluoro-5-((2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one.

In some embodiments, an A_{2A}R antagonist is (S)-5-amino-3-(2-(4-(2,4-difluoro-5-((2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one.

In another embodiment, an A_{2A}R antagonist is an A_{2A}R antagonist disclosed in WO2011/121418. Especially, an A_{2A}R antagonist is the compound of example 1 of WO2011/121418, namely 5-bromo-2,6-di-(1H-pyrazol-1-yl)pyrimidin-4-amine, also known as NIR178:

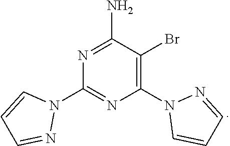 [SMILES: Nc1nc(-n2cccn2)nc(-n2cccn2)c1Br]

In another embodiment, an A_{2A}R antagonist is an A_{2A}R antagonist disclosed in WO2009/156737. Especially, an A_{2A}R antagonist is the compound of example 1S of WO2009/156737, namely (S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine, also known as CPI-444:

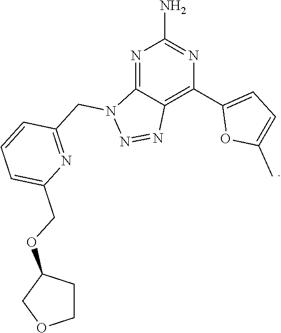 [SMILES: Cc1ccc(-c2nc(N)nc3c2nnn3Cc2cccc(CO[C@H]3CCOC3)n2)o1]

In another embodiment, an A_{2A}R antagonist is an A_{2A}R antagonist disclosed in WO2011/095626. Especially, the A_{2A}R antagonist is the compound (cxiv) of WO2011/095626, namely 6-(2-chloro-6-methylpyridin-4-yl)-5-(4-fluorophenyl)-1,2,4-triazin-3-amine, also known as AZD4635:

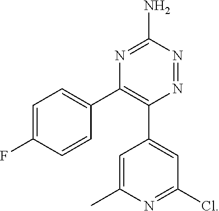 [SMILES: Cc1cc(-c2nnc(N)nc2-c2ccc(F)cc2)cc(Cl)n1]

In another embodiment, an A_{2A}R antagonist is an A_{2A}R antagonist disclosed in WO2018/136700. Especially, the A_{2A}R antagonist is the compound of example 1 of WO2018/136700, namely 3-(2-amino-6-(1-((6-(2-hydroxypropan-2-yl)pyridin-2-yl)methyl)-1H-1,2,3-triazol-4-yl)pyrimidin-4-yl)-2-methylbenzonitrile, also known as AB928:

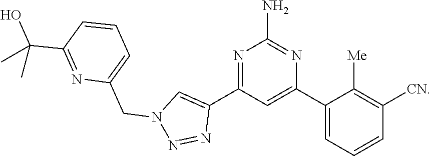 [SMILES: Cc1c(C#N)cccc1-c1cc(-c2cn(Cc3cccc(C(C)(C)O)n3)nn2)nc(N)n1]

In another embodiment, an A_{2A}R antagonist is Preladenant (SCH-420,814), namely 2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine:

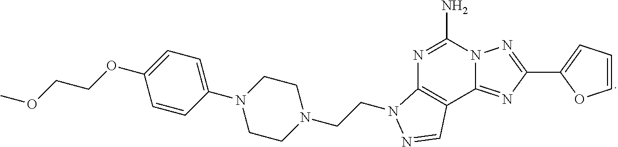 [SMILES: COCCOc1ccc(N2CCN(CCn3ncc4c3nc(N)n3nc(-c5ccco5)nc43)CC2)cc1]

In another embodiment, an A_{2A}R antagonist is Vipadenant (BIIB-014), namely 3-(4-amino-3-methylbenzyl)-7-(2-furyl)-3H-(1,2,3)triazolo(4,5-d)pyrimidine-5-amine:

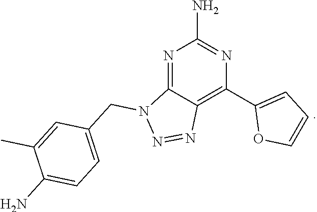 [SMILES: Cc1cc(Cn2nnc3c(-c4ccco4)nc(N)nc32)ccc1N]

In another embodiment, an A_{2A}R antagonist is Tozadenant (SYK-115), namely 4-hydroxy-N-(4-methoxy-7-morpholinobenzo[d]thiazol-2-yl)-4-methylpiperidine-1-carboxamide:

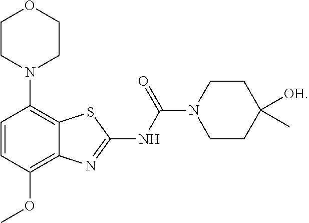 [SMILES: COc1ccc(N2CCOCC2)c2sc(NC(=O)N3CCC(C)(O)CC3)nc12]

In one embodiment, an adenosine receptor antagonist is selected from:
5-bromo-2,6-di-(1H-pyrazol-1-yl)pyrimidin-4-amine;
(S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine;
6-(2-chloro-6-methylpyridin-4-yl)-5-(4-fluorophenyl)-1,2,4-triazin-3-amine;
3-(2-amino-6-(1-((6-(2-hydroxypropan-2-yl)pyridin-2-yl)methyl)-1H-1,2,3-triazol-4-yl)pyrimidin-4-yl)-2-methylbenzonitrile;
2-(2-furanyl)-7-(2-(4-(4-(2-methoxyethoxy)phenyl)-1-piperazinyl)ethyl)-7H-pyrazolo(4,3-e)(1,2,4)triazolo(1,5-c)pyrimidine-5-amine;
3-(4-amino-3-methylbenzyl)-7-(2-furyl)-3H-(1,2,3)triazolo(4,5-d)pyrimidine-5-amine; and
4-hydroxy-N-(4-methoxy-7-morpholinobenzo[d]thiazol-2-yl)-4-methylpiperidine-1-carboxamide;
(R,S)-5-amino-3-(2-(4-(2,4-difluoro-5-((2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(R)-5-amino-3-(2-(4-(2,4-difluoro-5-((2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
(S)-5-amino-3-(2-(4-(2,4-difluoro-5-((2-(methylsulfinyl)ethoxy)phenyl)piperazin-1-yl)ethyl)-8-(furan-2-yl)thiazolo[5,4-e][1,2,4]triazolo[1,5-c]pyrimidin-2(3H)-one;
   or a pharmaceutically acceptable salts thereof.

In one embodiment, an adenosine receptor antagonist is 5-bromo-2,6-di-(1H-pyrazol-1-yl)pyrimidin-4-amine. In one embodiment, an adenosine receptor antagonist is (S)-7-(5-methylfuran-2-yl)-3-((6-(([tetrahydrofuran-3-yl]oxy)methyl)pyridin-2-yl)methyl)-3H-[1,2,3]triazolo[4,5-d]pyrimidin-5-amine. In one embodiment, an adenosine receptor antagonist is 6-(2-chloro-6-methylpyridin-4-yl)-5-(4-fluorophenyl)-1,2,4-triazin-3-amine. In one embodiment, an adenosine receptor antagonist is 3-(2-amino-6-(1-((6-(2-hydroxypropan-2-yl)pyridin-2-yl)methyl)-1H-1,2,3-triazol-4-yl)pyrimidin-4-yl)-2-methylbenzonitrile.

2. A_{2B} and A₃ Receptor Antagonists

An “A_{2B}R antagonist” refers to a compound that, upon administration to a patient, results in inhibition or down-regulation of a biological activity associated with activation of A_{2B} receptor in the patient, including any of the downstream biological effects otherwise resulting from the binding to A_{2B} receptor of its natural ligand. Such A_{2B}R antagonists include any agent that can block activation of A_{2B} receptor or any of the downstream biological effects of A_{2B} receptor activation.

Examples of A_{2B}R antagonists include: Vipadenant (BIIB-014), CVT-6883, MRS-1706, MRS-1754, PSB-603, PSB-0788, PSB-1115, OSIP-339,391, ATL-801, theophylline, or Caffeine.

Examples of inhibitors of A_{2B} receptor include ATL-801, CVT-6883, MRS-1706, MRS-1754, OSIP-339,391, PSB-603, PSB-0788 and PSB-1115.

Examples of inhibitors of A₃ receptor include KF-26777, MRS-545, MRS-1191, MRS-1220, MRS-1334, MRS-1523, MRS-3777, MRE-3005-F20, MRE-3008-F20, PSB-11, OT-7999, VUF-5574 and SSR161421.

### D. Combinations, Compositions, and Kits

In some embodiments, disclosed herein are combinations and compositions comprising an ENT1 inhibitor and a checkpoint inhibitor. In some embodiments, the combinations and compositions further comprise an adenosine receptor antagonist, optionally an A_{2A}R antagonist. In some embodiments, the combinations and compositions further comprise a pharmaceutically acceptable excipient.

By means of non-limiting examples, the ENT1 inhibitors, checkpoint inhibitors, adenosine receptor antagonists, combinations and compositions disclosed herein may each independently be in a form suitable for oral administration, for parenteral administration (such as by intravenous, intramuscular or subcutaneous injection or intravenous infusion), for topical administration (including ocular), for administration by inhalation, by a skin patch, by an implant, by a suppository, etc. Such suitable administration forms—which may be solid, semi-solid or liquid, depending on the manner of administration—as well as the methods and carriers, diluents and excipients for use in the preparation thereof, will be clear to the skilled person; reference is made to the latest edition of Remington's Pharmaceutical Sciences.

In some embodiments, such preparations include tablets, pills, powders, lozenges, sachets, cachets, elixirs, suspensions, emulsions, solutions, syrups, aerosols, ointments, cremes, lotions, soft and hard gelatin capsules, suppositories, drops, sterile injectable solutions and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propyl-hydroxybenzoates, talc, magnesium stearate, edible oils, vegetable oils and mineral oils or suitable mixtures thereof.

The ENT1 inhibitors, checkpoint inhibitors, adenosine receptor antagonists, combinations, and compositions described herein can optionally contain or be mixed with other substances that are commonly used in pharmaceutical compositions, such as lubricating agents, wetting agents, emulsifying and suspending agents, dispersing agents, disintegrants, bulking agents, fillers, preserving agents, sweetening agents, flavoring agents, flow regulators, release agents, etc. They may also be formulated so as to provide rapid, sustained or delayed release of the active compound(s) contained therein.

In general, the ENT1 inhibitors, checkpoint inhibitors, adenosine receptor antagonists, combinations, and compositions herein may be prepared according to known methods in pharmaceutical chemistry. Capsules can be prepared by mixing, for example, an ENT1 inhibitor with a suitable carrier or diluent and filling the proper amount of the mixture in capsules. The usual carriers and diluents include, but are not limited to, inert powdered substances such as starch of many different kinds, powdered cellulose, including crystalline and microcrystalline cellulose, sugars such as fructose, mannitol and sucrose, grain flours and similar edible powders.

Tablets can be prepared by direct compression, by wet granulation, or by dry granulation. Their formulations may incorporate diluents, binders, lubricants and disintegrators as well as the compound. Diluents include, for example, various types of starch, lactose, mannitol, kaolin, calcium phosphate or sulfate, inorganic salts such as sodium chloride and powdered sugar. Powdered cellulose derivatives are also useful. Tablet binders may be substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the dye. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. Corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The ENT1 inhibitors, checkpoint inhibitors, adenosine receptor antagonists, combinations, and compositions can also be formulated as chewable tablets, for example, by using substances such as mannitol in the formulation.

When it is desired to administer an ENT1 inhibitor, a checkpoint inhibitor, an adenosine receptor antagonist, a combination, or a composition of the present disclosure as a suppository, a base can be used. Cocoa butter may be used as a suppository base, which can be modified by addition of waxes to raise its melting point slightly. Water-miscible suppository bases comprising, for example, polyethylene glycols of various molecular weights are in wide use.

The effect of the ENT1 inhibitors, checkpoint inhibitors, adenosine receptor antagonists, combinations, and compositions of the present disclosure can be delayed or prolonged by proper formulation. For example, a slowly soluble pellet can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the compound of the present disclosure in oily or emulsified vehicles that allow it to disperse slowly in the serum.

In some embodiments, the ENT1 inhibitors, checkpoint inhibitors, adenosine receptor antagonists, combinations, and compositions of the present disclosure are in a unit dosage form, and may be suitably packaged, for example in a box, blister, vial, bottle, sachet, ampoule or in any other suitable single-dose or multi-dose holder or container (which may be properly labeled), optionally with one or more leaflets containing product information and/or instructions for use.

In some embodiments, the ENT1 inhibitor is administered in a daily dose from about 2.5 mg to about 160 mg. In some embodiments, the ENT inhibitor is administered in a daily dose from about 2.5 mg to about 160 mg, from about 2.5 mg to about 155 mg, from about 2.5 mg to about 150 mg, from about 2.5 mg to about 145 mg, from 2.5 mg to about 140 mg, from about 2.5 mg to about 135 mg, from about 2.5 mg to about 130 mg, from about 2.5 mg to about 125 mg, from about 2.5 mg to about 120 mg, from about 2.5 mg to about 115 mg, from 2.5 mg to about 110 mg, from about 2.5 mg to about 105 mg, about 2.5 mg to about 100 mg, from about 2.5 mg to about 95 mg, from 2.5 mg to about 90 mg, from about 2.5 mg to about 85 mg, from about 2.5 mg to about 80 mg, from about 2.5 mg to about 75 mg, from about 2.5 mg to about 70 mg, from about 2.5 mg to about 65 mg, from about 2.5 mg to about 60 mg, from about 2.5 mg to about 55 mg, from about 2.5 mg to about 50 mg, from about 2.5 mg to about 45 mg, from about 2.5 mg to about 40 mg, from about 2.5 mg to about 35 mg, from about 2.5 mg to about 30 mg, from about 2.5 mg to about 25 mg, from about 2.5 mg to about 20 mg, from about 2.5 mg to about 15 mg, from about 2.5 mg to about 10 mg, or from about 2.5 mg to about 5 mg.

In some embodiments, the ENT inhibitor is administered in a daily dose of about 2.5 mg, 5 mg, 10 mg, 15 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 145 mg, 150 mg, 155 mg, or 160 mg. In some embodiments, the ENT inhibitor is administered in a daily dose of about 2.5 mg. In some embodiments, the ENT inhibitor is administered in a daily dose of about 5 mg. In some embodiments, the ENT inhibitor is administered in a daily dose of about 10 mg. In some embodiments, the ENT inhibitor is administered in a daily dose of about 20 mg. In some embodiments, the ENT inhibitor is administered in a daily dose of about 30 mg. In some embodiments, the ENT inhibitor is administered in a daily dose of about 40 mg. In some embodiments, the ENT inhibitor is administered in a daily dose of about 45 mg. In some embodiments, the ENT inhibitor is administered in a daily dose of about 50 mg. In some embodiments, the ENT inhibitor is administered in a daily dose of about 60 mg. In some embodiments, the ENT inhibitor is administered in a daily dose of about 70 mg. In some embodiments, the ENT inhibitor is administered in a daily dose of about 80 mg. In some embodiments, the ENT inhibitor is administered in a daily dose of about 90 mg. In some embodiments, the ENT inhibitor is administered in a daily dose of about 100 mg. In some embodiments, the ENT inhibitor is administered in a daily dose of about 110 mg. In some embodiments, the ENT inhibitor is administered in a daily dose of about 120 mg. In some embodiments, the ENT inhibitor is administered in a daily dose of about 130 mg. In some embodiments, the ENT inhibitor is administered in a daily dose of about 140 mg. In some embodiments, the ENT inhibitor is administered in a daily dose of about 150 mg. In some embodiments, the ENT inhibitor is administered in a daily dose of about 160 mg.

In some embodiments, the ENT1 inhibitor is Compound 1 or a salt, hydrate, or solvate thereof. In some embodiments, the Compound 1 or a salt, hydrate, or solvate thereof is administered in a daily dose (free base) of about 2.5 mg, 5 mg, 10 mg, 15 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 145 mg, 150 mg, 155 mg, or 160 mg.

In some embodiments, Compound 1 or a salt, hydrate, or solvate thereof is administered in a daily dose of about 2.5 mg (free base). In some embodiments, Compound 1 or a salt, hydrate, or solvate thereof is administered in a daily dose of about 5 mg (free base). In some embodiments, Compound 1 or a salt, hydrate, or solvate thereof is administered in a daily dose of about 10 mg (free base). In some embodiments, Compound 1 or a salt, hydrate, or solvate thereof is administered in a daily dose of about 20 mg (free base). In some embodiments, Compound 1 or a salt, hydrate, or solvate thereof is administered in a daily dose of about 30 mg (free base). In some embodiments, Compound 1 or a salt, hydrate, or solvate thereof is administered in a daily dose of about 40 mg (free base). In some embodiments, Compound 1 or a salt, hydrate, or solvate thereof is administered in a daily dose of about 45 mg (free base). In some embodiments, Compound 1 or a salt, hydrate, or solvate thereof is administered in a daily dose of about 50 mg (free base). In some embodiments, Compound 1 or a salt, hydrate, or solvate thereof is administered in a daily dose of about 60 mg (free base). In some embodiments, Compound 1 or a salt, hydrate, or solvate thereof is administered in a daily dose of about 70 mg (free base). In some embodiments, Compound 1 or a salt, hydrate, or solvate thereof is administered in a daily dose of about 80 mg (free base). In some embodiments, Compound 1 or a salt, hydrate, or solvate thereof is administered in a daily dose of about 90 mg (free base). In some embodiments, Compound 1 or a salt, hydrate, or solvate thereof is administered in a daily dose of about 100 mg (free base). In some embodiments, Compound 1 or a salt, hydrate, or solvate thereof is administered in a daily dose of about 110 mg (free base). In some embodiments, Compound 1 or a salt, hydrate, or solvate thereof is administered in a daily dose of about 120 mg (free base). In some embodiments, Compound 1 or a salt, hydrate, or solvate thereof is administered in a daily dose of about 130 mg (free base). In some embodiments, Compound 1 or a salt, hydrate, or solvate thereof is administered in a daily dose of about 140 mg (free base). In some embodiments, Compound 1 or a salt, hydrate, or solvate thereof is administered in a daily dose of about 150 mg (free base). In some embodiments, Compound 1 or a salt, hydrate, or solvate thereof is administered in a daily dose of about 160 mg (free base).

In some embodiments, the ENT1 inhibitor is a Compound 1 hydrogen sulfate or hydrate or solvate thereof, such as crystalline Form 2 Compound 1 di(hydrogen sulfate) trihydrate. In some embodiments, the Compound 1 hydrogen sulfate or hydrate or solvate thereof, for example crystalline Form 2 Compound 1 di(hydrogen sulfate) trihydrate, is administered in a daily dose (free base) of about 2.5 mg, 5 mg, 10 mg, 15 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 145 mg, 150 mg, 155 mg, or 160 mg.

In some embodiments, the Compound 1 hydrogen sulfate or hydrate or solvate thereof, for example crystalline Form 2 Compound 1 di(hydrogen sulfate) trihydrate, is administered in a daily dose of about 2.5 mg (free base). In some embodiments, the Compound 1 hydrogen sulfate or hydrate or solvate thereof, for example crystalline Form 2 Compound 1 di(hydrogen sulfate) trihydrate, is administered in a daily dose of about 5 mg (free base). In some embodiments, the Compound 1 hydrogen sulfate or hydrate or solvate thereof, for example crystalline Form 2 Compound 1 di(hydrogen sulfate) trihydrate, is administered in a daily dose of about 10 mg (free base). In some embodiments, the Compound 1 hydrogen sulfate or hydrate or solvate thereof, for example crystalline Form 2 Compound 1 di(hydrogen sulfate) trihydrate, is administered in a daily dose of about 20 mg (free base). In some embodiments, the Compound 1 hydrogen sulfate or hydrate or solvate thereof, for example crystalline Form 2 Compound 1 di(hydrogen sulfate) trihydrate, is administered in a daily dose of about 30 mg (free base). In some embodiments, the Compound 1 hydrogen sulfate or hydrate or solvate thereof, for example crystalline Form 2 Compound 1 di(hydrogen sulfate) trihydrate, is administered in a daily dose of about 40 mg (free base). In some embodiments, the Compound 1 hydrogen sulfate or hydrate or solvate thereof, for example crystalline Form 2 Compound 1 di(hydrogen sulfate) trihydrate, is administered in a daily dose of about 45 mg (free base). In some embodiments, the Compound 1 hydrogen sulfate or hydrate or solvate thereof, for example crystalline Form 2 Compound 1 di(hydrogen sulfate) trihydrate, is administered in a daily dose of about 50 mg (free base). In some embodiments, the Compound 1 hydrogen sulfate or hydrate or solvate thereof, for example crystalline Form 2 Compound 1 di(hydrogen sulfate) trihydrate, is administered in a daily dose of about 60 mg (free base). In some embodiments, the Compound 1 hydrogen sulfate or hydrate or solvate thereof, for example crystalline Form 2 Compound 1 di(hydrogen sulfate) trihydrate, is administered in a daily dose of about 70 mg (free base). In some embodiments, the Compound 1 hydrogen sulfate or hydrate or solvate thereof, for example crystalline Form 2 Compound 1 di(hydrogen sulfate) trihydrate, is administered in a daily dose of about 80 mg (free base). In some embodiments, the Compound 1 hydrogen sulfate or hydrate or solvate thereof, for example crystalline Form 2 Compound 1 di(hydrogen sulfate) trihydrate, is administered in a daily dose of about 90 mg (free base). In some embodiments, the Compound 1 hydrogen sulfate or hydrate or solvate thereof, for example crystalline Form 2 Compound 1 di(hydrogen sulfate) trihydrate, is administered in a daily dose of about 100 mg (free base). In some embodiments, the Compound 1 hydrogen sulfate or hydrate or solvate thereof, for example crystalline Form 2 Compound 1 di(hydrogen sulfate) trihydrate, is administered in a daily dose of about 110 mg (free base). In some embodiments, the Compound 1 hydrogen sulfate or hydrate or solvate thereof, for example crystalline Form 2 Compound 1 di(hydrogen sulfate) trihydrate, is administered in a daily dose of about 120 mg (free base). In some embodiments, the Compound 1 hydrogen sulfate or hydrate or solvate thereof, for example crystalline Form 2 Compound 1 di(hydrogen sulfate) trihydrate, is administered in a daily dose of about 130 mg (free base). In some embodiments, the Compound 1 hydrogen sulfate or hydrate or solvate thereof, for example crystalline Form 2 Compound 1 di(hydrogen sulfate) trihydrate, is administered in a daily dose of about 140 mg (free base). In some embodiments, the Compound 1 hydrogen sulfate or hydrate or solvate thereof, for example crystalline Form 2 Compound 1 di(hydrogen sulfate) trihydrate, is administered in a daily dose of about 150 mg (free base). In some embodiments, the Compound 1 hydrogen sulfate or hydrate or solvate thereof, for example crystalline Form 2 Compound 1 di(hydrogen sulfate) trihydrate, is administered in a daily dose of about 160 mg (free base).

In some embodiments, the ENT inhibitor is administered once daily during a treatment cycle. In some embodiments, the ENT inhibitor is administered twice daily (BID) during a treatment cycle.

In some embodiments, the treatment comprises at least 1, 2, 3, 4, 5, 6, 7, 8, 9, or 10 treatment cycles. In some embodiments, the treatment comprises at least one treatment cycle of 21-28 days. In some embodiments, the treatment cycle is 21, 22, 23, 24, 25, 26, 27, or 28 days. In some embodiments, the treatment cycle is 21 days. In some embodiments, the treatment cycle is 28 days.

In some embodiments, the ENT inhibitor is administered continuously during the treatment cycle. In some embodiments, the ENT inhibitor is administered intermittently during the treatment cycle. In some embodiments, the intermittent dosing comprises 5 days of ENT inhibitor treatment and 2 days of no ENT inhibitor treatment in a 21-day cycle. In some embodiments, the intermittent dosing comprises 3 weeks of ENT inhibitor treatment and 1 week of no ENT inhibitor treatment in 28-day cycle.

In some embodiments, the ENT inhibitor is administered at the same dose during the treatment cycle. In some embodiments, the ENT inhibitor is administered at doses of escalating concentration (i.e., increasing doses). In this case, a subsequent dose can be increased by a particular increment, or by variable increments, until a maximum dose is reached, at which point administration may cease or may continue at the maximum dose. In some embodiments, the ENT inhibitor is administered at about 5 mg for the first week, 10 mg for the second week, etc.

In some embodiments, the ENT inhibitor is administered orally. In some embodiments, the ENT inhibitor is administered as a tablet and/or capsule.

In some embodiments, in the event that the patient develops, or is at risk of developing, an adverse event associated with the administration of the ENT1 inhibitor, the treatment further comprises the administration of an agent capable of treating, preventing, delaying, reducing or attenuating the development or risk of development of the adverse event. The agent may be administered to the patient prior to the initiation of the treatment with the ENT1 inhibitor (e.g., as a prophylaxis in order to prevent or reduce the risk of an adverse event developing) or during or subsequent to treatment with the ENT1 inhibitor (e.g., in response to the development of an adverse event). In some embodiments, the adverse event comprises headache, pain, nausea, and/or vomiting. In some embodiments, the agent is a medication administered to combat headache, pain, nausea, and/or vomiting.

In some embodiments, the agent capable of treating, preventing, delaying, reducing or attenuating the development or risk of development of the adverse event is administered as one or more doses to the patient prior to the initiation of the treatment with the ENT1 inhibitor as a prophylactic treatment for the adverse event.

In some embodiments, the agent capable of treating, preventing, delaying, reducing or attenuating the development or risk of development of the adverse event is administered to the patient in combination with one or more dose of the ENT1 inhibitor as a prophylactic treatment for the adverse event. The agent may be administered as one or more doses consecutively (before and/or after), and/or concurrently with the ENT1 inhibitor.

In some embodiments, the agent capable of treating, preventing, delaying, reducing or attenuating the development or risk of development of the adverse event is administered to the patient in the event that the patient develops an adverse event associated with the administration of the ENT1 inhibitor. In some embodiments, the treatment comprises administration of the agent at a therapeutic amount, or an amount sufficient to partially or completely alleviate or ameliorate the adverse event (e.g. CRS) or symptoms thereof.

In some embodiments, agent is administered prior to, concomitant with, and/or subsequent to at least one, two, three, four, five, six, seven, eight, nine, ten, eleven, twelve, thirteen, fourteen, fifteen, sixteen, seventeen, eighteen, nineteen, twenty, etc. doses of ENT inhibitor. In some embodiments, the agent is administered prior to, concomitant with, and/or subsequent to all doses of ENT inhibitor. In some embodiments, the agent is administered at least 30 minutes before and/or after at least one dose of ENT inhibitor. In some embodiments, the agent is administered 30 minutes before at least one dose of ENT inhibitor.

In some embodiments, the agent comprises an antiemetic, analgesic, and/or caffeine.

In some embodiments, the analgesic comprises a non-steroidal anti-inflammatory drug (NSAID) (e.g., diclofenac, diflunisal, etodolac, fenoprofen, flurbiprofen, ibuprofen, indomethacin, ketoprofen, ketorolac, mefenamic acid, meloxicam, nabumetone, naproxen, oxaprozin, piroxicam, sulindac, tolmetin, celecoxib, rofecoxib, valdecoxib), aspirin, acetaminophen, antidepressive medications (e.g., amitriptyline, duloxetine), antiepileptic medication (e.g., gabapentin, prefabalin), and/or lidocaine.

In some embodiments the antiemetic comprises anticholinergic agents such as hyoscyamine, methscopolamine, scopolamine; antihistamines, such as cyclizine, dimenhydrinate, doxylamine, hydroxyzine, meclizine, promethazine; cannabinoid receptor agonists, such as dronabinol, nabilone, tetrahydrocannabinol; dopamine receptor antagonists, such as chlorpromazine, prochlorperazine; serotonin 5-HT3 receptor antagonists, such as alosetron, dolasetron, granisetron, ondansetron, palonosetron; substance P/neurokinin 1 receptor antagonists, such as aprepitant, fosaprepitant, fosnetupitant, rolapitant; or miscellaneous antiemetic agents, such as amisulpride, dexamethasone, metoclopramide, trimethobenzamide.

In some embodiments, the agent is administered orally. In some embodiments, the agent is administered as a tablet and/or a capsule. In some embodiments, the agent is caffeine. In some embodiments, the caffeine is administered orally as a tablet or capsule. In some embodiments, the caffeine is administered in a beverage or in food.

In some embodiments, the checkpoint inhibitor is administered in a dose of from about 200 mg to about 400 mg. In some embodiments, the checkpoint inhibitor is administered in an amount of about 200 mg. In some embodiments, the checkpoint inhibitor is administered daily, weekly, every two weeks, every three weeks, or every four weeks. In some embodiment, the dosing interval for the checkpoint inhibitor is once every three weeks.

In some embodiments, the checkpoint inhibitor is administered as IV infusion.

In some embodiments, the checkpoint inhibitor is pembrolizumab.

In some embodiments, disclosed herein is a kit of parts comprising an ENT1 inhibitor and a checkpoint inhibitor. In some embodiments, the kit of parts further comprises a product insert. In some embodiments, the insert instructs one to administer the ENT1 inhibitor prior to, concomitant with, or subsequent to the administration of the checkpoint inhibitor. In some embodiments, disclosed herein is a kit of parts comprising an ENT1 inhibitor, a checkpoint inhibitor, and an adenosine receptor antagonist. In some embodiments, the kit of parts further comprises a product insert. In some embodiments, the insert instructs one to administer the ENT1 inhibitor prior to, concomitant with, or subsequent to the administration of the checkpoint inhibitor and/or adenosine receptor antagonist.

In one embodiment, the disclosure provides for a kit of parts as described herein for use in any method described herein. In some embodiments, the kit of parts is for use in the treatment of cancer. The disclosure further provides for a use of the kit of parts as described herein for the manufacture of a medicament for treating cancer. The disclosure further provides a method of treating cancer, which comprises administering to a patient in need thereof an ENT1 inhibitor and a checkpoint inhibitor, and optionally an adenosine receptor antagonist, contained within a kit of parts as described herein.

### E. Methods and Uses

In some embodiments, disclosed herein is a method of reversing adenosine-mediated suppression of T cell proliferation and/or viability, comprising administering an ENT1 inhibitor and a checkpoint inhibitor. In some embodiments, disclosed herein is a method of reversing adenosine-mediated suppression of antigen-specific killing activity of T cells, comprising administering an ENT1 inhibitor and a checkpoint inhibitor. In some embodiments, disclosed herein is a method of reversing adenosine-mediated suppression of production of cytokines comprising administering an ENT1 inhibitor and a checkpoint inhibitor. In some embodiments, the reversal of adenosine-mediated suppression occurs in a cancer.

In some embodiments, disclosed herein is a method of treating cancer comprising administering to a subject in need thereof an ENT1 inhibitor and a checkpoint inhibitor. In some embodiments, disclosed herein is a use of an ENT1 inhibitor and a checkpoint inhibitor in the manufacture of a medicament for the treatment of cancer. In some embodiments, the methods and use disclosed herein further comprises administration or use of an adenosine receptor antagonist.

Various cancers are known in the art. Cancers that can be treated using the methods described herein include solid cancers and non-solid cancers, especially benign and malignant solid tumors and benign and malignant non-solid tumors. The cancer may be metastatic or non-metastatic. The cancer may be familial or sporadic.

In some embodiments, the cancer is a solid-tumor cancer. As used herein, the term “solid cancer” encompasses any cancer (also referred to as malignancy) that forms a discrete tumor mass, as opposed to cancers (or malignancies) that diffusely infiltrate a tissue without forming a mass.

Examples of solid tumors include, but are not limited to: biliary tract cancer, brain cancer (including glioblastomas and medulloblastomas), breast cancer, carcinoid, cervical cancer, choriocarcinoma, colon cancer, colorectal cancer, endometrial cancer, esophageal cancer, gastric cancer, glioma, head and neck cancer, intraepithelial neoplasms (including Bowen's disease and Paget's disease), liver cancer, lung cancer, neuroblastomas, oral cancer (including squamous cell carcinoma), ovarian cancer (including those arising from epithelial cells, stromal cells, germ cells and mesenchymal cells), pancreatic cancer, prostate cancer, rectal cancer, renal cancer (including adenocarcinoma and Wilms tumor), sarcomas (including leiomyosarcoma, rhabdomyosarcoma, liposarcoma, fibrosarcoma and osteosarcoma), skin cancer (including melanoma, Kaposi's sarcoma, basocellular cancer and squamous cell cancer), testicular cancer including germinal tumors (seminomas, and non-seminomas such as teratomas and choriocarcinomas), stromal tumors, germ cell tumors, thyroid cancer (including thyroid adenocarcinoma and medullary carcinoma) and urothelial cancer.

In another embodiment, the cancer is a non-solid cancer. Examples of non-solid tumors include but are not limited to hematological neoplasms. As used herein, a hematologic neoplasm is a term of art which includes lymphoid disorders, myeloid disorders, and AIDS associated leukemias.

Lymphoid disorders include but are not limited to acute lymphocytic leukemia and chronic lymphoproliferative disorders (e.g., lymphomas, myelomas, and chronic lymphoid leukemias). Lymphomas include, for example, Hodgkin's disease, non-Hodgkin's lymphoma lymphomas, and lymphocytic lymphomas. Chronic lymphoid leukemias include, for example, T cell chronic lymphoid leukemias and B cell chronic lymphoid leukemias.

In some embodiments, the cancer is characterized by a high concentration of adenosine in the tumor microenvironment (TME). In some embodiments, the cancer is selected from breast, carcinoid, cervical, colorectal, endometrial, glioma, head and neck, liver, lung, melanoma, ovarian, pancreatic, prostate, renal, gastric, thyroid and urothelial cancers. In some embodiments, the cancer is breast cancer. In some embodiments, the breast cancer is triple negative breast cancer.

In some embodiments, the ENT1 inhibitor is administered prior to, concomitant with, or subsequent to the administration of the checkpoint inhibitor and optionally the adenosine receptor antagonist. In some embodiments, the ENT1 inhibitor is administered prior to administration of the checkpoint inhibitor and optionally the adenosine receptor antagonist. In some embodiments, the ENT1 inhibitor is administered concomitant with administration of the checkpoint inhibitor and optionally the adenosine receptor antagonist. In some embodiments, the ENT1 inhibitor is administered subsequent to administration of the checkpoint inhibitor, and optionally the adenosine receptor antagonist. In some embodiments, the ENT1 inhibitor is administered about 30 minutes to about 1 hour subsequent to administration of the checkpoint inhibitor, for example, on day 1 of a 21-day cycle.

Depending on the condition to be treated and the route of administration, the active compound(s) may be administered as a single daily dose, divided over one or more daily doses, or essentially continuously, e.g. using a drip infusion.

The ENT1 inhibitor, checkpoint inhibitor, the adenosine receptor antagonist, combination or composition of the present disclosure can be administered orally. For example, in some embodiments, the ENT inhibitor is administered orally. In one embodiment, when administered orally, the combination or composition of the present disclosure is administered without food. In one embodiment, when administered orally, the combination or composition of the present disclosure is administered with a meal and/or water. In another embodiment, the combination or composition of the present disclosure is dispersed in water or juice (e.g., apple juice or orange juice) or any other liquid and administered orally as a solution or a suspension. In some embodiments, the ENT inhibitor is administered within about 30 minutes to about 1 hour of a meal, i.e., breakfast and/or dinner.

The ENT1 inhibitor, checkpoint inhibitor, the adenosine receptor antagonist, combination or composition disclosed herein can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. For example, in some embodiments, the checkpoint inhibitor is administered intravenously. The mode of administration is left to the discretion of the health-care practitioner and can depend in-part upon the site of the medical condition.

In one embodiment, the subject has previously received at least one prior therapeutic treatment prior to administration of the ENT1 inhibitor and checkpoint inhibitor, and optionally the adenosine receptor antagonist. In one embodiment, the subject has previously received at least one prior therapeutic treatment and has progressed subsequent to the administration of the at least one prior therapeutic treatment and prior to administration of the ENT1 inhibitor and checkpoint inhibitor, and optionally the adenosine receptor antagonist. In one embodiment, the prior therapeutic treatment is selected from the group consisting of chemotherapy, immunotherapy, radiation therapy, stem cell transplant, hormone therapy, and surgery.

### ENUMERATED EMBODIMENTS

Embodiment 1. A combination of an ENT1 inhibitor and a checkpoint inhibitor.

Embodiment 2. A pharmaceutical composition comprising an ENT1 inhibitor and a checkpoint inhibitor.

Embodiment 3. A kit of parts comprising an ENT1 inhibitor and a checkpoint inhibitor.

Embodiment 4. A method of reversing adenosine-mediated suppression of T cell proliferation and/or viability comprising administering an ENT1 inhibitor and a checkpoint inhibitor.

Embodiment 5. A method of reversing adenosine-mediated suppression of antigen-specific killing activity of T cells comprising administering an ENT1 inhibitor and a checkpoint inhibitor.

Embodiment 6. A method of reversing adenosine-mediated suppression of production of cytokines comprising administering an ENT1 inhibitor and a checkpoint inhibitor.

Embodiment 7. A method of treating cancer comprising administering to a subject in need thereof an ENT1 inhibitor and a checkpoint inhibitor.

Embodiment 8. A use of an ENT1 inhibitor and a checkpoint inhibitor in the manufacture of a medicament for the treatment of cancer.

Embodiment 9. The combination, composition, kit, method or use according to any one of Embodiments 1-8, wherein the checkpoint inhibitor is not an anti-TIGIT antibody.

Embodiment 10. The combination, composition, kit, method or use according to any one of Embodiments 1-9, wherein the checkpoint inhibitor is chosen from a PD1 inhibitor, a PD-L1 inhibitor, and a CTLA-4 inhibitor.

Embodiment 11. The combination, composition, kit, method or use according to any one of Embodiments 1-10, wherein the checkpoint inhibitor is a PD1 inhibitor.

Embodiment 12. The combination, composition, kit, method or use according to any one of Embodiments 1-11, wherein the checkpoint inhibitor is selected from pembrolizumab, nivolumab, atezolizumab, dostarlimab, durvalumab, avelumab, and opdualag.

Embodiment 13. The combination, composition, kit, method or use according to any one of Embodiments 1-12, wherein the ENT1 inhibitor is a compound of

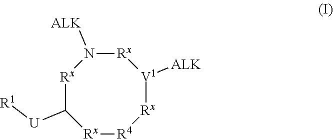

or a pharmaceutically acceptable salt, hydrate, or solvate thereof,
   wherein
R¹ is selected from the group consisting of

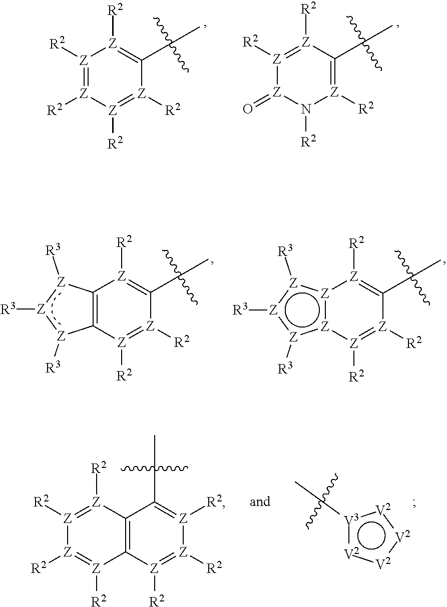

each R² is independently selected from the group consisting of absent, halogen, —NHR³, —OR³, —R³, —C(O)R³, —CO₂R³, C(O)N(R³)₂, —CH₂C(O)N(R³)₂, —S(O)₂R³, and —CN;
or two instances of R² are taken together with the atoms on which they are attached to form a heterocyclyl or heteroaryl ring;
each R³ is independently selected from absent, —H, oxo, ALK, phenyl, heterocyclyl, and heteroaryl;
R⁴ is selected from the group consisting of

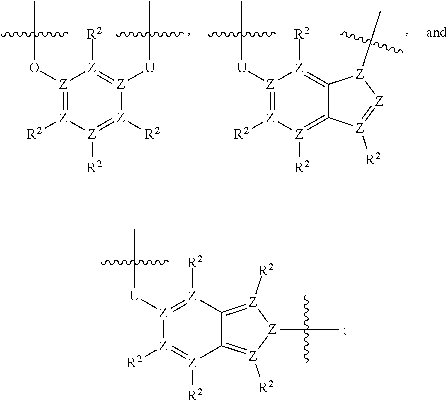

each U is independently selected from the group consisting of —C(O)—, alkylene, —O—, —N(R³)—, —C(O)O—, —C(O)N(R³)—, and

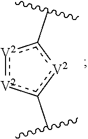

each R^{x} is independently selected from alkylene;
V¹ is selected from —C(R³)— and —N—;
each V² is independently selected from —C(R³)═, —N(R³)—, —N═, and —O—;
V³ is selected from —C═ and —N—; and
Z is C or N,
   wherein ALK is unsubstituted alkyl or substituted alkyl, or two instances of ALK may be joined together with their intervening atoms to form a cycloalkyl or heterocyclyl ring.

Embodiment 14. The combination, composition, kit, method or use according to any one of Embodiments 1-12, wherein the ENT1 inhibitor is a compound of Formula (II):

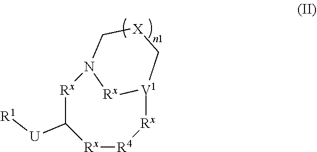

or a pharmaceutically acceptable salt, hydrate, or solvate thereof,
   wherein
R¹ is selected from the group consisting of ALK, cycloalkyl, heterocyclyl,

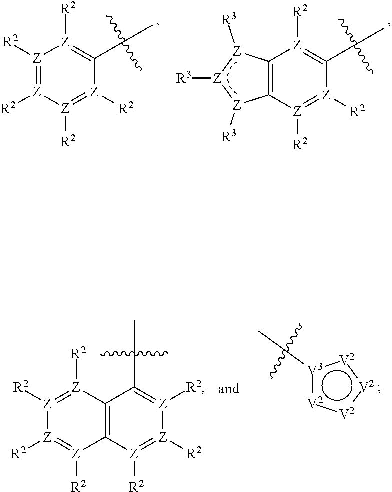

each R² is independently selected from the group consisting of absent, halogen, —OR³, —R³, —CO₂R³, C(O)N(R³)₂, —CH₂C(O)N(R³)₂, —S(O)₂R³, and —CN;
or two instances of R² are taken together with the atoms on which they are attached to form a heterocyclyl or heteroaryl ring;
each R³ is independently selected from absent, —H, ALK, phenyl, and heteroaryl;
R⁴ is

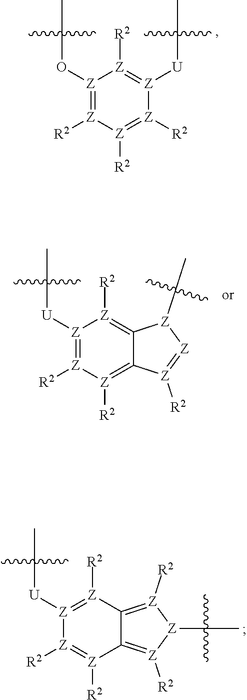

X is selected from the group consisting of —CH₂—, —CHF—, and —CF₂—;
each U is independently selected from the group consisting of —O—, —N(R³)—, —C(O)O—, —C(O)N(R³)—,

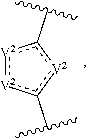

—C(O)—, —O—N═C(H)— and alkylene;
each R is independently selected from alkylene;
V¹ is selected from —C(R³)— and —N—;
each V² is independently selected from —C(R³)═, —N(R³)—, —N═, and —O—;
V³ is selected from —C═ and —N—;
each Z is independently C or N; and
n¹ is a number of 0 or 1,
wherein ALK is unsubstituted alkyl or substituted alkyl, or two instances of ALK may be joined together with their intervening atoms to form a cycloalkyl or heterocyclyl ring.

Embodiment 15. The combination, composition, kit, method or use according to Embodiment 14, wherein the ENT1 inhibitor is a compound of Formula (IIa):

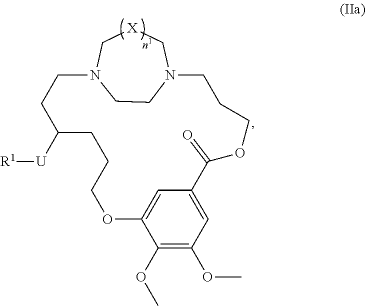

or a pharmaceutically acceptable salt, hydrate, or solvate thereof,
   wherein X is CH₂, CHF, or CF₂.

Embodiment 16. The combination, composition, kit, method or use according to any one of Embodiments 13-15, wherein R¹ is

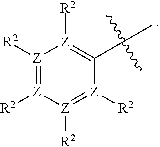

Embodiment 17. The combination, composition, kit, method or use according to Embodiment 16, wherein R¹ is

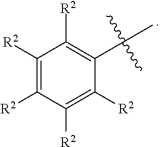

Embodiment 18. The combination, composition, kit, method or use according to any one of Embodiments 14-17, wherein the compound is a compound of Formula (IIb):

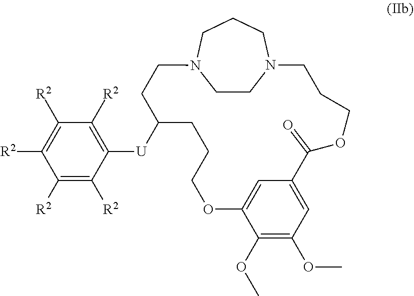

or a pharmaceutically acceptable salt, hydrate, or solvate thereof.

Embodiment 19. The combination, composition, kit, method or use according to any one of Embodiments 13-18, wherein U is —C(O)O—.

Embodiment 20. The combination, composition, kit, method or use according to any one of Embodiments 13, 14, 16, and 17, wherein R⁴ is

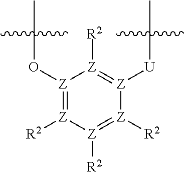

and the U in R⁴ is —C(O)O— or —C(O)NR³—.

Embodiment 21. The combination, composition, kit, method or use according to Embodiment 14, wherein the compound is a compound of Formula (IIa1):

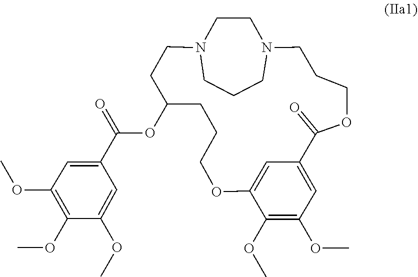

or a pharmaceutically acceptable salt, hydrate, or solvate thereof.

Embodiment 22. The combination, composition, kit, method or use according to any one of Embodiments 1-14, wherein the ENT1 inhibitor is selected from:
(12S)-7⁴,7⁵-dimethoxy-6-oxo-5,8-dioxa-1(1,4)-diazepana-7(1,3)-benzenacyclotetradecaphane-12-yl 3,4,5-trimethoxybenzoate
(12R)-7⁴,7⁵-dimethoxy-6-oxo-5,8-dioxa-1(1,4)-diazepana-7(1,3)-benzenacyclotetradecaphane-12-yl 3,4,5-trimethoxybenzoate
16,16-difluoro-7⁴,7⁵-dimethoxy-6-oxo-5,8-dioxa-1(1,4)-diazepana-7(1,3)-benzenacyclotetradecaphane-12-yl 3,4,5-trimethoxybenzoate
(12S)-16,16-difluoro-7⁴,7⁵-dimethoxy-6-oxo-5,8-dioxa-1(1,4)-diazepana-7(1,3)-benzenacyclotetradecaphane-12-yl 3,4,5-trimethoxybenzoate
(12R)-16,16-difluoro-7⁴,7⁵-dimethoxy-6-oxo-5,8-dioxa-1(1,4)-diazepana-7(1,3)-benzenacyclotetradecaphane-12-yl 3,4,5-trimethoxybenzoate
N-(7⁴,7⁵-dimethoxy-6-oxo-5,8-dioxa-1(1,4)-diazepana-7(1,3)-benzenacyclotetradecaphane-12-yl)-3,4,5-trimethoxybenzamide
7⁴,7⁵-dimethoxy-6-oxo-8-oxa-5-aza-1(1,4)-diazepana-7(1,3)-benzenacyclotetradecaphane-12-yl 3,4,5-trimethoxybenzoate
(12S)-7⁴,7⁵-dimethoxy-6-oxo-8-oxa-5-aza-1(1,4)-diazepana-7(1,3)-benzenacyclotetradecaphane-12-yl 3,4,5-trimethoxybenzoate
(12R)-7⁴,7⁵-dimethoxy-6-oxo-8-oxa-5-aza-1(1,4)-diazepana-7(1,3)-benzenacyclotetradecaphane-12-yl 3,4,5-trimethoxybenzoate
7⁴,7⁵-dimethoxy-5-methyl-6-oxo-8-oxa-5-aza-1(1,4)-diazepana-7(1,3)-benzenacyclotetradecaphane-12-yl 3,4,5-trimethoxybenzoate
(12S)-7⁴,7⁵-dimethoxy-5-methyl-6-oxo-8-oxa-5-aza-1(1,4)-diazepana-7(1,3)-benzenacyclotetradecaphane-12-yl 3,4,5-trimethoxybenzoate
(12R)-7⁴,7⁵-dimethoxy-5-methyl-6-oxo-8-oxa-5-aza-1(1,4)-diazepana-7(1,3)-benzenacyclotetradecaphane-12-yl 3,4,5-trimethoxybenzoate
(11R)-7⁴,7⁵-dimethoxy-6-oxo-5-aza-1(1,4)-diazepana-7(1,3)-benzenacyclotridecaphane-11-yl 3,4,5-trimethoxybenzoate
(10S)-14-chloro-2-oxo-11H-3-aza-1(6,1)-indazola-7(1,4)-diazepanacyclotridecaphane-10-yl 3,4,5-trimethoxybenzoate
(10R)-14-chloro-2-oxo-11H-3-aza-1(6,1)-indazola-7(1,4)-diazepanacyclotridecaphane-10-yl 3,4,5-trimethoxybenzoate
(12S)-6-oxo-5,8-dioxa-1(1,4)-diazepana-7(1,3)-benzenacyclotetradecaphane-12-yl 3,4,5-trimethoxybenzoate
(12R)-6-oxo-5,8-dioxa-1(1,4)-diazepana-7(1,3)-benzenacyclotetradecaphane-12-yl 3,4,5-trimethoxybenzoate
(12S)-6-oxo-8-oxa-5-aza-1(1,4)-diazepana-7(1,3)-benzenacyclotetradecaphane-12-yl benzoate
(12R)-6-oxo-8-oxa-5-aza-1(1,4)-diazepana-7(1,3)-benzenacyclotetradecaphane-12-yl benzoate
7⁴,7⁵dichloro-6-oxo-5,8-dioxa-1(1,4)-diazepana-7(1,3)-benzenacyclotetradecaphane-12-yl 3,4,5-trimethoxybenzoate
(12S)-7⁴,7⁵-dichloro-6-oxo-5,8-dioxa-1(1,4)-diazepana-7(1,3)-benzenacyclotetradecaphane-12-yl 3,4,5-trimethoxybenzoate
(12R)-7⁴,7⁵-dichloro-6-oxo-5,8-dioxa-1(1,4)-diazepana-7(1,3)-benzenacyclotetradecaphane-12-yl 3,4,5-trimethoxybenzoate
7⁵-carbamoyl-7⁴-chloro-6-oxo-5,8-dioxa-1(1,4)-diazepana-7(1,3)-benzenacyclotetradecaphane-12-yl 3,4,5-trimethoxybenzoate
(11Z,16E,10S)-14-chloro-2-oxo-12H-3-aza-1(6,2)-indazola-7(1,4)-diazepanacyclotridecaphane-10-yl 3,4,5-trimethoxybenzoate
(11Z,16E,10R)-14-chloro-2-oxo-12H-3-aza-1(6,2)-indazola-7(1,4)-diazepanacyclotridecaphane-10-yl 3,4,5-trimethoxybenzoate
(12S)-7⁴-carbamoyl-7⁵-chloro-6-oxo-8-oxa-5-aza-1(1,4)-diazepana-7(1,3)-benzenacyclotetradecaphane-12-yl 3,4,5-trimethoxybenzoate
(12R)-7⁴-carbamoyl-7⁵-chloro-6-oxo-8-oxa-5-aza-1(1,4)-diazepana-7(1,3)-benzenacyclotetradecaphane-12-yl 3,4,5-trimethoxybenzoate
7⁴-bromo-7⁵-chloro-6-oxo-8-oxa-5-aza-1(1,4)-diazepana-7(1,3)-benzenacyclotetradecaphane-12-yl 3,4,5-trimethoxybenzoate
7⁵-chloro-7⁴-cyano-6-oxo-8-oxa-5-aza-1(1,4)-diazepana-7(1,3)-benzenacyclotetradecaphane-12-yl 3,4,5-trimethoxybenzoate
(12R)-7⁴,7⁵-dimethoxy-6-oxo-5,8-dioxa-1(1,4)-diazepana-7(1,3)-benzenacyclotetradecaphane-12-yl benzoate
(12R)-7⁴,7⁵-dimethoxy-6-oxo-5,8-dioxa-1(1,4)-diazepana-7(1,3)-benzenacyclotetradecaphane-12-yl benzoate
(12S)-7⁴,7⁵-dimethoxy-6-oxo-5,8-dioxa-1(1,4)-diazepana-7(1,3)-benzenacyclotetradecaphane-12-yl benzoate
(Z)-benzaldehyde O-(7⁴,7⁵-dimethoxy-6-oxo-5,8-dioxa-1(1,4)-diazepana-7(1,3)-benzenacyclotetradecaphane-12-yl) oxime
12-hydroxy-7⁴,7⁵-dimethoxy-5,8-dioxa-1(1,4)-diazepana-7(1,3)-benzenacyclotetradecaphan-6-one
7⁴,7⁵-dimethoxy-6-oxo-5,8-dioxa-1(1,4)-diazepana-7(1,3)-benzenacyclotetradecaphane-12-yl 4-hydroxybenzoate
7⁴,7⁵-dimethoxy-6-oxo-5,8-dioxa-1(1,4)-diazepana-7(1,3)-benzenacyclotetradecaphane-12-yl 4-fluorobenzoate
7⁴,7⁵-dimethoxy-6-oxo-5,8-dioxa-1(1,4)-diazepana-7(1,3)-benzenacyclotetradecaphane-12-yl 4-isopropoxybenzoate
7⁴,7⁵-dimethoxy-6-oxo-5,8-dioxa-1(1,4)-diazepana-7(1,3)-benzenacyclotetradecaphane-12-yl 3-(trifluoromethyl)benzoate
7⁴,7⁵-dimethoxy-6-oxo-5,8-dioxa-1(1,4)-diazepana-7(1,3)-benzenacyclotetradecaphane-12-yl 3-(methylsulfonyl)benzoate
7⁴,7⁵-dimethoxy-6-oxo-5,8-dioxa-1(1,4)-diazepana-7(1,3)-benzenacyclotetradecaphane-12-yl 3-phenoxybenzoate
7⁴,7⁵-dimethoxy-6-oxo-5,8-dioxa-1(1,4)-diazepana-7(1,3)-benzenacyclotetradecaphane-12-yl 2-fluorobenzoate
7⁴,7⁵-dimethoxy-6-oxo-5,8-dioxa-1(1,4)-diazepana-7(1,3)-benzenacyclotetradecaphane-12-yl 4-bromo-3-cyanobenzoate
7⁴,7⁵-dimethoxy-6-oxo-5,8-dioxa-1(1,4)-diazepana-7(1,3)-benzenacyclotetradecaphane-12-yl 3-methyl-5-(trifluoromethyl)benzoate
7⁴,7⁵-dimethoxy-6-oxo-5,8-dioxa-1(1,4)-diazepana-7(1,3)-benzenacyclotetradecaphane-12-yl 2-fluoro-4-methoxybenzoate
7⁴,7⁵-dimethoxy-6-oxo-5,8-dioxa-1(1,4)-diazepana-7(1,3)-benzenacyclotetradecaphane-12-yl 4-methoxy-2-(trifluoromethoxy)benzoate
7⁴,7⁵-dimethoxy-6-oxo-5,8-dioxa-1(1,4)-diazepana-7(1,3)-benzenacyclotetradecaphane-12-yl picolinate
7⁴,7⁵-dimethoxy-6-oxo-5,8-dioxa-1(1,4)-diazepana-7(1,3)-benzenacyclotetradecaphane-12-yl nicotinate
7⁴,7⁵-dimethoxy-6-oxo-5,8-dioxa-1(1,4)-diazepana-7(1,3)-benzenacyclotetradecaphane-12-yl pyrazine-2-carboxylate
7⁴,7⁵-dimethoxy-6-oxo-5,8-dioxa-1(1,4)-diazepana-7(1,3)-benzenacyclotetradecaphane-12-yl 6-hydroxynicotinate
7⁴,7⁵-dimethoxy-6-oxo-5,8-dioxa-1(1,4)-diazepana-7(1,3)-benzenacyclotetradecaphane-12-yl quinoline-5-carboxylate
7⁴,7⁵-dimethoxy-6-oxo-5,8-dioxa-1(1,4)-diazepana-7(1,3)-benzenacyclotetradecaphane-12-yl oxazole-4-carboxylate
7⁴,7⁵-dimethoxy-6-oxo-5,8-dioxa-1(1,4)-diazepana-7(1,3)-benzenacyclotetradecaphane-12-yl 1H-1,2,3-triazole-4-carboxylate
7⁴,7⁵-dimethoxy-6-oxo-5,8-dioxa-1(1,4)-diazepana-7(1,3)-benzenacyclotetradecaphane-12-yl acetate
7⁴,7⁵-dimethoxy-6-oxo-5,8-dioxa-1(1,4)-diazepana-7(1,3)-benzenacyclotetradecaphane-12-yl cyclopropanecarboxylate
7⁴,7⁵-dimethoxy-6-oxo-5,8-dioxa-1(1,4)-diazepana-7(1,3)-benzenacyclotetradecaphane-12-yl 3-methylbutanoate
7⁴,7⁵-dimethoxy-6-oxo-5,8-dioxa-1(1,4)-diazepana-7(1,3)-benzenacyclotetradecaphane-12-yl 4,4,4-trifluorobutanoate
7⁴,7⁵-dimethoxy-6-oxo-5,8-dioxa-1(1,4)-diazepana-7(1,3)-benzenacyclotetradecaphane-12-yl cyclohexanecarboxylate
7⁴,7⁵-dimethoxy-6-oxo-5,8-dioxa-1(1,4)-diazepana-7(1,3)-benzenacyclotetradecaphane-12-yl 1-methylpiperidine-4-carboxylate
7⁴,7⁵-dimethoxy-6-oxo-5,8-dioxa-1(1,4)-diazepana-7(1,3)-benzenacyclotetradecaphane-12-yl 3,3-dimethylcyclobutane-1-carboxylate
7⁴,7⁵-dimethoxy-6-oxo-5,8-dioxa-1(1,4)-diazepana-7(1,3)-benzenacyclotetradecaphane-12-yl 2-(oxetan-3-yl)acetate
7⁴,7⁵-dimethoxy-6-oxo-5,8-dioxa-1(1,4)-diazepana-7(1,3)-benzenacyclotetradecaphane-12-yl (1R,5S,6R)-3-oxabicyclo[3.1.0]hexane-6-carboxylate
7⁴,7⁵-dimethoxy-6-oxo-5,8-dioxa-1(1,4)-diazepana-7(1,3)-benzenacyclotetradecaphane-12-yl 5-oxopyrrolidine-3-carboxylate
7⁴,7⁵-dimethoxy-6-oxo-5,8-dioxa-1(1,4)-diazepana-7(1,3)-benzenacyclotetradecaphane-12-yl 1-benzyl-5-oxopyrrolidine-3-carboxylate
7⁴,7⁵-dimethoxy-6-oxo-5,8-dioxa-1(1,4)-diazepana-7(1,3)-benzenacyclotetradecaphane-12-yl 4-methoxycyclohexane-1-carboxylate
7⁴,7⁵-dimethoxy-6-oxo-5,8-dioxa-1(1,4)-diazepana-7(1,3)-benzenacyclotetradecaphane-12-yl 2,6-difluorobenzoate
7⁴,7⁵-dimethoxy-6-oxo-5,8-dioxa-1(1,4)-diazepana-7(1,3)-benzenacyclotetradecaphane-12-yl 4-(trifluoromethoxy)benzoate
7⁴,7⁵-dimethoxy-6-oxo-5,8-dioxa-1(1,4)-diazepana-7(1,3)-benzenacyclotetradecaphane-12-yl 3-cyanobenzoate
7⁴,7⁵-dimethoxy-6-oxo-5,8-dioxa-1(1,4)-diazepana-7(1,3)-benzenacyclotetradecaphane-12-yl 2-oxo-1,2,3,4-tetrahydroquinoline-6-carboxylate
7⁴,7⁵-dimethoxy-6-oxo-5,8-dioxa-1(1,4)-diazepana-7(1,3)-benzenacyclotetradecaphane-12-yl 3-(difluoromethoxy)benzoate
7⁴,7⁵-dimethoxy-6-oxo-5,8-dioxa-1(1,4)-diazepana-7(1,3)-benzenacyclotetradecaphane-12-yl 3,5-dichlorobenzoate
7⁴,7⁵-dimethoxy-6-oxo-5,8-dioxa-1(1,4)-diazepana-7(1,3)-benzenacyclotetradecaphane-12-yl 3,4-dichlorobenzoate
7⁴,7⁵-dimethoxy-6-oxo-5,8-dioxa-1(1,4)-diazepana-7(1,3)-benzenacyclotetradecaphane-12-yl 2,3-dichlorobenzoate
7⁴,7⁵-dimethoxy-6-oxo-5,8-dioxa-1(1,4)-diazepana-7(1,3)-benzenacyclotetradecaphane-12-yl 2-chloro-6-fluoro-3-methylbenzoate
7⁴,7⁵-dimethoxy-6-oxo-5,8-dioxa-1(1,4)-diazepana-7(1,3)-benzenacyclotetradecaphane-12-yl 3-fluoro-5-(trifluoromethyl)benzoate
7⁴,7⁵-dimethoxy-6-oxo-5,8-dioxa-1(1,4)-diazepana-7(1,3)-benzenacyclotetradecaphane-12-yl 4-fluoro-3-(trifluoromethyl)benzoate
7⁴,7⁵-dimethoxy-6-oxo-5,8-dioxa-1(1,4)-diazepana-7(1,3)-benzenacyclotetradecaphane-12-yl 4-cyano-3-fluorobenzoate
7⁴,7⁵-dimethoxy-6-oxo-5,8-dioxa-1(1,4)-diazepana-7(1,3)-benzenacyclotetradecaphane-12-yl 4-(trifluoromethyl)benzoate
7⁴,7⁵-dimethoxy-6-oxo-5,8-dioxa-1(1,4)-diazepana-7(1,3)-benzenacyclotetradecaphane-12-yl 3,5-difluorobenzoate
7⁴,7⁵-dimethoxy-6-oxo-5,8-dioxa-1(1,4)-diazepana-7(1,3)-benzenacyclotetradecaphane-12-yl 3,4-difluorobenzoate
7⁴,7⁵-dimethoxy-6-oxo-5,8-dioxa-1(1,4)-diazepana-7(1,3)-benzenacyclotetradecaphane-12-yl 3-cyano-4-fluorobenzoate
7⁴,7⁵-dimethoxy-6-oxo-5,8-dioxa-1(1,4)-diazepana-7(1,3)-benzenacyclotetradecaphane-12-yl 4-cyanobenzoate
7⁴,7⁵-dimethoxy-6-oxo-5,8-dioxa-1(1,4)-diazepana-7(1,3)-benzenacyclotetradecaphane-12-yl 3-chloro-4-fluorobenzoate
7⁴,7⁵-dimethoxy-6-oxo-5,8-dioxa-1(1,4)-diazepana-7(1,3)-benzenacyclotetradecaphane-12-yl 1-methyl-1H-benzo[d]imidazole-5-carboxylate
7⁴,7⁵-dimethoxy-6-oxo-5,8-dioxa-1(1,4)-diazepana-7(1,3)-benzenacyclotetradecaphane-12-yl 4-(oxazol-5-yl)benzoate
7⁴,7⁵-dimethoxy-6-oxo-5,8-dioxa-1(1,4)-diazepana-7(1,3)-benzenacyclotetradecaphane-12-yl 4,5-dichloro-2-fluorobenzoate
7⁴,7⁵-dimethoxy-6-oxo-5,8-dioxa-1(1,4)-diazepana-7(1,3)-benzenacyclotetradecaphane-12-yl 3,4,5-triethoxybenzoate
7⁴,7⁵-dimethoxy-6-oxo-5,8-dioxa-1(1,4)-diazepana-7(1,3)-benzenacyclotetradecaphane-12-yl 3-methoxypropanoate
7⁴,7⁵-dimethoxy-6-oxo-5,8-dioxa-1(1,4)-diazepana-7(1,3)-benzenacyclotetradecaphane-12-yl 3-(1H-pyrazol-1-yl)propanoate
7⁴,7⁵-dimethoxy-6-oxo-5,8-dioxa-1(1,4)-diazepana-7(1,3)-benzenacyclotetradecaphane-12-yl 3-cyanopropanoate
7⁴,7⁵-dimethoxy-6-oxo-5,8-dioxa-1(1,4)-diazepana-7(1,3)-benzenacyclotetradecaphane-12-yl 4-cyanobutanoate
7⁴,7⁵-dimethoxy-6-oxo-5,8-dioxa-1(1,4)-diazepana-7(1,3)-benzenacyclotetradecaphane-12-yl 4-acetamidobutanoate
7⁴,7⁵-dimethoxy-6-oxo-5,8-dioxa-1(1,4)-diazepana-7(1,3)-benzenacyclotetradecaphane-12-yl 3-(1H-tetrazol-1-yl)propanoate
7⁴,7⁵-dimethoxy-6-oxo-5,8-dioxa-1(1,4)-diazepana-7(1,3)-benzenacyclotetradecaphane-12-yl 4-(dimethylamino)-4-oxobutanoate
7⁴,7⁵-dimethoxy-6-oxo-5,8-dioxa-1(1,4)-diazepana-7(1,3)-benzenacyclotetradecaphane-12-yl 3-acetamidopropanoate
7⁴,7⁵-dimethoxy-6-oxo-5,8-dioxa-1(1,4)-diazepana-7(1,3)-benzenacyclotetradecaphane-12-yl 4-(methylamino)-4-oxobutanoate
7⁴,7⁵-dimethoxy-6-oxo-5,8-dioxa-1(1,4)-diazepana-7(1,3)-benzenacyclotetradecaphane-12-yl 3-(1H-1,2,4-triazol-1-yl)propanoate
7⁴,7⁵-dimethoxy-6-oxo-5,8-dioxa-1(1,4)-diazepana-7(1,3)-benzenacyclotetradecaphane-12-yl 4-morpholino-4-oxobutanoate
7⁴,7⁵-dimethoxy-6-oxo-5,8-dioxa-1(1,4)-diazepana-7(1,3)-benzenacyclotetradecaphane-12-yl 3-(4-fluorophenoxy)propanoate
7⁴,7⁵-dimethoxy-6-oxo-5,8-dioxa-1(1,4)-diazepana-7(1,3)-benzenacyclotetradecaphane-12-yl 4,4-difluorocyclohexane-1-carboxylate
7⁴,7⁵-dimethoxy-6-oxo-5,8-dioxa-1(1,4)-diazepana-7(1,3)-benzenacyclotetradecaphane-12-yl 4-(trifluoromethyl)cyclohexane-1-carboxylate
7⁴,7⁵-dimethoxy-6-oxo-5,8-dioxa-1(1,4)-diazepana-7(1,3)-benzenacyclotetradecaphane-12-yl 3-(2,5-dioxopyrrolidin-1-yl)propanoate
7⁴,7⁵-dimethoxy-6-oxo-5,8-dioxa-1(1,4)-diazepana-7(1,3)-benzenacyclotetradecaphane-12-yl 3-methoxycyclohexane-1-carboxylate
7⁴,7⁵-dimethoxy-6-oxo-8-oxa-5-aza-1(1,4)-diazepana-7(1,3)-benzenacyclotetradecaphane-12-yl benzoate
(E)-benzaldehyde O-(7⁴,7⁵-dimethoxy-6-oxo-8-oxa-5-aza-1(1,4)-diazepana-7(1,3)-benzenacyclotetradecaphane-12-yl) oxime
(E)-benzaldehyde O-((12R)-7⁴,7⁵-dimethoxy-6-oxo-8-oxa-5-aza-1(1,4)-diazepana-7(1,3)-benzenacyclotetradecaphane-12-yl) oxime
(E)-benzaldehyde O-((12S)-7⁴,7⁵-dimethoxy-6-oxo-8-oxa-5-aza-1(1,4)-diazepana-7(1,3)-benzenacyclotetradecaphane-12-yl) oxime
12-hydroxy-7⁴,7⁵-dimethoxy-8-oxa-5-aza-1(1,4)-diazepana-7(1,3)-benzenacyclotetradecaphan-6-one
(12R)-12-hydroxy-7⁴,7⁵-dimethoxy-5,8-dioxa-1(1,4)-diazepana-7(1,3)-benzenacyclotetradecaphan-6-one
(12S)-12-hydroxy-7⁴,7⁵-dimethoxy-5,8-dioxa-1(1,4)-diazepana-7(1,3)-benzenacyclotetradecaphan-6-one
7⁴,7⁵-dimethoxy-12-(5-phenyl-2H-tetrazol-2-yl)-5,8-dioxa-1(1,4)-diazepana-7(1,3)-benzenacyclotetradecaphan-6-one
7⁴,7⁵-dimethoxy-12-(4-phenyl-1H-1,2,3-triazol-1-yl)-5,8-dioxa-1(1,4)-diazepana-7(1,3)-benzenacyclotetradecaphan-6-one
7⁴,7⁵-dimethoxy-12-(5-phenyl-1H-tetrazol-1-yl)-5,8-dioxa-1(1,4)-diazepana-7(1,3)-benzenacyclotetradecaphan-6-one,
   and pharmaceutically acceptable salts, hydrates, or solvates thereof.

Embodiment 23. The combination, composition, kit, method or use according to any one of Embodiments 1-14, wherein the ENT1 inhibitor is selected from Compound 1

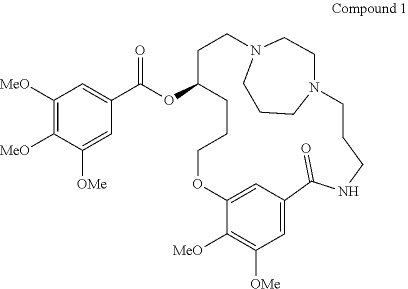 [SMILES: COc1cc(C(=O)O[C@@H]2CCCOc3cc(cc(OC)c3OC)C(=O)NCCCN3CCCN(CC2)CC3)cc(OC)c1OC]

and pharmaceutically acceptable salts, hydrates, and solvates thereof.

Embodiment 24. The combination, composition, kit, method or use according to any one of Embodiments 1-14, wherein the ENT1 inhibitor is selected from Compound 1

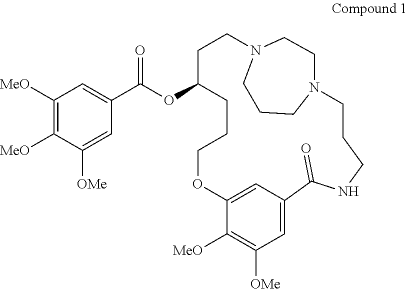 [SMILES: COc1cc(C(=O)O[C@@H]2CCCOc3cc(cc(OC)c3OC)C(=O)NCCCN3CCCN(CC2)CC3)cc(OC)c1OC]

and pharmaceutically acceptable salts thereof.

Embodiment 25. The combination, composition, kit, method or use according to Embodiment 24, wherein the ENT1 inhibitor is a Compound 1 hydrogen sulfate or a hydrate or solvate thereof.

Embodiment 26. The combination, composition, kit, method or use according to Embodiment 25, wherein the ENT1 inhibitor is a Compound 1 di(hydrogen sulfate) or a hydrate or solvate thereof.

Embodiment 27. The combination, composition, kit, method or use according to Embodiment 26, wherein the Compound 1 di(hydrogen sulfate) or a hydrate or solvate thereof is crystalline.

Embodiment 28. The combination, composition, kit, method or use according to Embodiment 27, wherein the crystalline Compound 1 di(hydrogen sulfate) or a hydrate or solvate thereof is crystalline Form 2 Compound 1 di(hydrogen sulfate) trihydrate.

Embodiment 29. The method according to any one of Embodiments 4-6, wherein the reversal of adenosine-mediated suppression occurs in a cancer.

Embodiment 30. The method or use according to any one of Embodiments 4-29, wherein the ENT1 inhibitor is administered prior to, concomitant with, or subsequent to the administration of the checkpoint inhibitor.

Embodiment 31. The method or use according to any one of embodiments 4-30, wherein the ENT1 inhibitor is administered orally, optionally as a tablet or capsule.

Embodiment 32. The method or use according to any one of embodiments 4-31, wherein the ENT1 inhibitor is administered daily during a treatment cycle.

Embodiment 33. The method or use according to any one of embodiments 4-32, wherein the ENT1 inhibitor is administered once or twice daily (BID).

Embodiment 34. The method or use according to any one of embodiments 4-31, wherein the ENT1 inhibitor is administered intermittently during a treatment cycle.

Embodiment 35. The method or use according to any one of embodiments 4-34, wherein the ENT1 inhibitor is administered at the same dose during the treatment cycle.

Embodiment 36. The method or use according to any one of embodiments 4-34, wherein the ENT1 inhibitor is administered at doses of escalating concentration.

Embodiment 37. The method or use according to any one of embodiments 4-36, further comprising administering an agent capable of treating, preventing, delaying, reducing or attenuating the development or risk of development of an adverse event.

Embodiment 38. The method or use according to embodiment 37, wherein the agent is administered prior to treatment with the ENT1 inhibitor, concomitant with, and/or subsequent to treatment with the ENT1 inhibitor.

Embodiment 39. The method or use according to embodiment 37 or embodiment 38, wherein the agent is administered prior to, concomitant with, and/or subsequent to at least one, two, three, four, five, six, seven, eight, nine, or ten doses of ENT inhibitor.

Embodiment 40. The method or use according to any one of embodiments 37-39, wherein the agent is administered at least 30 minutes prior to and/or subsequent to at least one dose of ENT1 inhibitor.

Embodiment 41. The method or use according to any one of embodiments 37-40, wherein the agent comprises an antiemetic, an analgesic, and/or caffeine.

Embodiment 42. The method or use according to embodiment 41, wherein the analgesic comprises a non-steroidal anti-inflammatory drug (NSAID), aspirin, acetaminophen, antidepressive medications, antiepileptic medication, and/or lidocaine; and the antiemetic comprises anticholinergic agents, antihistamines, cannabinoid receptor agonists, dopamine receptor antagonists, serotonin 5-HT3 receptor antagonists, and/or substance P/neurokinin 1 receptor antagonists.

Embodiment 43. The method or use according to embodiment 41, wherein the agent is caffeine.

Embodiment 44. The method or use according to any one of embodiments 37-43, wherein the agent is administered orally, optionally as a tablet or a capsule.

Embodiment 45. The method or use according to any one of embodiments 37-43, wherein the agent is administered in a beverage or in food.

Embodiment 46. The method or use according to any one of embodiments 4-45, wherein the ENT1 inhibitor is administered with a meal and/or a beverage, such as water.

Embodiment 47. The method or use according to any one of embodiments 7-46, wherein the cancer is characterized by a high concentration of adenosine in the tumor microenvironment (TME).

Embodiment 48. The method or use according to any one of embodiments 7-47, wherein the cancer is selected from breast, carcinoid, cervical, colorectal, endometrial, glioma, head and neck, liver, lung, melanoma, ovarian, pancreatic, prostate, renal, gastric, thyroid and urothelial cancers.

Embodiment 49. The method or use according to any one of embodiments 7-48, wherein the cancer is breast cancer.

Embodiment 50. The method or use according to embodiment 49, wherein the breast cancer is triple negative breast cancer.

Embodiment 51. The method or use according to embodiments 7-49, wherein the cancer is a solid-tumor cancer.

Embodiment 52. The combination, composition, or kit according to any one of embodiments 1-3 or 9-28, further comprising an adenosine receptor antagonist.

Embodiment 53. The method or use according to any one of embodiments 4-51, further comprising administering an adenosine receptor antagonist.

### EXAMPLES

### Preparation of Certain ENT1 Inhibitors

### Analytical Methods.

### (1) X-ray Powder Diffraction (XRPD)

In the examples below, unless otherwise stated, XRPD analysis was carried out on a PANalytical X'pert pro with PIXcel detector (128 channels), scanning the samples between 3 and 35° 2θ. The material was gently ground (where required) to release any agglomerates and loaded onto a multi-well plate with Mylar polymer film to support the sample. The multi-well plate was then placed into the diffractometer and analyzed using Cu K radiation (α1λ=1.54060 Å; α2=1.54443 Å; β=1.39225 Å; α1:α2 ratio=0.5; most, if not all, β radiation is removed from the beam using an X-ray mirror) running in transmission mode (step size 0.0130° 2θ, step time 18.87 s) using 40 kV/40 mA generator settings. Data were visualized and images generated using the HighScore Plus 4.7 desktop application (PANalytical, 2017).

### (2) Single Crystal X-ray Diffraction (SCXRD)

In Example 4, a suitable crystal was selected and mounted in a nylon loop while protected in a protective layer of paratone oil. Two data sets were collected at temperatures: 295K and 100K. In both cases, data were collected using a Bruker D8 Venture diffractometer equipped with a Photon III detector operating in shutterless mode with Cu-Kα radiation (1.54178 Å). All nonhydrogen atoms were located in the Fourier map and their positions refined prior to describing the thermal movement of all non-hydrogen atoms anisotropically.

The structures were solved in the Olex2 software package with the ShelXT (intrinsic phasing) structure solution program and refined with the ShelXL refinement package using Least Squares minimization. See Dolomanov, O. V. et al, J. Appl. Cryst., 2009, 42, 339-341; Sheldrick, G. M., Acta Cryst., 2015, A71, 3-8; and Sheldrick, G. M., Acta Cryst., 2015, C71, 3-8. Data was collected, solved, and refined in triclinic space-group P1.

A series of restraints were applied to the final refinement. All CH and CH₂ groups were refined with fixed Uiso of 1.2 times, and all CH₃ groups with fixed Uiso of 1.5 times. The anions were disordered in the structure collected at 100K and they were modelled as follows:—S4 was disordered over two positions and modelled at 0.44 occupancy; O300, O302 and O303 were disordered over two positions and modelled at 0.55 occupancy.

For the structure collected at 295 K, the highest residual Fourier peak was found to be 0.75 e.Å⁻³ approx. 2.43 Å from O302 and the deepest Fourier hole was found to be −0.72 e.Å⁻³ approx. 0.62 Å from S2.

For the structure collected at 100 K, the highest residual Fourier peak was found to be 0.63 e.Å⁻³ approx. 0.51 Å from H4WA and the deepest Fourier hole was found to be −1.17 e.Å⁻³ approx. 0.61 Å from S2.

Crystal data for the structure at 295K was obtained with the following parameters: C₃₃H₅₅N₃O₁₉S₂* (M=858.89 g/mol): triclinic, space group P1 (no. 1), a=9.669(2) Å, b=12.439(3) Å, c=18.717(4) Å, α=102.143(13)°, β=94.505(14)°, γ=110.746(12)°, V=2029.0(8) Å³, Z=2 T=295(2) K, μ(CuKα)=1.896 mm⁻¹, Dcalc=1.406 g/cm³, 59429 reflections measured (4.898≤2Θ≤133.718), 13898 unique (Rᵢₙₜ=0.0790, R_{sigma}=0.0782) which were used in all calculations. The final R₁ was 0.0835 (>2σ(I)) and wR2 was 0.2767 (all data). *the calculated formula differs from the reported formula by two hydrogen atoms and one oxygen atom that were not located

Crystal data for the structure at 100K was obtained with the following parameters: C₃₃H₅₇N₃O₂₀S₂ (M=879.93 g/mol): triclinic, space group P1 (no. 1), a=9.6794(2) Å, b=12.3886(2) Å, c=18.4016(4) Å, α=101.6708(8)⁰, f=94.7448(8)⁰, y=110.7514(8)°, V=1991.88(7) Å³, Z=2, T=100.0 K, μ(CuKα)=1.962 mm-1, Dcalc=1.467 g/cm3, 97841 reflections measured (4.974°≤2Θ≤144.29°), 15198 unique (Rᵢₙₜ=0.0299, R_{sigma}=0.0202) which were used in all calculations. The final R₁ was 0.0586 (I>2σ(I)) and wR₂ was 0.1597 (all data).

(3) Thermogravimetric Analysis/Differential Scanning Calorimetry (TGA/DSC), a.k.a. Thermogravimetric/Differential Scanning Calorimetry (TG/DSC)

In the examples below, to measure TGA/DSC, 2-10 mg of material was added into a pre-tared open aluminum pan and loaded into a TA Instruments Discovery SDT 650 Auto-Simultaneous DSC and held at room temperature. The sample was then heated at a rate of 10° C./min from 30° C. to 400° C. during which time the change in sample weight was recorded along with the heat flow response (DSC). Nitrogen was used as the sample purge gas, at a flow rate of 200 cm³/min.

### (4) Differential Scanning Calorimetry (DSC)

METHOD A: In Example 1, 1-5 mg of material was weighed into an aluminum DSC pan and sealed non-hermetically with a pierced aluminum lid. The sample pan was then loaded into a TA DSC2500 and held at 20° C. Once a stable heat-flow response was obtained, the sample and reference were heated to an upper temperature of 200° C. at a scan rate of 10° C./min and the resulting heat flow response monitored. The sample was held at the upper temperature for 3 minutes, before it was cooled at 10° C./min to 20° C., reheated to the upper temperature at 10° C./min, and then cooled again to 20° C. at the same rate. Nitrogen was used as the purge gas, at a flow rate of 50 cm³/min.

METHOD B: In Example 3, 1-5 mg of a sample were weighed into an aluminum DSC pan and sealed non-hermetically with an aluminum lid. The sample pan was then loaded into a TA Instruments Discovery DSC 2500 differential scanning calorimeter equipped with a RC90 cooler. The sample and reference were heated to 190° C. at a scan rate of 10° C./min and the resulting heat flow response monitored. The sample was re-cooled to −80° C. and then reheated again to 190° C. all at 10° C./min. Nitrogen was used as the purge gas, at a flow rate of 50 cm³/min.

### (5) Nuclear Magnetic Resonance Spectroscopy (NMR)

METHOD A: In Example 1, ¹H NMR experiments were performed on a Bruker AV500 (frequency: 500 MHz for protons). Experiments were performed in d₄-methanol and samples were prepared to ca. 5 mM concentration.

METHOD B: In Example 3, NMR experiments were performed on a Bruker AVIIIHD spectrometer equipped with a DCH cryoprobe operating at 500.12 MHz for protons. Experiments were performed in d4-methanol and each sample was prepared to ca. 10 mM concentration. Quantitative NMR analysis was carried out using 1,3,5-trimethoxybenzene as the external standard; experiments were carried out in d₄-methanol for Compound 1, which is a free base, samples and in DMSO-d₆ for the di(hydrogen sulfate) samples.

### (6) Infrared Spectroscopy (IR)

In the examples below, Infrared spectroscopy was carried out on a Bruker ALPHA P spectrometer. Sufficient material was placed onto the center of the plate of the spectrometer and the spectra were obtained using the following parameters:
Resolution: 4 cm⁻¹
Background Scan Time: 16 scans
Sample Scan Time: 16 scans
Data Collection: 4000 to 400 cm⁻¹
Result Spectrum: Transmittance

Software: OPUS version 6

### (7) Polarized Light Microscopy (PLM)

In Example 3, the presence of crystallinity (birefringence) was determined using an Olympus BX50 microscope, equipped with cross-polarizing lenses and a Motic camera. Images were captured using Motic Images Plus 2.0. All images were recorded using the 20× objective, unless otherwise stated.

### (8) Hot Stage Light Microscopy (HSM)

In Example 5, thermal events were monitored visually using a calibrated Linkam THM600 hotstage with connected controller unit coupled to an Olympus BH2 microscope equipped with a Motic camera and image capture software (Motic Images Plus 3.0). Approximately 0.5 mg of material was placed onto a microscope coverslip and heated at a rate of 10° C./min with images taken at routine intervals to document any thermal transitions. All images were recorded using the 20×objective, unless otherwise stated.

### (9) Karl Fischer Coulometric Titration (KF)

In the examples below, KF titration was measured using either an In Motion KF Autosampler or Direct Addition Method. The In Motion KF Autosampler was carried out via the following procedure. 15-20 mg of solid was weighed into a 10 mL glass vial and tightly sealed with a screw cap. Samples were analyzed using a Mettler Toledo C30SX and an InMotion KFOven Autosampler at 130° C. Samples were analyzed in duplicate and an average moisture content reported. See table below for further details.

| | | |
|---|---|---|
| Blank | Oven Temperature/° C. | 130 |
| | Source for Drift | Determination |
| | Max. Start Drift/μg/min | 10 |
| | Carrier Gas Flow Rate/mL/min | 80 |
| | Transfer Tube Heating | No |
| | Mix Time/s | 60 |
| | Stir Speed/% | 45 |
| | Drift Termination/s | 10 (Delay Time) |
| | Max. Titration Time/s | 600 |
| Sample | Oven Temperature/° C. | 130 |
| | Source for Drift | Determination |
| | Max. Start Drift/μg/min | 10 |
| | Carrier Gas Flow Rate/mL/min | 80 |
| | Mix Time/s | 60 |
| | Stir Speed/% | 45 |
| | Drift Termination/s | 10 (Delay Time) |
| | Max. Titration Time/s | 600 |

The Direct Addition Method was carried out via the following procedure. 15-20 mg of solid material was accurately weighed into a vial. The solid was then manually introduced into the titration cell of a Mettler Toledo C30 Compact Titrator. The vial was back-weighed after the addition of the solid and the weight of the added solid entered on the instrument. Titration was initiated once the sample had fully dissolved in the cell. The water content was calculated automatically by the instrument as a percentage and the data printed.

### (10) Dynamic Vapor Sorption (DVS)

METHOD A: In Example 5, approximately 5-15 mg of sample was placed into a mesh vapor sorption balance pan and loaded into either a DVS Intrinsic or DVS Advantage dynamic vapor sorption balance by Surface Measurement Systems. The sample was subjected to a ramping profile from 40-90% relative humidity (RH) at 10% increments, maintaining the sample at each step until a stable weight had been achieved (dm/dt 0.004%, minimum step length 30 minutes, maximum step length 500 minutes) at 25° C. After completion of the sorption cycle, the sample was dried using the same procedure to 0% RH and then a second sorption cycle back to 40% RH was carried out. Two cycles were performed. The weight change during the sorption/desorption cycles were plotted, allowing the hygroscopic nature of the sample to be determined. XRPD analysis was then carried out on the residual solid.

METHOD B: In Example 1, approximately 10-20 mg of sample was placed into a mesh vapor sorption balance pan and loaded into either a DVS Intrinsic or DVS Advantage dynamic vapor sorption balance by Surface Measurement Systems. The sample was subjected to a ramping profile from 40-90% relative humidity (RH) at 10% increments, maintaining the sample at each step until a stable weight had been achieved (dm/dt 0.004%, minimum step length 30 minutes, maximum step length 500 minutes) at 25° C. After completion of the sorption cycle, the sample was dried using the same procedure to 0% RH and then a second sorption cycle back to 40% RH was carried out. Two cycles were performed. The weight change during the sorption/desorption cycles were plotted, allowing the hygroscopic nature of the sample to be determined.

### (11) High Performance Liquid Chromatography-Ultraviolet Detection (HPLC-UV)

Unless otherwise indicated in the examples below, HPLC-UV was performed with the follow experimental parameters and conditions:
Column: Waters XSelect CSH Fluoro-Phenyl 150×4.6 mm, 2.5 μm
Mobile Phase A: 0.1% TFA in water
Mobile Phase B: 0.1% TFA in acetonitrile
Diluent: Methanol
Autosampler Temperature: 5° C.
Flow Rate: 1.0 mL/min
Runtime: 30 minutes
Column Temperature: 30° C. (±1° C.)
Column Pressure: 110 bar (at start of run)
Injection Volume: 5 μL
Sample Concentration: 0.5 mg/mL
Detection: 266 nm
Sampling Rate: 50 Hz

Gradient Program:

| Time (min) | Mobile Phase A (%) | Mobile Phase B (%) |
|---|---|---|
| 0.0 | 95 | 5 |
| 5.0 | 75 | 25 |
| 18.0 | 65 | 35 |
| 20.0 | 50 | 50 |
| 22.0 | 5 | 95 |
| 25.0 | 5 | 95 |
| 25.1 | 95 | 5 |
| 30.0 | 95 | 5 |

### (12) Gas Chromatography (GC)

In Example 3, gas chromatography was conducted using a Thermo Trace 1300 GC equipped with a Tri-Plus 300 Headspace sampler. The following parameters were used:
Column: Agilent J&W DB-624 30 m×0.32 mm, 1.8 μm d.f. or equivalent
Oven Temperature: 35° C. (hold 0.5 min) to 45° C. @16.5° C./min to 70° C. @5.0° C./min to 220° C.@30.0° C./min
Flow Rate: 2.2 mL/min (constant flow)
Carrier Gas: Hydrogen
Diluent: DMA
Injection Mode: Split
Injection Temperature: 225° C.
Injection Split Ratio: 20:1
Detector Temperature: 250° C.
Detector Hydrogen: 30.0 mL/min
Detector Air: 400 mL/min
Make-up Flow: 40.0 mL/min
Make-up Gas: Nitrogen
The following Headspace Parameters were used:
Oven Temperature: 100° C.
Loop Temperature: 110° C.
Transfer Line Temperature: 150° C.
Vial Equilibration Time: 10.0 min
Pressurization Mode: Pressure
Auxiliary Pressure: 100 kPa (Nitrogen)
Pressurization Time: 0.2 min
Loop Fill Mode: Pressure
Loop Pressure: 50 kPa
Loop Equilibration Time: 0.2 min
Loop Volume: 1 mL
Inject Time: 0.5 min
Vial Shaking: High
GC Cycle Time: 21 min.

### (13) Raman Spectroscopy

In Example 2, Raman spectroscopy was carried out using a Wasatch 785 nm spectrometer and a BlazeMetrics 900 probe. The material to be analyzed was brought in contact with the probe window and measurements were taken with the following parameters:
Wavenumber: ca. 2021-218 cm⁻¹
Accumulations: 2
Integration time: 2000 ms
Care was taken to protect the sample from external light sources.

### (14) Particle Size Distribution

In Example 3, the particle size distribution of solids was measured using a Mastersizer 2000 instrument and the method is detailed below.

| Dispersant | 0.05% w/v span 85 in Heptane | |
|---|---|---|
| Concentration | 100 mg in 10 mL dispersant | |
| Stirrer Speed | 2000 | rpm |
| Sonication time | 30 | seconds |
| Pre-measurement delay | 1 | minute |
| Measurement time | 10 | seconds |
| Background measurement time | 10 | seconds |

| | |
|---|---|
| Measurement cycles | 3 |
| Obscuration | 5-20% |
| Analysis model | General model |
| Sensitivity | Normal |
| Dispersant Refractive Index | 1.39 |
| Material size absorption | 0.01 |
| Material R.I. | 1.58 |

### Example 1. Preparation 1 of Crystalline Form 2 Compound 1 Di(Hydrogen Sulfate) Trihydrate

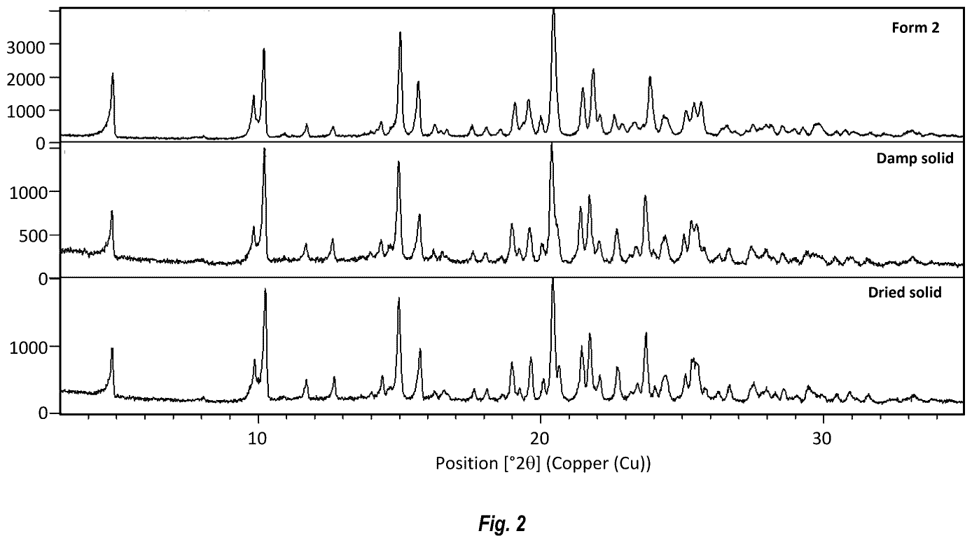

In this preparation method, a sample of Compound 1 (which is a free base) and 6 vol. of IPA/water (75:25% v/v) were added to a 20 mL scintillation vial. 2.05 mole equivalents of sulfuric acid were added as a solution in 2 vol. of IPA/water (75:35% v/v). The solution was stirred for about one hour at 40° C. The temperature was then cycled between 40° C. and 5° C. with a 0.1° C./min ramp and a one hour hold between each step. After about 48 hours of cycling, a clear solution was still observed. Up to 6 vol. of anti-solvent (tBME) was added to 40° C. to facilitate precipitation. The experiment (now a slurry) was further temperature cycled for 24 hours. The solids were then isolated via vacuum filtration and dried under vacuum at about 40° C. for 48 hours. The damp and dried solids were subsampled and analyzed by XRPD and shown to be crystalline Form 2 Compound 1 di(hydrogen sulfate) trihydrate as shown in FIG. 2. TG/DSC data show a mass loss of 7.3% corresponding to an endothermic event with an onset of 67° C. which is likely due to water loss (approximating 3.6 mole equivalents of water) (FIG. 3). DSC shows an endotherm at an onset of 144° C., which is consistent with a melting event (FIG. 4) whereas a small melting event was observed in the TG/DSC data with an onset of 130° C.

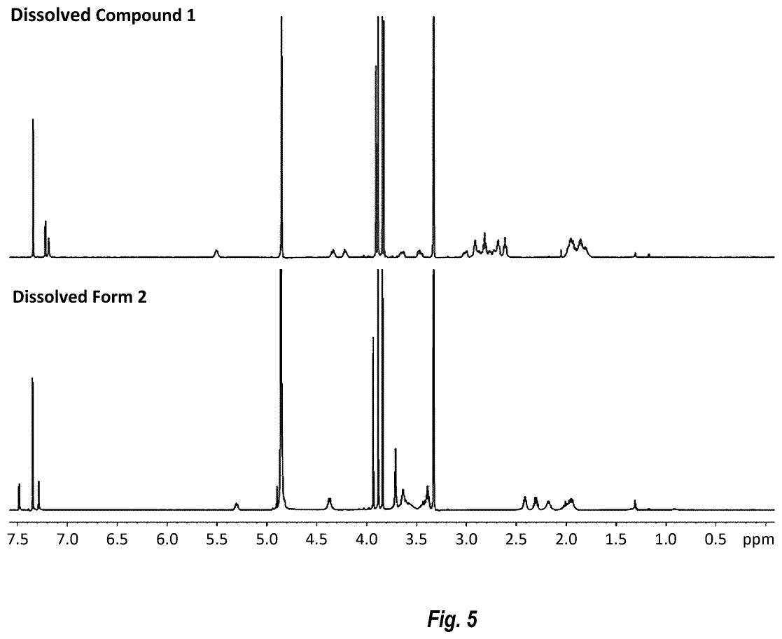

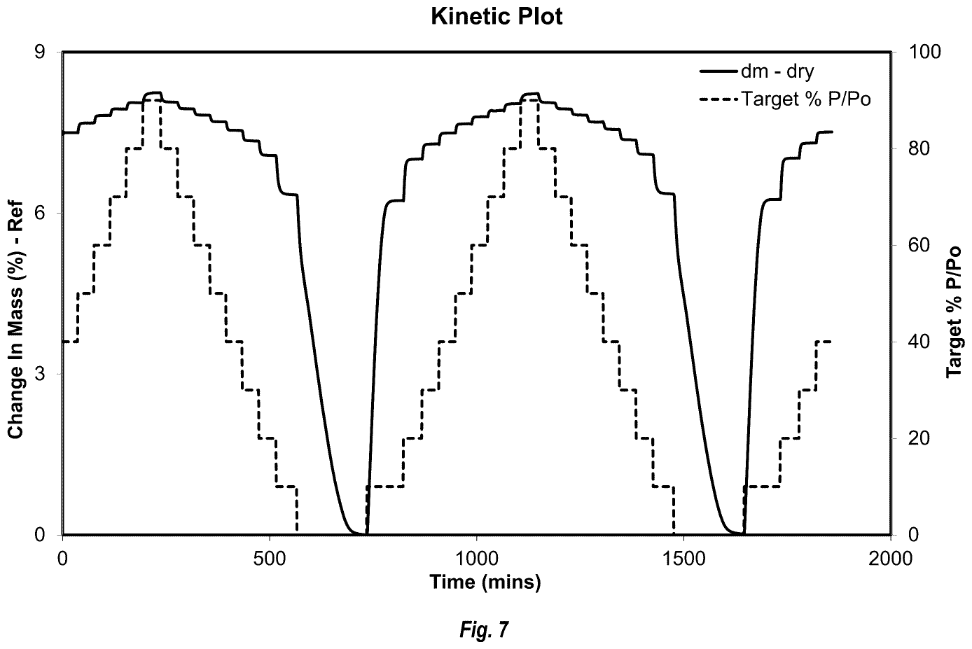

KF analysis shows a water content of 6.74%. ¹H-NMR is consistent with the Compound 1 (free base) structure with peak shifts indicating salt formation (FIG. 5). A DVS experiment (FIG. 6 and FIG. 7) shows a moisture uptake of about 6.3% between 0 and 10% RH with a more gradual uptake of 1.7% as RH is raised to 80% from 10%.

### Example 2. Preparation 2 of Crystalline Form 2 Compound 1 Di(Hydrogen Sulfate) Trihydrate

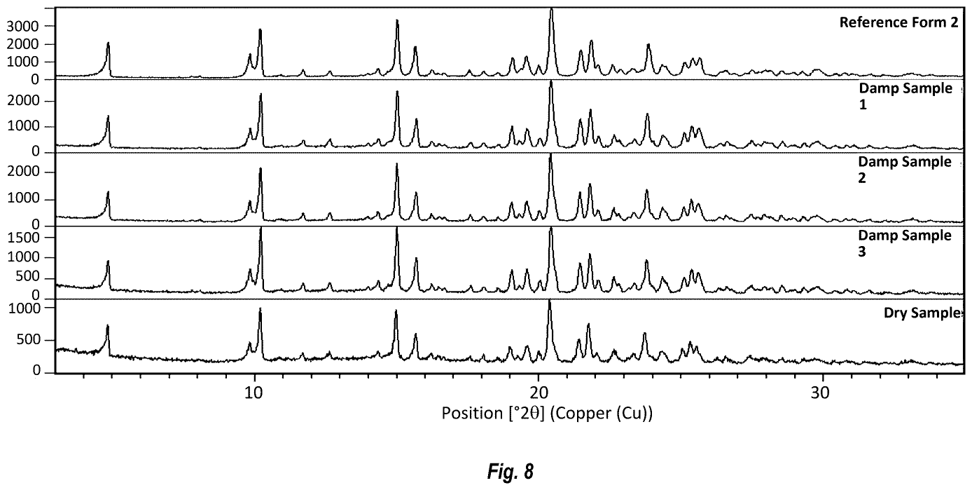

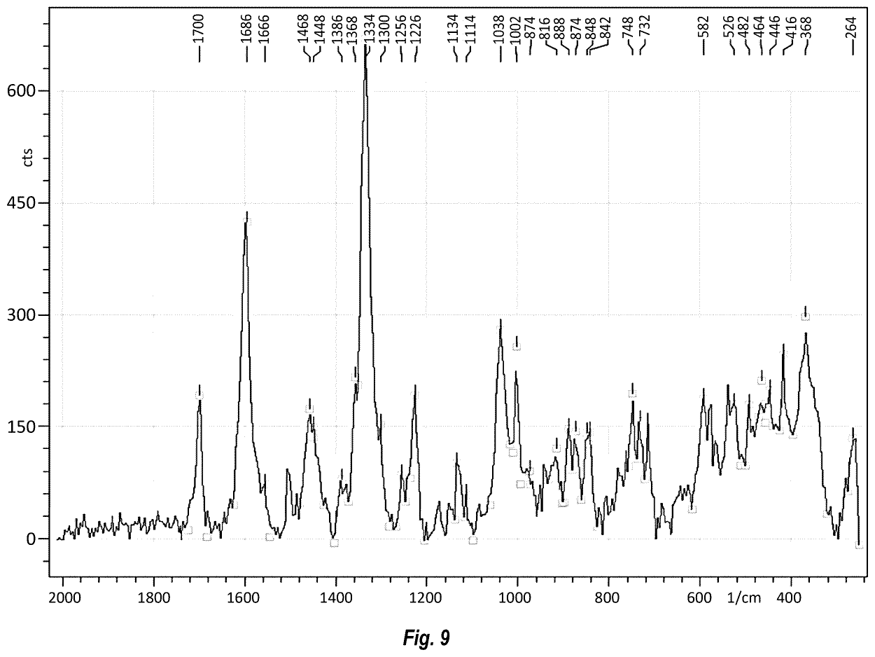

Approximately 1.5 g of Compound 1 was added to three 20 mL scintillation vials. 5 mL of 2-propanol:water (75:25% v/v) was added to each vial, and the samples were stirred at 40° C. 2.05 mole equiv. of H₂SO₄ was added to each sample as solutions in 5 mL of propanol:water (75:25% v/v). Clear solutions were observed in all samples. Stirring continued at 40° C. for about 1.5 hours after addition of H₂SO₄. tBME was then added to each sample, also at 40° C., as an anti-solvent in 1 mL aliquots until the solutions turned cloudy. The samples were then temperature-cycled between 40° C. and 5° C. for about 20 hours with 0.1° C./min ramp and a 1 hour hold between steps. The resultant slurries were subsampled, and solids isolated via centrifugation. The isolated solids were analyzed by XRPD. The remainder of the slurries were vacuum filtered as one sample using a Buchner funnel. The filter cake was dried under vacuum at 40° C. for 48 hours. 4.87 g of crystalline Form 2 Compound 1 di(hydrogen sulfate) trihydrate was obtained; isolated yield=81%. An XRPD pattern was collected confirming crystalline Form 2 Compound 1 di(hydrogen sulfate) trihydrate (FIG. 8). A Raman spectrum was also collected (FIG. 9).

### Example 3. Preparation 3 of Crystalline Form 2 Compound 1 Di(Hydrogen Sulfate) Trihydrate

253.75 g of a mixture of crystalline Form 1 Compound 1 (free base) and crystalline Form 2 Compound 1 (free base) (see Example 5 below) was added to a 5 L temperature-controlled reactor. 1.10 L of 1-propanol:water (80:20% v/v) was added to the reactor, followed by stirring at 50° C. at 100 RPM. 2.5 equivalents of sulfuric acid (95% wt.) were added to the reactor as a solution in 0.17 L of the solvent system, achieving a concentration of about 200 mg/mL. The experiment was equilibrated at 50° C., cooled to 40° C. and seeded with 1% crystalline Form 2 Compound 1 di(hydrogen sulfate) trihydrate. The seed crystal can be prepared methods such as those disclosed herein. Post-seeding, equilibration of ca. 6 hr was applied. The stirring speed was increased to 120 RPM during the equilibration period. The experiment was cooled to 20° C. at 0.2° C./min and equilibrated at 20° C. for 1 hour. At 20° C., anti-solvent addition with 1-propanol was carried out at 0.25 L/hr. 2.96 L of 1-propanol was added to reach a final ratio of 94:6% v/v. The stirring speed was increased to 190 RPM during the addition. Equilibration at 20° C. for about 1 hour was applied post-addition followed by cooling to 5° C. at 0.2° C./min and stirring at 5° C. for about 20 hours. The resulting slurry was vacuum filtered and the resulting isolated cake was washed with 0.5 L of the precooled resulting solvent system. The filter cake was dried under vacuum at 40° C. for 4 days. The dried solids were exposed to ambient conditions for about 20 hours to allow time for moisture equilibration. The moisture content of the solids after about 20 hours exposure to ambient condition was measured as 4.33% w/w. The solids were then re-exposed to ambient conditions for 2 days to allow time for further moisture uptake and equilibration during which time the solids were manually mixed intermittently.

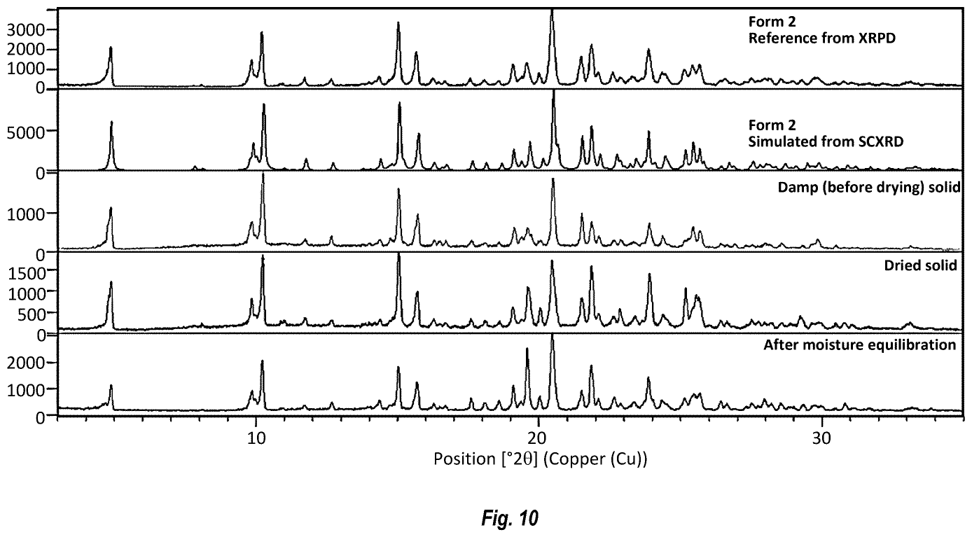

The resulting solids were shown to be crystalline Form 2 Compound 1 di(hydrogen sulfate) trihydrate by XRPD. FIG. 10 shows the material before drying, after drying, and after drying and moisture equilibration. All diffractograms are that of crystalline Form 2 Compound 1 di(hydrogen sulfate) trihydrate. An isolated yield of 80.4% was obtained, with the theoretical yield estimated as 80.3% (based on the concentrations of the extracted mother liquor and wash). The purity of the dried solids was determined as >99.99% area by HPLC and GC indicated about 0.12% by weight residual 1-propanol.

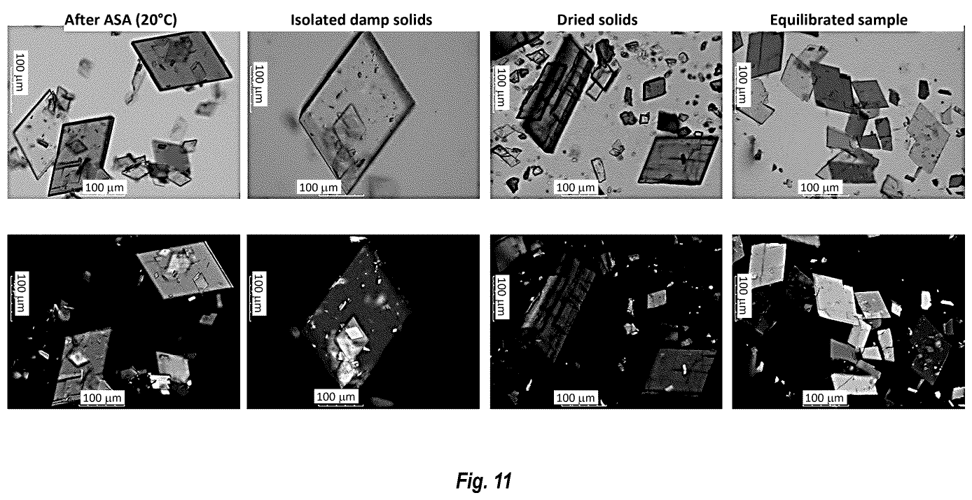

PLM showed the particle to be birefringent after anti-solvent addition at 20° C., as damp solids, after drying, and after moisture equilibration (FIG. 11). By Karl Fischer measurements, a water content on average of 4.33% w/w was measured after about 21 hours of re-exposure to ambient conditions, which increased to 6.07% after another 48 hours. A water content of 6.14% is the theoretical content for a trihydrate.

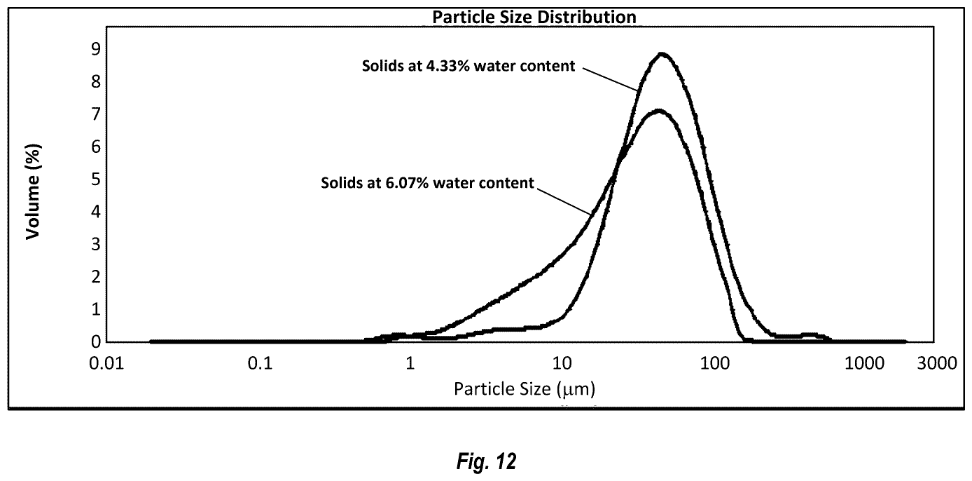

Particle size-measurements were taken before and after complete equilibration (moisture content of 4.33% and then later 6.07%). FIG. 12 is an overlay of the two measurements showing the difference in particle size distributions. The data are summarized in Table 11 below:

TABLE 11
Particle Size Measurements after Equilibration

| | Before complete | After complete |
|---|---|---|
| | equilibration; | equilibration; |
| | 24 hr post-drying | 3 days post-drying |
| | (moisture content = | (moisture content = |
| | 4.33%) | 6.07%) |
| D₁₀ | 17.596 μm | 6.32 μm |
| D₅₀ | 46.785 μm | 32.90 μm |
| D₉₀ | 109.799 μm | 84.73 μm |

These data show that the average particle size decreased with longer equilibration times and higher water contents.

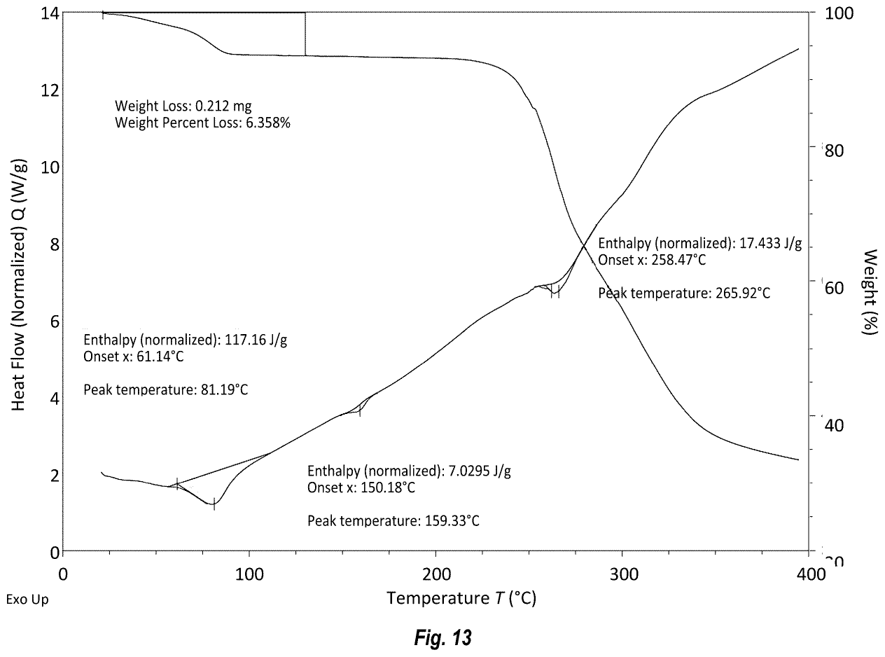

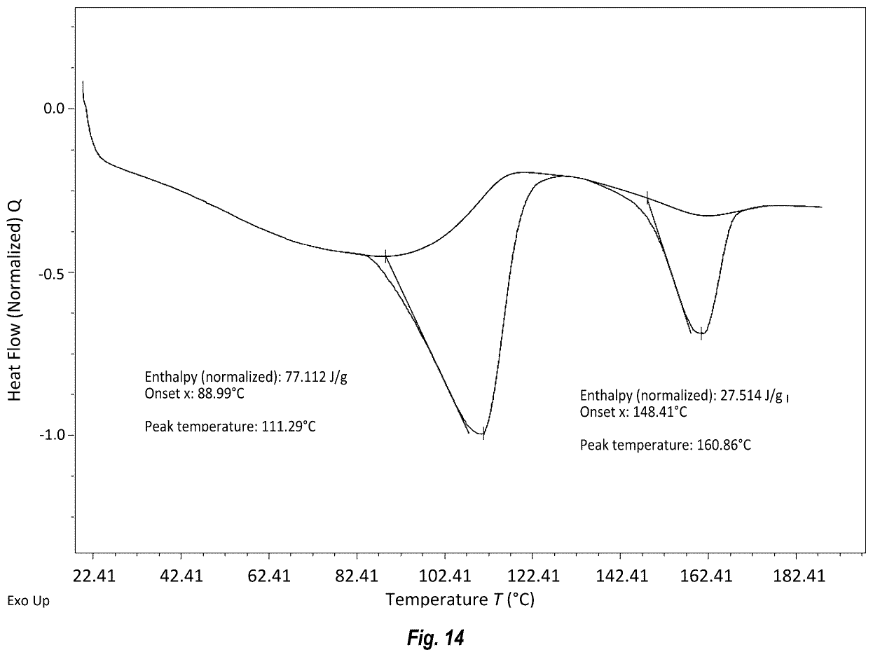

TG/DSC thermograms are presented in FIG. 13 of a fully equilibrated sample of crystalline Form 2 Compound 1 di(hydrogen sulfate) trihydrate. The data reveal a mass loss of about 6.4% between about 20° C. and 130° C., which corresponded with an endothermic event at about 61° C., both of which are likely due to solvent/water loss. A small endothermic event with a peak onset of about 150° C. may be due to melting and the thermal events above 200° C. are likely due to degradation. A DSC thermogram is provided in FIG. 14 showing thermal events at onsets of about 89° C. and about 148° C. The lower one is likely dehydration whereas the higher one is a melting event.

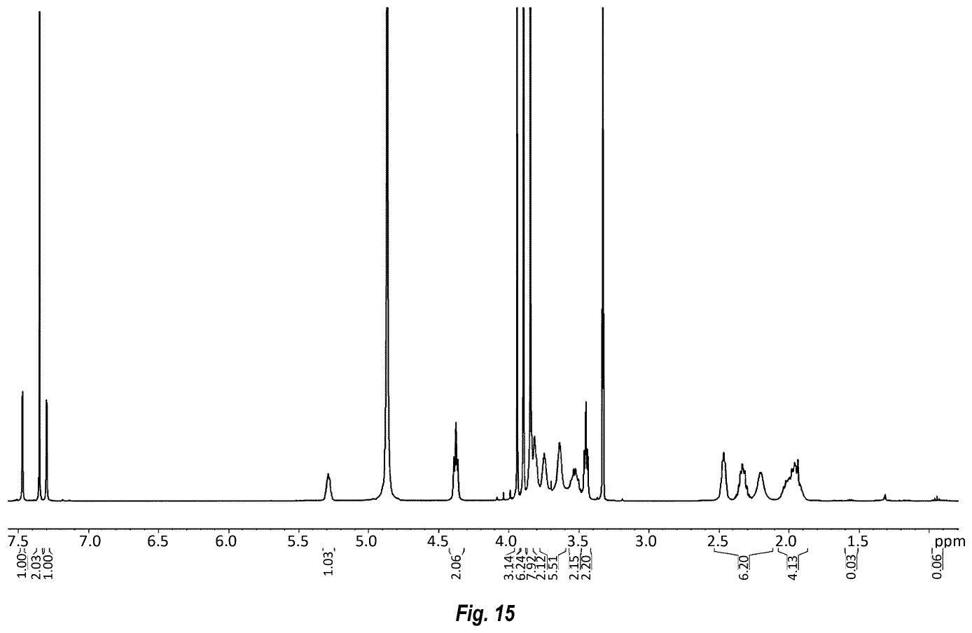

¹H-NMR spectra were collected on crystalline Form 2 Compound 1 di(hydrogen sulfate) trihydrate and the spectrum was consistent with the di(hydrogen sulfate) structure (FIG. 15).

An FT-IR spectrum of crystalline Form 2 Compound 1 di(hydrogen sulfate) trihydrate are shown in FIG. 16.

### Example 4. Single Crystal Study of Crystalline Form 2 Compound 1 Di(Hydrogen Sulfate) Trihydrate

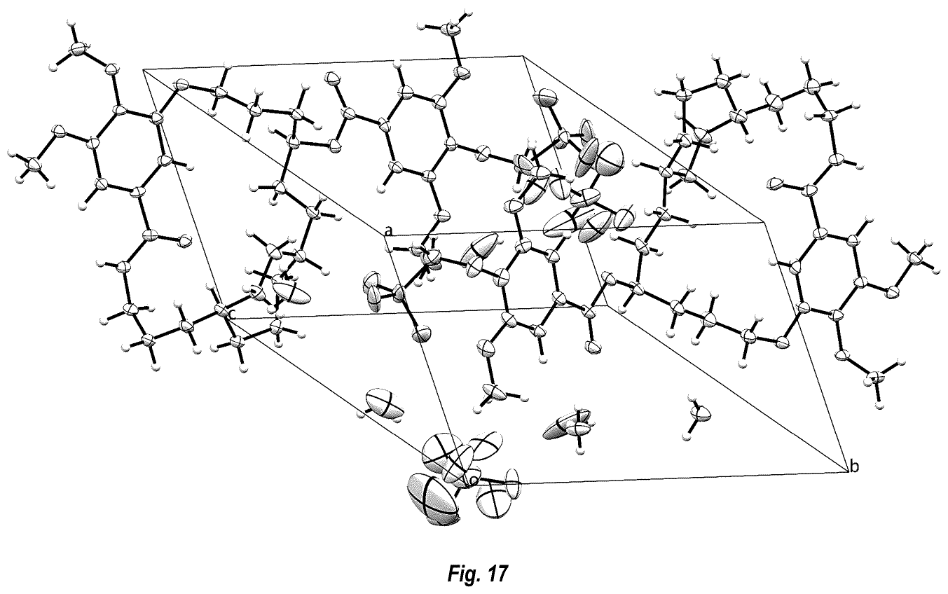

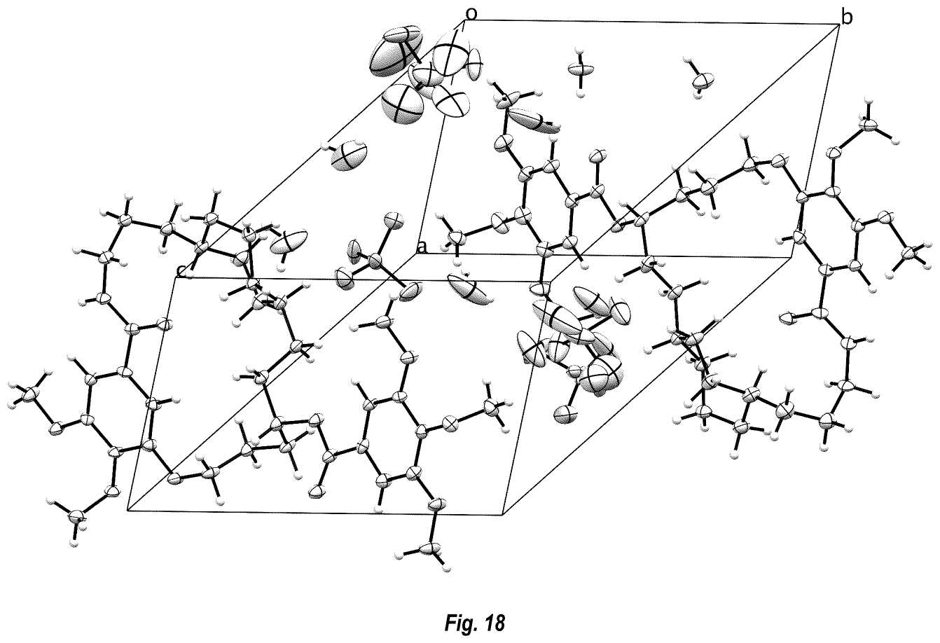

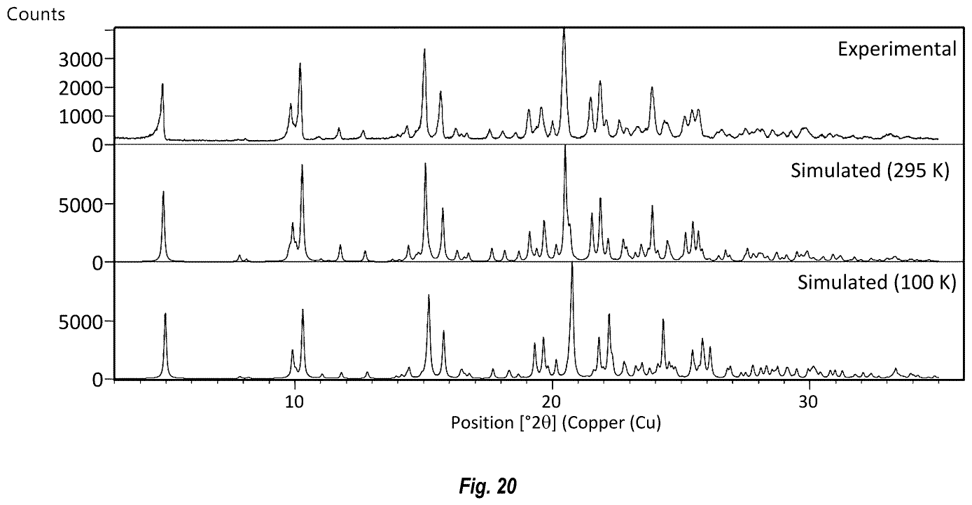

A single crystal of crystalline Form 2 Compound 1 di(hydrogen sulfate) trihydrate was prepared in the following manner. Approximately 10 mg of amorphous Compound 1 di(hydrogen sulfate) (prepared as described in Example 6B). was added to a 2 mL vial, into which 100 μL of 2-ethoxyethanol was added. Complete dissolution was observed at ambient temperature (ca. 20° C.). The solvent was then allowed to evaporate at ambient conditions for 3 days. Post-evaporation, the residual solid was observed to be clear block-like particles, which were analyzed by single crystal X-ray diffraction. FIGS. 17 and 18 show a single crystal structure drawing of crystalline Form 2 Compound 1 di(hydrogen sulfate) trihydrate at 100K in two different orientations. Simulated XRPD diffractograms were calculated for structures collected at 295 K and 100 K, respectively (FIG. 19), and compared to an experimental (room temperature) diffractogram (FIG. 20) with detailed peak list in Table 3 and Table 6 and the 20 most intense peaks listed in Table 4 and Table 7. The simulated diffractogram showed good correlation with the experimental diffractogram indicating that the structure presented in this data is representative of the bulk material.

### Example 5. Preparation of Crystalline Form 1 Compound 1 (Free Base) and Crystalline Form 2 Compound 1 (Free Base)

Approximately 100 mg of amorphous Compound 1 (free base) (see Example 6A) was weighed into a 1.5 mL screw-cap vial. Acetonitrile was added in 100 μL aliquots while stirring at 40° C. until the material dissolved (500 μL). The solution was stirred at 40° C. for 0.25 hr and then cooled to 5° C. at 0.1° C./min. A thick slurry (poorly mixed) was obtained at ca. 30° C. and so 200 μL of tBME was added as anti-solvent, but the slurry dissolved. After 11 hr at 5° C., a slurry was obtained. A portion of the slurry was isolated by centrifugation (0.22 μm nylon filter) and analyzed by XRPD. The isolated solids were consistent with a mixture of crystalline Form 1 Compound 1 (free base) and crystalline Form 2 Compound 1 (free base), which dried to crystalline Form 1 Compound 1 (free base).

Further aliquots of tBME were added to the remaining slurry (total of 600 μL), but dissolution was observed. Aliquots of heptane (3×100 μL) were added, but solution remained. Material was recovered by rotary evaporation and re-slurried in acetonitrile, then isolated by centrifugation (0.22 μm nylon filter) and analyzed by XRPD and found to be crystalline Form 1 Compound 1 (free base).

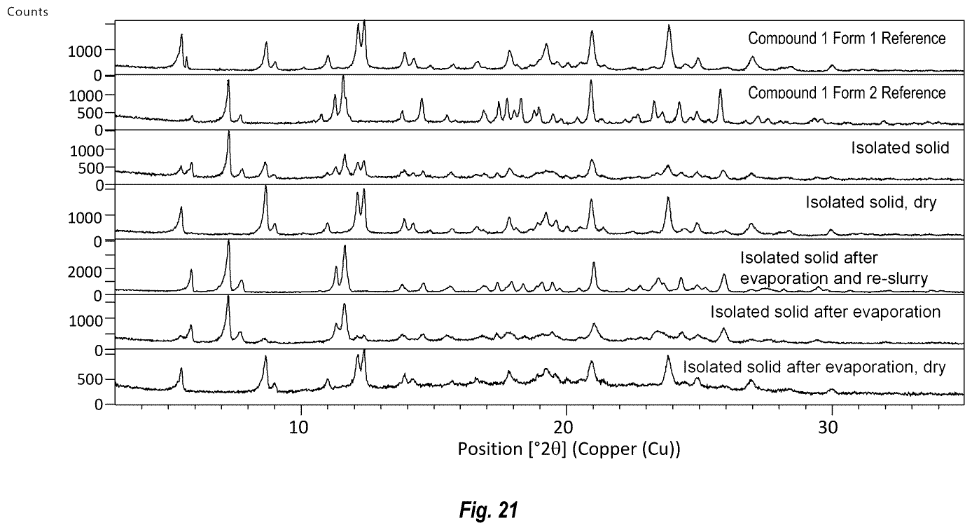

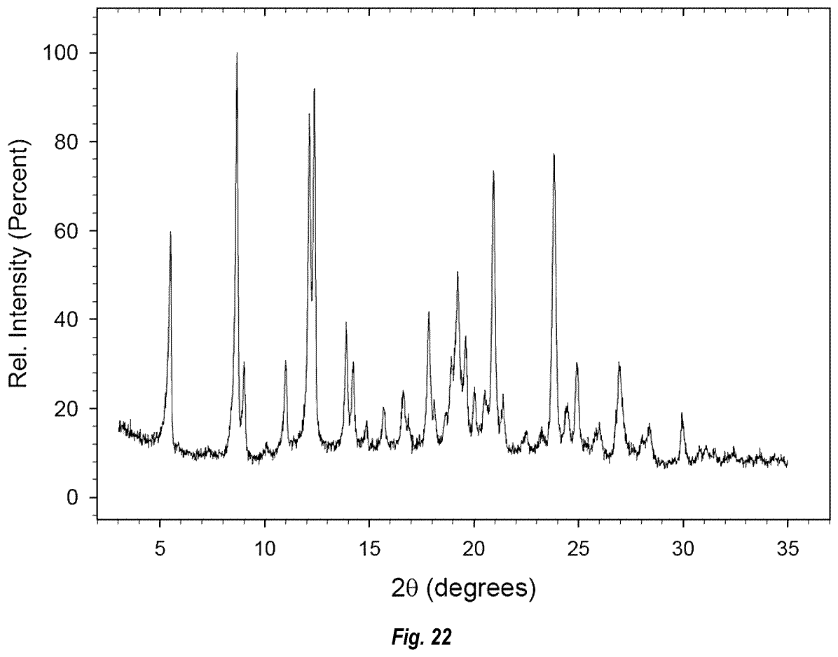

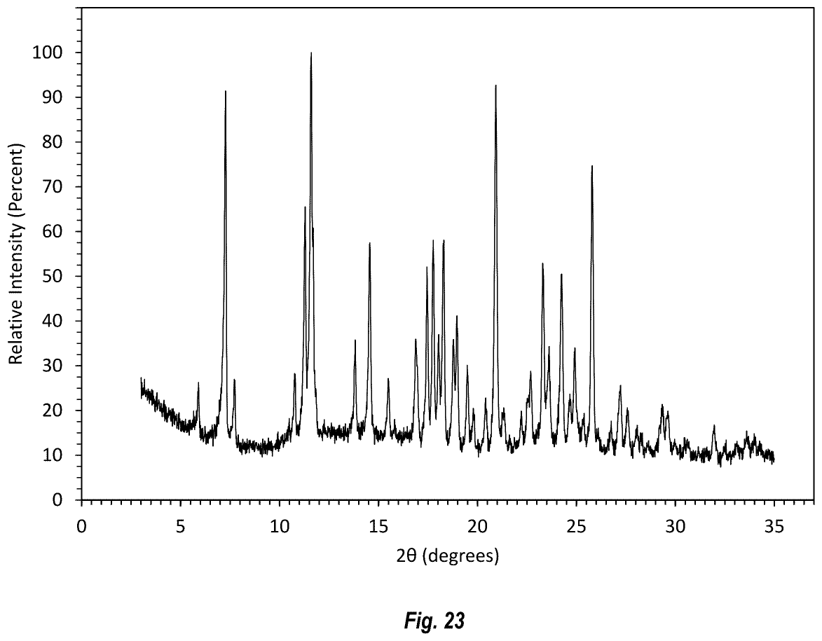

All liquors were evaporated to recover material. Solids were dried under vacuum at 35° C. for ca. 21 hr and then re-analyzed by XRPD. The solids were found to be crystalline Form 2 Compound 1 (free base), which dried to crystalline Form 1 Compound 1 (free base) with some loss of crystallinity. FIG. 21 is a stackplot of XRPD patterns taken during this experiment including crystalline Form 1 Compound 1 (free base) and crystalline Form 2 Compound 1 (free base) reference patterns. FIG. 22 is a separate XRPD pattern of crystalline Form 1 Compound 1 (free base) and FIG. 23 is a separate XRPD Pattern of crystalline Form 2 Compound 1 (free base), each prepared separately.

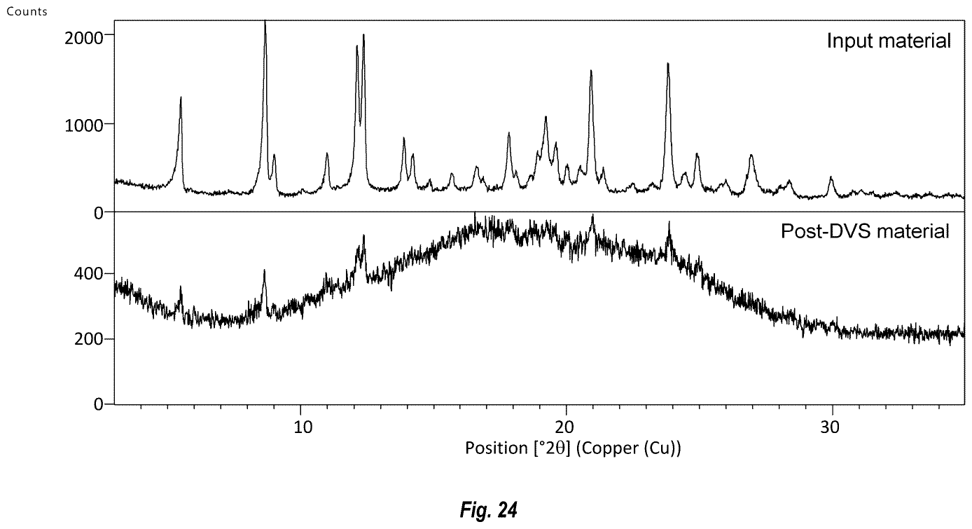

Hot stage microscopy showed that crystalline Form 1 Compound 1 (free base) started to melt at 65° C., with melting complete at 80° C. DVS analysis indicated that crystalline Form 1 Compound 1 (free base) was slightly hygroscopic with water uptake of 0.9% between 40-80% RH in the first sorption cycle. A further uptake of 3.8% between 80-90% RH likely resulted in formation of amorphous material, which was then moderately hygroscopic (uptake of 3.3% at 80% RH) in the second sorption cycle. Post-DVS XRPD analysis indicated that predominantly amorphous material was recovered (traces of crystalline Form 1 Compound 1 (free base)) as shown in FIG. 24.

### Example 6. Preparation of Amorphous Compound 1 (Free Base) and Amorphous Compound 1 Di(Hydrogen Sulfate)

(A) Amorphous Compound 1 (free base) was used to prepare different crystalline forms. Compound 1 (free base) can be prepared as described by, for example, WO 2021/204896 or as described by WO 2023/056910, for example, as well.

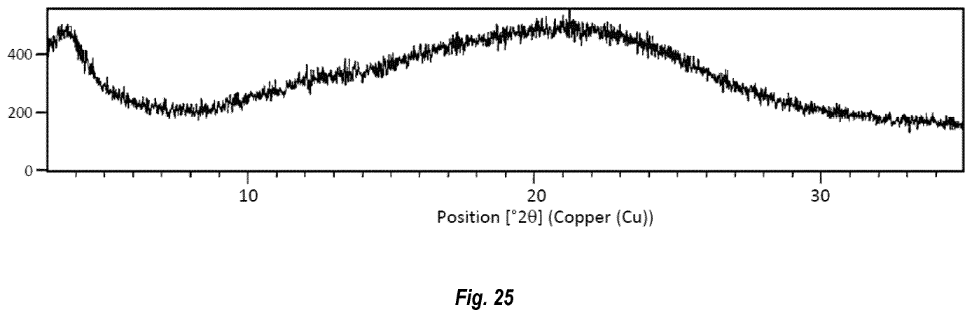

(B) Amorphous Compound 1 di(hydrogen sulfate) was used to prepare the single crystal that was used in the Single Crystal Study (see Example 4 above). The amorphous Compound 1 di(hydrogen sulfate) was prepared as follows. Approximately 2.5 g of crystalline Form 2 Compound 1 di(hydrogen sulfate) trihydrate (see, e.g., Example 4) was added to a 250 mL conical flask. 100 mL of methanol was added to the flask to dissolve the solid at ambient temperature. When complete dissolution was observed, the solvent was removed under vacuum via rotary evaporation. An amorphous solid resulted as determined by XRPD (FIG. 25). ¹H-NMR analysis of a solution of the compound prepared from the solid material was consistent with the ¹H-NMR spectrum of Compound 1.

### BIOLOGICAL EXAMPLES

### Example 7. Mouse Tumor Model Experiment

The anti-tumor therapeutic efficacy of Compound 1 monotherapy, nivolumab monotherapy, and Compound 1-nivolumab combination therapy was assessed in ‘humanized’ mice with subcutaneous tumors resulting from the subcutaneous injection of MDA-MB-231 cells, a human triple-negative breast cancer cell line.

Human cell line xenograft experiments were performed at Transcure (France) and all associated animal procedures were reviewed and approved by the local ethics committee. The experiment was carried out with the NOD-Prkdc^{em26cd5}2Il2rg^{em26cd22}/NjuCrl immunodeficient mouse strain (NCG from Charles River Laboratories, Wilmington, MA, USA). Four-week-old immunodeficient NCG mice were engrafted with cord-blood-derived human CD34⁺ hematopoietic stem and progenitor cells (French Blood Institute, Paris, France) 2 days after chemical myeloablative treatment. Fourteen weeks after cell injection, engraftment level was monitored with the analysis of human CD45⁺ cells among total blood leukocytes by flow cytometry (Attune, Life Technologies/Thermo Fisher Scientific, Waltham, MA, USA). Engraftment was confirmed if human CD45⁺ cells represented 25% or more of total blood leukocytes.

MDA-MB-231 cells were obtained from The American Type Culture Collection (ATCC) and were cultured in L-15 medium supplemented with 10% FBS and 1% Glutamine (Gibco) in culture flasks (Greiner) and incubated at 37° C. with 5% CO₂.

After engraftment confirmation as described above (hCD45/total CD45>25%), the now ‘humanized’ mice were inoculated subcutaneously with 5×10⁶ MDA-MB-231 cells resuspended in 50% Matrigel (Corning). When the tumors reached approximately 60 mm³ (day 19 after tumor cell inoculation), the animals were randomized into 4 treatment groups by manual randomization based on their humanization rate, tumor volume, and the cord blood donor ID.

The groups were then treated as follows:
Group 1—‘Vehicle’—mice received a 100 μL solution of 2.5% dimethyl sulfoxide (DMSO), 10% solutol HS15 (Sigma-Aldrich), and 87.5% phosphate-buffered saline (PBS) orally twice a day.
Group 2—‘Compound 1’—mice received 200 μL solution of Compound 1 (30 mg drug per kg of mouse body weight—i.e. 30 mg/kg) dissolved in 2.5% DMSO, 10% solutol HS15, and 87.5% PBS by intraperitoneal (i.p.) injection twice a day.
Group 3—‘anti-PD-1’—mice received 200 μL of a nivolumab solution at 10 mg/kg in PBS via i.p. injection once every 5 days.
Group 4—‘Compound 1+anti-PD-1’—mice received a 200 μL solution of Compound 1 (30 mg/kg) dissolved in 2.5% DMSO, 10% solutol HS15, and 87.5% PBS by i.p. injection twice a day and 200 μL of a nivolumab solution at 10 mg/kg in PBS via i.p. injection once every 5 days.

All the animals used were monitored daily, checking their clinical signs of distress and body weight and tumors were measured three times a week using an electronic caliper. The tumor volume was determined by applying the formula Length×(Width)²×0.5.

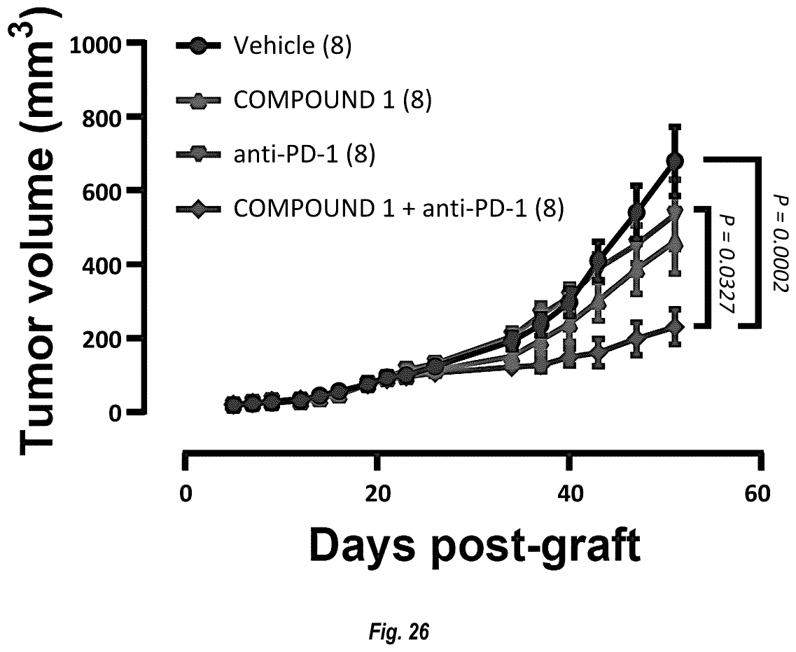

In FIG. 26, mean tumor volumes in mm³ are depicted±standard error of the mean (s.e.m.) from 1 experiment, with n=8 mice per group. Statistical analysis was performed with GraphPad Prism (version 10.0.2) using ordinary two-way analysis of variance (ANOVA) of log-transformed tumor volumes with Šidák's multiple comparisons test, p value displayed for day 51.

Tumor volumes in both the Compound 1 monotherapy and anti-PD-1 monotherapy groups were not statistically significantly different from the vehicle group as assessed on day 51 of the experiment. Tumor volumes of the Compound 1+anti-PD-1 group were statistically significantly smaller than the vehicle group (P=0.0002) and the anti-PD-1 group (P=0.0327) on day 51 of the experiment.

### Example 8. Adenosine Uptake and T-cell Proliferation

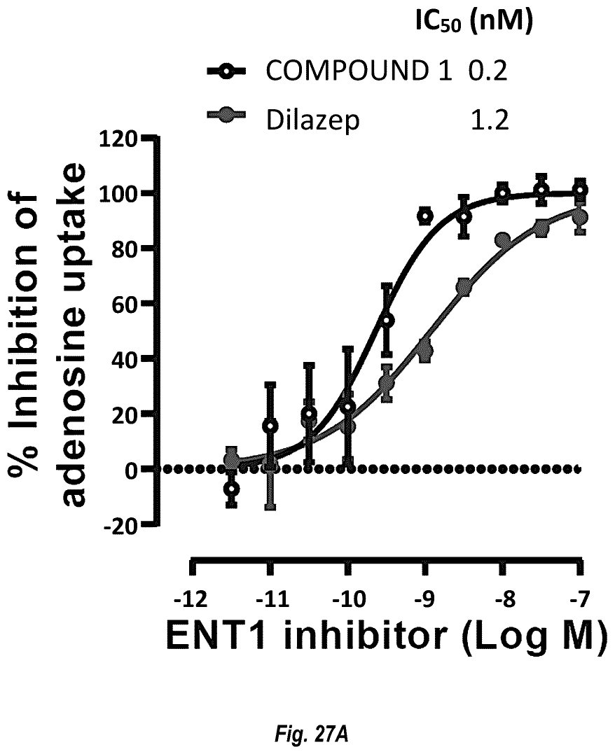

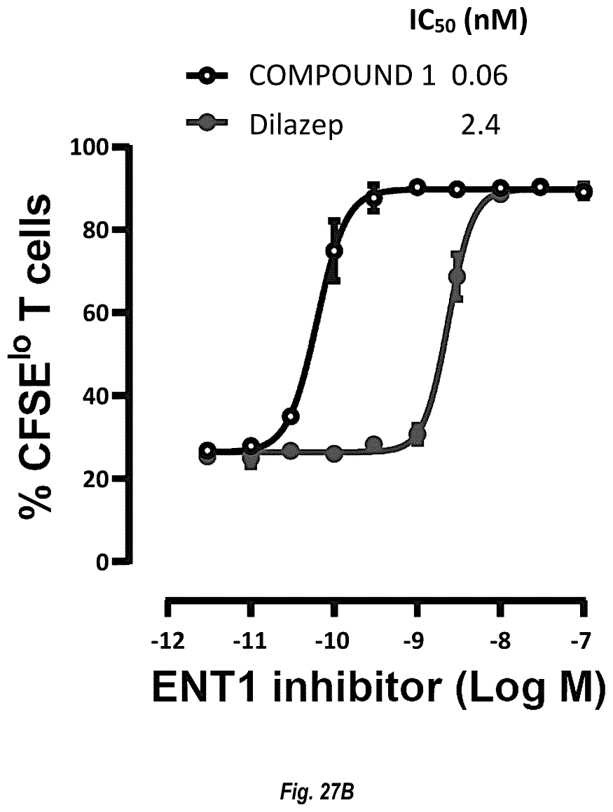

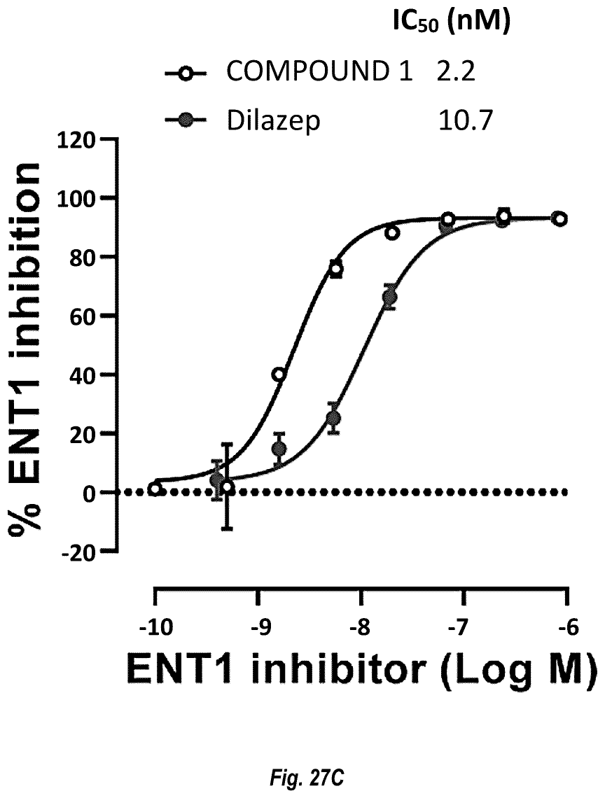

Human T cells were activated for 2 days in the presence of Compound 1 or dilazep. The cells were then washed and used in a [³H]adenosine uptake experiment. The results indicate that Compound 1 inhibits the uptake of adenosine into human T cells with an IC50 value of 0.2 nM as compared with 1.2 nM for dilazep. (See FIG. 27A.)

Human T cells were activated in the presence of 100 μM ATP and dilazep or Compound 1 and proliferation was monitored by CarboxyFluoroscein Succinimidyl Ester (CFSE) dilution. The results indicate that Compound 1 restores T cell proliferation under these conditions with an IC50 value of 0.06 nM as compared with 2.4 nM for dilazep under these conditions. (See FIG. 27B.)

ENT1 transport inhibition assay demonstrates that Compound 1 is a potent ENT1 antagonist and inhibits ENT1-mediated uridine transport with nanomolar potency. The results indicate that Compound 1 inhibits ENT1 with an IC50 value of 2.2 nM as compared with 10.7 nM for dilazep. (See FIG. 27C.)

### Example 9: Compound 1 Protects Tumor-Infiltrating T Cells from Adenosine

Human tumors were dissociated, and cells were CFSE-labelled and stimulated with CD3/CD28 microbeads and IL-2 in the presence of 500 μM of ATP as a source of adenosine.

Human tumor pieces were rinsed with cold PBS and cut into 1 to 2 mm fragments with a scalpel blade and transferred into GentleMACS C tubes. Dissociation of the tissue was completed with the Tumor Dissociation kit (Miltenyi Biotec) according to the manufacturer's instruction. Briefly, 5 mL of dissociation solution—containing 200 μL of enzyme H, 20 μL of enzyme R and 25 μL of enzyme A and RPMI until 5 mL—was added per gram of tumor. The tube was placed in the gentleMACS dissociator and the 1st program was run. An incubation step of 30 minutes at 37° C. was applied with rotation (MACSmix tube rotator). After this incubation, a quick centrifugation (30×rcf, 1 min) was performed, the supernatant containing dissociated cells was collected in a new tube containing FBS (30% of the total volume with supernatant) and placed at 4° C. A 2nd round of dissociation was performed on the remaining tissue fragments: after the addition of dissociation solution (same volume and content as the 1st round), the 2nd gentleMACS program was run and the tube was placed for 30 min incubation at 37° C. with rotation. A short centrifugation was performed, the cells in supernatant were pooled with the 1st collection and filtered through a 100 μm cell strainer which was washed with 10 mL RPMI 10% FBS 2% P/S. After centrifugation at 350×rcf for 10 min, cells were resuspended in 20 mL RPMI 10% FBS 2% P/S and counted with a haemocytometer. If cells agglomerates were visible, the cell suspension was filtered through a 100 μm cell strainer before counting. Dissociated tumour cells (DTCs) were either used directly for FACS analysis or else were washed into FBS with 10% DMSO and cryopreserved in liquid nitrogen until required.

Cryopreserved human DTCs were thawed and washed twice with RPMI1640 medium (Lonza, Verviers, Belgium) containing 10% hiFBS. Cells were resuspended in PBS with 10% FBS at a concentration of 107 cells/ml. One volume of cells was combined with 1 volume of CFSE solution in PBS to give a final CFSE concentration of 1 μM. The cell suspension was mixed and incubated at 37° C. for 5 minutes before cells were topped up with PBS with FBS (10%) to quench remaining excess CFSE, before centrifugation at 450×rcf for 5 minutes. The cells were resuspended at 4×106 cells/mL in X-VIVO15 medium containing 5% (v/v) HS (Biowest), 1 mM Na-Pyr and 2% P/S (Westburg (LONZA)). Cell suspension (50 μl, 2×105 cells) was added to wells of sterile round-bottom 96-well plates. Cells were left unstimulated, or activated by adding 50 μL of 50 U/mL IL-2 (Proleukin-Novartis) and anti-CD3 anti-CD28 coated microbeads (Dynabeads human T-activator CD3/CD28; Life Technologies, Paisley, UK) at a ratio of one microbead per five cells. ENT1 inhibitor Compound 1 (300 nM, iTeos Therapeutics; stock solution of 10 mM in DMSO), and anti-PD-1 (10 μg/mL) OPDIVO®; nivolumab, Bristol-Myers Squibb) was prepared and distributed to the relevant wells of the culture plate. A concentration-matched isotype control antibody was added to all wells not containing nivolumab and DMSO concentrations were matched across all samples to 0.1%. Cells were cultured in the presence or absence of ATP (Sigma-Aldrich) at a final concentration of 500 μM. The final well volume was 200 μl in all cases. Experiments were performed at a minimum in technical in duplicates. Cells were mixed by pipetting up and down and incubated for 3 days in a 37° C. humidified tissue culture incubator with 5% CO2. An additional dose of ATP at a final concentration of 500 μM was added 18 hours after the start of culture.

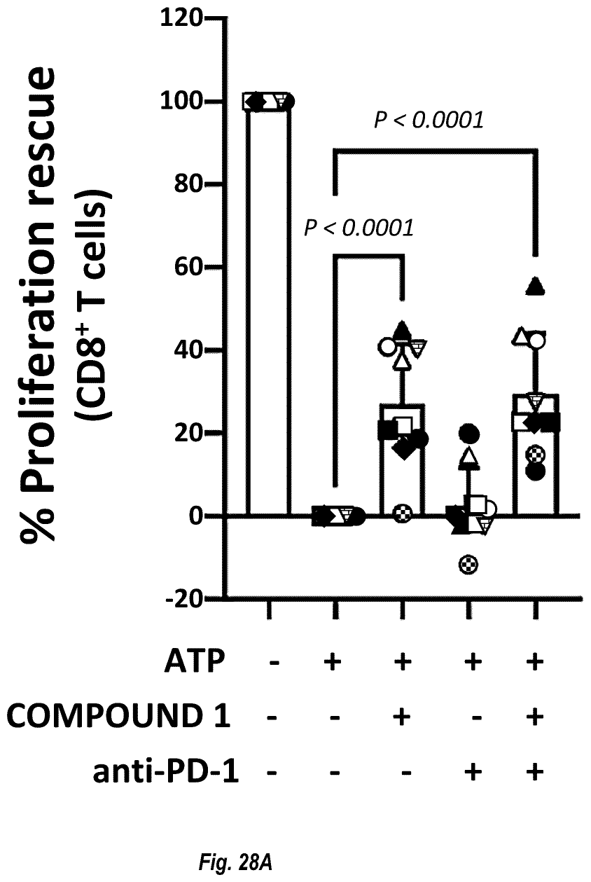

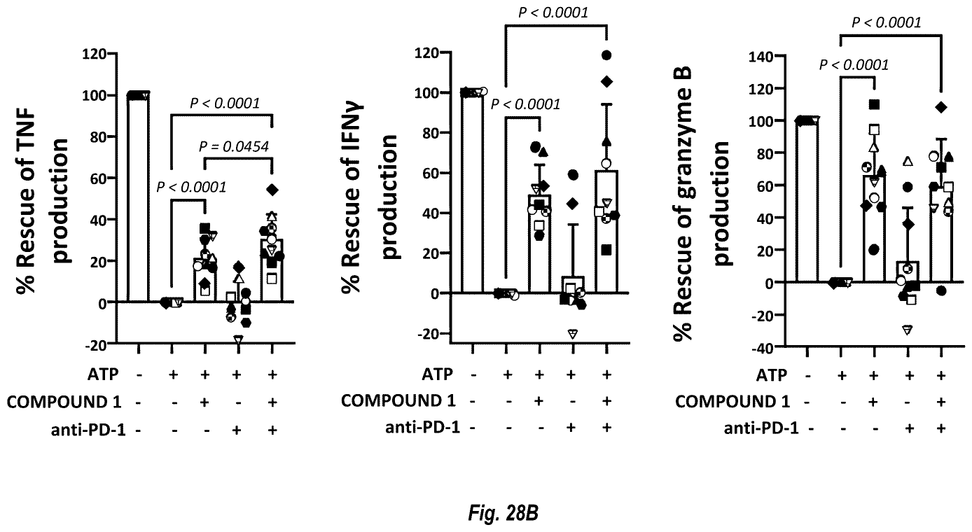

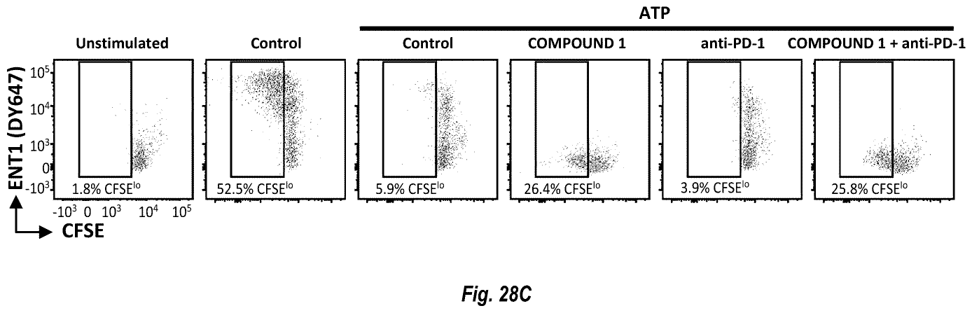

Summary data of Compound 1-mediated rescue of CD8+ T cell proliferation and TNF, IFNγ, and granzyme B, respectively, from dissociated tumor cell suspension cultures was shown in FIGS. 28A and 28B. For the summary analysis of T cell proliferation and cytokine production, the mean value of the readout recorded in the presence of ATP but with no other treatments was subtracted from all values. These were then expressed as a percentage of the mean value of control samples stimulated in the absence of ATP. This generated a scale of rescue from 0 to 100 in which 0 represents no difference from the ATP level and 100 represents a full rescue.

In FIG. 28A, the data evaluated whether chronic adenosine exposure would suppress the expansion and function of tumor infiltrating T cells upon activation and if this could be prevented with Compound 1. Dissociation of resected tumors from cancer patients into a cell suspension followed by CFSE labelling allowed T cell proliferation to be monitored over 3 days in culture. While the presence of anti-CD3/CD28 microbeads induced robust proliferation of CD8+ and CD4+ T cells, this was strongly suppressed when ATP was supplemented into the cultures to model intratumoral adenosine generation. Inclusion of Compound 1 restored T cell proliferation in the presence of ATP, which was not further enhanced by the presence of anti-PD-1. The results indicate that ATP restricted T cell proliferation was partially reversed by Compound 1 alone and in combination with anti-PD-1. (See FIG. 28A.)

TNF, IFNγ, and granzyme B levels were also assessed in culture supernatants. Production of cytokines associated with cytotoxic T cell function were also suppressed by the inclusion of ATP and all were restored, to varying extents, by Compound 1 (see FIG. 28B). Production of TNF and IFNγ were further enhanced when Compound 1 was combined with anti-PD-1 (see FIG. 28B).

FIG. 28C shows Compound 1 suppresses ENT1 expression.

### Example 10: Compound 1 Restores T Cell Expansion and Antigen-Specific Killing Activity

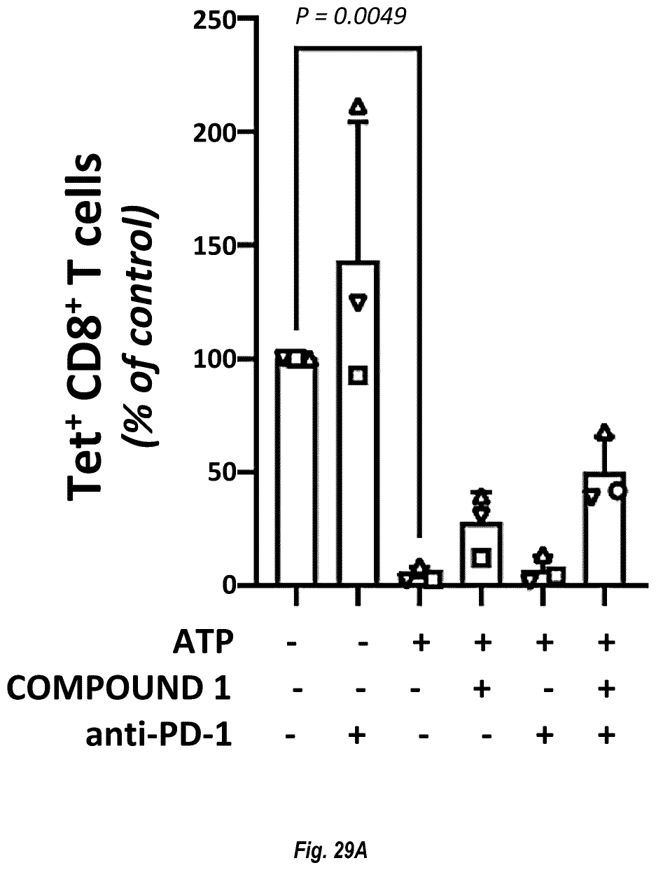

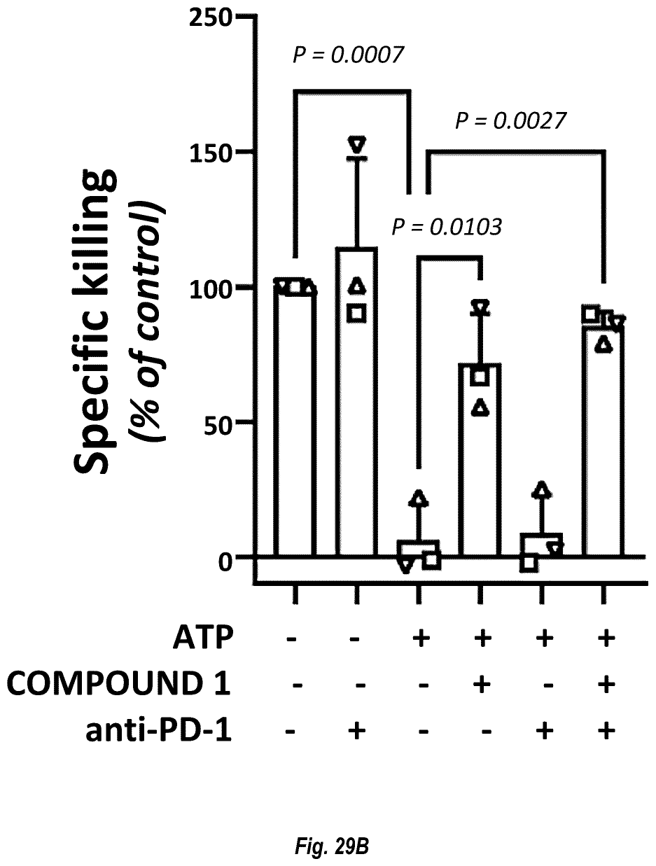

Human T cells were activated in the presence of 300 μM ATP and Compound 1, anti-PD-1, or Compound 1 and anti-PD-1. CD8+ T cell population was monitored by tetramer-positive (Tet+) staining data. The results indicate that Compound 1 restores CMV pp65 peptide-specific T cell expansion in the presence of ATP (300 μM) as a source of adenosine, resulting in a restoration of antigen-specific killing activity, respectively. (See FIGS. 29A-29B).

### Example 11: Human Studies Trial

In this study, patients with solid tumors are treated with crystalline Form 2 Compound 1 di(hydrogen sulfate) trihydrate, alone (“Part 1A”) or in combination with the checkpoint inhibitor pembrolizumab (“Part 1”).

In Part 1A of the study, patients receive crystalline Form 2 Compound 1 di(hydrogen sulfate) trihydrate in one of the following dosing scheme: (1) a single daily oral dose of 5 mg of Compound 1 (free base); (2) twice a day oral doses of 5 mg of Compound 1 (free base)—i.e., 10 mg of Compound 1 (free base) total per day; (3) twice a day oral doses of 10 mg of Compound 1 (free base)—i.e., 20 mg of Compound 1 (free base) total per day; (4) twice a day oral doses of 15 mg of Compound 1 (free base)—i.e., 30 mg of Compound 1 (free base) total per day; (5) twice a day oral doses of 22.5 mg of Compound 1 (free base)—i.e., 45 mg of Compound 1 (free base) total per day; or (6) twice a day oral doses of 30 mg of Compound 1 (free base)—i.e., 60 mg of Compound 1 (free base) total per day.

The aim of Part 1A is to evaluate the safety, tolerability, pharmacokinetics (PK), pharmacodynamics (PD), and preliminary antitumor efficacy of crystalline Form 2 Compound 1 di(hydrogen sulfate) trihydrate as a monotherapy and to define the maximum tolerated dose (MTD) (if applicable) and the tentative Recommended Phase 2 dose(s) (RP2D) for the crystalline Form 2 Compound 1 di(hydrogen sulfate) trihydrate when given alone.

Part 1A will consist of sequential dose-escalation cohorts with at least 3 and up to 6 dose-limiting toxicity (DLT)-evaluable participants each. There are 5 planned dose level cohorts in Part 1A, as discussed earlier. The planned starting human dose of crystalline Form 2 Compound 1 di(hydrogen sulfate) trihydrate in Cohort 1 (Dose Level 1) is 5 mg (expressed as free-base equivalent) twice daily (BID) oral dosing (PO). The first dose-escalation cohort (Cohort 1) will start with a single-dose administration of 5 mg (free base) in Cycle 0 followed by the washout period of at least 3 and approximately 7 days before Day 1 of Cycle 1. On Day 1 of Cycle 1, participants in Cohort 1 will start taking crystalline Form 2 Compound 1 di(hydrogen sulfate) trihydrate at 5 mg (free base) BID continuously for 21 days of each 21-day cycle. Subsequent dose-escalation cohorts may also include the single-dose Cycle 0 until sufficient PK/PD information is collected to support the prediction of PK and PD at higher BID dose levels.

The initial dosing schedule is BID (every 12 hours±2 hours) orally continuously for 21 days of each 21-day treatment cycle.

The dosing schedule may be modified at any time at the Sponsor's discretion and with the Safety Review Committee (SRC) approval in response to emerging data. Further, intermittent dosing (e.g., 5 days on and 2 days off in 21-day cycles, or 3 weeks on and 1 week off in 28-day cycles) may be used if treatment holiday is deemed necessary to manage the incidence or severity of treatment-emergent adverse events (TEAEs).

Based on the evaluation of all available study data from Part 1A, the Sponsor will recommend the dose(s) and dosing schedule to be used as the tentative RP2D(s) for the crystalline Form 2 Compound 1 di(hydrogen sulfate) trihydrate as monotherapy in Part 1A. At least 6 and approximately 9 DLT-evaluable participants must be evaluated at a dose level before it could be considered as the tentative RP2D. The tentative RP2D(s) for crystalline Form 2 Compound 1 di(hydrogen sulfate) trihydrate as monotherapy may be evaluated further in the Phase 1b expansion cohort(s) in Part 2 according to a future protocol amendment.

The aim of Part 1B is to evaluate the safety, tolerability, PK, PD, and preliminary antitumor efficacy of crystalline Form 2 Compound 1 di(hydrogen sulfate) trihydrate in combination with pembrolizumab and to define the MTD (if applicable) and the tentative RP2D(s) for crystalline Form 2 Compound 1 di(hydrogen sulfate) trihydrate when given with pembrolizumab.

Part 1B involves sequential dose-escalation cohorts of participants with advanced solid tumors. Part 1B will be initiated after the MTD (if applicable) and tentative RP2D for crystalline Form 2 Compound 1 di(hydrogen sulfate) trihydrate as monotherapy have been identified in Part 1A. It is estimated that there could be 3 planned crystalline Form 2 Compound 1 di(hydrogen sulfate) trihydrate dose-escalation cohorts in Part 1B.

The starting dose of crystalline Form 2 Compound 1 di(hydrogen sulfate) trihydrate in Part 1B will be at least 1 dose level below the highest dose that has been shown to be safe and well tolerated as monotherapy in Part 1A at that time. The Sponsor will determine the specific starting dose of crystalline Form 2 Compound 1 di(hydrogen sulfate) trihydrate in Part 1B based on the review of all available safety, PK, PD, and tumor response data for crystalline Form 2 Compound 1 di(hydrogen sulfate) trihydrate as monotherapy across all dose levels evaluated in Part 1A at that time.

The SRC must approve the starting dose of crystalline Form 2 Compound 1 di(hydrogen sulfate) trihydrate in Part 1B before the first dose cohort in Part 1B is opened.

The initial dosing schedule for crystalline Form 2 Compound 1 di(hydrogen sulfate) trihydrate is BID (every 12 hours±2 hours) orally continuously for 21 days of each 21-day treatment cycle. In all dose cohorts in Part 1B, the planned dose of pembrolizumab is fixed at 200 mg once every 3 weeks (Q3W) given by a 30-minute intravenous (IV) infusion on Day 1 of each 21-day cycle.

Treatment with both study drugs would start on Day 1 of Cycle 1, where the crystalline Form 2 Compound 1 di(hydrogen sulfate) trihydrate will be administered 30 (±5) minutes before the start of pembrolizumab infusion.

### EQUIVALENTS

The foregoing written specification is considered to be sufficient to enable one skilled in the art to practice the embodiments. The foregoing description and Examples detail certain embodiments and describe the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

What is claimed is:

1. A combination of an ENT1 inhibitor and a checkpoint inhibitor.

2. A pharmaceutical composition comprising an ENT1 inhibitor and a checkpoint inhibitor.

3. A kit of parts comprising an ENT1 inhibitor and a checkpoint inhibitor.

4. A method of reversing adenosine-mediated suppression of T cell proliferation and/or viability, comprising administering an ENT1 inhibitor and a checkpoint inhibitor.

5. A method of reversing adenosine-mediated suppression of antigen-specific killing activity of T cells, comprising administering an ENT1 inhibitor and a checkpoint inhibitor.

6. A method of reversing adenosine-mediated suppression of production of cytokines, comprising administering an ENT1 inhibitor and a checkpoint inhibitor.

7. A method of treating cancer comprising administering to a subject in need thereof an ENT1 inhibitor and a checkpoint inhibitor.

8. A use of an ENT1 inhibitor and a checkpoint inhibitor in the manufacture of a medicament for the treatment of cancer.

9. The combination, composition, kit, method or use according to any one of claims 1-8, wherein the checkpoint inhibitor is not an anti-TIGIT antibody.

10. The combination, composition, kit, method or use according to any one of claims 1-9, wherein the checkpoint inhibitor is chosen from a PD1 inhibitor, a PD-L1 inhibitor, and a CTLA-4 inhibitor.

11. The combination, composition, kit, method or use according to any one of claims 1-10, wherein the checkpoint inhibitor is a PD1 inhibitor.

12. The combination, composition, kit, method or use according to any one of claims 1-11, wherein the checkpoint inhibitor is selected from pembrolizumab, nivolumab, atezolizumab, dostarlimab, durvalumab, avelumab, and opdualag.

13. The combination, composition, kit, method or use according to any one of claims 1-12, wherein the ENT1 inhibitor is a compound of Formula (I): 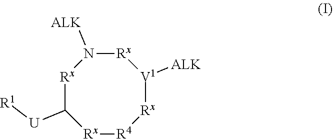
or a pharmaceutically acceptable salt, hydrate, or solvate thereof,
wherein
R¹ is selected from the group consisting of 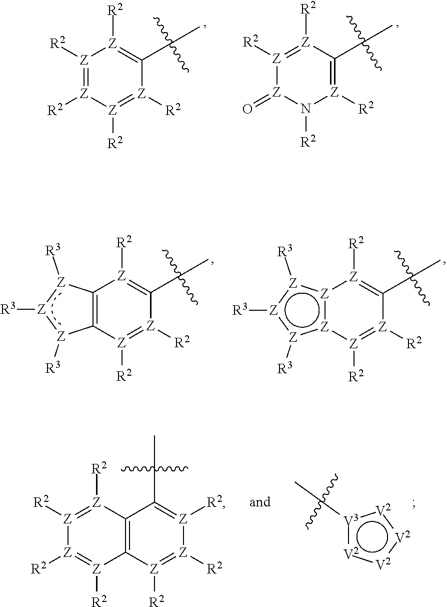
each R² is independently selected from the group consisting of absent, halogen, —NHR³, —OR³, —R³, —C(O)R³, —CO₂R³, C(O)N(R³)₂, —CH₂C(O)N(R³)₂, —S(O)₂R³, and —CN;
or two instances of R² are taken together with the atoms on which they are attached to form a heterocyclyl or heteroaryl ring;
each R³ is independently selected from absent, —H, oxo, ALK, phenyl, heterocyclyl, and heteroaryl;
R⁴ is selected from the group consisting of 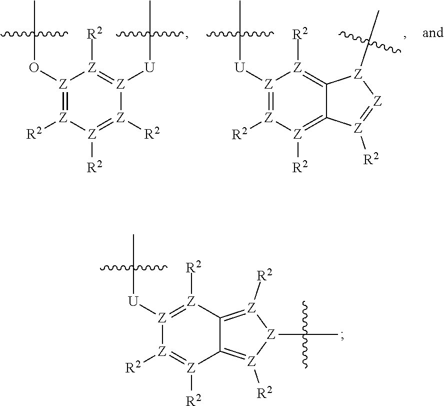
each U is independently selected from the group consisting of —C(O)—, alkylene, —O—, —N(R³)—, —C(O)O—, —C(O)N(R³)—, and 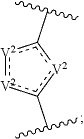
each R^{x} is independently selected from alkylene;
V¹ is selected from —C(R³)— and —N—;
each V² is independently selected from —C(R³)═, —N(R³)—, —N═, and —O—;
V³ is selected from —C═ and —N—; and
Z is C or N,
wherein ALK is unsubstituted alkyl or substituted alkyl, or two instances of ALK may be joined together with their intervening atoms to form a cycloalkyl or heterocyclyl ring.

14. The combination, composition, kit, method or use according to any one of claims 1-12, wherein the ENT1 inhibitor is a compound of Formula (II): 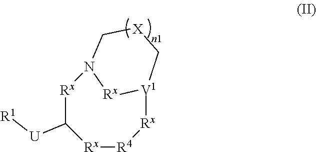
or a pharmaceutically acceptable salt, hydrate, or solvate thereof,
wherein
R¹ is selected from the group consisting of ALK, cycloalkyl, heterocyclyl, 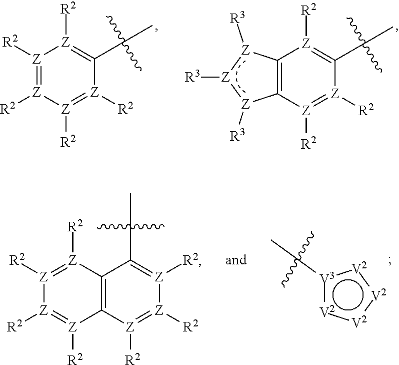
each R² is independently selected from the group consisting of absent, halogen, —OR³, —R³, —CO₂R³, C(O)N(R³)₂, —CH₂C(O)N(R³)₂, —S(O)₂R³, and —CN;
or two instances of R² are taken together with the atoms on which they are attached to form a heterocyclyl or heteroaryl ring;
each R³ is independently selected from absent, —H, ALK, phenyl, and heteroaryl;
R⁴ is 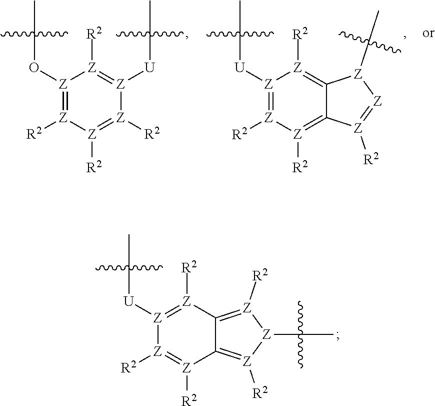
X is selected from the group consisting of —CH₂—, —CHF—, and —CF₂—;
each U is independently selected from the group consisting of —O—, —N(R³)—, —C(O)O—, —C(O)N(R³)—, 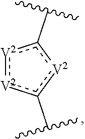
—C(O)—, —O—N═C(H)— and alkylene;
each R^{x} is independently selected from alkylene;
V¹ is selected from —C(R³)— and —N—;
each V² is independently selected from —C(R³)═, —N(R³)—, —N═, and —O—;
V³ is selected from —C═ and —N—;
each Z is independently C or N; and
n¹ is a number of 0 or 1,
wherein ALK is unsubstituted alkyl or substituted alkyl, or two instances of ALK may be joined together with their intervening atoms to form a cycloalkyl or heterocyclyl ring.

15. The combination, composition, kit, method or use according to claim 14, wherein the ENT1 inhibitor is a compound of Formula (IIa): 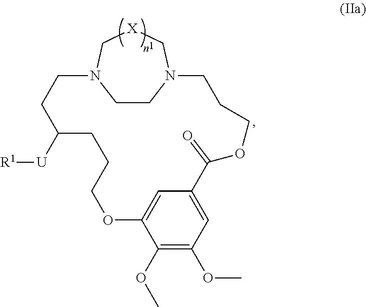
or a pharmaceutically acceptable salt, hydrate, or solvate thereof,
wherein X is CH₂, CHF, or CF₂.

16. The combination, composition, kit, method or use according to any one of claims 13-15, wherein R¹ is

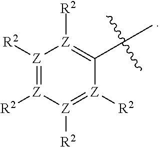

17. The combination, composition, kit, method or use according to claim 16, wherein R¹ is

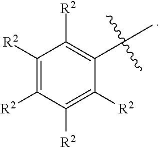

18. The combination, composition, kit, method or use according to any one of claims 14-17, wherein the compound is a compound of Formula (IIb): 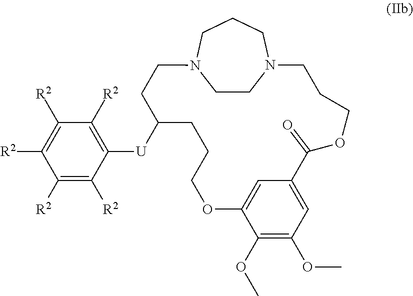
or a pharmaceutically acceptable salt, hydrate, or solvate thereof.

19. The combination, composition, kit, method or use according to any one of claims 13-18, wherein U is —C(O)O—.

20. The combination, composition, kit, method or use according to any one of claims 13, 14, 16, and 17, wherein R⁴ is 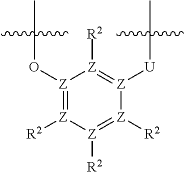
and the U in R⁴ is —C(O)O— or —C(O)NR³—.

21. The combination, composition, kit, method or use according to claim 14, wherein the compound is a compound of Formula (IIa1): 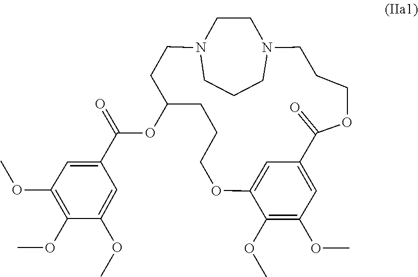
or a pharmaceutically acceptable salt, hydrate, or solvate thereof.

22. The combination, composition, kit, method or use according to any one of claims 1-14, wherein the ENT1 inhibitor is selected from:
(12S)-7⁴,7⁵-dimethoxy-6-oxo-5,8-dioxa-1(1,4)-diazepana-7(1,3)-benzenacyclotetradecaphane-12-yl 3,4,5-trimethoxybenzoate
(12R)-7⁴,7⁵-dimethoxy-6-oxo-5,8-dioxa-1(1,4)-diazepana-7(1,3)-benzenacyclotetradecaphane-12-yl 3,4,5-trimethoxybenzoate
16,16-difluoro-7⁴,7⁵-dimethoxy-6-oxo-5,8-dioxa-1(1,4)-diazepana-7(1,3)-benzenacyclotetradecaphane-12-yl 3,4,5-trimethoxybenzoate
(12S)-16,16-difluoro-7⁴,7⁵-dimethoxy-6-oxo-5,8-dioxa-1(1,4)-diazepana-7(1,3)-benzenacyclotetradecaphane-12-yl 3,4,5-trimethoxybenzoate
(12R)-16,16-difluoro-7⁴,7⁵-dimethoxy-6-oxo-5,8-dioxa-1(1,4)-diazepana-7(1,3)-benzenacyclotetradecaphane-12-yl 3,4,5-trimethoxybenzoate
N-(7⁴,7⁵-dimethoxy-6-oxo-5,8-dioxa-1(1,4)-diazepana-7(1,3)-benzenacyclotetradecaphane-12-yl)-3,4,5-trimethoxybenzamide
7⁴,7⁵-dimethoxy-6-oxo-8-oxa-5-aza-1(1,4)-diazepana-7(1,3)-benzenacyclotetradecaphane-12-yl 3,4,5-trimethoxybenzoate
(12S)-7⁴,7⁵-dimethoxy-6-oxo-8-oxa-5-aza-1(1,4)-diazepana-7(1,3)-benzenacyclotetradecaphane-12-yl 3,4,5-trimethoxybenzoate
(12R)-7⁴,7⁵-dimethoxy-6-oxo-8-oxa-5-aza-1(1,4)-diazepana-7(1,3)-benzenacyclotetradecaphane-12-yl 3,4,5-trimethoxybenzoate
7⁴,7⁵-dimethoxy-5-methyl-6-oxo-8-oxa-5-aza-1(1,4)-diazepana-7(1,3)-benzenacyclotetradecaphane-12-yl 3,4,5-trimethoxybenzoate
(12S)-7⁴,7⁵-dimethoxy-5-methyl-6-oxo-8-oxa-5-aza-1(1,4)-diazepana-7(1,3)-benzenacyclotetradecaphane-12-yl 3,4,5-trimethoxybenzoate
(12R)-7⁴,7⁵-dimethoxy-5-methyl-6-oxo-8-oxa-5-aza-1(1,4)-diazepana-7(1,3)-benzenacyclotetradecaphane-12-yl 3,4,5-trimethoxybenzoate
(11R)-7⁴,7⁵-dimethoxy-6-oxo-5-aza-1(1,4)-diazepana-7(1,3)-benzenacyclotridecaphane-11-yl 3,4,5-trimethoxybenzoate
(10S)-14-chloro-2-oxo-11H-3-aza-1(6,1)-indazola-7(1,4)-diazepanacyclotridecaphane-10-yl 3,4,5-trimethoxybenzoate
(10R)-14-chloro-2-oxo-11H-3-aza-1(6,1)-indazola-7(1,4)-diazepanacyclotridecaphane-10-yl 3,4,5-trimethoxybenzoate
(12S)-6-oxo-5,8-dioxa-1(1,4)-diazepana-7(1,3)-benzenacyclotetradecaphane-12-yl 3,4,5-trimethoxybenzoate
(12R)-6-oxo-5,8-dioxa-1(1,4)-diazepana-7(1,3)-benzenacyclotetradecaphane-12-yl 3,4,5-trimethoxybenzoate
(12S)-6-oxo-8-oxa-5-aza-1(1,4)-diazepana-7(1,3)-benzenacyclotetradecaphane-12-yl benzoate
(12R)-6-oxo-8-oxa-5-aza-1(1,4)-diazepana-7(1,3)-benzenacyclotetradecaphane-12-yl benzoate
7⁴,7⁵-dichloro-6-oxo-5,8-dioxa-1(1,4)-diazepana-7(1,3)-benzenacyclotetradecaphane-12-yl
3,4,5-trimethoxybenzoate
(12S)-7⁴,7⁵-dichloro-6-oxo-5,8-dioxa-1(1,4)-diazepana-7(1,3)-benzenacyclotetradecaphane-12-yl 3,4,5-trimethoxybenzoate
(12R)-7⁴,7⁵-dichloro-6-oxo-5,8-dioxa-1(1,4)-diazepana-7(1,3)-benzenacyclotetradecaphane-12-yl 3,4,5-trimethoxybenzoate
7⁵-carbamoyl-7⁴-chloro-6-oxo-5,8-dioxa-1(1,4)-diazepana-7(1,3)-benzenacyclotetradecaphane-12-yl 3,4,5-trimethoxybenzoate
(11Z,16E,10S)-14-chloro-2-oxo-12H-3-aza-1(6,2)-indazola-7(1,4)-diazepanacyclotridecaphane-10-yl 3,4,5-trimethoxybenzoate
(11Z,16E,10R)-14-chloro-2-oxo-12H-3-aza-1(6,2)-indazola-7(1,4)-diazepanacyclotridecaphane-10-yl 3,4,5-trimethoxybenzoate
(12S)-7⁴-carbamoyl-7⁵-chloro-6-oxo-8-oxa-5-aza-1(1,4)-diazepana-7(1,3)-benzenacyclotetradecaphane-12-yl 3,4,5-trimethoxybenzoate
(12R)-7⁴-carbamoyl-7⁵-chloro-6-oxo-8-oxa-5-aza-1(1,4)-diazepana-7(1,3)-benzenacyclotetradecaphane-12-yl 3,4,5-trimethoxybenzoate
7⁴-bromo-7⁵-chloro-6-oxo-8-oxa-5-aza-1(1,4)-diazepana-7(1,3)-benzenacyclotetradecaphane-12-yl 3,4,5-trimethoxybenzoate
7⁵-chloro-7⁴-cyano-6-oxo-8-oxa-5-aza-1(1,4)-diazepana-7(1,3)-benzenacyclotetradecaphane-12-yl 3,4,5-trimethoxybenzoate
(12R)-7⁴,7⁵-dimethoxy-6-oxo-5,8-dioxa-1(1,4)-diazepana-7(1,3)-benzenacyclotetradecaphane-12-yl benzoate
(12R)-7⁴,7⁵-dimethoxy-6-oxo-5,8-dioxa-1(1,4)-diazepana-7(1,3)-benzenacyclotetradecaphane-12-yl benzoate
(12S)-7⁴,7⁵-dimethoxy-6-oxo-5,8-dioxa-1(1,4)-diazepana-7(1,3)-benzenacyclotetradecaphane-12-yl benzoate
(Z)-benzaldehyde O-(7⁴,7⁵-dimethoxy-6-oxo-5,8-dioxa-1(1,4)-diazepana-7(1,3)-benzenacyclotetradecaphane-12-yl) oxime
12-hydroxy-7⁴,7⁵-dimethoxy-5,8-dioxa-1(1,4)-diazepana-7(1,3)-benzenacyclotetradecaphan-6-one
7⁴,7⁵-dimethoxy-6-oxo-5,8-dioxa-1(1,4)-diazepana-7(1,3)-benzenacyclotetradecaphane-12-yl 4-hydroxybenzoate
7⁴,7⁵-dimethoxy-6-oxo-5,8-dioxa-1(1,4)-diazepana-7(1,3)-benzenacyclotetradecaphane-12-yl 4-fluorobenzoate
7⁴,7⁵-dimethoxy-6-oxo-5,8-dioxa-1(1,4)-diazepana-7(1,3)-benzenacyclotetradecaphane-12-yl 4-isopropoxybenzoate
7⁴,7⁵-dimethoxy-6-oxo-5,8-dioxa-1(1,4)-diazepana-7(1,3)-benzenacyclotetradecaphane-12-yl 3-(trifluoromethyl)benzoate
7⁴,7⁵-dimethoxy-6-oxo-5,8-dioxa-1(1,4)-diazepana-7(1,3)-benzenacyclotetradecaphane-12-yl 3-(methylsulfonyl)benzoate
7⁴,7⁵-dimethoxy-6-oxo-5,8-dioxa-1(1,4)-diazepana-7(1,3)-benzenacyclotetradecaphane-12-yl 3-phenoxybenzoate
7⁴,7⁵-dimethoxy-6-oxo-5,8-dioxa-1(1,4)-diazepana-7(1,3)-benzenacyclotetradecaphane-12-yl 2-fluorobenzoate
7⁴,7⁵-dimethoxy-6-oxo-5,8-dioxa-1(1,4)-diazepana-7(1,3)-benzenacyclotetradecaphane-12-yl 4-bromo-3-cyanobenzoate
7⁴,7⁵-dimethoxy-6-oxo-5,8-dioxa-1(1,4)-diazepana-7(1,3)-benzenacyclotetradecaphane-12-yl 3-methyl-5-(trifluoromethyl)benzoate
7⁴,7⁵-dimethoxy-6-oxo-5,8-dioxa-1(1,4)-diazepana-7(1,3)-benzenacyclotetradecaphane-12-yl 2-fluoro-4-methoxybenzoate
7⁴,7⁵-dimethoxy-6-oxo-5,8-dioxa-1(1,4)-diazepana-7(1,3)-benzenacyclotetradecaphane-12-yl 4-methoxy-2-(trifluoromethoxy)benzoate
7⁴,7⁵-dimethoxy-6-oxo-5,8-dioxa-1(1,4)-diazepana-7(1,3)-benzenacyclotetradecaphane-12-yl picolinate
7⁴,7⁵-dimethoxy-6-oxo-5,8-dioxa-1(1,4)-diazepana-7(1,3)-benzenacyclotetradecaphane-12-yl nicotinate
7⁴,7⁵-dimethoxy-6-oxo-5,8-dioxa-1(1,4)-diazepana-7(1,3)-benzenacyclotetradecaphane-12-yl pyrazine-2-carboxylate
7⁴,7⁵-dimethoxy-6-oxo-5,8-dioxa-1(1,4)-diazepana-7(1,3)-benzenacyclotetradecaphane-12-yl 6-hydroxynicotinate
7⁴,7⁵-dimethoxy-6-oxo-5,8-dioxa-1(1,4)-diazepana-7(1,3)-benzenacyclotetradecaphane-12-yl quinoline-5-carboxylate
7⁴,7⁵-dimethoxy-6-oxo-5,8-dioxa-1(1,4)-diazepana-7(1,3)-benzenacyclotetradecaphane-12-yl oxazole-4-carboxylate
7⁴,7⁵-dimethoxy-6-oxo-5,8-dioxa-1(1,4)-diazepana-7(1,3)-benzenacyclotetradecaphane-12-yl 1H-1,2,3-triazole-4-carboxylate
7⁴,7⁵-dimethoxy-6-oxo-5,8-dioxa-1(1,4)-diazepana-7(1,3)-benzenacyclotetradecaphane-12-yl acetate
7⁴,7⁵-dimethoxy-6-oxo-5,8-dioxa-1(1,4)-diazepana-7(1,3)-benzenacyclotetradecaphane-12-yl cyclopropanecarboxylate
7⁴,7⁵-dimethoxy-6-oxo-5,8-dioxa-1(1,4)-diazepana-7(1,3)-benzenacyclotetradecaphane-12-yl 3-methylbutanoate
7⁴,7⁵-dimethoxy-6-oxo-5,8-dioxa-1(1,4)-diazepana-7(1,3)-benzenacyclotetradecaphane-12-yl 4,4,4-trifluorobutanoate
7⁴,7⁵-dimethoxy-6-oxo-5,8-dioxa-1(1,4)-diazepana-7(1,3)-benzenacyclotetradecaphane-12-yl cyclohexanecarboxylate
7⁴,7⁵-dimethoxy-6-oxo-5,8-dioxa-1(1,4)-diazepana-7(1,3)-benzenacyclotetradecaphane-12-yl 1-methylpiperidine-4-carboxylate
7⁴,7⁵-dimethoxy-6-oxo-5,8-dioxa-1(1,4)-diazepana-7(1,3)-benzenacyclotetradecaphane-12-yl 3,3-dimethylcyclobutane-1-carboxylate
7⁴,7⁵-dimethoxy-6-oxo-5,8-dioxa-1(1,4)-diazepana-7(1,3)-benzenacyclotetradecaphane-12-yl 2-(oxetan-3-yl)acetate
7⁴,7⁵-dimethoxy-6-oxo-5,8-dioxa-1(1,4)-diazepana-7(1,3)-benzenacyclotetradecaphane-12-yl (1R,5S,6R)-3-oxabicyclo[3.1.0]hexane-6-carboxylate
7⁴,7⁵-dimethoxy-6-oxo-5,8-dioxa-1(1,4)-diazepana-7(1,3)-benzenacyclotetradecaphane-12-yl 5-oxopyrrolidine-3-carboxylate
7⁴,7⁵-dimethoxy-6-oxo-5,8-dioxa-1(1,4)-diazepana-7(1,3)-benzenacyclotetradecaphane-12-yl 1-benzyl-5-oxopyrrolidine-3-carboxylate
7⁴,7⁵-dimethoxy-6-oxo-5,8-dioxa-1(1,4)-diazepana-7(1,3)-benzenacyclotetradecaphane-12-yl 4-methoxycyclohexane-1-carboxylate
7⁴,7⁵-dimethoxy-6-oxo-5,8-dioxa-1(1,4)-diazepana-7(1,3)-benzenacyclotetradecaphane-12-yl 2,6-difluorobenzoate
7⁴,7⁵-dimethoxy-6-oxo-5,8-dioxa-1(1,4)-diazepana-7(1,3)-benzenacyclotetradecaphane-12-yl 4-(trifluoromethoxy)benzoate
7⁴,7⁵-dimethoxy-6-oxo-5,8-dioxa-1(1,4)-diazepana-7(1,3)-benzenacyclotetradecaphane-12-yl 3-cyanobenzoate
7⁴,7⁵-dimethoxy-6-oxo-5,8-dioxa-1(1,4)-diazepana-7(1,3)-benzenacyclotetradecaphane-12-yl 2-oxo-1,2,3,4-tetrahydroquinoline-6-carboxylate
7⁴,7⁵-dimethoxy-6-oxo-5,8-dioxa-1(1,4)-diazepana-7(1,3)-benzenacyclotetradecaphane-12-yl 3-(difluoromethoxy)benzoate
7⁴,7⁵-dimethoxy-6-oxo-5,8-dioxa-1(1,4)-diazepana-7(1,3)-benzenacyclotetradecaphane-12-yl 3,5-dichlorobenzoate
7⁴,7⁵-dimethoxy-6-oxo-5,8-dioxa-1(1,4)-diazepana-7(1,3)-benzenacyclotetradecaphane-12-yl 3,4-dichlorobenzoate
7⁴,7⁵-dimethoxy-6-oxo-5,8-dioxa-1(1,4)-diazepana-7(1,3)-benzenacyclotetradecaphane-12-yl 2,3-dichlorobenzoate
7⁴,7⁵-dimethoxy-6-oxo-5,8-dioxa-1(1,4)-diazepana-7(1,3)-benzenacyclotetradecaphane-12-yl 2-chloro-6-fluoro-3-methylbenzoate
7⁴,7⁵-dimethoxy-6-oxo-5,8-dioxa-1(1,4)-diazepana-7(1,3)-benzenacyclotetradecaphane-12-yl 3-fluoro-5-(trifluoromethyl)benzoate
7⁴,7⁵-dimethoxy-6-oxo-5,8-dioxa-1(1,4)-diazepana-7(1,3)-benzenacyclotetradecaphane-12-yl 4-fluoro-3-(trifluoromethyl)benzoate
7⁴,7⁵-dimethoxy-6-oxo-5,8-dioxa-1(1,4)-diazepana-7(1,3)-benzenacyclotetradecaphane-12-yl 4-cyano-3-fluorobenzoate
7⁴,7⁵-dimethoxy-6-oxo-5,8-dioxa-1(1,4)-diazepana-7(1,3)-benzenacyclotetradecaphane-12-yl 4-(trifluoromethyl)benzoate
7⁴,7⁵-dimethoxy-6-oxo-5,8-dioxa-1(1,4)-diazepana-7(1,3)-benzenacyclotetradecaphane-12-yl 3,5-difluorobenzoate
7⁴,7⁵-dimethoxy-6-oxo-5,8-dioxa-1(1,4)-diazepana-7(1,3)-benzenacyclotetradecaphane-12-yl 3,4-difluorobenzoate
7⁴,7⁵-dimethoxy-6-oxo-5,8-dioxa-1(1,4)-diazepana-7(1,3)-benzenacyclotetradecaphane-12-yl 3-cyano-4-fluorobenzoate
7⁴,7⁵-dimethoxy-6-oxo-5,8-dioxa-1(1,4)-diazepana-7(1,3)-benzenacyclotetradecaphane-12-yl 4-cyanobenzoate
7⁴,7⁵-dimethoxy-6-oxo-5,8-dioxa-1(1,4)-diazepana-7(1,3)-benzenacyclotetradecaphane-12-yl 3-chloro-4-fluorobenzoate
7⁴,7⁵-dimethoxy-6-oxo-5,8-dioxa-1(1,4)-diazepana-7(1,3)-benzenacyclotetradecaphane-12-yl 1-methyl-1H-benzo[d]imidazole-5-carboxylate
7⁴,7⁵-dimethoxy-6-oxo-5,8-dioxa-1(1,4)-diazepana-7(1,3)-benzenacyclotetradecaphane-12-yl 4-(oxazol-5-yl)benzoate
7⁴,7⁵-dimethoxy-6-oxo-5,8-dioxa-1(1,4)-diazepana-7(1,3)-benzenacyclotetradecaphane-12-yl 4,5-dichloro-2-fluorobenzoate
7⁴,7⁵-dimethoxy-6-oxo-5,8-dioxa-1(1,4)-diazepana-7(1,3)-benzenacyclotetradecaphane-12-yl 3,4,5-triethoxybenzoate
7⁴,7⁵-dimethoxy-6-oxo-5,8-dioxa-1(1,4)-diazepana-7(1,3)-benzenacyclotetradecaphane-12-yl 3-methoxypropanoate
7⁴,7⁵-dimethoxy-6-oxo-5,8-dioxa-1(1,4)-diazepana-7(1,3)-benzenacyclotetradecaphane-12-yl 3-(1H-pyrazol-1-yl)propanoate
7⁴,7⁵-dimethoxy-6-oxo-5,8-dioxa-1(1,4)-diazepana-7(1,3)-benzenacyclotetradecaphane-12-yl 3-cyanopropanoate
7⁴,7⁵-dimethoxy-6-oxo-5,8-dioxa-1(1,4)-diazepana-7(1,3)-benzenacyclotetradecaphane-12-yl 4-cyanobutanoate
7⁴,7⁵-dimethoxy-6-oxo-5,8-dioxa-1(1,4)-diazepana-7(1,3)-benzenacyclotetradecaphane-12-yl 4-acetamidobutanoate
7⁴,7⁵-dimethoxy-6-oxo-5,8-dioxa-1(1,4)-diazepana-7(1,3)-benzenacyclotetradecaphane-12-yl 3-(1H-tetrazol-1-yl)propanoate
7⁴,7⁵-dimethoxy-6-oxo-5,8-dioxa-1(1,4)-diazepana-7(1,3)-benzenacyclotetradecaphane-12-yl 4-(dimethylamino)-4-oxobutanoate
7⁴,7⁵-dimethoxy-6-oxo-5,8-dioxa-1(1,4)-diazepana-7(1,3)-benzenacyclotetradecaphane-12-yl 3-acetamidopropanoate
7⁴,7⁵-dimethoxy-6-oxo-5,8-dioxa-1(1,4)-diazepana-7(1,3)-benzenacyclotetradecaphane-12-yl 4-(methylamino)-4-oxobutanoate
7⁴,7⁵-dimethoxy-6-oxo-5,8-dioxa-1(1,4)-diazepana-7(1,3)-benzenacyclotetradecaphane-12-yl 3-(1H-1,2,4-triazol-1-yl)propanoate
7⁴,7⁵-dimethoxy-6-oxo-5,8-dioxa-1(1,4)-diazepana-7(1,3)-benzenacyclotetradecaphane-12-yl 4-morpholino-4-oxobutanoate
7⁴,7⁵-dimethoxy-6-oxo-5,8-dioxa-1(1,4)-diazepana-7(1,3)-benzenacyclotetradecaphane-12-yl 3-(4-fluorophenoxy)propanoate
7⁴,7⁵-dimethoxy-6-oxo-5,8-dioxa-1(1,4)-diazepana-7(1,3)-benzenacyclotetradecaphane-12-yl 4,4-difluorocyclohexane-1-carboxylate
7⁴,7⁵-dimethoxy-6-oxo-5,8-dioxa-1(1,4)-diazepana-7(1,3)-benzenacyclotetradecaphane-12-yl 4-(trifluoromethyl)cyclohexane-1-carboxylate
7⁴,7⁵-dimethoxy-6-oxo-5,8-dioxa-1(1,4)-diazepana-7(1,3)-benzenacyclotetradecaphane-12-yl 3-(2,5-dioxopyrrolidin-1-yl)propanoate
7⁴,7⁵-dimethoxy-6-oxo-5,8-dioxa-1(1,4)-diazepana-7(1,3)-benzenacyclotetradecaphane-12-yl 3-methoxycyclohexane-1-carboxylate
7⁴,7⁵-dimethoxy-6-oxo-8-oxa-5-aza-1(1,4)-diazepana-7(1,3)-benzenacyclotetradecaphane-12-yl benzoate
(E)-benzaldehyde O-(7⁴,7⁵-dimethoxy-6-oxo-8-oxa-5-aza-1(1,4)-diazepana-7(1,3)-benzenacyclotetradecaphane-12-yl) oxime
(E)-benzaldehyde O-((12R)-7⁴,7⁵-dimethoxy-6-oxo-8-oxa-5-aza-1(1,4)-diazepana-7(1,3)-benzenacyclotetradecaphane-12-yl) oxime
(E)-benzaldehyde O-((12S)-7⁴,7⁵-dimethoxy-6-oxo-8-oxa-5-aza-1(1,4)-diazepana-7(1,3)-benzenacyclotetradecaphane-12-yl) oxime
12-hydroxy-7⁴,7⁵-dimethoxy-8-oxa-5-aza-1(1,4)-diazepana-7(1,3)-benzenacyclotetradecaphan-6-one
(12R)-12-hydroxy-7⁴,7⁵-dimethoxy-5,8-dioxa-1(1,4)-diazepana-7(1,3)-benzenacyclotetradecaphan-6-one
(12S)-12-hydroxy-7⁴,7⁵-dimethoxy-5,8-dioxa-1(1,4)-diazepana-7(1,3)-benzenacyclotetradecaphan-6-one
7⁴,7⁵-dimethoxy-12-(5-phenyl-2H-tetrazol-2-yl)-5,8-dioxa-1(1,4)-diazepana-7(1,3)-benzenacyclotetradecaphan-6-one
7⁴,7⁵-dimethoxy-12-(4-phenyl-1H-1,2,3-triazol-1-yl)-5,8-dioxa-1(1,4)-diazepana-7(1,3)-benzenacyclotetradecaphan-6-one
7⁴,7⁵-dimethoxy-12-(5-phenyl-1H-tetrazol-1-yl)-5,8-dioxa-1(1,4)-diazepana-7(1,3)-benzenacyclotetradecaphan-6-one,
and pharmaceutically acceptable salts, hydrates, or solvates thereof.

23. The combination, composition, kit, method or use according to any one of claims 1-14, wherein the ENT1 inhibitor is selected from Compound 1 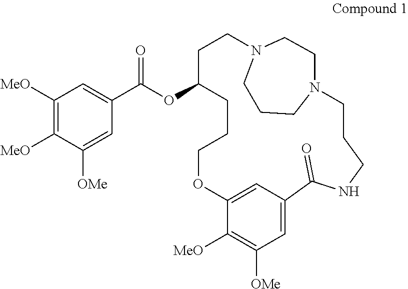 [SMILES: COc1cc(C(=O)O[C@@H]2CCCOc3cc(cc(OC)c3OC)C(=O)NCCCN3CCCN(CC2)CC3)cc(OC)c1OC]
and pharmaceutically acceptable salts, hydrates, and solvates thereof.

24. The combination, composition, kit, method or use according to any one of claims 1-14, wherein the ENT1 inhibitor is selected from Compound 1 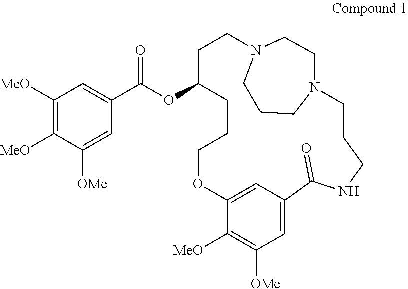 [SMILES: COc1cc(C(=O)O[C@@H]2CCCOc3cc(cc(OC)c3OC)C(=O)NCCCN3CCCN(CC2)CC3)cc(OC)c1OC]
and pharmaceutically acceptable salts thereof.

25. The combination, composition, kit, method or use according to claim 24, wherein the ENT1 inhibitor is a Compound 1 hydrogen sulfate or a hydrate or solvate thereof.

26. The combination, composition, kit, method or use according to claim 25, wherein the ENT1 inhibitor is a Compound 1 di(hydrogen sulfate) or a hydrate or solvate thereof.

27. The combination, composition, kit, method or use according to claim 26, wherein the Compound 1 di(hydrogen sulfate) or a hydrate or solvate thereof is crystalline.

28. The combination, composition, kit, method or use according to claim 27, wherein the crystalline Compound 1 di(hydrogen sulfate) or a hydrate or solvate thereof is crystalline Form 2 Compound 1 di(hydrogen sulfate) trihydrate.

29. The method according to any one of claims 4-6, wherein the reversal of adenosine-mediated suppression occurs in a cancer.

30. The method or use according to any one of claims 4-29, wherein the ENT1 inhibitor is administered prior to, concomitant with, or subsequent to the administration of the checkpoint inhibitor.

31. The method or use according to any one of claims 4-30, wherein the ENT1 inhibitor is administered orally, optionally as a tablet or capsule.

32. The method or use according to any one of claims 4-31, wherein the ENT1 inhibitor is administered daily during a treatment cycle.

33. The method or use according to any one of claims 4-32, wherein the ENT1 inhibitor is administered once or twice daily (BID).

34. The method or use according to any one of claims 4-31, wherein the ENT1 inhibitor is administered intermittently during a treatment cycle.

35. The method or use according to any one of claims 4-34, wherein the ENT1 inhibitor is administered at the same dose during the treatment cycle.

36. The method or use according to any one of claims 4-34, wherein the ENT1 inhibitor is administered at doses of escalating concentration.

37. The method or use according to any one of claims 4-36, further comprising administering an agent capable of treating, preventing, delaying, reducing or attenuating the development or risk of development of an adverse event.

38. The method or use according to claim 37, wherein the agent is administered prior to treatment with the ENT1 inhibitor, concomitant with, and/or subsequent to treatment with the ENT1 inhibitor.

39. The method or use according to claim 37 or claim 38, wherein the agent is administered prior to, concomitant with, and/or subsequent to at least one, two, three, four, five, six, seven, eight, nine, or ten doses of ENT inhibitor.

40. The method or use according to any one of claims 37-39, wherein the agent is administered at least 30 minutes prior to and/or subsequent to at least one dose of ENT1 inhibitor.

41. The method or use according to any one of claims 37-40, wherein the agent comprises an antiemetic, an analgesic, and/or caffeine.

42. The method or use according to claim 41, wherein the analgesic comprises a non-steroidal anti-inflammatory drug (NSAID), aspirin, acetaminophen, antidepressive medications, antiepileptic medication, and/or lidocaine; and the antiemetic comprises anticholinergic agents, antihistamines, cannabinoid receptor agonists, dopamine receptor antagonists, serotonin 5-HT3 receptor antagonists, and/or substance P/neurokinin 1 receptor antagonists.

43. The method or use according to claim 41, wherein the agent is caffeine.

44. The method or use according to any one of claims 37-43, wherein the agent is administered orally, optionally as a tablet or a capsule.

45. The method or use according to any one of claims 37-43, wherein the agent is administered in a beverage or in food.

46. The method or use according to any one of claims 4-45, wherein the ENT1 inhibitor is administered with a meal and/or a beverage, such as water.

47. The method or use according to any one of claims 7-46, wherein the cancer is characterized by a high concentration of adenosine in the tumor microenvironment (TME).

48. The method or use according to any one of claims 7-47, wherein the cancer is selected from breast, carcinoid, cervical, colorectal, endometrial, glioma, head and neck, liver, lung, melanoma, ovarian, pancreatic, prostate, renal, gastric, thyroid and urothelial cancers.

49. The method or use according to any one of claims 7-48, wherein the cancer is breast cancer.

50. The method or use according to claim 49, wherein the breast cancer is triple negative breast cancer.

51. The method or use according to claims 7-49, wherein the cancer is a solid-tumor cancer.
